(19) United States
(12) Patent Application Publication
(10) Pub. No.: US 2026/0079145 A1
(43) Pub. Date: Mar. 19, 2026

(54) FAT AND OIL DETERIORATION DEGREE DETECTION DEVICE, FAT AND OIL DETERIORATION DEGREE DETECTION SYSTEM, FAT AND OIL DETERIORATION DEGREE DETECTION METHOD, AND FAT AND OIL DETERIORATION DEGREE DETECTION PROGRAM

(71) Applicant: J-OIL MILLS, Inc., Tokyo (JP)

(72) Inventors: Eri NUTAHARA, Tokyo (JP); Ryohei WATANABE, Tokyo (JP); Kenichi KAKIMOTO, Tokyo (JP); Moeka ONO, Tokyo (JP); Shinsuke HARUGUCHI, Tokyo (JP)

(21) Appl. No.: 19/109,994

(22) PCT Filed: Nov. 20, 2023
(86) PCT No.: PCT/JP2023/041651

§ 371 Date: Mar. 7, 2025

(30) Foreign Application Priority Data
Nov. 21, 2022 (JP) 2022-186021

(51) Int. Cl. G01N 33/03; G06F 17/15

(52) U.S. Cl. CPC G01N 33/03; G06F 17/15

(57) ABSTRACT

A cloud 8, as a fat and oil deterioration degree detection device for detecting the deterioration degree of the frying oil P, includes a storage section 82 configured to retain a correlation between the total polar compounds PCn of the frying oil P and a predetermined deterioration indicator DIn other than the total polar compounds, a data acquisition section 81 configured to acquire a measured value of the total polar compounds, a deterioration indicator calculation section 83 configured to calculate the predetermined deterioration indicator DIn based on the measured value of the total polar compounds acquired by the data acquisition section 81 and the correlation stored in the storage section 82; and a detection result output section 84 configured to output the predetermined deterioration indicator DIn calculated by the deterioration indicator calculation section 83 as a result of detection of the deterioration degree of the frying oil P.

### TECHNICAL FIELD

The present invention relates to a fat and oil deterioration degree detection device, a fat and oil deterioration degree detection system, a fat and oil deterioration degree detection method, and a fat and oil deterioration degree detection program, which are configured to detect the degree of deterioration of fat and oil.

### BACKGROUND ART

Deep fry cooking, in which ingredients are deep fried using edible oil that is one type of fat and oil, is widely known as one of a number of cooking methods. For providing fried foods obtained by deep fry cooking with stable qualities, it is necessary to appropriately control the quality of edible oil (hereinafter, referred to as “frying oil”) used in the deep fry cooking. Frying oil gets deteriorated by its oxidation which progresses with increased time and increased frequency of use in deep fry cooking. Accordingly, especially in restaurants and stores which provide customers with fried foods, the degree of deterioration of the frying oil (hereinafter, simply referred to as “deterioration degree”) is grasped using an appropriate indicator, so that the frying oil that has reached the level for disposal of oil is disposed to be replaced with new frying oil.

An indicator indicative of the deterioration degree of fat and oil (hereinafter, referred to as “deterioration indicator”) includes, for example, the color, the acid value (AV), the total polar materials (TPM), the rate of increase in viscosity, the anisidine value, the carbonyl value, the smoke point, the content of tocopherol, the iodine value, the refractive indicator, the amount of volatile components, the volatile component composition, and the like. Each of these deterioration indicators can be measured using various sensors, imaging apparatuses, and the like.

For example, Patent Literature 1 discloses a method in which an image of a color test piece immersed in the target fat and oil and an image of a color bar including the colors corresponding to the acid values are simultaneously captured using a camera, the RGB color information on the color test piece and the RGB color information on the color bar are calculated based on the captured images, and the acid value of the target fat and oil is measured based on the RGB color information on the color test piece calculated by referring to the acid value corresponding to the calculated RGB color information of the color bar as calculated.

However, a person who measures the “acid value” of the fat and oil by the method according to Patent Literature 1 has to firstly immerse the color test piece in the target fat and oil, thereafter, let the color test piece that has been immersed in the target fat and oil stand for about 30 seconds, and then capture an image of the color test piece and an image of the color bar using a camera in such a manner that they are included in the same angle of view. Thus, an operation for measurement according to the method of Patent Literature 1 has to be complicated. Furthermore, difference in the measurement (for example, how to capture an image using the camera) among the persons who are in charge of measurement may cause errors in the result of measurement.

For this problem, for example, the “total polar compounds” can be used as the deterioration indicator of fat and oil which can be measured more easily and accurately than measuring the “acid value”. Patent Literature 2 discloses a method for detecting a TPM value indicative of the total polar compounds (polar molecular weight) contained in the fat and oil based on the result of measurement of capacitance in the fat and oil obtained by immersing a sensor having an electrode portion in the fat and oil. A person who measures the total polar compounds of the fat and oil by the method according to Patent Literature 2 is only required to immerse the sensor in the fat and oil, which makes the operation for measurement very simple. Furthermore, this can prevent errors in the result of measurement among the persons who perform the measurement from occurring, and thus can improve the accuracy in the measurement.

### CITATION LIST

### Patent Literature

Patent Literature 1: JP-A-2020-38207
Patent Literature 2: JP-B-6395243

### SUMMARY OF INVENTION

### Technical Problem

In Japan, an “acid value” has been conventionally used as an indicator indicative of a criteria for disposing oil. For example, according to the hygiene standards established by Ministry of Health, Labour and Welfare of Japan, edible oil for commercial use such as the one for prepared meals or school lunches is required to be replaced when it exceeds a standard value such as “acid value 2.5”. Under these circumstances, restaurants, stores, and the like rarely adopt the method of measuring “total polar compounds” according to Patent Literature 2, but mostly adopt the method of measuring “acid value” according to Patent Literature 1. In some cases, “color” is used as a deterioration indicator since it is easy to judge by appearance, and in other cases, various deterioration indicators, such as “viscosity increase rate”, may be adopted.

In the case of adopting an indicator of deterioration of edible oil which is likely to cause an error in the result of measurement, in many cases, a store sets a value which is slightly lower than a standard value (for example, in the case of adopting “acid value”, setting to “acid value 2.0”) as a threshold for disposing oil, considering that the measured value includes an error. This may cause even such edible oil that can be still used to be wastefully discarded. Furthermore, in the case of adopting an indicator of deterioration of edible oil which involves a complicated measurement operation, a store needs to secure a certain amount of time to detect the deterioration degree of the edible oil, which may cause the reduction in the work efficiency.

Therefore, an object of the present invention is to provide a fat and oil deterioration degree detection device, a fat and oil deterioration degree detection system, a fat and oil deterioration degree detection method, and a fat and oil deterioration degree detection program, which are capable of simply and accurately detecting various deterioration indicators of fat and oil.

### Solution to Problem

[1] The present invention provides a fat and oil deterioration degree detection device for detecting a deterioration degree of a fat and oil based on total polar compounds of the fat and oil which is one of fat and oil deterioration indicators, the device comprising: a storage section configured to retain a correlation between the total polar compounds and a predetermined deterioration indicator other than the total polar compounds; a data acquisition section configured to acquire a measured value of the total polar compounds; a deterioration indicator calculation section configured to calculate the predetermined deterioration indicator based on the measured value of the total polar compounds acquired by the data acquisition section and the correlation stored in the storage section; and a detection result output section configured to output the predetermined deterioration indicator calculated by the deterioration indicator calculation section as a result of detection of the deterioration degree.

[2] According to the fat and oil deterioration degree detection device described in [1], preferably, the correlation is a correlation equation in a form of a polynomial in which the predetermined deterioration indicator is expressed with the total polar compounds.

[3] According to the fat and oil deterioration degree detection device described in [2], preferably, the correlation equation is at least one of a linear equation expressed with a following equation (1) or a quadratic equation expressed with a following equation (2), where the total polar compounds are defined as PC, the predetermined deterioration indicator is defined as DI, and an arbitrary heating time of the fat and oil is defined as n.

$$DIn = α \times ( PCn ) + β \qquad ( 1 )$$

α: first-order coefficient of PCn
β: constant

$$DIn = γ \times {( PCn )}^{2} + δ \times ( PCn ) + ε \qquad ( 2 )$$

γ: second-order coefficient of PCn
δ: first-order coefficient of PCn
ε: constant

[4] According to the fat and oil deterioration degree detection device described in [3], preferably, the fat and oil are edible oil used for deep frying an ingredient, and the first-order coefficient α and the constant β included in the equation (1) and the second-order coefficient γ, the first-order coefficient δ, and the constant β included in the equation (2) are set to values corresponding to a deep-frying weight per unit time of a deep-frying material to be cooked using the edible oil, respectively.

[5] According to the fat and oil deterioration degree detection device described in [3], preferably, the fat and oil are edible oil used for deep frying an ingredient, the storage section retains, as the correlation equation, a linear equation expressed with a following equation (3) obtained by subtracting an empty heating variable EH1 from the equation (1) or a quadratic equation expressed with a following equation (4) obtained by subtracting an empty heating variable EH2 from the equation (2), the empty heating variable EH1 being set considering empty heating in which only the fat and oil are heated without cooking the ingredient, and the empty heating variable EH2 being set considering the empty heating, and

$$DIn = α \times ( PCn ) + β - EH 1 \qquad ( 3 )$$

α: first-order coefficient of PCn
β: constant
EH1: empty heating variable

$$DIn = γ \times {( PCn )}^{2} + δ \times ( PCn ) + ε - EH 2 \qquad ( 4 )$$

γ: second-order coefficient of PCn
δ: first-order coefficient of PCn
ε: constant
EH2: empty heating variable
in a case where the empty heating for the fat and oil has been performed, the deterioration indicator calculation section uses the equation (3) or the equation (4) stored in the storage section to calculate the predetermined deterioration indicator.

[6] According to the fat and oil deterioration degree detection device described in [3], preferably, the first-order coefficient α and the constant β included in the equation (1) and the second-order coefficient γ, the first-order coefficient δ, and the constant β included in the equation (2) are set to values corresponding to a type of the fat and oil, respectively.

[7] According to the fat and oil deterioration degree detection device described in [6], preferably, the type of the fat and oil is classified into a first oil type and a second oil type depending on a fatty acid composition of the fat and oil, the first oil type is an oil type indicative of a composition of the fat and oil in which a content of oleic acid is more than a content of linoleic acid, the second oil type is an oil type indicative of a composition of the fat and oil in which the content of oleic acid is equal to or less than the content of linoleic acid, the storage section retains, as the correlation equation, a linear equation expressed with a following equation (5) including each of α1 set to a value corresponding to the first oil type as the first-order coefficient α in the equation (1) and β1 set to a value corresponding to the first oil type as the constant β in the equation (1), or a quadratic equation expressed with a following equation (6) including each of γ1 set to a value corresponding to the first oil type as the second-order coefficient γ in the equation (2), δ1 set to a value corresponding to the first oil type as the first-order coefficient δ in the equation (2), and ε1 set to a value corresponding to the first oil type as the constant ε in the equation (2),

$$DIn = α1 \times ( PCn ) + β 1 \qquad ( 5 )$$

α1: first-order coefficient of PCn
β1: constant

$$DIn = γ1 \times {( PCn )}^{2} + δ1 \times ( PCn ) + ε1 \qquad ( 6 )$$

γ1: second-order coefficient of PCn
δ1: first-order coefficient of PCn
ε1: constant
the storage section retains, as the correlation equation, a linear equation expressed with a following equation (7) including each of α2 set to a value corresponding to the second oil type as the first-order coefficient α in the equation (1) and β2 set to a value corresponding to the second oil type as the constant β in the equation (1), or a quadratic equation expressed with a following equation (8) including each of γ2 set to a value corresponding to the second oil type as the second-order coefficient γ in the equation (2), δ2 set to a value corresponding to the second oil type as the first-order coefficient δ in the equation (2), and ε2 set to a value corresponding to the second oil type as the constant ε in the equation (2),

$$DIn = α2 \times ( PCn ) + β 2 \qquad ( 7 )$$

α2: first-order coefficient of PCn
β2: constant

$$DIn = γ2 \times {( PCn )}^{2} + δ2 \times ( PCn ) + ε2 \qquad ( 8 )$$

γ2: second-order coefficient of PCn
δ2: first-order coefficient of PCn
ε2: constant
in a case where the type of the oil and fat is the first oil type, the deterioration indicator calculation section uses the equation (5) or the equation (6) stored in the storage section to calculate the predetermined deterioration indicator, and in a case where the type of the oil and fat is the second oil type, the deterioration indicator calculation section uses the equation (7) or the equation (8) stored in the storage section to calculate the predetermined deterioration indicator.

[8] According to the fat and oil deterioration degree detection device described in [6], preferably, the type of the fat and oil is classified into a third oil type and a fourth oil type depending on an iodine value of the fat and oil, the third oil type is an oil type for which the iodine value of the fat and oil is less than a predetermined iodine value threshold, the fourth oil type is an oil type for which the iodine value of the fat and oil is equal to or more than the predetermined iodine value threshold, the storage section retains, as the correlation equation, a linear equation expressed with a following equation (9) including each of a3 set to a value corresponding to the third oil type as the first-order coefficient α in the equation (1) and β3 set to a value corresponding to the third oil type as the constant β in the equation (1), or a quadratic equation expressed with a following equation (10) including each of γ3 set to a value corresponding to the third oil type as the second-order coefficient γ in the equation (2), δ3 set to a value corresponding to the third oil type as the first-order coefficient δ in the equation (2), and ε3 set to a value corresponding to the third oil type as the constant ε in the equation (2),

$$DIn = α3 \times ( PCn ) + β 3 \qquad ( 9 )$$

α3: first-order coefficient of PCn
β3: constant

$$DIn = γ3 \times {( PCn )}^{2} + δ3 \times ( PCn ) + ε3 \qquad ( 10 )$$

γ3: second-order coefficient of PCn
δ3: first-order coefficient of PCn
ε3: constant
the storage section retains, as the correlation equation, a linear equation expressed with a following equation (11) including each of α4 set to a value corresponding to the fourth oil type as the first-order coefficient α in the equation (1) and β4 set to a value corresponding to the fourth oil type as the constant β in the equation (1), or a quadratic equation expressed with a following equation (12) including each of γ4 set to a value corresponding to the fourth oil type as the second-order coefficient γ in the equation (2), 64 set to a value corresponding to the fourth oil type as the first-order coefficient δ in the equation (2), and ε4 set to a value corresponding to the fourth oil type as the constant ε in the equation (2),

$$DIn = α4 \times ( PCn ) + β4 \qquad ( 11 )$$

α4: first-order coefficient of PCn
β4: constant

$$DIn = γ4 \times {( PCn )}^{2} + δ4 \times ( PCn ) + ε 4 \qquad ( 12 )$$

γ4: second-order coefficient of PCn
δ4: first-order coefficient of PCn
ε4: constant
in a case where the type of the oil and fat is the third oil type, the deterioration indicator calculation section uses the equation (9) or the equation (10) to calculate the predetermined deterioration indicator, and in a case where the type of the oil and fat is the fourth oil type, the deterioration indicator calculation section uses the equation (11) or the equation (12) to calculate the predetermined deterioration indicator.

[9] According to the fat and oil deterioration degree detection device described in [6], preferably, the type of the fat and oil is classified into a fifth oil type and a sixth oil type depending on a CDM value of the fat and oil, the fifth oil type is an oil type for which the CDM value of the fat and oil is equal to or more than a predetermined CDM threshold, the sixth oil type is an oil type for which the CDM value of the fat and oil is less than the predetermined CDM threshold, the storage section retains, as the correlation equation, a linear equation expressed with a following equation (13) including each of α5 set to a value corresponding to the fifth oil type as the first-order coefficient α in the equation (1) and β5 set to a value corresponding to the fifth oil type as the constant R in the equation (1), or a quadratic equation expressed with a following equation (14) including each of γ5 set to a value corresponding to the fifth oil type as the second-order coefficient γ in the equation (2), δ5 set to a value corresponding to the fifth oil type as the first-order coefficient δ in the equation (2), and ε5 set to a value corresponding to the fifth oil type as the constant ε in the equation (2),

$$DIn = α5 \times ( PCn ) + β 5 \qquad ( 13 )$$

α5: first-order coefficient of PCn
β5: constant

$$DIn = γ5 \times {( PCn )}^{2} + δ5 \times ( PCn ) + ε5 \qquad ( 14 )$$

γ5: second-order coefficient of PCn
δ5: first-order coefficient of PCn
ε5: constant
the storage section retains, as the correlation equation, a linear equation expressed with a following equation (15) including each of α6 set to a value corresponding to the sixth oil type as the first-order coefficient α in the equation (1) and β6 set to a value corresponding to the sixth oil type as the constant β in the equation (1), or a quadratic equation expressed with a following equation (16) including each of γ6 set to a value corresponding to the sixth oil type as the second-order coefficient γ in the equation (2), δ6 set to a value corresponding to the sixth oil type as the first-order coefficient δ in the equation (2), and ε6 set to a value corresponding to the sixth oil type as the constant ε in the equation (2),

$$DIn = α6 \times ( PCn ) + β 6 \qquad ( 15 )$$

α6: first-order coefficient of PCn
β6: constant

$$DIn = γ6 \times {( PCn )}^{2} + δ6 \times ( PCn ) + ε6 \qquad ( 16 )$$

γ6: second-order coefficient of PCn
δ6: first-order coefficient of PCn
ε6: constant
in a case where the type of the oil and fat is the fifth oil type, the deterioration indicator calculation section uses the equation (13) or the equation (14) to calculate the predetermined deterioration indicator, and in a case where the type of the oil and fat is the sixth oil type, the deterioration indicator calculation section uses the equation (15) or the equation (16) to calculate the predetermined deterioration indicator.

[10] According to the fat and oil deterioration degree detection device described in [6], preferably, the type of the fat and oil is classified into a seventh oil type and an eighth oil type depending on lipid molecular species in the fat and oil, the seventh oil type is an oil type for which a content of the lipid molecular species in the fat and oil is more than a predetermined content threshold, a rate of increase in a content of diacylglycerol in the fat and oil due to heating is equal to or less than a predetermined first increase rate threshold, a rate of increase in a content of free fatty acid in the fat and oil due to heating is equal to or less than a predetermined second increase rate threshold, and a rate of decrease in a content of triacylglycerol in the fat and oil due to heating is equal to or less than a predetermined decrease rate threshold, the eighth oil type is an oil type for which the content of the lipid molecular species in the fat and oil is equal to or less than the predetermined content threshold, the rate of increase in the content of diacylglycerol in the fat and oil due to heating is more than the predetermined first increase rate threshold, the rate of increase in the content of free fatty acid in the fat and oil due to heating is more than the predetermined second increase rate threshold, and the rate of decrease in the content of triacylglycerol in the fat and oil due to heating is more than the predetermined decrease rate threshold, the storage section retains, as the correlation equation, a linear equation expressed with a following equation (17) including each of α7 set to a value corresponding to the seventh oil type as the first-order coefficient α in the equation (1) and β7 set to a value corresponding to the seventh oil type as the constant β in the equation (1), or a quadratic equation expressed with a following equation (18) including each of γ7 set to a value corresponding to the seventh oil type as the second-order coefficient γ in the equation (2), 67 set to a value corresponding to the seventh oil type as the first-order coefficient δ in the equation (2), and ε7 set to a value corresponding to the seventh oil type as the constant ε in the equation (2),

$$DIn = α7 \times ( PCn ) + β 7 \qquad ( 17 )$$

α7: first-order coefficient of PCn
β7: constant

$$DIn = γ7 \times {( PCn )}^{2} + δ7 \times ( PCn ) + ε7 \qquad ( 18 )$$

γ7: second-order coefficient of PCn
δ7: first-order coefficient of PCn
ε7: constant
the storage section retains, as the correlation equation, a linear equation expressed with a following equation (19) including each of α8 set to a value corresponding to the eighth oil type as the first-order coefficient α in the equation (1) and β8 set to a value corresponding to the eighth oil type as the constant β in the equation (1), or a quadratic equation expressed with a following equation (20) including each of γ8 set to a value corresponding to the eighth oil type as the second-order coefficient γ in the equation (2), 68 set to a value corresponding to the eighth oil type as the first-order coefficient δ in the equation (2), and ε8 set to a value corresponding to the eighth oil type as the constant ε in the equation (2),

$$DIn = α8 \times ( PCn ) + β8 \qquad ( 19 )$$

α8: first-order coefficient of PCn
β8: constant

$$DIn = γ8 \times {( PCn )}^{2} + δ8 \times ( PCn ) + ε8 \qquad ( 20 )$$

γ8: second-order coefficient of PCn
δ8: first-order coefficient of PCn
ε8: constant
in a case where the type of the oil and fat is the seventh oil type, the deterioration indicator calculation section uses the equation (17) or the equation (18) to calculate the predetermined deterioration indicator, and in a case where the type of the oil and fat is the eighth oil type, the deterioration indicator calculation section uses the equation (19) or the equation (20) to calculate the predetermined deterioration indicator.

[11] Furthermore, the present invention provides a fat and oil deterioration degree detection system for detecting a deterioration degree of a fat and oil based on total polar compounds of the fat and oil which is one of fat and oil deterioration indicators, the system comprising: a measurement device configured to measure the total polar compounds contained in the fat and oil; and a fat and oil deterioration degree detection device configured to detect the deterioration degree of the fat and oil based on a measured value of the total polar compounds measured by the measurement device, and the fat and oil deterioration degree detection device being configured to: retain a correlation between the total polar compounds and a predetermined deterioration indicator other than the total polar compounds; acquire the measured value of the total polar compounds measured by the measurement device; calculate the predetermined deterioration indicator based on the measured value of the total polar compounds as acquired and the correlation as stored; and output the predetermined deterioration indicator as calculated as a result of detection of the deterioration degree.

[12] According to the fat and oil deterioration degree detection system described in [11], preferably, the correlation is a correlation equation in a form of a polynomial in which the predetermined deterioration indicator is expressed with the total polar compounds.

[13] According to the fat and oil deterioration degree detection system described in [12], preferably, the correlation equation is at least one of a linear equation expressed with a following equation (1) or a quadratic equation expressed with a following equation (2), where the total polar compounds are defined as PC, the predetermined deterioration indicator is defined as DI, and an arbitrary heating time of the fat and oil is defined as n.

$$DIn = α \times ( PCn ) + β \qquad ( 1 )$$

α: first-order coefficient of PCn
β: constant

$$DIn = γ \times {( PCn )}^{2} + δ \times ( PCn ) + ε \qquad ( 2 )$$

γ: second-order coefficient of PCn
δ: first-order coefficient of PCn
ε: constant

[14] Furthermore, the present invention provides a fat and oil deterioration degree detection method for detecting a deterioration degree of a fat and oil based on total polar compounds of the fat and oil which is one of fat and oil deterioration indicators, the method using: a measurement device configured to measure the total polar compounds contained in the fat and oil; and a fat and oil deterioration degree detection device configured to retain a correlation between the total polar compounds and a predetermined deterioration indicator other than the total polar compounds, and the method comprising: a measurement step of measuring, by the measurement device, the total polar compounds contained in the fat and oil; a data acquisition step of acquiring, by the fat and oil deterioration degree detection device, a measured value of the total polar compounds measured in the measurement step; a deterioration indicator calculation step of calculating, by the fat and oil deterioration degree detection device, the predetermined deterioration indicator based on the measured value of the total polar compounds acquired in the data acquisition step and the correlation as stored; and a detection result output step of outputting, by the fat and oil deterioration degree detection device, the predetermined deterioration indicator calculated in the deterioration indicator calculation step as a result of detection of the deterioration degree.

[15] According to the fat and oil deterioration degree detection method described in [14], preferably, the correlation is a correlation equation in a form of a polynomial in which the predetermined deterioration indicator is expressed with the total polar compounds.

[16] According to the fat and oil deterioration degree detection method described in [15], preferably, the correlation equation is at least one of a linear equation expressed with a following equation (1) or a quadratic equation expressed with a following equation (2), where the total polar compounds are defined as PC, the predetermined deterioration indicator is defined as DI, and an arbitrary heating time of the fat and oil is defined as n.

$$DIn = α \times ( PCn ) + β \qquad ( 1 )$$

α: first-order coefficient of PCn
β: constant

$$DIn = γ \times {( PCn )}^{2} + δ \times ( PCn ) + ε \qquad ( 2 )$$

γ: second-order coefficient of PCn
δ: first-order coefficient of PCn
ε: constant

[17] Furthermore, the present invention provides a fat and oil deterioration degree detection program for detecting a deterioration degree of a fat and oil, the program causing a computer to execute processing comprising: a data acquisition process of acquiring a measured value of total polar compounds contained in the fat and oil; a deterioration indicator calculation process of calculating, using a correlation between the total polar compounds and a predetermined deterioration indicator other than the total polar compounds, the predetermined deterioration indicator based on the measured value of the total polar compounds acquired by the data acquisition process; and a detection result output process of outputting the predetermined deterioration indicator calculated by the deterioration indicator calculation process as a result of detection of the deterioration degree of the fat and oil.

[18] According to the fat and oil deterioration degree detection program described in [17], preferably, the correlation is a correlation equation in a form of a polynomial in which the predetermined deterioration indicator is expressed with the total polar compounds.

[19] According to the fat and oil deterioration degree detection program described in [18], preferably, the correlation equation is at least one of a linear equation expressed with a following equation (1) or a quadratic equation expressed with a following equation (2), where the total polar compounds are defined as PC, the predetermined deterioration indicator is defined as DI, and an arbitrary heating time of the fat and oil is defined as n.

$$DIn = α \times ( PCn ) + β \qquad ( 1 )$$

α: first-order coefficient of PCn
β: constant

$$DIn = γ \times {( PCn )}^{2} + δ \times ( PCn ) + ε \qquad ( 2 )$$

γ: second-order coefficient of PCn
δ: first-order coefficient of PCn
ε: constant

[20] Furthermore, the present invention provides a fat and oil deterioration degree detection device for detecting a deterioration degree of a fat and oil, the device comprising: a storage section configured to retain a correlation between a first deterioration indicator and a second deterioration indicator, the first deterioration indicator being a deterioration indicator of the fat and oil and defined based on a substance produced by heating the fat and oil, and the second deterioration indicator being a deterioration indicator of the fat and oil other than the first deterioration indicator; a data acquisition section configured to acquire a measured value of the first deterioration indicator; a deterioration indicator calculation section configured to calculate the second deterioration indicator based on the measured value of the first deterioration indicator acquired by the data acquisition section and the correlation stored in the storage section; and a detection result output section configured to output the second deterioration indicator calculated by the deterioration indicator calculation section as a result of detection of the deterioration degree.

[21] According to the fat and oil deterioration degree detection device described in [20], preferably, the correlation is a correlation equation in a form of a polynomial in which the second deterioration indicator is expressed with the first deterioration indicator.

[22] According to the fat and oil deterioration degree detection device described in [21], preferably, the correlation equation is at least one of a linear equation expressed with a following equation (31) or a quadratic equation expressed with a following equation (32), where the first deterioration indicator is Di1, the second deterioration indicator is Di2, and an arbitrary heating time of the fat and oil is n.

$$Di 2 n = α \times ( Di 1 n ) + β \qquad ( 31 )$$

α: first-order coefficient of Di1n
β: constant

$$Di 2 n = γ \times {( Di 1 n )}^{2} + δ \times ( Di 1 n ) + ε \qquad ( 32 )$$

γ: second-order coefficient of Di1n
δ: first-order coefficient of Di1n
ε: constant

[23] According to the fat and oil deterioration degree detection device described in [22], preferably, the fat and oil are edible oil used for deep frying an ingredient, and the first-order coefficient α and the constant β included in the equation (31) and the second-order coefficient γ, the first-order coefficient δ, and the constant β included in the equation (32) are set to values corresponding to a deep-frying weight per unit time of a deep-frying material to be cooked using the edible oil, respectively.

[24] According to the fat and oil deterioration degree detection device described in [22], preferably, the fat and oil are edible oil used for deep frying an ingredient, the storage section retains, as the correlation equation, a linear equation expressed with a following equation (33) obtained by adding a member of an empty heating variable EH1 to the equation (31) or a quadratic equation expressed with a following equation (34) obtained by adding a member of an empty heating variable EH2 to the equation (32), the empty heating variable EH1 being set considering empty heating in which only the fat and oil are heated without cooking the ingredient, and the empty heating variable EH2 being set considering the empty heating, and

$$Di 2 n = α \times ( Di 1 n ) + β + EH 1 \qquad ( 33 )$$

α: first-order coefficient of Di1n
β: constant
EH1: empty heating variable

$$Di 2 n = γ \times {( Di 1 n )}^{2} + δ \times ( Di 1 n ) + ε + EH 2 \qquad ( 34 )$$

γ: second-order coefficient of Di1n
δ: first-order coefficient of Di1n
ε: constant
EH2: empty heating variable
in a case where the empty heating for the fat and oil has been performed, the deterioration indicator calculation section uses the equation (33) or the equation (34) stored in the storage section to calculate the second deterioration indicator.

[25] According to the fat and oil deterioration degree detection device described in [22], preferably, the first-order coefficient α and the constant β included in the equation (31) and the second-order coefficient γ, the first-order coefficient δ, and the constant β included in the equation (32) are set to values corresponding to a type of the fat and oil, respectively.

[26] According to the fat and oil deterioration degree detection device described in [25], preferably, the type of the fat and oil is classified into a first oil type and a second oil type depending on a fatty acid composition of the fat and oil, the first oil type is an oil type indicative of a composition of the fat and oil in which a content of oleic acid is more than a content of linoleic acid, the second oil type is an oil type indicative of a composition of the fat and oil in which the content of oleic acid is equal to or less than the content of linoleic acid, the storage section retains, as the correlation equation, a linear equation expressed with a following equation (35) including each of α1 set to a value corresponding to the first oil type as the first-order coefficient α in the equation (31) and β1 set to a value corresponding to the first oil type as the constant β in the equation (31), or a quadratic equation expressed with a following equation (36) including each of γ1 set to a value corresponding to the first oil type as the second-order coefficient γ in the equation (32), 61 set to a value corresponding to the first oil type as the first-order coefficient δ in the equation (32), and ε1 set to a value corresponding to the first oil type as the constant ε in the equation (32),

$$Di 2 n = α 1 \times ( Di 1 n ) + β 1 \qquad ( 35 )$$

α1: first-order coefficient of Di1n
β1: constant

$$Di 2 n = γ 1 \times {( Di 1 n )}^{2} + δ 1 \times ( Di 1 n ) + ε 1 \qquad ( 36 )$$

γ1: second-order coefficient of Di1n
δ1: first-order coefficient of Di1n
ε1: constant
the storage section retains, as the correlation equation, a linear equation expressed with a following equation (37) including each of α2 set to a value corresponding to the second oil type as the first-order coefficient α in the equation (31) and β2 set to a value corresponding to the second oil type as the constant β in the equation (31), or a quadratic equation expressed with a following equation (38) including each of γ2 set to a value corresponding to the second oil type as the second-order coefficient γ in the equation (32), δ2 set to a value corresponding to the second oil type as the first-order coefficient δ in the equation (32), and ε2 set to a value corresponding to the second oil type as the constant ε in the equation (32),

$$Di 2 n = α 2 \times ( Di 1 n ) + β 2 \qquad ( 37 )$$

α2: first-order coefficient of Di1n
β2: constant

$$Di 2 n = γ 2 \times {( Di 1 n )}^{2} + δ 2 \times ( Di 1 n ) + ε 2 \qquad ( 38 )$$

γ2: second-order coefficient of Di1n
δ2: first-order coefficient of Di1n
ε2: constant
in a case where the type of the oil and fat is the first oil type, the deterioration indicator calculation section uses the equation (35) or the equation (36) stored in the storage section to calculate the second deterioration indicator, and in a case where the type of the oil and fat is the second oil type, the deterioration indicator calculation section uses the equation (37) or the equation (38) stored in the storage section to calculate the second deterioration indicator.

[27] According to the fat and oil deterioration degree detection device described in [25], preferably, the type of the fat and oil is classified into a third oil type and a fourth oil type depending on an iodine value of the fat and oil, the third oil type is an oil type for which the iodine value of the fat and oil is less than a predetermined iodine value threshold, the fourth oil type is an oil type for which the iodine value of the fat and oil is equal to or more than the predetermined iodine value threshold, the storage section retains, as the correlation equation, a linear equation expressed with a following equation (39) including each of α3 set to a value corresponding to the third oil type as the first-order coefficient α in the equation (31) and β3 set to a value corresponding to the third oil type as the constant β in the equation (31), or a quadratic equation expressed with a following equation (40) including each of γ3 set to a value corresponding to the third oil type as the second-order coefficient γ in the equation (32), δ3 set to a value corresponding to the third oil type as the first-order coefficient δ in the equation (32), and ε3 set to a value corresponding to the third oil type as the constant ε in the equation (32),

$$Di 2 n = α 3 \times ( Di 1 n ) + β 3 \qquad ( 39 )$$

α3: first-order coefficient of Di1n
β3: constant

$$Di 2 n = γ 3 \times {( Di 1 n )}^{2} + δ 3 \times ( Di 1 n ) + ε 3 \qquad ( 40 )$$

γ3: second-order coefficient of Di1n
δ3: first-order coefficient of Di1n
ε3: constant
the storage section retains, as the correlation equation, a linear equation expressed with a following equation (41) including each of α4 set to a value corresponding to the fourth oil type as the first-order coefficient α in the equation (31) and β4 set to a value corresponding to the fourth oil type as the constant β in the equation (31), or a quadratic equation expressed with a following equation (42) including each of γ4 set to a value corresponding to the fourth oil type as the second-order coefficient γ in the equation (32), δ4 set to a value corresponding to the fourth oil type as the first-order coefficient δ in the equation (32), and ε4 set to a value corresponding to the fourth oil type as the constant ε in the equation (32),

$$Di 2 n = α4 \times ( Di 1 n ) + β 4 \qquad ( 41 )$$

α4: first-order coefficient of Di1n
β4: constant

$$Di 2 n = γ 4 \times {( Di 1 n )}^{2} + δ 4 \times ( Di 1 n ) + ε 4 \qquad ( 42 )$$

γ4: second-order coefficient of Di1n
δ4: first-order coefficient of Di1n
ε4: constant
in a case where the type of the oil and fat is the third oil type, the deterioration indicator calculation section uses the equation (39) or the equation (40) to calculate the second deterioration indicator, and in a case where the type of the oil and fat is the fourth oil type, the deterioration indicator calculation section uses the equation (41) or the equation (42) to calculate the second deterioration indicator.

[28] According to the fat and oil deterioration degree detection device described in [25], preferably, the type of the fat and oil is classified into a fifth oil type and a sixth oil type depending on a CDM value of the fat and oil, the fifth oil type is an oil type for which the CDM value of the fat and oil is equal to or more than a predetermined CDM threshold, the sixth oil type is an oil type for which the CDM value of the fat and oil is less than the predetermined CDM threshold, the storage section retains, as the correlation equation, a linear equation expressed with a following equation (43) including each of α5 set to a value corresponding to the fifth oil type as the first-order coefficient α in the equation (31) and β5 set to a value corresponding to the fifth oil type as the constant R in the equation (31), or a quadratic equation expressed with a following equation (44) including each of γ5 set to a value corresponding to the fifth oil type as the second-order coefficient γ in the equation (32), 65 set to a value corresponding to the fifth oil type as the first-order coefficient δ in the equation (32), and ε5 set to a value corresponding to the fifth oil type as the constant ε in the equation (32),

$$Di 2 n = α5 \times ( Di 1 n ) + β 5 \qquad ( 43 )$$

α5: first-order coefficient of Di1n
β5: constant

$$Di 2 n = γ 5 \times {( Di 1 n )}^{2} + δ5 \times ( Di 1 n ) + ε 5 \qquad ( 44 )$$

γ5: second-order coefficient of Di1n
δ5: first-order coefficient of Di1n
ε5: constant
the storage section retains, as the correlation equation, a linear equation expressed with a following equation (45) including each of α6 set to a value corresponding to the sixth oil type as the first-order coefficient α in the equation (31) and β6 set to a value corresponding to the sixth oil type as the constant β in the equation (31), or a quadratic equation expressed with a following equation (46) including each of γ6 set to a value corresponding to the sixth oil type as the second-order coefficient γ in the equation (32), δ6 set to a value corresponding to the sixth oil type as the first-order coefficient δ in the equation (32), and ε6 set to a value corresponding to the sixth oil type as the constant ε in the equation (32),

$$Di 2 n = α 6 \times ( Di 1 n ) + β 6 \qquad ( 45 )$$

α6: first-order coefficient of Di1n
β6: constant

$$Di 2 n = γ 6 \times {( Di 1 n )}^{2} + δ 6 \times ( Di 1 n ) + ε 6 \qquad ( 46 )$$

γ6: second-order coefficient of Di1n
δ6: first-order coefficient of Di1n
ε6: constant
in a case where the type of the oil and fat is the fifth oil type, the deterioration indicator calculation section uses the equation (43) or the equation (44) to calculate the second deterioration indicator, and in a case where the type of the oil and fat is the sixth oil type, the deterioration indicator calculation section uses the equation (45) or the equation (46) to calculate the second deterioration indicator.

[29] According to the fat and oil deterioration degree detection device described in [25], preferably, the type of the fat and oil is classified into a seventh oil type and an eighth oil type depending on lipid molecular species in the fat and oil, the seventh oil type is an oil type for which a content of the lipid molecular species in the fat and oil is more than a predetermined content threshold, a rate of increase in a content of diacylglycerol in the fat and oil due to heating is equal to or less than a predetermined first increase rate threshold, a rate of increase in a content of free fatty acid in the fat and oil due to heating is equal to or less than a predetermined second increase rate threshold, and a rate of decrease in a content of triacylglycerol in the fat and oil due to heating is equal to or less than a predetermined decrease rate threshold, the eighth oil type is an oil type for which the content of the lipid molecular species in the fat and oil is equal to or less than the predetermined content threshold, the rate of increase in the content of diacylglycerol in the fat and oil due to heating is more than the predetermined first increase rate threshold, the rate of increase in the content of free fatty acid in the fat and oil due to heating is more than the predetermined second increase rate threshold, and the rate of decrease in the content of triacylglycerol in the fat and oil due to heating is more than the predetermined decrease rate threshold, the storage section retains, as the correlation equation, a linear equation expressed with a following equation (47) including each of α7 set to a value corresponding to the seventh oil type as the first-order coefficient α in the equation (31) and β7 set to a value corresponding to the seventh oil type as the constant β in the equation (31), or a quadratic equation expressed with a following equation (48) including each of γ7 set to a value corresponding to the seventh oil type as the second-order coefficient γ in the equation (32), 67 set to a value corresponding to the seventh oil type as the first-order coefficient δ in the equation (2), and ε7 set to a value corresponding to the seventh oil type as the constant ε in the equation (32),

$$Di 2 n = α 7 \times ( Di 1 n ) + β 7 \qquad ( 47 )$$

α7: first-order coefficient of Di1n
β7: constant

$$Di 2 n = γ 7 \times {( Di 1 n )}^{2} + δ 7 \times ( Di 1 n ) + ε 7 \qquad ( 48 )$$

γ7: second-order coefficient of Di1n
δ7: first-order coefficient of Di1n
ε7: constant
the storage section retains, as the correlation equation, a linear equation expressed with a following equation (49) including each of α8 set to a value corresponding to the eighth oil type as the first-order coefficient α in the equation (31) and β8 set to a value corresponding to the eighth oil type as the constant β in the equation (31), or a quadratic equation expressed with a following equation (50) including each of γ8 set to a value corresponding to the eighth oil type as the second-order coefficient γ in the equation (32), 68 set to a value corresponding to the eighth oil type as the first-order coefficient δ in the equation (32), and ε8 set to a value corresponding to the eighth oil type as the constant ε in the equation (32),

$$Di 2 n = α 8 \times ( Di 1 n ) + β 8 \qquad ( 49 )$$

α8: first-order coefficient of Di1n
β8: constant

$$Di 2 n = γ 8 \times {( Di 1 n )}^{2} + δ 8 \times ( Di 1 n ) + ε 8 \qquad ( 50 )$$

γ8: second-order coefficient of Di1n
δ8: first-order coefficient of Di1n
ε8: constant
in a case where the type of the oil and fat is the seventh oil type, the deterioration indicator calculation section uses the equation (47) or the equation (48) to calculate the second deterioration indicator, and in a case where the type of the oil and fat is the eighth oil type, the deterioration indicator calculation section uses the equation (49) or the equation (50) to calculate the second deterioration indicator.

[30] According to the fat and oil deterioration degree detection device described in [22], preferably, the fat and oil are edible oil used for deep frying an ingredient, and in a case where the deterioration indicator calculation section calculates the color of the edible oil as the second deterioration indicator, the first-order coefficient α and the constant β included in the equation (31) and the second-order coefficient γ, the first-order coefficient δ, and the constant β included in the equation (32) are set to values corresponding to a type of a deep-frying material to be cooked using the edible oil, respectively.

[31] According to the fat and oil deterioration degree detection device described in [20], preferably, the first deterioration indicator is at least one of an acid value of the fat and oil, total polar compounds of the fat and oil, a color of the fat and oil, or a rate of increase in viscosity of the fat and oil.

[32] Furthermore, the present invention provides a fat and oil deterioration degree detection system for detecting a deterioration degree of a fat and oil, the system comprising: a measurement device configured to measure a first deterioration indicator which is a deterioration indicator of the fat and oil and defined based on a substance produced by heating the fat and oil; and a fat and oil deterioration degree detection device configured to detect the deterioration degree of the fat and oil based on a measured value of the first deterioration indicator measured by the measurement device, and the fat and oil deterioration degree detection device being configured to: retain a correlation between the first deterioration indicator and a second deterioration indicator which is a deterioration indicator of the fat and oil other than the first deterioration indicator; acquire the measured value of the first deterioration indicator measured by the measurement device; calculate the second deterioration indicator based on the measured value of the first deterioration indicator as acquired and the correlation as stored; and output the second deterioration indicator as calculated as a result of detection of the deterioration degree.

[33] According to the fat and oil deterioration degree detection system described in [32], preferably, the correlation is a correlation equation in a form of a polynomial in which the second deterioration indicator is expressed with the first deterioration indicator.

[34] According to the fat and oil deterioration degree detection system described in [33], preferably, the correlation equation is at least one of a linear equation expressed with a following equation (31) or a quadratic equation expressed with a following equation (32), where the first deterioration indicator is defined as Di1, the second deterioration indicator is defined as Di2, and an arbitrary heating time of the fat and oil is defined as n.

$$Di 2 n = α \times ( Di 1 n ) + β \qquad ( 31 )$$

α: first-order coefficient of Di1n
β: constant

$$Di 2 n = γ \times {( Di 1 n )}^{2} + δ \times ( Di 1 n ) + ε \qquad ( 32 )$$

γ: second-order coefficient of Di1n
δ: first-order coefficient of Di1n
ε: constant

[35] Furthermore, the present invention provides a fat and oil deterioration degree detection method for detecting a deterioration degree of a fat and oil, the method using: a measurement device configured to measure a first deterioration indicator which is a deterioration indicator of the fat and oil and defined based on a substance produced by heating the fat and oil; and a fat and oil deterioration degree detection device configured to retain a correlation between the first deterioration indicator and a second deterioration indicator which is a deterioration indicator of the fat and oil other than the first deterioration indicator, and the method comprising: a measurement step of measuring, by the measurement device, the first deterioration indicator; a data acquisition step of acquiring, by the fat and oil deterioration degree detection device, a measured value of the first deterioration indicator measured in the measurement step; a deterioration indicator calculation step of calculating, by the fat and oil deterioration degree detection device, the second deterioration indicator based on the measured value of the first deterioration indicator acquired in the data acquisition step and the correlation as stored; and a detection result output step of outputting, by the fat and oil deterioration degree detection device, the second deterioration indicator calculated in the deterioration indicator calculation step as a result of detection of the deterioration degree.

[36] According to the fat and oil deterioration degree detection method described in [35], preferably, the correlation is a correlation equation in a form of a polynomial in which the second deterioration indicator is expressed with the first deterioration indicator.

[37] According to the fat and oil deterioration degree detection method described in [36], preferably, the correlation equation is at least one of a linear equation expressed with a following equation (31) or a quadratic equation expressed with a following equation (32), where the first deterioration indicator is defined as Di1, the second deterioration indicator is defined as Di2, and an arbitrary heating time of the fat and oil is defined as n.

$$Di 2 n = α \times ( Di 1 n ) + β \qquad ( 31 )$$

a: first-order coefficient of Di1n
β: constant

$$Di 2 n = γ \times {( Di 1 n )}^{2} + δ \times ( Di 1 n ) + ε \qquad ( 32 )$$

γ: second-order coefficient of Di1n
δ: first-order coefficient of Di1n
ε: constant

[38] According to the fat and oil deterioration degree detection program for detecting a deterioration degree of a fat and oil, the program causing a computer to execute processing comprising: a data acquisition process of acquiring a measured value of a first deterioration indicator which is a deterioration indicator of the fat and oil and defined based on a substance produced by heating the fat and oil; a deterioration indicator calculation process of calculating, using a correlation between the first deterioration indicator and a second deterioration indicator which is a deterioration indicator of the fat and oil other than the first deterioration indicator, the second deterioration indicator based on the measured value of the first deterioration indicator acquired by the data acquisition process; and a detection result output process of outputting the second deterioration indicator calculated by the deterioration indicator calculation process as a result of detection of the deterioration degree of the fat and oil.

[39] According to the fat and oil deterioration degree detection program described in [38], preferably, the correlation is a correlation equation in a form of a polynomial in which the first deterioration indicator is expressed with the second deterioration indicator.

[40] According to the fat and oil deterioration degree detection program described in [39], preferably, the correlation equation is at least one of a linear equation expressed with a following equation (31) or a quadratic equation expressed with a following equation (32), where the first deterioration indicator is defined as Di1, the second deterioration indicator is defined as Di2, and an arbitrary heating time of the fat and oil is defined as n.

$$Di 2 n = α \times ( Di 1 n ) + β \qquad ( 31 )$$

a: first-order coefficient of Di1n
β: constant

$$Di 2 n = γ \times {( Di 1 n )}^{2} + δ \times ( Di 1 n ) + ε \qquad ( 32 )$$

γ: second-order coefficient of Di1n
δ: first-order coefficient of Di1n
ε: constant

### Advantageous Effects of Invention

According to the present invention, it is possible to simply and accurately detect various deterioration indicators of fat and oil. The problems, configurations, and advantageous effects other than those described above will be clarified by explanation of the embodiments below.

### BRIEF DESCRIPTION OF DRAWINGS

FIG. 1 illustrates a part of a cooking area in which deep frying is performed.

FIG. 2 is a system configuration diagram illustrating an example of a configuration of a fat and oil deterioration level detection system according to each embodiment of the present invention.

FIG. 3 illustrates a graph of a linear function showing the correlation of the acid value of frying oil relative to the total polar compounds contained in the frying oil.

FIG. 4 illustrates a graph of a quadratic function showing the correlation of the acid value of frying oil relative to the total polar compounds contained in the frying oil.

FIG. 5 is a functional block diagram illustrating functions provided in a cloud according to the first embodiment.

FIG. 6 illustrates a flowchart of a flow of the processing to be executed in a cloud according to the first embodiment.

FIG. 7 illustrates a graph of a linear function showing the correlation of the acid value of frying oil relative to the total polar compounds contained in the frying oil, in which deep-frying weight per unit time is considered.

FIG. 8 illustrates a graph of a quadratic function showing the correlation of the acid value of frying oil relative to the total polar compounds contained in the frying oil, in which deep-frying weight per unit time is considered.

FIG. 9 is a functional block diagram illustrating functions provided in a cloud according to the second embodiment.

FIG. 10 illustrates a flowchart of a flow of the processing to be executed in a cloud according to the second embodiment.

FIG. 11 illustrates a graph of a linear function showing the correlation of the acid value of frying oil relative to the total polar compounds contained in the frying oil, in which whether empty heating has been performed is considered.

FIG. 12 illustrates a graph of a quadratic function showing the correlation of the acid value of frying oil relative to the total polar compounds contained in the frying oil, in which whether empty heating has been performed is considered.

FIG. 13 is a functional block diagram illustrating functions provided in a cloud according to the third embodiment.

FIG. 14 illustrates a flowchart of a flow of the processing to be executed in a cloud according to the third embodiment.

FIG. 15 illustrates a graph showing the correlation of the acid value of frying oil relative to the total polar compounds contained in the frying oil relating to the first oil type and the second oil type.

FIG. 16 is a functional block diagram illustrating functions provided in a cloud according to the fourth embodiment.

FIG. 17 illustrates a flowchart of a flow of the processing to be executed in a cloud according to the fourth embodiment.

FIG. 18 illustrates a graph showing the correlation of the acid value of frying oil relating to the first oil type relative to the total polar compounds contained in the frying oil, in which the actually measured acid values are compared with the acid values calculated using Equation (1), Equation (2), Equation (5), and Equation (6), respectively.

FIG. 19 illustrates a graph showing the correlation of the acid value of frying oil relating to the second oil type relative to the total polar compounds contained in the frying oil, in which the actually measured acid values are compared with the acid values calculated using Equation (1), Equation (2), Equation (7), and Equation (8), respectively.

FIG. 20 illustrates a graph showing the correlation of the acid value of frying oil relating to the third oil type and the fourth oil type relative to the total polar compounds contained in the frying oil.

FIG. 21 illustrates a flowchart of a flow of the processing to be executed in a cloud according to the fifth embodiment.

FIG. 22 illustrates a graph showing the correlation of the acid value of frying oil relating to the third oil type relative to the total polar compounds contained in the frying oil, in which the actually measured acid values are compared with the acid values calculated using Equation (1), Equation (2), Equation (9), and Equation (10), respectively.

FIG. 23 illustrates a graph showing the correlation of the acid value of frying oil relating to the fourth oil type relative to the total polar compounds contained in the frying oil, in which the actually measured acid values are compared with the acid values calculated using Equation (1), Equation (2), Equation (11), and Equation (12), respectively.

FIG. 24 illustrates a graph showing the correlation of the acid value of the frying oil P relative to the total polar compounds contained in the frying oil P relating to the fifth oil type and the sixth oil type.

FIG. 25 illustrates a flowchart of a flow of the processing to be executed in a cloud according to the sixth embodiment.

FIG. 26 illustrates a graph showing the correlation of the acid value of frying oil relating to the fifth oil type relative to the total polar compounds contained in the frying oil, in which the actually measured acid values are compared with the acid values calculated using Equation (1), Equation (2), Equation (13), and Equation (14), respectively.

FIG. 27 illustrates a graph showing the correlation of the acid value of frying oil relating to the sixth oil type relative to the total polar compounds contained in the frying oil, in which the actually measured acid values are compared with the acid values calculated using Equation (1), Equation (2), Equation (15), and Equation (16), respectively.

FIG. 28 illustrates a graph showing the correlation of the acid value of frying oil relating to the seventh oil type and the eighth oil type, which are classified based on the MG content in new oil, relative to the total polar compounds contained in the frying oil.

FIG. 29 illustrates a graph showing the correlation of the acid value of frying oil relating to the seventh oil type and the eighth oil type, which are classified based on the FFA content in new oil, relative to the total polar compounds contained in the frying oil.

FIG. 30 illustrates a graph showing the correlation of the acid value of frying oil relating to the seventh oil type and the eighth oil type, which are classified based on the MG content in heated oil, relative to the total polar compounds contained in the frying oil.

FIG. 31 illustrates a graph showing the correlation of the acid value of frying oil relating to the seventh oil type and the eighth oil type, which are classified based on the TG content in heated oil, relative to the total polar compounds contained in the frying oil.

FIG. 32 illustrates a graph showing the correlation of the acid value of frying oil relating to the seventh oil type and the eighth oil type, which are classified based on the rate of increase in the DG content due to heating, relative to the total polar compounds contained in the frying oil.

FIG. 33 illustrates a graph showing the correlation of the acid value of frying oil relating to the seventh oil type and the eighth oil type, which are classified based on the rate of increase in the FFA content due to heating, relative to the total polar compounds contained in the frying oil.

FIG. 34 illustrates a graph showing the correlation of the acid value of frying oil relating to the seventh oil type and the eighth oil type, which are classified based on the rate of decrease in the TG content due to heating, relative to the total polar compounds contained in the frying oil.

FIG. 35 illustrates a flowchart of a flow of the processing to be executed in a cloud according to the seventh embodiment.

FIG. 36 illustrates a graph showing the correlation of the acid value of frying oil relating to the seventh oil type, which is classified based on the MG content in new oil, relative to the total polar compounds contained in the frying oil, in which the actually measured acid values are compared with the acid values calculated using Equation (1), Equation (2), Equation (17), and Equation (18), respectively.

FIG. 37 illustrates a graph showing the correlation of the acid value of frying oil relating to the eighth oil type, which is classified based on the MG content in new oil, relative to the total polar compounds contained in the frying oil, in which the actually measured acid values are compared with the acid values calculated using Equation (1), Equation (2), Equation (19), and Equation (20), respectively.

FIG. 38 illustrates a graph showing the correlation of the acid value of frying oil relating to the seventh oil type, which is classified based on the FFA content in new oil, relative to the total polar compounds contained in the frying oil, in which the actually measured acid values are compared with the acid values calculated using Equation (1), Equation (2), Equation (17), and Equation (18), respectively.

FIG. 39 illustrates a graph showing the correlation of the acid value of frying oil relating to the eighth oil type, which is classified based on the FFA content in new oil, relative to the total polar compounds contained in the frying oil, in which the actually measured acid values are compared with the acid values calculated using Equation (1), Equation (2), Equation (19), and Equation (20), respectively.

FIG. 40 illustrates a graph showing the correlation of the acid value of frying oil relating to the seventh oil type, which is classified based on the MG content in heated oil, relative to the total polar compounds contained in the frying oil, in which the actually measured acid values are compared with the acid values calculated using Equation (1), Equation (2), Equation (17), and Equation (18), respectively.

FIG. 41 illustrates a graph showing the correlation of the acid value of frying oil relating to the eighth oil type, which is classified based on the MG content in heated oil, relative to the total polar compounds contained in the frying oil, in which the actually measured acid values are compared with the acid values calculated using Equation (1), Equation (2), Equation (19), and Equation (20), respectively.

FIG. 42 illustrates a graph showing the correlation of the acid value of frying oil relating to the seventh oil type, which is classified based on the TG content in heated oil, relative to the total polar compounds contained in the frying oil, in which the actually measured acid values are compared with the acid values calculated using Equation (1), Equation (2), Equation (17), and Equation (18), respectively.

FIG. 43 illustrates a graph showing the correlation of the acid value of frying oil relating to the eighth oil type, which is classified based on the TG content in heated oil, relative to the total polar compounds contained in the frying oil, in which the actually measured acid values are compared with the acid values calculated using Equation (1), Equation (2), Equation (19), and Equation (20), respectively.

FIG. 44 illustrates a graph showing the correlation of the acid value of frying oil relating to the seventh oil type, which is classified based on the rate of increase in the DG content due to heating, relative to the total polar compounds contained in the frying oil, in which the actually measured acid values are compared with the acid values calculated using Equation (1), Equation (2), Equation (17), and Equation (18), respectively.

FIG. 45 illustrates a graph showing the correlation of the acid value of frying oil relating to the eighth oil type, which is classified based on the rate of increase in the DG content due to heating, relative to the total polar compounds contained in the frying oil, in which the actually measured acid values are compared with the acid values calculated using Equation (1), Equation (2), Equation (19), and Equation (20), respectively.

FIG. 46 illustrates a graph showing the correlation of the acid value of frying oil relating to the seventh oil type, which is classified based on the rate of increase in the FFA content due to heating, relative to the total polar compounds contained in the frying oil, in which the actually measured acid values are compared with the acid values calculated using Equation (1), Equation (2), Equation (17), and Equation (18), respectively.

FIG. 47 illustrates a graph showing the correlation of the acid value of frying oil relating to the eighth oil type, which is classified based on the rate of increase in the FFA content due to heating, relative to the total polar compounds contained in the frying oil, in which the actually measured acid values are compared with the acid values calculated using Equation (1), Equation (2), Equation (19), and Equation (20), respectively.

FIG. 48 illustrates a graph showing the correlation of the acid value of frying oil relating to the seventh oil type, which is classified based on the rate of decrease in the TG content due to heating, relative to the total polar compounds contained in the frying oil, in which the actually measured acid values are compared with the acid values calculated using Equation (1), Equation (2), Equation (17), and Equation (18), respectively.

FIG. 49 illustrates a graph showing the correlation of the acid value of frying oil relating to the eighth oil type, which is classified based on the rate of decrease in the TG content due to heating, relative to the total polar compounds contained in the frying oil, in which the actually measured acid values are compared with the acid values calculated using Equation (1), Equation (2), Equation (19), and Equation (20), respectively.

FIG. 50 illustrates a graph of a linear function showing the correlation of the viscosity increase rate of frying oil relative to the total polar compounds contained in the frying oil.

FIG. 51 illustrates a graph of a linear function showing the correlation of the color of frying oil relative to the total polar compounds contained in the frying oil.

FIG. 52 illustrates a graph of a quadratic function showing the correlation of the viscosity increase rate of frying oil relative to the total polar compounds contained in the frying oil.

FIG. 53 illustrates a graph of a quadratic function showing the correlation of the color of frying oil relative to the total polar compounds contained in the frying oil.

FIG. 54 illustrates a graph of a linear function showing the correlation of the total polar compounds contained in frying oil relative to the acid value of the frying oil.

FIG. 55 illustrates a graph of a linear function showing the correlation of the viscosity increase rate of frying oil relative to the acid value of the frying oil.

FIG. 56 illustrates a graph of a linear function showing the correlation of the color of frying oil relative to the acid value of the frying oil.

FIG. 57 illustrates a graph of a quadratic function showing the correlation of the total polar compounds contained in frying oil relative to the acid value of the frying oil.

FIG. 58 illustrates a graph of a quadratic function showing the correlation of the viscosity increase rate of frying oil relative to the acid value of the frying oil.

FIG. 59 illustrates a graph of a quadratic function showing the correlation of the color of frying oil relative to the acid value of the frying oil.

FIG. 60 illustrates a graph of a linear function showing the correlation of the total polar compounds contained in frying oil relative to the viscosity increase rate of the frying oil.

FIG. 61 illustrates a graph of a linear function showing the correlation of the acid value of frying oil relative to the viscosity increase rate of the frying oil.

FIG. 62 illustrates a graph of a linear function showing the correlation of the color of frying oil relative to the viscosity increase rate of the frying oil.

FIG. 63 illustrates a graph of a quadratic function showing the correlation of the total polar compounds contained in frying oil relative to the viscosity increase rate of the frying oil.

FIG. 64 illustrates a graph of a quadratic function showing the correlation of the acid value of frying oil relative to the viscosity increase rate of the frying oil.

FIG. 65 illustrates a graph of a quadratic function showing the correlation of the color of frying oil relative to the viscosity increase rate of the frying oil.

FIG. 66 illustrates a graph of a linear function showing the correlation of the total polar compounds contained in frying oil relative to the color of the frying oil.

FIG. 67 illustrates a graph of a linear function showing the correlation of the acid value of frying oil relative to the color of the frying oil.

FIG. 68 illustrates a graph of a linear function showing the correlation of the viscosity increase rate of frying oil relative to the color of the frying oil.

FIG. 69 illustrates a graph of a quadratic function showing the correlation of the total polar compounds contained in frying oil relative to the color of the frying oil.

FIG. 70 illustrates a graph of a quadratic function showing the correlation of the acid value of frying oil relative to the color of the frying oil.

FIG. 71 illustrates a graph of a quadratic function showing the correlation of the viscosity increase rate of frying oil relative to the color of the frying oil.

FIG. 72 is a functional block diagram illustrating functions provided in a cloud according to the eighth embodiment.

FIG. 73 illustrates a flowchart of a flow of the processing to be executed in a cloud according to the eighth embodiment.

FIG. 74 illustrates a graph of a linear function showing the correlation of the total polar compounds contained in frying oil relative to the acid value of the frying oil, in which the deep-frying weight per unit time is considered.

FIG. 75 illustrates a graph of a quadratic function showing the correlation of the total polar compounds contained in frying oil relative to the acid value of the frying oil, in which the deep-frying weight per unit time is considered.

FIG. 76 illustrates a graph of a linear function showing the correlation of the viscosity increase rate of frying oil relative to the acid value of the frying oil, in which the deep-frying weight per unit time is considered.

FIG. 77 illustrates a graph of a quadratic function showing the correlation of the viscosity increase rate of frying oil relative to the acid value of the frying oil, in which the deep-frying weight per unit time is considered.

FIG. 78 illustrates a graph of a linear function showing the correlation of the acid value of frying oil relative to the viscosity increase rate of the frying oil, in which the deep-frying weight per unit time is considered.

FIG. 79 illustrates a graph of a quadratic function showing the correlation of the acid value of frying oil relative to the viscosity increase rate of the frying oil, in which the deep-frying weight per unit time is considered.

FIG. 80 is a functional block diagram illustrating functions provided in a cloud according to the ninth embodiment.

FIG. 81 illustrates a flowchart of a flow of the processing to be executed in a cloud according to the ninth embodiment.

FIG. 82 illustrates a graph of a linear function showing the correlation of the total polar compounds contained in frying oil relative to the acid value of the frying oil, in which whether empty heating has been performed is considered.

FIG. 83 illustrates a graph of a linear function showing the correlation of the viscosity increase rate of frying oil relative to the acid value of the frying oil, in which whether empty heating has been performed is considered.

FIG. 84 illustrates a graph of a linear function showing the correlation of the color of frying oil relative to the acid value of the frying oil, in which whether empty heating has been performed is considered.

FIG. 85 illustrates a graph of a quadratic function showing the correlation of the total polar compounds contained in frying oil relative to the acid value of the frying oil, in which whether empty heating has been performed is considered.

FIG. 86 illustrates a graph of a quadratic function showing the correlation of the viscosity increase rate of frying oil relative to the acid value of the frying oil, in which whether empty heating has been performed is considered.

FIG. 87 illustrates a graph of a quadratic function showing the correlation of the color of frying oil relative to the acid value of the frying oil, in which whether empty heating has been performed is considered.

FIG. 88 illustrates a graph of a linear function showing the correlation of the acid value of frying oil relative to the viscosity increase rate of the frying oil, in which whether empty heating has been performed is considered.

FIG. 89 illustrates a graph of a quadratic function showing the correlation of the acid value of frying oil relative to the viscosity increase rate of the frying oil, in which whether empty heating has been performed is considered.

FIG. 90 illustrates a graph of a linear function showing the correlation of the acid value of frying oil relative to the color of the frying oil, in which whether empty heating has been performed is considered.

FIG. 91 illustrates a graph of a quadratic function showing the correlation of the acid value of frying oil relative to the color of the frying oil, in which whether empty heating has been performed is considered.

FIG. 92 is a functional block diagram illustrating functions provided in a cloud according to the tenth embodiment.

FIG. 93 illustrates a flowchart of a flow of the processing to be executed in a cloud according to the tenth embodiment.

FIG. 94 illustrates a graph showing the correlation of the viscosity increase rate of frying oil relating to the first oil type and the second oil type relative to the total polar compounds contained in the frying oil.

FIG. 95 illustrates a graph showing the correlation of the color of frying oil relating to the first oil type and the second oil type relative to the total polar compounds contained in the frying oil.

FIG. 96 illustrates a graph showing the correlation of the total polar compounds contained in frying oil relating to the first oil type and the second oil type relative to the acid value of the frying oil.

FIG. 97 illustrates a graph showing the correlation of the viscosity increase rate of frying oil relating to the first oil type and the second oil type relative to the acid value of the frying oil.

FIG. 98 illustrates a graph showing the correlation of the total polar compounds contained in frying oil relating to the first oil type and the second oil type relative to the viscosity increase rate of the frying oil.

FIG. 99 illustrates a graph showing the correlation of the acid value of frying oil relating to the first oil type and the second oil type relative to the viscosity increase rate of the frying oil.

FIG. 100 illustrates a graph showing the correlation of the color of frying oil relating to the first oil type and the second oil type relative to the viscosity increase rate of the frying oil.

FIG. 101 illustrates a graph showing the correlation of the total polar compounds contained in frying oil relating to the first oil type and the second oil type relative to the color of the frying oil.

FIG. 102 illustrates a graph showing the correlation of the viscosity increase rate of frying oil relating to the first oil type and the second oil type. relative to the color of the frying oil

FIG. 103 is a functional block diagram illustrating functions provided in a cloud according to the eleventh embodiment.

FIG. 104 illustrates a flowchart of a flow of the processing to be executed in a cloud according to the eleventh embodiment.

FIG. 105 illustrates a graph showing the correlation of the viscosity increase rate of frying oil relating to the first oil type relative to the total polar compounds contained in the frying oil, in which the actually measured viscosity increase rates are compared with the viscosity increase rates calculated using Equation (31), Equation (32), Equation (35), and Equation (36), respectively.

FIG. 106 illustrates a graph showing the correlation of the color of frying oil relating to the first oil type relative to the total polar compounds contained in the frying oil, in which the actually measured values of the color are compared with the colors calculated using Equation (31), Equation (32), Equation (35), and Equation (36), respectively.

FIG. 107 illustrates a graph showing the correlation of the total polar compounds contained in frying oil relating to the first oil type relative to the acid value of the frying oil, in which the actually measured total polar compounds are compared with the total polar compounds calculated using Equation (31), Equation (32), Equation (35), and Equation (36), respectively.

FIG. 108 illustrates a graph showing the correlation of the viscosity increase rate of frying oil relating to the first oil type relative to the acid value of the frying oil, in which the actually measured viscosity increase rates are compared with the viscosity increase rates calculated using Equation (31), Equation (32), Equation (35), and Equation (36), respectively.

FIG. 109 illustrates a graph showing the correlation of the total polar compounds contained in frying oil relating to the first oil type relative to the viscosity increase rate of the frying oil, in which the actually measured total polar compounds are compared with the total polar compounds calculated using Equation (31), Equation (32), Equation (35), and Equation (36), respectively.

FIG. 110 illustrates a graph showing the correlation of the acid value of frying oil relating to the first oil type relative to the viscosity increase rate of the frying oil, in which the actually measured acid values are compared with the acid values calculated using Equation (31), Equation (32), Equation (35), and Equation (36), respectively.

FIG. 111 illustrates a graph showing the correlation of the color of the frying oil relating to the first oil type relative to the viscosity increase rate of the frying oil, in which the actually measured values of the color are compared with the colors calculated using Equation (31), Equation (32), Equation (35), and Equation (36), respectively.

FIG. 112 illustrates a graph showing the correlation of the total polar compounds contained in frying oil relating to the first oil type relative to the color of the frying oil, in which the actually measured total polar compounds are compared with the total polar compounds calculated using Equation (31), Equation (32), Equation (35), and Equation (36), respectively.

FIG. 113 illustrates a graph showing the correlation of the viscosity increase rate of frying oil relating to the first oil type relative to the color of the frying oil, in which the actually measured viscosity increase rates are compared with the viscosity increase rates calculated using Equation (31), Equation (32), Equation (35), and Equation (36), respectively.

FIG. 114 illustrates a graph showing the correlation of the viscosity increase rate of frying oil relating to the second oil type relative to the total polar compounds contained in the frying oil, in which the actually measured viscosity increase rates are compared with the viscosity increase rates calculated using Equation (31), Equation (32), Equation (37), and Equation (38), respectively.

FIG. 115 illustrates a graph showing the correlation of the color of frying oil relating to the second oil type relative to the total polar compounds contained in the frying oil, in which the actually measured values of the color are compared with the colors calculated using Equation (31), Equation (32), Equation (37), and Equation (38), respectively.

FIG. 116 illustrates a graph showing the correlation of the total polar compounds contained in frying oil relating to the second oil type relative to the acid value of the frying oil, in which the actually measured total polar compounds are compared with the total polar compounds calculated using Equation (31), Equation (32), Equation (37), and Equation (38), respectively.

FIG. 117 illustrates a graph showing the correlation of the viscosity increase rate of frying oil relating to the second oil type relative to the acid value of the frying oil, in which the actually measured viscosity increase rates are compared with the viscosity increase rates calculated using Equation (31), Equation (32), Equation (37), and Equation (38), respectively.

FIG. 118 illustrates a graph showing the correlation of the total polar compounds contained in frying oil relating to the second oil type relative to the viscosity increase rate of the frying oil, in which the actually measured total polar compounds are compared with the total polar compounds calculated using Equation (31), Equation (32), Equation (37), and Equation (38), respectively.

FIG. 119 illustrates a graph showing the correlation of the acid value of frying oil relating to the second oil type relative to the viscosity increase rate of the frying oil, in which the actually measured acid values are compared with the acid values calculated using Equation (31), Equation (32), Equation (37), and Equation (38), respectively.

FIG. 120 illustrates a graph showing the correlation of the color of frying oil relating to the second oil type relative to the viscosity increase rate of the frying oil, in which the actually measured values of the color are compared with the colors calculated using Equation (31), Equation (32), Equation (37), and Equation (38), respectively.

FIG. 121 illustrates a graph showing the correlation of the total polar compounds contained in frying oil relating to the second oil type relative to the color of the frying oil, in which the actually measured total polar compounds are compared with the total polar compounds calculated using Equation (31), Equation (32), Equation (37), and Equation (38), respectively.

FIG. 122 illustrates a graph showing the correlation of the viscosity increase rate of frying oil relating to the second oil type relative to the color of the frying oil, in which the actually measured viscosity increase rates are compared with the viscosity increase rates calculated using Equation (31), Equation (32), Equation (37), and Equation (38), respectively.

FIG. 123 illustrates a graph showing the correlation of the viscosity increase rate of frying oil relating to the third oil type and the fourth oil type relative to the total polar compounds contained in the frying oil.

FIG. 124 illustrates a graph showing the correlation of the color of frying oil relating to the third oil type and the fourth oil type relative to the total polar compounds contained in the frying oil.

FIG. 125 illustrates a graph showing the correlation of the total polar compounds contained in frying oil relating to the third oil type and the fourth oil type relative to the acid value of the frying oil.

FIG. 126 illustrates a graph showing the correlation of the viscosity increase rate of frying oil relating to the third oil type and the fourth oil type relative to the acid value of the frying oil.

FIG. 127 illustrates a graph showing the correlation of the total polar compounds contained in frying oil relating to the third oil type and the fourth oil type relative to the viscosity increase rate of the frying oil.

FIG. 128 illustrates a graph showing the correlation of the acid value of frying oil relating to the third oil type and the fourth oil type relative to the viscosity increase rate of the frying oil.

FIG. 129 illustrates a graph showing the correlation of the color of frying oil relating to the third oil type and the fourth oil type relative to the viscosity increase rate of the frying oil.

FIG. 130 illustrates a graph showing the correlation of the total polar compounds contained in frying oil relating to the third oil type and the fourth oil type relative to the color of the frying oil.

FIG. 131 illustrates a graph showing the correlation of the viscosity increase rate of frying oil relating to the third oil type and the fourth oil type relative to the color of the frying oil.

FIG. 132 illustrates a flowchart of a flow of the processing to be executed in a cloud according to the twelfth embodiment.

FIG. 133 illustrates a graph showing the correlation of the viscosity increase rate of frying oil relating to the third oil type relative to the total polar compounds contained in the frying oil, in which the actually measured viscosity increase rates are compared with the viscosity increase rates calculated using Equation (31), Equation (32), Equation (39), and Equation (40), respectively.

FIG. 134 illustrates a graph showing the correlation of the color of frying oil relating to the third oil type relative to the total polar compounds contained in the frying oil, in which the actually measured values of the color are compared with the colors calculated using Equation (31), Equation (32), Equation (39), and Equation (40), respectively.

FIG. 135 illustrates a graph showing the correlation of the total polar compounds contained in frying oil relating to the third oil type relative to the acid value of the frying oil, in which the actually measured total polar compounds are compared with the total polar compounds calculated using Equation (31), Equation (32), Equation (39), and Equation (40), respectively.

FIG. 136 illustrates a graph showing the correlation of the viscosity increase rate of frying oil relating to the third oil type relative to the acid value of the frying oil, in which the actually measured viscosity increase rates are compared with the viscosity increase rates calculated using Equation (31), Equation (32), Equation (39), and Equation (40), respectively.

FIG. 137 illustrates a graph showing the correlation of the total polar compounds contained in frying oil relating to the third oil type relative to the viscosity increase rate of the frying oil, in which the actually measured total polar compounds are compared with the total polar compounds calculated using Equation (31), Equation (32), Equation (39), and Equation (40), respectively.

FIG. 138 illustrates a graph showing the correlation of the acid value of frying oil relating to the third oil type relative to the viscosity increase rate of the frying oil, in which the actually measured acid values are compared with the acid values calculated using Equation (31), Equation (32), Equation (39), and Equation (40), respectively.

FIG. 139 illustrates a graph showing the correlation of the color of frying oil relating to the third oil type relative to the viscosity increase rate of the frying oil, in which the actually measured values of the color are compared with the colors calculated using Equation (31), Equation (32), Equation (39), and Equation (40), respectively.

FIG. 140 illustrates a graph showing the correlation of the total polar compounds contained in frying oil relating to the third oil type relative to the color of the frying oil, in which the actually measured total polar compounds are compared with the total polar compounds calculated using Equation (31), Equation (32), Equation (39), and Equation (40), respectively.

FIG. 141 illustrates a graph showing the correlation of the viscosity increase rate of frying oil relating to the third oil type relative to the color of the frying oil, in which the actually measured viscosity increase rates are compared with the viscosity increase rates calculated using Equation (31), Equation (32), Equation (39), and Equation (40), respectively.

FIG. 142 illustrates a graph showing the correlation of the viscosity increase rate of frying oil relating to the fourth oil type relative to the total polar compounds contained in the frying oil, in which the actually measured viscosity increase rates are compared with the viscosity increase rates calculated using Equation (31), Equation (32), Equation (41), and Equation (42), respectively.

FIG. 143 illustrates a graph showing the correlation of the color of frying oil relating to the fourth oil type relative to the total polar compounds contained in the frying oil, in which the actually measured values of the color are compared with the colors calculated using Equation (31), Equation (32), Equation (41), and Equation (42), respectively.

FIG. 144 illustrates a graph showing the correlation of the total polar compounds contained in frying oil relating to the fourth oil type relative to the acid value of the frying oil, in which the actually measured total polar compounds are compared with the total polar compounds calculated using Equation (31), Equation (32), Equation (41), and Equation (42), respectively.

FIG. 145 illustrates a graph showing the correlation of the viscosity increase rate of frying oil relating to the fourth oil type relative to the acid value of the frying oil, in which the actually measured viscosity increase rates are compared with the viscosity increase rates calculated using Equation (31), Equation (32), Equation (41), and Equation (42), respectively.

FIG. 146 illustrates a graph showing the correlation of the total polar compounds contained in frying oil relating to the fourth oil type relative to the viscosity increase rate of the frying oil, in which the actually measured total polar compounds are compared with the total polar compounds calculated using Equation (31), Equation (32), Equation (41), and Equation (42), respectively.

FIG. 147 illustrates a graph showing the correlation of the acid value of frying oil relating to the fourth oil type relative to the viscosity increase rate of the frying oil, in which the actually measured acid values are compared with the acid values calculated using Equation (31), Equation (32), Equation (41), and Equation (42), respectively.

FIG. 148 illustrates a graph showing the correlation of the color of frying oil relating to the fourth oil type relative to the viscosity increase rate of the frying oil, in which the actually measured values of the color are compared with the colors calculated using Equation (31), Equation (32), Equation (41), and Equation (42), respectively.

FIG. 149 illustrates a graph showing the correlation of the total polar compounds contained in frying oil relating to the fourth oil type relative to the color of the frying oil, in which the actually measured total polar compounds are compared with the total polar compounds calculated using Equation (31), Equation (32), Equation (41), and Equation (42), respectively.

FIG. 150 illustrates a graph showing the correlation of the viscosity increase rate of frying oil relating to the fourth oil type relative to the color of the frying oil, in which the actually measured viscosity increase rates are compared with the viscosity increase rates calculated using Equation (31), Equation (32), Equation (41), and Equation (42), respectively.

FIG. 151 illustrates a graph showing the correlation of the viscosity increase rate of frying oil relating to the fifth oil type and the sixth oil type relative to the total polar compounds contained in the frying oil.

FIG. 152 illustrates a graph showing the correlation of the color of frying oil relating to the fifth oil type and the sixth oil type relative to the total polar compounds contained in the frying oil.

FIG. 153 illustrates a graph showing the correlation of the total polar compounds contained in frying oil relating to the fifth oil type and the sixth oil type relative to the acid value of the frying oil.

FIG. 154 illustrates a graph showing the correlation of the viscosity increase rate of frying oil relating to the fifth oil type and the sixth oil type relative to the acid value of the frying oil.

FIG. 155 illustrates a graph showing the correlation of the total polar compounds contained in frying oil relating to the fifth oil type and the sixth oil type relative to the viscosity increase rate of the frying oil.

FIG. 156 illustrates a graph showing the correlation of the acid value of frying oil relating to the fifth oil type and the sixth oil type relative to the viscosity increase rate of the frying oil.

FIG. 157 illustrates a graph showing the correlation of the color of frying oil relating to the fifth oil type and the sixth oil type relative to the viscosity increase rate of the frying oil.

FIG. 158 illustrates a graph showing the correlation of the total polar compounds contained in frying oil relating to the fifth oil type and the sixth oil type relative to the color of the frying oil.

FIG. 159 illustrates a graph showing the correlation of the viscosity increase rate of frying oil relating to the fifth oil type and the sixth oil type relative to the color of the frying oil.

FIG. 160 illustrates a flowchart of a flow of the processing to be executed in a cloud according to the thirteenth embodiment.

FIG. 161 illustrates a graph showing the correlation of the viscosity increase rate of frying oil relating to the fifth oil type relative to the total polar compounds contained in the frying oil, in which the actually measured viscosity increase rates are compared with the viscosity increase rates calculated using Equation (31), Equation (32), Equation (43), and Equation (44), respectively.

FIG. 162 illustrates a graph showing the correlation of the color of frying oil relating to the fifth oil type relative to the total polar compounds contained in the frying oil, in which the actually measured values of the color are compared with the colors calculated using Equation (31), Equation (32), Equation (43), and Equation (44), respectively.

FIG. 163 illustrates a graph showing the correlation of the total polar compounds contained in frying oil relating to the fifth oil type relative to the acid value of the frying oil, in which the actually measured total polar compounds are compared with the total polar compounds calculated using Equation (31), Equation (32), Equation (43), and Equation (44), respectively.

FIG. 164 illustrates a graph showing the correlation of the viscosity increase rate of frying oil relating to the fifth oil type relative to the acid value of the frying oil, in which the actually measured viscosity increase rates are compared with the viscosity increase rates calculated using Equation (31), Equation (32), Equation (43), and Equation (44), respectively.

FIG. 165 illustrates a graph showing the correlation of the total polar compounds contained in frying oil relating to the fifth oil type relative to the viscosity increase rate of the frying oil, in which the actually measured total polar compounds are compared with the total polar compounds calculated using Equation (31), Equation (32), Equation (43), and Equation (44), respectively.

FIG. 166 illustrates a graph showing the correlation of the acid value of frying oil relating to the fifth oil type relative to the viscosity increase rate of the frying oil, in which the actually measured acid values are compared with the acid values calculated using Equation (31), Equation (32), Equation (43), and Equation (44), respectively.

FIG. 167 illustrates a graph showing the correlation of the color of frying oil relating to the fifth oil type relative to the viscosity increase rate of the frying oil, in which the actually measured values of the color are compared with the colors calculated using Equation (31), Equation (32), Equation (43), and Equation (44), respectively.

FIG. 168 illustrates a graph showing the correlation of the total polar compounds contained in frying oil relating to the fifth oil type relative to the color of the frying oil, in which the actually measured total polar compounds are compared with the total polar compounds calculated using Equation (31), Equation (32), Equation (43), and Equation (44), respectively.

FIG. 169 illustrates a graph showing the correlation of the viscosity increase rate of frying oil relating to the fifth oil type relative to the color of the frying oil, in which the actually measured viscosity increase rates are compared with the viscosity increase rates calculated using Equation (31), Equation (32), Equation (43), and Equation (44), respectively.

FIG. 170 illustrates a graph showing the correlation of the viscosity increase rate of frying oil relating to the sixth oil type relative to the total polar compounds contained in the frying oil, in which the actually measured viscosity increase rates are compared with the viscosity increase rates calculated using Equation (31), Equation (32), Equation (45), and Equation (46), respectively.

FIG. 171 illustrates a graph showing the correlation of the color of frying oil relating to the sixth oil type relative to the total polar compounds contained in the frying oil, in which the actually measured values of the color are compared with the colors calculated using Equation (31), Equation (32), Equation (45), and Equation (46), respectively.

FIG. 172 illustrates a graph showing the correlation of the total polar compounds contained in frying oil relating to the sixth oil type relative to the acid value of the frying oil, in which the actually measured total polar compounds are compared with the total polar compounds calculated using Equation (31), Equation (32), Equation (45), and Equation (46), respectively.

FIG. 173 illustrates a graph showing the correlation of the viscosity increase rate of frying oil relating to the sixth oil type relative to the acid value of the frying oil, in which the actually measured viscosity increase rates are compared with the viscosity increase rates calculated using Equation (31), Equation (32), Equation (45), and Equation (46), respectively.

FIG. 174 illustrates a graph showing the correlation of the total polar compounds contained in frying oil relating to the sixth oil type relative to the viscosity increase rate of the frying oil, in which the actually measured total polar compounds are compared with the total polar compounds calculated using Equation (31), Equation (32), Equation (45), and Equation (46), respectively.

FIG. 175 illustrates a graph showing the correlation of the acid value of frying oil relating to the sixth oil type relative to the viscosity increase rate of the frying oil, in which the actually measured acid values are compared with the acid values calculated using Equation (31), Equation (32), Equation (45), and Equation (46), respectively.

FIG. 176 illustrates a graph showing the correlation of the color of frying oil relating to the sixth oil type relative to the viscosity increase rate of the frying oil, in which the actually measured values of the color are compared with the colors calculated using Equation (31), Equation (32), Equation (45), and Equation (46), respectively.

FIG. 177 illustrates a graph showing the correlation of the total polar compounds contained in frying oil relating to the sixth oil type relative to the color of the frying oil, in which the actually measured total polar compounds are compared with the total polar compounds calculated using Equation (31), Equation (32), Equation (45), and Equation (46), respectively.

FIG. 178 illustrates a graph showing the correlation of the viscosity increase rate of frying oil relating to the sixth oil type relative to the color of the frying oil, in which the actually measured viscosity increase rates are compared with the viscosity increase rates calculated using Equation (31), Equation (32), Equation (45), and Equation (46), respectively.

FIG. 179 illustrates a graph showing the correlation of the viscosity increase rate of frying oil relating to the seventh oil type and the eighth oil type, which are classified based on the MG content in new oil, relative to the total polar compounds contained in the frying oil.

FIG. 180 illustrates a graph showing the correlation of the color of frying oil relating to the seventh oil type and the eighth oil type, which are classified based on the MG content in new oil, relative to the total polar compounds contained in the frying oil.

FIG. 181 illustrates a graph showing the correlation of the total polar compounds contained in frying oil relating to the seventh oil type and the eighth oil type, which are classified based on the MG content in new oil, relative to the acid value of the frying oil.

FIG. 182 illustrates a graph showing the correlation of the viscosity increase rate of frying oil relating to the seventh oil type and the eighth oil type, which are classified based on the MG content in new oil, relative to the acid value of the frying oil.

FIG. 183 illustrates a graph showing the correlation of the total polar compounds contained in frying oil relating to the seventh oil type and the eighth oil type, which are classified based on the MG content in new oil, relative to the viscosity increase rate of the frying oil.

FIG. 184 illustrates a graph showing the correlation of the acid value of frying oil relating to the seventh oil type and the eighth oil type, which are classified based on the MG content in new oil, relative to the viscosity increase rate of the frying oil.

FIG. 185 illustrates a graph showing the correlation of the color of frying oil relating to the seventh oil type and the eighth oil type, which are classified based on the MG content in new oil, relative to the viscosity increase rate of the frying oil.

FIG. 186 illustrates a graph showing the correlation of the total polar compounds contained in frying oil relating to the seventh oil type and the eighth oil type, which are classified based on the MG content in new oil, relative to the color of the frying oil.

FIG. 187 illustrates a graph showing the correlation of the viscosity increase rate of frying oil relating to the seventh oil type and the eighth oil type, which are classified based on the MG content in new oil, relative to the color of the frying oil.

FIG. 188 illustrates a graph showing the correlation of the viscosity increase rate of frying oil relating to the seventh oil type and the eighth oil type, which are classified based on the FFA content in new oil, relative to the total polar compounds contained in the frying oil.

FIG. 189 illustrates a graph showing the correlation of the color of frying oil relating to the seventh oil type and the eighth oil type, which are classified based on the FFA content in new oil, relative to the total polar compounds contained in the frying oil.

FIG. 190 illustrates a graph showing the correlation of the total polar compounds contained in frying oil relating to the seventh oil type and the eighth oil type, which are classified based on the FFA content in new oil, relative to the acid value of the frying oil.

FIG. 191 illustrates a graph showing the correlation of the viscosity increase rate of frying oil relating to the seventh oil type and the eighth oil type, which are classified based on the FFA content in new oil, relative to the acid value of the frying oil.

FIG. 192 illustrates a graph showing the correlation of the total polar compounds contained in frying oil relating to the seventh oil type and the eighth oil type, which are classified based on the FFA content in new oil, relative to the viscosity increase rate of the frying oil.

FIG. 193 illustrates a graph showing the correlation of the acid value of frying oil relating to the seventh oil type and the eighth oil type, which are classified based on the FFA content in new oil, relative to the viscosity increase rate of the frying oil.

FIG. 194 illustrates a graph showing the correlation of the color of frying oil relating to the seventh oil type and the eighth oil type, which are classified based on the FFA content in new oil, relative to the viscosity increase rate of the frying oil.

FIG. 195 illustrates a graph showing the correlation of the total polar compounds contained in frying oil relating to the seventh oil type and the eighth oil type, which are classified based on the FFA content in new oil, relative to the color of the frying oil.

FIG. 196 illustrates a graph showing the correlation of the viscosity increase rate of frying oil relating to the seventh oil type and the eighth oil type, which are classified based on the FFA content in new oil, relative to the color of the frying oil.

FIG. 197 illustrates a graph showing the correlation of the viscosity increase rate of frying oil relating to the seventh oil type and the eighth oil type, which are classified based on the MG content in heated oil, relative to the total polar compounds contained in the frying oil.

FIG. 198 illustrates a graph showing the correlation of the color of frying oil relating to the seventh oil type and the eighth oil type, which are classified based on the MG content in heated oil, relative to the total polar compounds contained in the frying oil.

FIG. 199 illustrates a graph showing the correlation of the total polar compounds contained in frying oil relating to the seventh oil type and the eighth oil type, which are classified based on the MG content in heated oil, relative to the acid value of the frying oil.

FIG. 200 illustrates a graph showing the correlation of the viscosity increase rate of frying oil relating to the seventh oil type and the eighth oil type, which are classified based on the MG content in heated oil, relative to the acid value of the frying oil.

FIG. 201 illustrates a graph showing the correlation of the total polar compounds contained in frying oil relating to the seventh oil type and the eighth oil type, which are classified based on the MG content in heated oil, relative to the viscosity increase rate of the frying oil.

FIG. 202 illustrates a graph showing the correlation of the acid value of frying oil relating to the seventh oil type and the eighth oil type, which are classified based on the MG content in heated oil, relative to the viscosity increase rate of the frying oil.

FIG. 203 illustrates a graph showing the correlation of the color of frying oil relating to the seventh oil type and the eighth oil type, which are classified based on the MG content in heated oil, relative to the viscosity increase rate of the frying oil.

FIG. 204 illustrates a graph showing the correlation of the total polar compounds contained in frying oil relating to the seventh oil type and the eighth oil type, which are classified based on the MG content in heated oil, relative to the color of the frying oil.

FIG. 205 illustrates a graph showing the correlation of the viscosity increase rate of frying oil relating to the seventh oil type and the eighth oil type, which are classified based on the MG content in heated oil, relative to the color of the frying oil.

FIG. 206 illustrates a graph showing the correlation of the viscosity increase rate of frying oil relating to the seventh oil type and the eighth oil type, which are classified based on the TG content in heated oil, relative to the total polar compounds contained in the frying oil.

FIG. 207 illustrates a graph showing the correlation of the color of frying oil relating to the seventh oil type and the eighth oil type, which are classified based on the TG content in heated oil, relative to the total polar compounds contained in the frying oil.

FIG. 208 illustrates a graph showing the correlation of the total polar compounds contained in frying oil relating to the seventh oil type and the eighth oil type, which are classified based on the TG content in heated oil, relative to the acid value of the frying oil.

FIG. 209 illustrates a graph showing the correlation of the viscosity increase rate of frying oil relating to the seventh oil type and the eighth oil type, which are classified based on the TG content in heated oil, relative to the acid value of the frying oil.

FIG. 210 illustrates a graph showing the correlation of the total polar compounds contained in frying oil relating to the seventh oil type and the eighth oil type, which are classified based on the TG content in heated oil, relative to the viscosity increase rate of the frying oil.

FIG. 211 illustrates a graph showing the correlation of the acid value of frying oil relating to the seventh oil type and the eighth oil type, which are classified based on the TG content in heated oil, relative to the viscosity increase rate of the frying oil.

FIG. 212 illustrates a graph showing the correlation of the color of frying oil relating to the seventh oil type and the eighth oil type, which are classified based on the TG content in heated oil, relative to the viscosity increase rate of the frying oil.

FIG. 213 illustrates a graph showing the correlation of the total polar compounds contained in frying oil relating to the seventh oil type and the eighth oil type, which are classified based on the TG content in heated oil, relative to the color of the frying oil.

FIG. 214 illustrates a graph showing the correlation of the viscosity increase rate of frying oil relating to the seventh oil type and the eighth oil type, which are classified based on the TG content in heated oil, relative to the color of the frying oil.

FIG. 215 illustrates a graph showing the correlation of the viscosity increase rate of frying oil relating to the seventh oil type and the eighth oil type, which are classified based on the rate of increase in the DG content due to heating, relative to the total polar compounds contained in the frying oil.

FIG. 216 illustrates a graph showing the correlation of the color of frying oil relating to the seventh oil type and the eighth oil type, which are classified based on the rate of increase in the DG content due to heating, relative to the total polar compounds contained in the frying oil.

FIG. 217 illustrates a graph showing the correlation of the total polar compounds contained in frying oil relating to the seventh oil type and the eighth oil type, which are classified based on the rate of increase in the DG content due to heating, relative to the acid value of the frying oil.

FIG. 218 illustrates a graph showing the correlation of the viscosity increase rate of frying oil relating to the seventh oil type and the eighth oil type, which are classified based on the rate of increase in the DG content due to heating, relative to the acid value of the frying oil.

FIG. 219 illustrates a graph showing the correlation of the total polar compounds contained in frying oil relating to the seventh oil type and the eighth oil type, which are classified based on the rate of increase in the DG content due to heating, relative to the viscosity increase rate of the frying oil.

FIG. 220 illustrates a graph showing the correlation of the acid value of frying oil relating to the seventh oil type and the eighth oil type, which are classified based on the rate of increase in the DG content due to heating, relative to the viscosity increase rate of the frying oil.

FIG. 221 illustrates a graph showing the correlation of the color of frying oil relating to the seventh oil type and the eighth oil type, which are classified based on the rate of increase in the DG content due to heating, relative to the viscosity increase rate of the frying oil.

FIG. 222 illustrates a graph showing the correlation of the total polar compounds contained in frying oil relating to the seventh oil type and the eighth oil type, which are classified based on the rate of increase in the DG content due to heating, relative to the color of the frying oil.

FIG. 223 illustrates a graph showing the correlation of the viscosity increase rate of frying oil relating to the seventh oil type and the eighth oil type, which are classified based on the rate of increase in the DG content due to heating, relative to the color of the frying oil.

FIG. 224 illustrates a graph showing the correlation of the viscosity increase rate of frying oil relating to the seventh oil type and the eighth oil type, which are classified based on the rate of increase in the FFA content due to heating, relative to the total polar compounds contained in the frying oil.

FIG. 225 illustrates a graph showing the correlation of the color of frying oil relating to the seventh oil type and the eighth oil type, which are classified based on the rate of increase in the FFA content due to heating, relative to the total polar compounds contained in the frying oil.

FIG. 226 illustrates a graph showing the correlation of the total polar compounds contained in frying oil relating to the seventh oil type and the eighth oil type, which are classified based on the rate of increase in the FFA content due to heating, relative to the acid value of the frying oil.

FIG. 227 illustrates a graph showing the correlation of the viscosity increase rate of frying oil relating to the seventh oil type and the eighth oil type, which are classified based on the rate of increase in the FFA content due to heating, relative to the acid value of the frying oil.

FIG. 228 illustrates a graph showing the correlation of the total polar compounds contained in frying oil relating to the seventh oil type and the eighth oil type, which are classified based on the rate of increase in the FFA content due to heating, relative to the viscosity increase rate of the frying oil.

FIG. 229 illustrates a graph showing the correlation of the acid value of frying oil relating to the seventh oil type and the eighth oil type, which are classified based on the rate of increase in the FFA content due to heating, relative to the viscosity increase rate of the frying oil.

FIG. 230 illustrates a graph showing the correlation of the color of frying oil relating to the seventh oil type and the eighth oil type, which are classified based on the rate of increase in the FFA content due to heating, relative to the viscosity increase rate of the frying oil.

FIG. 231 illustrates a graph showing the correlation of the total polar compounds contained in frying oil relating to the seventh oil type and the eighth oil type, which are classified based on the rate of increase in the FFA content due to heating, relative to the color of the frying oil.

FIG. 232 illustrates a graph showing the correlation of the viscosity increase rate of frying oil relating to the seventh oil type and the eighth oil type, which are classified based on the rate of increase in the FFA content due to heating, relative to the color of the frying oil.

FIG. 233 illustrates a graph showing the correlation of the viscosity increase rate of frying oil relating to the seventh oil type and the eighth oil type, which are classified based on the rate of decrease in the TG content due to heating, relative to the total polar compounds contained in the frying oil.

FIG. 234 illustrates a graph showing the correlation of the color of frying oil relating to the seventh oil type and the eighth oil type, which are classified based on the rate of decrease in the TG content due to heating, relative to the total polar compounds contained in the frying oil.

FIG. 235 illustrates a graph showing the correlation of the total polar compounds contained in frying oil relating to the seventh oil type and the eighth oil type, which are classified based on the rate of decrease in the TG content due to heating, relative to the acid value of the frying oil.

FIG. 236 illustrates a graph showing the correlation of the viscosity increase rate of frying oil relating to the seventh oil type and the eighth oil type, which are classified based on the rate of decrease in the TG content due to heating, relative to the acid value of the frying oil.

FIG. 237 illustrates a graph showing the correlation of the total polar compounds contained in frying oil relating to the seventh oil type and the eighth oil type, which are classified based on the rate of decrease in the TG content due to heating, relative to the viscosity increase rate of the frying oil.

FIG. 238 illustrates a graph showing the correlation of the acid value of frying oil relating to the seventh oil type and the eighth oil type, which are classified based on the rate of decrease in the TG content due to heating, relative to the viscosity increase rate of the frying oil.

FIG. 239 illustrates a graph showing the correlation of the color of frying oil relating to the seventh oil type and the eighth oil type, which are classified based on the rate of decrease in the TG content due to heating, relative to the viscosity increase rate of the frying oil.

FIG. 240 illustrates a graph showing the correlation of the total polar compounds contained in frying oil relating to the seventh oil type and the eighth oil type, which are classified based on the rate of decrease in the TG content due to heating, relative to the color of the frying oil.

FIG. 241 illustrates a graph showing the correlation of the viscosity increase rate of frying oil relating to the seventh oil type and the eighth oil type, which are classified based on the rate of decrease in the TG content due to heating, relative to the color of the frying oil.

FIG. 242 illustrates a flowchart of a flow of the processing to be executed in a cloud according to the fourteenth embodiment.

FIG. 243 illustrates a graph showing the correlation of the viscosity increase rate of frying oil relating to the seventh oil type, which is classified based on the MG content in new oil, relative to the total polar compounds contained in the frying oil, in which the actually measured viscosity increase rates are compared with the viscosity increase rates calculated using Equation (31), Equation (32), Equation (47), and Equation (48), respectively.

FIG. 244 illustrates a graph showing the correlation of the color of frying oil relating to the seventh oil type, which is classified based on the MG content in new oil, relative to the total polar compounds contained in the frying oil, in which the actually measured values of the color are compared with the colors calculated using Equation (31), Equation (32), Equation (47), and Equation (48), respectively.

FIG. 245 illustrates a graph showing the correlation of the total polar compounds contained in frying oil relating to the seventh oil type, which is classified based on the MG content in new oil, relative to the acid value of the frying oil, in which the actually measured total polar compounds are compared with the total polar compounds calculated using Equation (31), Equation (32), Equation (47), and Equation (48), respectively.

FIG. 246 illustrates a graph showing the correlation of the viscosity increase rate of frying oil relating to the seventh oil type, which is classified based on the MG content in new oil, relative to the acid value of the frying oil, in which the actually measured viscosity increase rates are compared with the viscosity increase rates calculated using Equation (31), Equation (32), Equation (47), and Equation (48), respectively.

FIG. 247 illustrates a graph showing the correlation of the total polar compounds contained in frying oil relating to the seventh oil type, which is classified based on the MG content in new oil, relative to the viscosity increase rate of the frying oil, in which the actually measured total polar compounds are compared with the total polar compounds calculated using Equation (31), Equation (32), Equation (47), and Equation (48), respectively.

FIG. 248 illustrates a graph showing the correlation of the acid value of frying oil relating to the seventh oil type, which is classified based on the MG content in new oil, relative to the viscosity increase rate of the frying oil, in which the actually measured acid values are compared with the acid values calculated using Equation (31), Equation (32), Equation (47), and Equation (48), respectively.

FIG. 249 illustrates a graph showing the correlation of the color of frying oil relating to the seventh oil type, which is classified based on the MG content in new oil, relative to the viscosity increase rate of the frying oil, in which the actually measured values of the color are compared with the colors calculated using Equation (31), Equation (32), Equation (47), and Equation (48), respectively.

FIG. 250 illustrates a graph showing the correlation of the total polar compounds contained in frying oil relating to the seventh oil type, which is classified based on the MG content in new oil, relative to the color of the frying oil, in which the actually measured total polar compounds are compared with the total polar compounds calculated using Equation (31), Equation (32), Equation (47), and Equation (48), respectively.

FIG. 251 illustrates a graph showing the correlation of the viscosity increase rate of frying oil relating to the seventh oil type, which is classified based on the MG content in new oil, relative to the color of the frying oil, in which the actually measured viscosity increase rates are compared with the viscosity increase rates calculated using Equation (31), Equation (32), Equation (47), and Equation (48), respectively.

FIG. 252 illustrates a graph showing the correlation of the viscosity increase rate of frying oil relating to the eighth oil type, which is classified based on the MG content in new oil, relative to the total polar compounds contained in the frying oil, in which the actually measured viscosity increase rates are compared with the viscosity increase rates calculated using Equation (31), Equation (32), Equation (49), and Equation (50), respectively.

FIG. 253 illustrates a graph showing the correlation of the color of frying oil relating to the eighth oil type, which is classified based on the MG content in new oil, relative to the total polar compounds contained in the frying oil, in which the actually measured values of the color are compared with the colors calculated using Equation (31), Equation (32), Equation (49), and Equation (50), respectively.

FIG. 254 illustrates a graph showing the correlation of the total polar compounds contained in frying oil relating to the eighth oil type, which is classified based on the MG content in new oil, relative to the acid value of the frying oil, in which the actually measured total polar compounds are compared with the total polar compounds calculated using Equation (31), Equation (32), Equation (49), and Equation (50), respectively.

FIG. 255 illustrates a graph showing the correlation of the viscosity increase rate of frying oil relating to the eighth oil type, which is classified based on the MG content in new oil, relative to the acid value of the frying oil, in which the actually measured viscosity increase rates are compared with the viscosity increase rates calculated using Equation (31), Equation (32), Equation (49), and Equation (50), respectively.

FIG. 256 illustrates a graph showing the correlation of the total polar compounds contained in frying oil relating to the eighth oil type, which is classified based on the MG content in new oil, relative to the viscosity increase rate of the frying oil, in which the actually measured total polar compounds are compared with the total polar compounds calculated using Equation (31), Equation (32), Equation (49), and Equation (50), respectively.

FIG. 257 illustrates a graph showing the correlation of the acid value of frying oil relating to the eighth oil type, which is classified based on the MG content in new oil, relative to the viscosity increase rate of the frying oil, in which the actually measured acid values are compared with the acid values calculated using Equation (31), Equation (32), Equation (49), and Equation (50), respectively.

FIG. 258 illustrates a graph showing the correlation of the color of frying oil relating to the eighth oil type, which is classified based on the MG content in new oil, relative to the viscosity increase rate of the frying oil, in which the actually measured values of the color are compared with the colors calculated using Equation (31), Equation (32), Equation (49), and Equation (50), respectively.

FIG. 259 illustrates a graph showing the correlation of the total polar compounds contained in frying oil relating to the eighth oil type, which is classified based on the MG content in new oil, relative to the color of the frying oil, in which the actually measured total polar compounds are compared with the total polar compounds calculated using Equation (31), Equation (32), Equation (49), and Equation (50), respectively.

FIG. 260 illustrates a graph showing the correlation of the viscosity increase rate of frying oil relating to the eighth oil type, which is classified based on the MG content in new oil, relative to the color of the frying oil, in which the actually measured viscosity increase rates are compared with the viscosity increase rates calculated using Equation (31), Equation (32), Equation (49), and Equation (50), respectively.

FIG. 261 illustrates a graph showing the correlation of the viscosity increase rate of frying oil relating to the seventh oil type, which is classified based on the FFA content in new oil, relative to the total polar compounds contained in the frying oil, in which the actually measured viscosity increase rates are compared with the viscosity increase rates calculated using Equation (31), Equation (32), Equation (47), and Equation (48), respectively.

FIG. 262 illustrates a graph showing the correlation of the color of frying oil relating to the seventh oil type, which is classified based on the FFA content in new oil, relative to the total polar compounds contained in the frying oil, in which the actually measured values of the color are compared with the colors calculated using Equation (31), Equation (32), Equation (47), and Equation (48), respectively.

FIG. 263 illustrates a graph showing the correlation of the total polar compounds contained in frying oil relating to the seventh oil type, which is classified based on the FFA content in new oil, relative to the acid value of the frying oil, in which the actually measured total polar compounds are compared with the total polar compounds calculated using Equation (31), Equation (32), Equation (47), and Equation (48), respectively.

FIG. 264 illustrates a graph showing the correlation of the viscosity increase rate of frying oil relating to the seventh oil type, which is classified based on the FFA content in new oil, relative to the acid value of the frying oil, in which the actually measured viscosity increase rates are compared with the viscosity increase rates calculated using Equation (31), Equation (32), Equation (47), and Equation (48), respectively.

FIG. 265 illustrates a graph showing the correlation of the total polar compounds contained in frying oil relating to the seventh oil type, which is classified based on the FFA content in new oil, relative to the viscosity increase rate of the frying oil, in which the actually measured total polar compounds are compared with the total polar compounds calculated using Equation (31), Equation (32), Equation (47), and Equation (48), respectively.

FIG. 266 illustrates a graph showing the correlation of the acid value of frying oil relating to the seventh oil type, which is classified based on the FFA content in new oil, relative to the viscosity increase rate of the frying oil, in which the actually measured acid values are compared with the acid values calculated using Equation (31), Equation (32), Equation (47), and Equation (48), respectively.

FIG. 267 illustrates a graph showing the correlation of the color of frying oil relating to the seventh oil type, which is classified based on the FFA content in new oil, relative to the viscosity increase rate of the frying oil, in which the actually measured values of the color are compared with the colors calculated using Equation (31), Equation (32), Equation (47), and Equation (48), respectively.

FIG. 268 illustrates a graph showing the correlation of the total polar compounds contained in frying oil relating to the seventh oil type, which is classified based on the FFA content in new oil, relative to the color of the frying oil, in which the actually measured total polar compounds are compared with the total polar compounds calculated using Equation (31), Equation (32), Equation (47), and Equation (48), respectively.

FIG. 269 illustrates a graph showing the correlation of the viscosity increase rate of frying oil relating to the seventh oil type, which is classified based on the FFA content in new oil, relative to the color of the frying oil, in which the actually measured viscosity increase rates are compared with the viscosity increase rates calculated using Equation (31), Equation (32), Equation (47), and Equation (48), respectively.

FIG. 270 illustrates a graph showing the correlation of the viscosity increase rate of frying oil relating to the eighth oil type, which is classified based on the FFA content in new oil, relative to the total polar compounds contained in the frying oil, in which the actually measured viscosity increase rates are compared with the viscosity increase rates calculated using Equation (31), Equation (32), Equation (49), and Equation (50), respectively.

FIG. 271 illustrates a graph showing the correlation of the color of frying oil relating to the eighth oil type, which is classified based on the FFA content in new oil, relative to the total polar compounds contained in the frying oil, in which the actually measured values of the color are compared with the colors calculated using Equation (31), Equation (32), Equation (49), and Equation (50), respectively.

FIG. 272 illustrates a graph showing the correlation of the total polar compounds contained in frying oil relating to the eighth oil type, which is classified based on the FFA content in new oil, relative to the acid value of the frying oil, in which the actually measured total polar compounds are compared with the total polar compounds calculated using Equation (31), Equation (32), Equation (49), and Equation (50), respectively.

FIG. 273 illustrates a graph showing the correlation of the viscosity increase rate of frying oil relating to the eighth oil type, which is classified based on the FFA content in new oil, relative to the acid value of the frying oil, in which the actually measured viscosity increase rates are compared with the viscosity increase rates calculated using Equation (31), Equation (32), Equation (49), and Equation (50), respectively.

FIG. 274 illustrates a graph showing the correlation of the total polar compounds contained in frying oil relating to the eighth oil type, which is classified based on the FFA content in new oil, relative to the viscosity increase rate of the frying oil, in which the actually measured total polar compounds are compared with the total polar compounds calculated using Equation (31), Equation (32), Equation (49), and Equation (50), respectively.

FIG. 275 illustrates a graph showing the correlation of the acid value of frying oil relating to the eighth oil type, which is classified based on the FFA content in new oil, relative to the viscosity increase rate of the frying oil, in which the actually measured acid values are compared with the acid values calculated using Equation (31), Equation (32), Equation (49), and Equation (50), respectively.

FIG. 276 illustrates a graph showing the correlation of the color of frying oil relating to the eighth oil type, which is classified based on the FFA content in new oil, relative to the viscosity increase rate of the frying oil, in which the actually measured values of the color are compared with the colors calculated using Equation (31), Equation (32), Equation (49), and Equation (50), respectively.

FIG. 277 illustrates a graph showing the correlation of the total polar compounds contained in frying oil relating to the eighth oil type, which is classified based on the FFA content in new oil, relative to the color of the frying oil, in which the actually measured total polar compounds are compared with the total polar compounds calculated using Equation (31), Equation (32), Equation (49), and Equation (50), respectively.

FIG. 278 illustrates a graph showing the correlation of the viscosity increase rate of frying oil relating to the eighth oil type, which is classified based on the FFA content in new oil, relative to the color of the frying oil, in which the actually measured viscosity increase rates are compared with the viscosity increase rates calculated using Equation (31), Equation (32), Equation (49), and Equation (50), respectively.

FIG. 279 illustrates a graph showing the correlation of the viscosity increase rate of frying oil relating to the seventh oil type, which is classified based on the MG content in heated oil, relative to the total polar compounds contained in the frying oil, in which the actually measured viscosity increase rates are compared with the viscosity increase rates calculated using Equation (31), Equation (32), Equation (47), and Equation (48), respectively.

FIG. 280 illustrates a graph showing the correlation of the color of frying oil relating to the seventh oil type, which is classified based on the MG content in heated oil, relative to the total polar compounds contained in the frying oil, in which the actually measured values of the color are compared with the colors calculated using Equation (31), Equation (32), Equation (47), and Equation (48), respectively.

FIG. 281 illustrates a graph showing the correlation of the total polar compounds contained in frying oil relating to the seventh oil type, which is classified based on the MG content in heated oil, relative to the acid value of the frying oil, in which the actually measured total polar compounds are compared with the total polar compounds calculated using Equation (31), Equation (32), Equation (47), and Equation (48), respectively.

FIG. 282 illustrates a graph showing the correlation of the viscosity increase rate of frying oil relating to the seventh oil type, which is classified based on the MG content in heated oil, relative to the acid value of the frying oil, in which the actually measured viscosity increase rates are compared with the viscosity increase rates calculated using Equation (31), Equation (32), Equation (47), and Equation (48), respectively.

FIG. 283 illustrates a graph showing the correlation of the total polar compounds contained in frying oil relating to the seventh oil type, which is classified based on the MG content in heated oil, relative to the viscosity increase rate of the frying oil, in which the actually measured total polar compounds are compared with the total polar compounds calculated using Equation (31), Equation (32), Equation (47), and Equation (48), respectively.

FIG. 284 illustrates a graph showing the correlation of the acid value of frying oil relating to the seventh oil type, which is classified based on the MG content in heated oil, relative to the viscosity increase rate of the frying oil, in which the actually measured acid values are compared with the acid values calculated using Equation (31), Equation (32), Equation (47), and Equation (48), respectively.

FIG. 285 illustrates a graph showing the correlation of the color of frying oil relating to the seventh oil type, which is classified based on the MG content in heated oil, relative to the viscosity increase rate of the frying oil, in which the actually measured values of the color are compared with the colors calculated using Equation (31), Equation (32), Equation (47), and Equation (48), respectively.

FIG. 286 illustrates a graph showing the correlation of the total polar compounds contained in frying oil relating to the seventh oil type, which is classified based on the MG content in heated oil, relative to the color of the frying oil, in which the actually measured total polar compounds are compared with the total polar compounds calculated using Equation (31), Equation (32), Equation (47), and Equation (48), respectively.

FIG. 287 illustrates a graph showing the correlation of the viscosity increase rate of frying oil relating to the seventh oil type, which is classified based on the MG content in heated oil, relative to the color of the frying oil, in which the actually measured viscosity increase rates are compared with the viscosity increase rates calculated using Equation (31), Equation (32), Equation (47), and Equation (48), respectively.

FIG. 288 illustrates a graph showing the correlation of the viscosity increase rate of frying oil relating to the eighth oil type, which is classified based on the MG content in heated oil, relative to the total polar compounds contained in the frying oil, in which the actually measured viscosity increase rates are compared with the viscosity increase rates calculated using Equation (31), Equation (32), Equation (49), and Equation (50), respectively.

FIG. 289 illustrates a graph showing the correlation of the color of frying oil relating to the eighth oil type, which is classified based on the MG content in heated oil, relative to the total polar compounds contained in the frying oil, in which the actually measured values of the color are compared with the colors calculated using Equation (31), Equation (32), Equation (49), and Equation (50), respectively.

FIG. 290 illustrates a graph showing the correlation of the total polar compounds contained in frying oil relating to the eighth oil type, which is classified based on the MG content in heated oil, relative to the acid value of the frying oil, in which the actually measured total polar compounds are compared with the total polar compounds calculated using Equation (31), Equation (32), Equation (49), and Equation (50), respectively.

FIG. 291 illustrates a graph showing the correlation of the viscosity increase rate of frying oil relating to the eighth oil type, which is classified based on the MG content in heated oil, relative to the acid value of the frying oil, in which the actually measured viscosity increase rates are compared with the viscosity increase rates calculated using Equation (31), Equation (32), Equation (49), and Equation (50), respectively.

FIG. 292 illustrates a graph showing the correlation of the total polar compounds contained in frying oil relating to the eighth oil type, which is classified based on the MG content in heated oil, relative to the viscosity increase rate of the frying oil, in which the actually measured total polar compounds are compared with the total polar compounds calculated using Equation (31), Equation (32), Equation (49), and Equation (50), respectively.

FIG. 293 illustrates a graph showing the correlation of the acid value of frying oil relating to the eighth oil type, which is classified based on the MG content in heated oil, relative to the viscosity increase rate of the frying oil, in which the actually measured acid values are compared with the acid values calculated using Equation (31), Equation (32), Equation (49), and Equation (50), respectively.

FIG. 294 illustrates a graph showing the correlation of the color of frying oil relating to the eighth oil type, which is classified based on the MG content in heated oil, relative to the viscosity increase rate of the frying oil, in which the actually measured values of the color are compared with the colors calculated using Equation (31), Equation (32), Equation (49), and Equation (50), respectively.

FIG. 295 illustrates a graph showing the correlation of the total polar compounds contained in frying oil relating to the eighth oil type, which is classified based on the MG content in heated oil, relative to the color of the frying oil, in which the actually measured total polar compounds are compared with the total polar compounds calculated using Equation (31), Equation (32), Equation (49), and Equation (50), respectively.

FIG. 296 illustrates a graph showing the correlation of the viscosity increase rate of frying oil relating to the eighth oil type, which is classified based on the MG content in heated oil, relative to the color of the frying oil, in which the actually measured viscosity increase rates are compared with the viscosity increase rates calculated using Equation (31), Equation (32), Equation (49), and Equation (50), respectively.

FIG. 297 illustrates a graph showing the correlation of the viscosity increase rate of frying oil relating to the seventh oil type, which is classified based on the TG content in heated oil, relative to the total polar compounds contained in the frying oil, in which the actually measured viscosity increase rates are compared with the viscosity increase rates calculated using Equation (31), Equation (32), Equation (47), and Equation (48), respectively.

FIG. 298 illustrates a graph showing the correlation of the color of frying oil relating to the seventh oil type, which is classified based on the TG content in heated oil, relative to the total polar compounds contained in the frying oil, in which the actually measured values of the color are compared with the colors calculated using Equation (31), Equation (32), Equation (47), and Equation (48), respectively.

FIG. 299 illustrates a graph showing the correlation of the total polar compounds contained in frying oil relating to the seventh oil type, which is classified based on the TG content in heated oil, relative to the acid value of the frying oil, in which the actually measured total polar compounds are compared with the total polar compounds calculated using Equation (31), Equation (32), Equation (47), and Equation (48), respectively.

FIG. 300 illustrates a graph showing the correlation of the viscosity increase rate of frying oil relating to the seventh oil type, which is classified based on the TG content in heated oil, relative to the acid value of the frying oil, in which the actually measured viscosity increase rates are compared with the viscosity increase rates calculated using Equation (31), Equation (32), Equation (47), and Equation (48), respectively.

FIG. 301 illustrates a graph showing the correlation of the total polar compounds contained in frying oil relating to the seventh oil type, which is classified based on the TG content in heated oil, relative to the viscosity increase rate of the frying oil, in which the actually measured total polar compounds are compared with the total polar compounds calculated using Equation (31), Equation (32), Equation (47), and Equation (48), respectively.

FIG. 302 illustrates a graph showing the correlation of the acid value of frying oil relating to the seventh oil type, which is classified based on the TG content in heated oil, relative to the viscosity increase rate of the frying oil, in which the actually measured acid values are compared with the acid values calculated using Equation (31), Equation (32), Equation (47), and Equation (48), respectively.

FIG. 303 illustrates a graph showing the correlation of the color of frying oil relating to the seventh oil type, which is classified based on the TG content in heated oil, relative to the viscosity increase rate of the frying oil, in which the actually measured values of the color are compared with the colors calculated using Equation (31), Equation (32), Equation (47), and Equation (48), respectively.

FIG. 304 illustrates a graph showing the correlation of the total polar compounds contained in frying oil relating to the seventh oil type, which is classified based on the TG content in heated oil, relative to the color of the frying oil, in which the actually measured total polar compounds are compared with the total polar compounds calculated using Equation (31), Equation (32), Equation (47), and Equation (48), respectively.

FIG. 305 illustrates a graph showing the correlation of the viscosity increase rate of frying oil relating to the seventh oil type, which is classified based on the TG content in heated oil, relative to the color of the frying oil, in which the actually measured viscosity increase rates are compared with the viscosity increase rates calculated using Equation (31), Equation (32), Equation (47), and Equation (48), respectively.

FIG. 306 illustrates a graph showing the correlation of the viscosity increase rate of frying oil relating to the eighth oil type, which is classified based on the TG content in heated oil, relative to the total polar compounds contained in the frying oil, in which the actually measured viscosity increase rates are compared with the viscosity increase rates calculated using Equation (31), Equation (32), Equation (49), and Equation (50), respectively.

FIG. 307 illustrates a graph showing the correlation of the color of frying oil relating to the eighth oil type, which is classified based on the TG content in heated oil, relative to the total polar compounds contained in the frying oil, in which the actually measured values of the color are compared with the colors calculated using Equation (31), Equation (32), Equation (49), and Equation (50), respectively.

FIG. 308 illustrates a graph showing the correlation of the total polar compounds contained in frying oil relating to the eighth oil type, which is classified based on the TG content in heated oil, relative to the acid value of the frying oil, in which the actually measured total polar compounds are compared with the total polar compounds calculated using Equation (31), Equation (32), Equation (49), and Equation (50), respectively.

FIG. 309 illustrates a graph showing the correlation of the viscosity increase rate of frying oil relating to the eighth oil type, which is classified based on the TG content in heated oil, relative to the acid value of the frying oil, in which the actually measured viscosity increase rates are compared with the viscosity increase rates calculated using Equation (31), Equation (32), Equation (49), and Equation (50), respectively.

FIG. 310 illustrates a graph showing the correlation of the total polar compounds contained in frying oil relating to the eighth oil type, which is classified based on the TG content in heated oil, relative to the viscosity increase rate of the frying oil, in which the actually measured total polar compounds are compared with the total polar compounds calculated using Equation (31), Equation (32), Equation (49), and Equation (50), respectively.

FIG. 311 illustrates a graph showing the correlation of the acid value of frying oil relating to the eighth oil type, which is classified based on the TG content in heated oil, relative to the viscosity increase rate of the frying oil, in which the actually measured acid values are compared with the acid values calculated using Equation (31), Equation (32), Equation (49), and Equation (50), respectively.

FIG. 312 illustrates a graph showing the correlation of the color of frying oil relating to the eighth oil type, which is classified based on the TG content in heated oil, relative to the viscosity increase rate of the frying oil, in which the actually measured values of the color are compared with the colors calculated using Equation (31), Equation (32), Equation (49), and Equation (50), respectively.

FIG. 313 illustrates a graph showing the correlation of the total polar compounds contained in frying oil relating to the eighth oil type, which is classified based on the TG content in heated oil, relative to the color of the frying oil, in which the actually measured total polar compounds are compared with the total polar compounds calculated using Equation (31), Equation (32), Equation (49), and Equation (50), respectively.

FIG. 314 illustrates a graph showing the correlation of the viscosity increase rate of frying oil relating to the eighth oil type, which is classified based on the TG content in heated oil, relative to the color of the frying oil, in which the actually measured viscosity increase rates are compared with the viscosity increase rates calculated using Equation (31), Equation (32), Equation (49), and Equation (50), respectively.

FIG. 315 illustrates a graph showing the correlation of the viscosity increase rate of frying oil relating to the seventh oil type, which is classified based on the rate of increase in the DG content due to heating, relative to the total polar compounds contained in the frying oil, in which the actually measured viscosity increase rates are compared with the viscosity increase rates calculated using Equation (31), Equation (32), Equation (47), and Equation (48), respectively.

FIG. 316 illustrates a graph showing the correlation of the color of frying oil relating to the seventh oil type, which is classified based on the rate of increase in the DG content due to heating, relative to the total polar compounds contained in the frying oil, in which the actually measured values of the color are compared with the colors calculated using Equation (31), Equation (32), Equation (47), and Equation (48), respectively.

FIG. 317 illustrates a graph showing the correlation of the total polar compounds contained in frying oil relating to the seventh oil type, which is classified based on the rate of increase in the DG content due to heating, relative to the acid value of the frying oil, in which the actually measured total polar compounds are compared with the total polar compounds calculated using Equation (31), Equation (32), Equation (47), and Equation (48), respectively.

FIG. 318 illustrates a graph showing the correlation of the viscosity increase rate of frying oil relating to the seventh oil type, which is classified based on the rate of increase in the DG content due to heating, relative to the acid value of the frying oil, in which the actually measured viscosity increase rates are compared with the viscosity increase rates calculated using Equation (31), Equation (32), Equation (47), and Equation (48), respectively.

FIG. 319 illustrates a graph showing the correlation of the total polar compounds contained in frying oil relating to the seventh oil type, which is classified based on the rate of increase in the DG content due to heating, relative to the viscosity increase rate of the frying oil, in which the actually measured total polar compounds are compared with the total polar compounds calculated using Equation (31), Equation (32), Equation (47), and Equation (48), respectively.

FIG. 320 illustrates a graph showing the correlation of the acid value of frying oil relating to the seventh oil type, which is classified based on the rate of increase in the DG content due to heating, relative to the viscosity increase rate of the frying oil, in which the actually measured acid values are compared with the acid values calculated using Equation (31), Equation (32), Equation (47), and Equation (48), respectively.

FIG. 321 illustrates a graph showing the correlation of the color of frying oil relating to the seventh oil type, which is classified based on the rate of increase in the DG content due to heating, relative to the viscosity increase rate of the frying oil, in which the actually measured values of the color are compared with the colors calculated using Equation (31), Equation (32), Equation (47), and Equation (48), respectively.

FIG. 322 illustrates a graph showing the correlation of the total polar compounds contained in frying oil relating to the seventh oil type, which is classified based on the rate of increase in the DG content due to heating, relative to the color of the frying oil, in which the actually measured total polar compounds are compared with the total polar compounds calculated using Equation (31), Equation (32), Equation (47), and Equation (48), respectively.

FIG. 323 illustrates a graph showing the correlation of the viscosity increase rate of frying oil relating to the seventh oil type, which is classified based on the rate of increase in the DG content due to heating, relative to the color of the frying oil, in which the actually measured viscosity increase rates are compared with the viscosity increase rates calculated using Equation (31), Equation (32), Equation (47), and Equation (48), respectively.

FIG. 324 illustrates a graph showing the correlation of the viscosity increase rate of frying oil relating to the eighth oil type, which is classified based on the rate of increase in the DG content due to heating, relative to the total polar compounds contained in the frying oil, in which the actually measured viscosity increase rates are compared with the viscosity increase rates calculated using Equation (31), Equation (32), Equation (49), and Equation (50), respectively.

FIG. 325 illustrates a graph showing the correlation of the color of frying oil relating to the eighth oil type, which is classified based on the rate of increase in the DG content due to heating, relative to the total polar compounds contained in the frying oil, in which the actually measured values of the color are compared with the colors calculated using Equation (31), Equation (32), Equation (49), and Equation (50), respectively.

FIG. 326 illustrates a graph showing the correlation of the total polar compounds contained in frying oil relating to the eighth oil type, which is classified based on the rate of increase in the DG content due to heating, relative to the acid value of the frying oil, in which the actually measured total polar compounds are compared with the total polar compounds calculated using Equation (31), Equation (32), Equation (49), and Equation (50), respectively.

FIG. 327 illustrates a graph showing the correlation of the viscosity increase rate of frying oil relating to the eighth oil type, which is classified based on the rate of increase in the DG content due to heating, relative to the acid value of the frying oil, in which the actually measured viscosity increase rates are compared with the viscosity increase rates calculated using Equation (31), Equation (32), Equation (49), and Equation (50), respectively.

FIG. 328 illustrates a graph showing the correlation of the total polar compounds contained in frying oil relating to the eighth oil type, which is classified based on the rate of increase in the DG content due to heating, relative to the viscosity increase rate of the frying oil, in which the actually measured total polar compounds are compared with the total polar compounds calculated using Equation (31), Equation (32), Equation (49), and Equation (50), respectively.

FIG. 329 illustrates a graph showing the correlation of the acid value of frying oil relating to the eighth oil type, which is classified based on the rate of increase in the DG content due to heating, relative to the viscosity increase rate of the frying oil, in which the actually measured acid values are compared with the acid values calculated using Equation (31), Equation (32), Equation (49), and Equation (50), respectively.

FIG. 330 illustrates a graph showing the correlation of the color of frying oil relating to the eighth oil type, which is classified based on the rate of increase in the DG content due to heating, relative to the viscosity increase rate of the frying oil, in which the actually measured values of the color are compared with the colors calculated using Equation (31), Equation (32), Equation (49), and Equation (50), respectively.

FIG. 331 illustrates a graph showing the correlation of the total polar compounds contained in frying oil relating to the eighth oil type, which is classified based on the rate of increase in the DG content due to heating, relative to the color of the frying oil, in which the actually measured total polar compounds are compared with the total polar compounds calculated using Equation (31), Equation (32), Equation (49), and Equation (50), respectively.

FIG. 332 illustrates a graph showing the correlation of the viscosity increase rate of frying oil relating to the eighth oil type, which is classified based on the rate of increase in the DG content due to heating, relative to the color of the frying oil, in which the actually measured viscosity increase rates are compared with the viscosity increase rates calculated using Equation (31), Equation (32), Equation (49), and Equation (50), respectively.

FIG. 333 illustrates a graph showing the correlation of the viscosity increase rate of frying oil relating to the seventh oil type, which is classified based on the rate of increase in the FFA content due to heating, relative to the total polar compounds contained in the frying oil, in which the actually measured viscosity increase rates are compared with the viscosity increase rates calculated using Equation (31), Equation (32), Equation (47), and Equation (48), respectively.

FIG. 334 illustrates a graph showing the correlation of the color of frying oil relating to the seventh oil type, which is classified based on the rate of increase in the FFA content due to heating, relative to the total polar compounds contained in the frying oil, in which the actually measured values of the color are compared with the colors calculated using Equation (31), Equation (32), Equation (47), and Equation (48), respectively.

FIG. 335 illustrates a graph showing the correlation of the total polar compounds contained in frying oil relating to the seventh oil type, which is classified based on the rate of increase in the FFA content due to heating, relative to the acid value of the frying oil, in which the actually measured total polar compounds are compared with the total polar compounds calculated using Equation (31), Equation (32), Equation (47), and Equation (48), respectively.

FIG. 336 illustrates a graph showing the correlation of the viscosity increase rate of frying oil relating to the seventh oil type, which is classified based on the rate of increase in the FFA content due to heating, relative to the acid value of the frying oil, in which the actually measured viscosity increase rates are compared with the viscosity increase rates calculated using Equation (31), Equation (32), Equation (47), and Equation (48), respectively.

FIG. 337 illustrates a graph showing the correlation of the total polar compounds contained in frying oil relating to the seventh oil type, which is classified based on the rate of increase in the FFA content due to heating, relative to the viscosity increase rate of the frying oil, in which the actually measured total polar compounds are compared with the total polar compounds calculated using Equation (31), Equation (32), Equation (47), and Equation (48), respectively.

FIG. 338 illustrates a graph showing the correlation of the acid value of frying oil relating to the seventh oil type, which is classified based on the rate of increase in the FFA content due to heating, relative to the viscosity increase rate of the frying oil, in which the actually measured acid values are compared with the acid values calculated using Equation (31), Equation (32), Equation (47), and Equation (48), respectively.

FIG. 339 illustrates a graph showing the correlation of the color of frying oil relating to the seventh oil type, which is classified based on the rate of increase in the FFA content due to heating, relative to the viscosity increase rate of the frying oil, in which the actually measured values of the color are compared with the colors calculated using Equation (31), Equation (32), Equation (47), and Equation (48), respectively.

FIG. 340 illustrates a graph showing the correlation of the total polar compounds contained in frying oil relating to the seventh oil type, which is classified based on the rate of increase in the FFA content due to heating, relative to the color of the frying oil, in which the actually measured total polar compounds are compared with the total polar compounds calculated using Equation (31), Equation (32), Equation (47), and Equation (48), respectively.

FIG. 341 illustrates a graph showing the correlation of the viscosity increase rate of frying oil relating to the seventh oil type, which is classified based on the rate of increase in the FFA content due to heating, relative to the color of the frying oil, in which the actually measured viscosity increase rates are compared with the viscosity increase rates calculated using Equation (31), Equation (32), Equation (47), and Equation (48), respectively.

FIG. 342 illustrates a graph showing the correlation of the viscosity increase rate of frying oil relating to the eighth oil type, which is classified based on the rate of increase in the FFA content due to heating, relative to the total polar compounds contained in the frying oil, in which the actually measured viscosity increase rates are compared with the viscosity increase rates calculated using Equation (31), Equation (32), Equation (49), and Equation (50), respectively.

FIG. 343 illustrates a graph showing the correlation of the color of frying oil relating to the eighth oil type, which is classified based on the rate of increase in the FFA content due to heating, relative to the total polar compounds contained in the frying oil, in which the actually measured values of the color are compared with the colors calculated using Equation (31), Equation (32), Equation (49), and Equation (50), respectively.

FIG. 344 illustrates a graph showing the correlation of the total polar compounds contained in frying oil relating to the eighth oil type, which is classified based on the rate of increase in the FFA content due to heating, relative to the acid value of the frying oil, in which the actually measured total polar compounds are compared with the total polar compounds calculated using Equation (31), Equation (32), Equation (49), and Equation (50), respectively.

FIG. 345 illustrates a graph showing the correlation of the viscosity increase rate of frying oil relating to the eighth oil type, which is classified based on the rate of increase in the FFA content due to heating, relative to the acid value of the frying oil, in which the actually measured viscosity increase rates are compared with the viscosity increase rates calculated using Equation (31), Equation (32), Equation (49), and Equation (50), respectively.

FIG. 346 illustrates a graph showing the correlation of the total polar compounds contained in frying oil relating to the eighth oil type, which is classified based on the rate of increase in the FFA content due to heating, relative to the viscosity increase rate of the frying oil, in which the actually measured total polar compounds are compared with the total polar compounds calculated using Equation (31), Equation (32), Equation (49), and Equation (50), respectively.

FIG. 347 illustrates a graph showing the correlation of the acid value of frying oil relating to the eighth oil type, which is classified based on the rate of increase in the FFA content due to heating, relative to the viscosity increase rate of the frying oil, in which the actually measured acid values are compared with the acid values calculated using Equation (31), Equation (32), Equation (49), and Equation (50), respectively.

FIG. 348 illustrates a graph showing the correlation of the color of frying oil relating to the eighth oil type, which is classified based on the rate of increase in the FFA content due to heating, relative to the viscosity increase rate of the frying oil, in which the actually measured values of the color are compared with the colors calculated using Equation (31), Equation (32), Equation (49), and Equation (50), respectively.

FIG. 349 illustrates a graph showing the correlation of the total polar compounds contained in frying oil relating to the eighth oil type, which is classified based on the rate of increase in the FFA content due to heating, relative to the color of the frying oil, in which the actually measured total polar compounds are compared with the total polar compounds calculated using Equation (31), Equation (49), and Equation (50), respectively.

FIG. 350 illustrates a graph showing the correlation of the viscosity increase rate of frying oil relating to the eighth oil type, which is classified based on the rate of increase in the FFA content due to heating, relative to the color of the frying oil, in which the actually measured viscosity increase rates are compared with the viscosity increase rates calculated using Equation (31), Equation (32), Equation (49), and Equation (50), respectively.

FIG. 351 illustrates a graph showing the correlation of the viscosity increase rate of frying oil relating to the seventh oil type, which is classified based on the rate of decrease in the TG content due to heating, relative to the total polar compounds contained in the frying oil, in which the actually measured viscosity increase rates are compared with the viscosity increase rates calculated using Equation (31), Equation (32), Equation (47), and Equation (48), respectively.

FIG. 352 illustrates a graph showing the correlation of the color of frying oil relating to the seventh oil type, which is classified based on the rate of decrease in the TG content due to heating, relative to the total polar compounds contained in the frying oil, in which the actually measured values of the color are compared with the colors calculated using Equation (31), Equation (32), Equation (47), and Equation (48), respectively.

FIG. 353 illustrates a graph showing the correlation of the total polar compounds contained in frying oil relating to the seventh oil type, which is classified based on the rate of decrease in the TG content due to heating, relative to the acid value of the frying oil, in which the actually measured total polar compounds are compared with the total polar compounds calculated using Equation (31), Equation (32), Equation (47), and Equation (48), respectively.

FIG. 354 illustrates a graph showing the correlation of the viscosity increase rate of frying oil relating to the seventh oil type, which is classified based on the rate of decrease in the TG content due to heating, relative to the acid value of the frying oil, in which the actually measured viscosity increase rates are compared with the viscosity increase rates calculated using Equation (31), Equation (32), Equation (47), and Equation (48), respectively.

FIG. 355 illustrates a graph showing the correlation of the total polar compounds contained in frying oil relating to the seventh oil type, which is classified based on the rate of decrease in the TG content due to heating, relative to the viscosity increase rate of the frying oil, in which the actually measured total polar compounds are compared with the total polar compounds calculated using Equation (31), Equation (32), Equation (47), and Equation (48), respectively.

FIG. 356 illustrates a graph showing the correlation of the acid value of frying oil relating to the seventh oil type, which is classified based on the rate of decrease in the TG content due to heating, relative to the viscosity increase rate of the frying oil, in which the actually measured acid values are compared with the acid values calculated using Equation (31), Equation (32), Equation (47), and Equation (48), respectively.

FIG. 357 illustrates a graph showing the correlation of the color of frying oil relating to the seventh oil type, which is classified based on the rate of decrease in the TG content due to heating, relative to the viscosity increase rate of the frying oil, in which the actually measured values of the color are compared with the colors calculated using Equation (31), Equation (32), Equation (47), and Equation (48), respectively.

FIG. 358 illustrates a graph showing the correlation of the total polar compounds contained in frying oil relating to the seventh oil type, which is classified based on the rate of decrease in the TG content due to heating, relative to the color of the frying oil, in which the actually measured total polar compounds are compared with the total polar compounds calculated using Equation (31), Equation (32), Equation (47), and Equation (48), respectively.

FIG. 359 illustrates a graph showing the correlation of the viscosity increase rate of frying oil relating to the seventh oil type, which is classified based on the rate of decrease in the TG content due to heating, relative to the color of the frying oil, in which the actually measured viscosity increase rates are compared with the viscosity increase rates calculated using Equation (31), Equation (32), Equation (47), and Equation (48), respectively.

FIG. 360 illustrates a graph showing the correlation of the viscosity increase rate of frying oil relating to the eighth oil type, which is classified based on the rate of decrease in the TG content due to heating, relative to the total polar compounds contained in the frying oil, in which the actually measured viscosity increase rates are compared with the viscosity increase rates calculated using Equation (31), Equation (32), Equation (49), and Equation (50), respectively.

FIG. 361 illustrates a graph showing the correlation of the color of frying oil relating to the eighth oil type, which is classified based on the rate of decrease in the TG content due to heating, relative to the total polar compounds contained in the frying oil, in which the actually measured values of the color are compared with the colors calculated using Equation (31), Equation (32), Equation (49), and Equation (50), respectively.

FIG. 362 illustrates a graph showing the correlation of the total polar compounds contained in frying oil relating to the eighth oil type, which is classified based on the rate of decrease in the TG content due to heating, relative to the acid value of the frying oil, in which the actually measured total polar compounds are compared with the total polar compounds calculated using Equation (31), Equation (32), Equation (49), and Equation (50), respectively.

FIG. 363 illustrates a graph showing the correlation of the viscosity increase rate of frying oil relating to the eighth oil type, which is classified based on the rate of decrease in the TG content due to heating, relative to the acid value of the frying oil, in which the actually measured viscosity increase rates are compared with the viscosity increase rates calculated using Equation (31), Equation (32), Equation (49), and Equation (50), respectively.

FIG. 364 illustrates a graph showing the correlation of the total polar compounds contained in frying oil relating to the eighth oil type, which is classified based on the rate of decrease in the TG content due to heating, relative to the viscosity increase rate of the frying oil, in which the actually measured total polar compounds are compared with the total polar compounds calculated using Equation (31), Equation (32), Equation (49), and Equation (50), respectively.

FIG. 365 illustrates a graph showing the correlation of the acid value of frying oil relating to the eighth oil type, which is classified based on the rate of decrease in the TG content due to heating, relative to the viscosity increase rate of the frying oil, in which the actually measured acid values are compared with the acid values calculated using Equation (31), Equation (32), Equation (49), and Equation (50), respectively.

FIG. 366 illustrates a graph showing the correlation of the color of frying oil relating to the eighth oil type, which is classified based on the rate of decrease in the TG content due to heating, relative to the viscosity increase rate of the frying oil, in which the actually measured values of the color are compared with the colors calculated using Equation (31), Equation (32), Equation (49), and Equation (50), respectively.

FIG. 367 illustrates a graph showing the correlation of the total polar compounds contained in frying oil relating to the eighth oil type, which is classified based on the rate of decrease in the TG content due to heating, relative to the color of the frying oil, in which the actually measured total polar compounds are compared with the total polar compounds calculated using Equation (31), Equation (32), Equation (49), and Equation (50), respectively.

FIG. 368 illustrates a graph showing the correlation of the viscosity increase rate of frying oil relating to the eighth oil type, which is classified based on the rate of decrease in the TG content due to heating, relative to the color of the frying oil, in which the actually measured viscosity increase rates are compared with the viscosity increase rates calculated using Equation (31), Equation (32), Equation (49), and Equation (50), respectively.

FIG. 369 illustrates a graph of the correlation of the color of frying oil relative to the total polar compounds contained in the frying oil, in which the type of a deep-frying material is considered.

FIG. 370 illustrates a graph of the correlation of the color of frying oil relative to the acid value of the frying oil, in which the type of a deep-frying material is considered.

FIG. 371 illustrates a graph of the correlation of the color of frying oil relative to the viscosity increase rate of the frying oil, in which the type of a deep-frying material is considered.

FIG. 372 is a functional block diagram illustrating functions provided in a cloud according to the fifteenth embodiment.

FIG. 373 illustrates a flowchart of a flow of the processing to be executed in a cloud according to the fifteenth embodiment.

### DESCRIPTION OF EMBODIMENTS

Hereinafter, as an aspect of a fat and oil deterioration degree detection system according to each embodiment of the present invention, a system applicable to cooking of fried foods such as fried chickens, croquettes, and karaage, which is performed, for example, in convenience stores and supermarkets.

Cooking of fried foods is referred herein as “deep frying”, edible oil to be used in deep frying is referred herein as “frying oil”, and an ingredient to be deep fried is referred herein as “deep-frying material”.

### (Arrangement in Cooking Area 1)

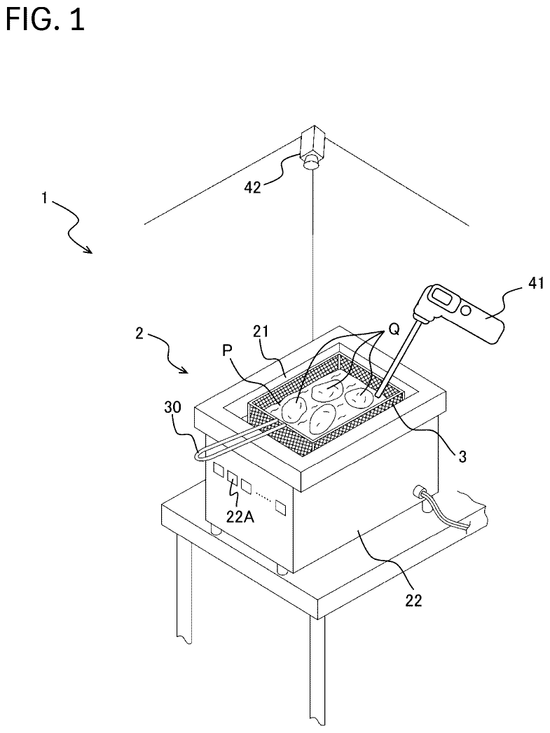

Firstly, an example of an environment in which deep frying is performed will be described with reference to FIG. 1.

FIG. 1 illustrates a part of a cooking area 1 in which deep frying is performed.

For example, in a store such as a convenience store or a supermarket, the cooking area 1 in which deep frying is performed is provided in the store so as to provide customers with freshly deep-fried foods. Within the cooking area 1, as a cooking tool to be used in deep frying, for example, an electric fryer 2 is installed. However, the fryer 2 does not necessarily have to be an electric fryer but may be a gas fryer.

The fryer 2 includes an oil vat 21 for holding frying oil P therein, and a housing 22 for accommodating the oil vat 21. On a side surface of the housing 22, various types of operation switches 22A are provided. The plurality of operation switches 22A includes setting switches for setting the temperature of the frying oil P and the details of the deep frying for each kind of materials Q for deep frying, a start switch for starting deep frying, and the like.

For deep frying, firstly, a cook places the deep-frying material Q in a fry basket 3 having a handle 30, and then hooks the handle 30 on an upper end portion of the housing 22 so as to immerse, in the frying oil P, the deep-frying material Q placed in the fry basket 3. At the same time, therebefore, or thereafter, the cook operates one of the operation switches 22A which corresponds to the kind of the deep-frying material Q to be cooked.

Subsequently, the fryer 2 identifies the one of the operation switches 22A which was operated by the cook, and when a deep-frying time, which is associated with the operated one of the switches 22A, passes, notifies the cook of the completion of deep frying. At the same time, the fry basket 3 holding the deep-fried food (deep-frying material Q after being deep fried) automatically rises from the oil vat 21 so that the deep-fried food that has been immersed in the frying oil P is pulled up.

As a technique of informing the completion of deep frying of a fried food, for example, a buzzer sound may be output from a speaker of the fryer 2 or completion of deep frying may be shown on a monitor installed near the fryer 2.

The cook who has noticed the completion of deep frying pulls up the fry basket 3 to take the fried food out therefrom. The operation of pulling up the fry basket 3 from the oil vat 21 may be automatically performed by a drive mechanism which can be provided in the fryer 2.

A user who uses the frying oil P (for example, a cook, a store staff, or the like) measures a deterioration indicator of the frying oil P using various types of measurement devices, and based on the deterioration degree of the frying oil P derived from the measured value of the deterioration indicator of the frying oil P, determines or predicts the deterioration of the frying oil P, so that the quality of the frying oil P and the quality of fried foods obtained by deep frying using the frying oil P can be maintained.

The deterioration indicators of the frying oil P are the ones which vary as the heating time progresses, and include, for example, the acid value (AV) of the frying oil P, the total polar compounds (PC) of the frying oil P, the color of the frying oil P, the viscosity of the frying oil P, the rate of increase in viscosity of the frying oil P, the anisidine value of the frying oil P, the carbonyl value of the frying oil P, the smoke point of the frying oil P, the tocopherol contents of the frying oil P, the iodine value of the frying oil P, the refractive indicator of the frying oil P, the amount of volatile components of the frying oil P, the volatile component composition of the frying oil P, the flavor of the frying oil P, the amount of volatile components of a fried food obtained by deep frying using the frying oil P, the volatile component composition of a fried food obtained by deep frying using the frying oil P, the flavor of a fried food obtained by deep frying using the frying oil P, and the like.

Among these deterioration indicators, in particular, the total polar compounds of the frying oil P can be directly measured by immersing a PC sensor 41 (see FIG. 1), which is configured to measure the total polar compounds contained in the frying oil P based on the capacitance of the frying oil P, in the frying oil P. Thus, using the total polar compounds enables a measured value with high accuracy to be obtained by a simple measurement method.

The cooking area 1 illustrated in FIG. 1 includes a camera 42 for capturing an image of the surface of the frying oil P in the oil vat 21, which is mounted to the ceiling which is positioned above the oil vat 21. However, the camera 42 does not necessarily have to be mounted to the ceiling positioned above the oil vat 21. The camera 42 may be mounted to any position, for example, a wall near the fryer 2, as long as it is held at a position allowing the state in the oil vat 21 to be captured.

The camera 42 is a video camera for capturing a video or a still camera for capturing a still image, and is used to detect a deep-frying material Q within the frying oil P. Thus, the image captured by the camera 42 needs to be an image at least allowing whether the deep-frying material Q is immersed within the oil vat 21 to be checked, however, it may include an image other than the image of the surface of the frying oil P, such as an image of a portion of the oil vat 21, an image of a portion of the fry basket 3 immersed in the frying oil P, or the like.

### (Configuration of Fat and Oil Deterioration Degree Detection System 5)

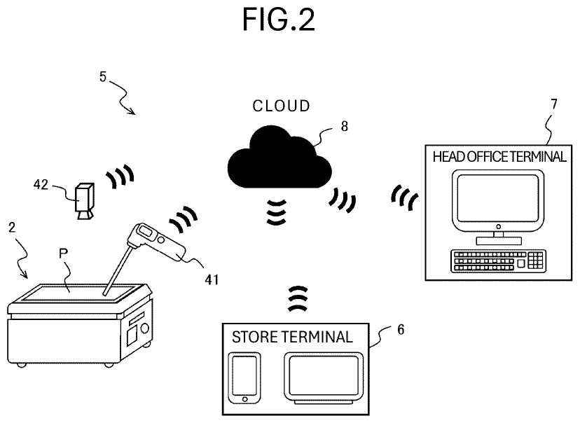

Next, a configuration of a fat and oil deterioration degree detection system 5 will be described with reference to FIG. 2.

FIG. 2 is a system configuration diagram illustrating a configuration example of the fat and oil deterioration degree detection system 5 according to each embodiment of the present invention.

The fat and oil deterioration degree detection system 5 is a system for detecting the deterioration degree of the frying oil P based on the total polar compounds contained in the frying oil P. In the following, the fat and oil deterioration degree detection system 5 detects the acid value (AV[mg KOH/g]) of the frying oil P derived based on the total polar compounds (PC[% TPM]) contained in the frying oil P as the deterioration degree of the frying oil P.

As illustrated in FIG. 2, the fat and oil deterioration degree detection system 5 is configured with, for example, store terminals 6, a head office terminal 7, and a cloud 8. The store terminals 6 are installed in stores included in a convenience store chain, a supermarket chain, or the like, respectively. The head office terminal 7 is installed in, for example, a head office center which manages the plurality of stores. The cloud 8 is configured to execute a fat and oil deterioration degree detection program for detecting the deterioration degree of the frying oil P used in each store.

The store terminal 6, the head office terminal 7, and the cloud 8 are connected to each other via a communication network such as Internet so as to realize the information communication among them. Furthermore, the PC sensor 41 and the camera 42 described above are also connected to the cloud 8, respectively, so as to realize the information communication therebetween. These configurations allow a measured value of the total polar compounds of the frying oil P measured by the PC sensor 41 and an image captured by the camera 42 to be directly transmitted to the cloud 8.

However, the PC sensor 41 and the camera 42 do not necessarily have to be connected to the cloud 8 for communication. For example, in the case where the PC sensor 41 and the camera 42 are connected to the store terminal 6 for communication, a measured value of the total polar compounds of the frying oil P measured by the PC sensor 41 and an image captured by the camera 42 may be transmitted to the cloud 8 via the store terminal 6, respectively.

In the fat and oil deterioration degree detection system 5 for the frying oil P, the store terminals 6 installed in the stores, respectively, have the same functions from each other, and accordingly, in the following, the store terminal 6 in any of the stores is exemplified while the store terminals 6 in other stores will not be described in detail.

The store terminal 6 is an input terminal to which information on a store and information on the frying oil P are input, and also serves as a notification device for notifying various kinds of information output from the head office terminal 7 and the cloud 8 (either by displaying texts or outputting sounds). In the store terminal 6, application software for controlling the frying oil P (frying oil control app) used in a store is installed.

The head office terminal 7 is configured to acquire information output from the store terminal 6 and the cloud 8 in each store to perform control of the amount of the frying oil P used in each store, hygiene of each store, and the like. In the same manner as the store terminal 6, the head office terminal 7 also serves as a notification device for notifying various kinds of information output from each store terminal 6 and the cloud 8 (either by displaying texts or outputting sounds).

The cloud 8 is one of the aspects of a fat and oil deterioration degree detection device for detecting the deterioration degree of the frying oil P based on the total polar compounds of the frying oil P. Specifically, the cloud 8 retains a correlation between the total polar compounds of the frying oil P and the acid value of the frying oil P, and is configured to execute the processes of acquiring a measured value of the total polar compounds of the frying oil P measured by the PC sensor 41 (data acquisition process), calculating the acid value of the frying oil P based on the measured value of the total polar compounds of the frying oil P as acquired and the correlation as stored (deterioration indicator calculation process), and outputting, as the deterioration degree of the frying oil P, the acid value of the frying oil P as calculated to the store terminal 6 and the head office terminal 7 (detection result output process).

A computer for implementing the cloud 8 (for example, a computer owned by a company which provides a cloud system or the like) includes, as a hardware configuration, a CPU (Central Processing Unit), a RAM (Random Access Memory), a ROM (Read Only Memory), an HDD (Hard Disk Drive), and an I/F (Interface). These components are connected to each other via a common bus.

The CPU is an arithmetic means and controls the whole operations of the cloud 8. The RAM is a volatile storage medium capable of reading and writing information at high speed, and is used, for example, as a working area where the CPU processes information. The ROM is a read-only and non-volatile storage medium, in which a program such as a firmware is stored.

The HDD is a nonvolatile storage medium capable of reading and writing information, and has a large storage capacity in which an OS (Operating System) and control programs and application programs for executing various kinds of information processing, which will be described later, are stored. Any type of device such as an SSD (Solid State Drive) may be used instead of the HDD as long as it realizes the functions of storing and managing information as a non-volatile storage medium.

The I/F is a connection interface for connecting to a communication network, to which each of the store terminals 6, the head office terminal 7, the PC sensor 41, the camera 42, and the like are connected.

A computer for implementing the cloud 8 described above is an information processing device that implements the processing functions of the control program stored in the ROM, the control program and application program loaded onto the RAM from a storage medium such as the HDD by means of an arithmetic function provided in the CPU.

By executing the information processing, a software control section including various functions in the cloud 8 are implemented. The functional block that realizes the functions of the cloud 8 is configured with a combination of the software control section thus configured and the hardware resources including the configurations described above.

The fat and oil deterioration degree detection device does not necessarily have to be configured with the cloud 8, but may be configured with a server device. In the case of the fat and oil deterioration degree detection device configured with a server device, the hardware configuration described above is provided in the server device.

Hereinafter, the functions provided in the cloud 8 and the processes to be executed by the cloud 8 will be described for each embodiment.

### First Embodiment

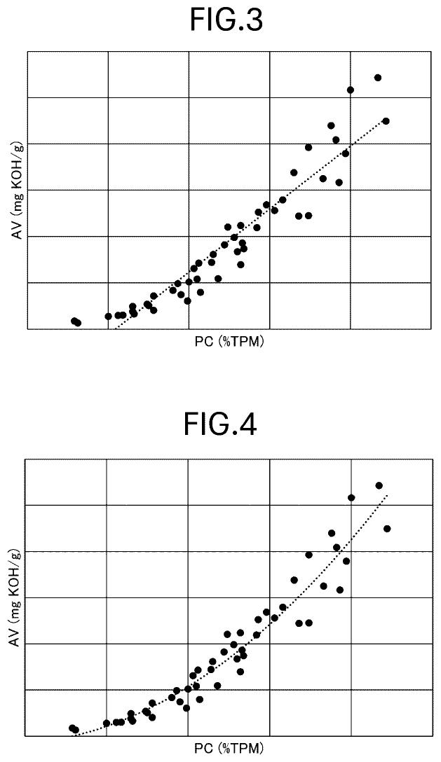

The cloud 8 according to the first embodiment of the present invention will be described with reference to FIG. 3 to FIG. 6.

### (Correlation of Acid Value Relative to Total Polar Materials)

Firstly, the correlation of the acid value of the frying oil P relative to the total polar compounds of the frying oil P, which is stored in the cloud 8, will be described with reference to FIG. 3 and FIG. 4.

FIG. 3 illustrates a graph of a linear function showing the correlation of the acid value of the frying oil P relative to the total polar compounds contained in the frying oil P. FIG. 4 illustrates a graph of a quadratic function showing the correlation of the acid value of the frying oil P relative to the total polar compounds contained in the frying oil P.

The total polar compounds contained in the frying oil P and the acid value of the frying oil P are correlated with each other as illustrated in the graphs of FIG. 3 and FIG. 4. Specifically, as the total polar compounds contained in the frying oil P increase, the acid value of the frying oil P increases. That is, the correlation of the acid value of the frying oil P relative to the total polar compounds contained in the frying oil P are expressed with a correlation equation in the form of a polynomial in which the acid value of the frying oil P is expressed with the total polar compounds.

Specifically, where the total polar compounds of the frying oil P is PC, the acid value of the frying oil P is AV, and the arbitrary heating time of the frying oil P is n, the correlation equation for the acid value AVn of the frying oil P relative to the total polar compounds PCn contained in the frying oil P at the arbitrary heating time n is expressed with a linear equation of the following Equation (1) or a quadratic equation of the following Equation (2).

$$AVn = α \times ( PCn ) + β \qquad ( 1 )$$

$$AVn = γ \times {( PCn )}^{2} + δ \times ( PCn ) + ε \qquad ( 2 )$$

Equation (1) is a correlation equation for the correlation graph illustrated in FIG. 3, and Equation (2) is a correlation equation for the correlation graph illustrated in FIG. 4. By substituting a measured value of the total polar compounds of the frying oil P at the arbitrary heating time n into PCn of each of Equation (1) and Equation (2), the acid value AVn of the frying oil P at the arbitrary heating time n can be calculated.

Each of a first-order coefficient α and a constant R for PCn of Equation (1) and each of a second-order coefficient γ for PCn, a first-order coefficient δ for PCn, and a constant ε of Equation (2) may be any fixed value set in advance, or may be a value that varies depending on the environment where the frying oil P is being used, the type of the frying oil P (oil type), and the like. The latter case will be described in detail in the second to seventh embodiments.

### (Functional Configuration of Cloud 8)

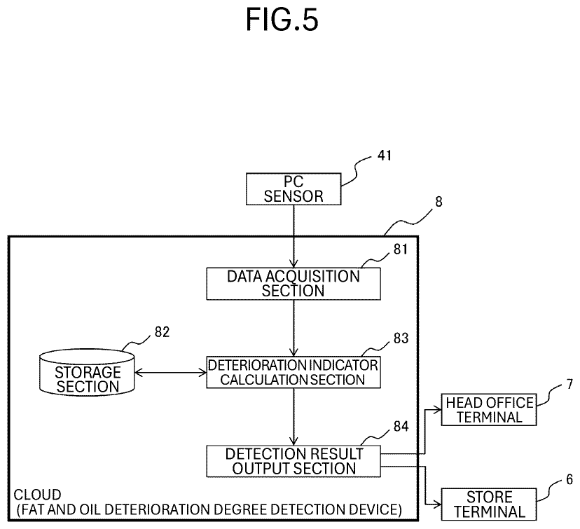

Next, a functional configuration of the cloud 8 will be described with reference to FIG. 5.

FIG. 5 is a functional block diagram illustrating functions provided in the cloud 8 according to the first embodiment.

The cloud 8 includes a data acquisition section 81, a storage section 82, a deterioration indicator calculation section 83, and a detection result output section 84.

The data acquisition section 81 is configured to acquire a measured value of the total polar compounds of the frying oil P output from the PC sensor 41. In the present embodiment, the measured value of the total polar compounds of the frying oil P is measured by the PC sensor 41, however, it may be measured in accordance with other measurement methods or analysis methods using various measurement devices other than the PC sensor 41, for example, by the method using the total polar compounds (column chromatography method) according to 2.5.5-2013 of the JOCS (Japan Oil Chemists' Society) Standard methods for the analysis of fats, oils and related materials.

The storage section 82 retains the correlation equation of the acid value of the frying oil P relative to the total polar compounds of the frying oil P which has been described above, specifically, Equation (1) or Equation (2). The storage section 82 may retain both Equation (1) and Equation (2), or only one of Equation (1) and Equation (2).

The deterioration indicator calculation section 83 is configured to calculate the acid value of the frying oil P based on the measured value of the total polar compounds of the frying oil P acquired by the data acquisition section 81 and the correlation equation for the acid value of the frying oil P relative to the total polar compounds of the frying oil P read from the storage section 82.

Specifically, the deterioration indicator calculation section 83 substitutes the measured value of the total polar compounds of the frying oil P acquired by the data acquisition section 81 into PCn of Equation (1) or Equation (2) read out from the storage section 82 to calculate the acid value AVn of the frying oil P.

In the case where both Equations (1) and (2) are stored in the storage section 82, the deterioration indicator calculation section 83 selects one of Equations (1) and (2), substitutes the measured value of the total polar compounds of the frying oil P into PCn of the selected equation to calculate the acid value AVn of the frying oil P.

The detection result output section 84 is configured to output the acid value of the frying oil P calculated by the deterioration indicator calculation section 83 to each of the store terminal 6 and the head office terminal 7 as the result of detection of the deterioration degree of the frying oil P. In the present embodiment, the detection result output section 84 outputs the result of detection of the deterioration degree of the frying oil P to both the store terminal 6 and the head office terminal 7, respectively, however, may output it to only one of the store terminal 6 and the head office terminal 7.

### (Processing to be Executed in Cloud 8)

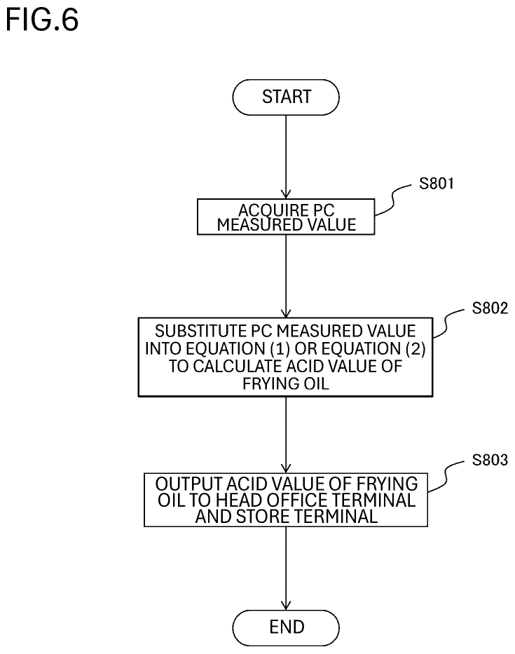

Next, a flow of the processing to be executed in the cloud 8 will be described with reference to FIG. 6.

FIG. 6 illustrates a flowchart of a flow of the processing to be executed in the cloud 8 according to the first embodiment.

As illustrated in FIG. 6, in the cloud 8, firstly, the data acquisition section 81 acquires a measured value of the total polar compounds of the frying oil P measured by the PC sensor 41 in a measurement step (step S801; data acquisition step).

Next, the deterioration indicator calculation section 83 substitutes the measured value of the total polar compounds of the frying oil P acquired in step S801 into PCn of the correlation equation for the acid value of the frying oil P relative to the total polar compounds of the frying oil P stored in the storage section 82, that is, Equation (1) or Equation (2), so as to calculate the acid value AVn of the frying oil P (step S802; deterioration indicator calculation step).

Then, the detection result output section 84 outputs the acid value AVn of the frying oil P calculated in step S802 to the store terminal 6 and the head office terminal 7, respectively, as the result of detection of the deterioration degree of the frying oil P (step S803; detection result output step), whereby the processing in the cloud 8 is ended.

As described above, the cloud 8 enables conversion of the total polar compounds of the frying oil P into the acid value of the frying oil P using the correlation equation for the acid value of the frying oil P relative to the total polar compounds of the frying oil P. This enables a staff of a store to obtain a result of detection of the deterioration degree of the frying oil P which is expressed with the acid value merely by measuring the total polar compounds of the frying oil P using the PC sensor 41.

For detecting the deterioration degree of the frying oil P, in the case where a staff of a store measures the acid value of the frying oil P without using the cloud 8, he or she has to immerse, for example, a color test piece in the frying oil P to identify a measured value of the acid value of the frying oil P based on the change in the color of the color test piece immersed in the frying oil P. This complicated measurement method is likely to cause errors in a result of measurement. On the other hand, the cloud 8 is configured to calculate the acid value based on the total polar compounds that can be easily and accurately measured using the PC sensor 41, which enables the acid value of the frying oil P to be obtained with high accuracy.

### Second Embodiment

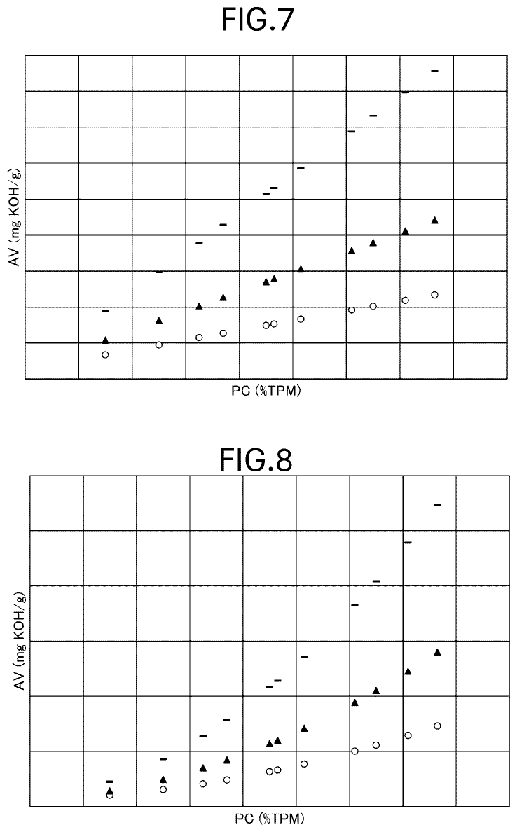

Next, a cloud 8A according to the second embodiment of the present invention will be described with reference to FIG. 7 to FIG. 10. In the following, the same components as those described for the cloud 8 according to the first embodiment are provided with the same reference signs, and repetitive explanation therefor will be omitted. The same applies to the second to fifteenth embodiments.

In the present embodiment, the coefficient and constant included in the correlation equation for the acid value of the frying oil P relative to the total polar compounds of the frying oil P are set to values corresponding to a weight W per unit heating time (hereinafter, referred to as “deep-frying weight W per unit time”) of the deep-frying material Q (ingredient) to be deep fried using the frying oil P.

FIG. 7 illustrates a graph of a linear function showing the correlation of the acid value of the frying oil relative to the total polar compounds contained in the frying oil P, in which the deep-frying weight W per unit time is considered. FIG. 8 illustrates a graph of a quadratic function showing the correlation of the acid value of the frying oil relative to the total polar compounds contained in the frying oil P, in which the deep-frying weight W per unit time is considered.

As illustrated in FIG. 7 and FIG. 8, the slopes of the graphs, each of which shows the correlation of the acid value of the frying oil P relative to the total polar compounds of the frying oil P, vary depending on the deep-frying weight W per unit time of the deep-frying material Q to be cooked using the frying oil P. Accordingly, the first-order coefficient α of PCn in Equation (1) and the second-order coefficient γ and the first-order coefficient δ of PCn in Equation (2) are set to values corresponding to the deep-frying weight W per unit time of the deep-frying material Q to be cooked using the frying oil P, respectively.

Here, the deep-frying weight W of the deep-frying material Q per unit time at a store can also be calculated based on the sales per day at the store in which the frying oil P is used, and thus values corresponding to the sales per day at the store in which the frying oil P is used may be employed for the first-order coefficient α of PCn in Equation (1) and the second-order coefficient γ and the first-order coefficient δ of PCn in Equation (2). The sales per day at the store are obtained, for example, by calculating an average value per day based on the total sales at the store in the past year, or by calculating an average value per day based on the total sales at the store in a predetermined period (for example, every season). The sales per day at the store is preferably the amount obtained by extracting only the sales of the fried food cooked using the frying oil P.

In FIG. 7 and FIG. 8, the deep-frying weight W per unit time at a store is categorized into the three ranges of “large”, “medium”, and “small”, and a correlation graph including a plurality of “∘” is indicative of the case where the deep-frying weight W per unit time is less than 2,000 g (W<2000), a correlation graph including a plurality of “▴” is indicative of the case where the deep-frying weight W per unit time is equal to or more than 2,000 g and less than 12,000 g (20005W<12000), and a correlation graph including a plurality of “-” is indicative of the case where the deep-frying weight W per unit time is equal to or more than 12,000 g (WZ12000), respectively.

The slope of the correlation graph in the case where the deep-frying weight W per unit time is “small” is the smallest among those of the three correlation graphs. The slope of the correlation graph in the case where the deep-frying weight W per unit time is “medium” is greater than the slope of the correlation graph in the case where the deep-frying weight W per unit time is “small”. The slope of the correlation graph in the case where the deep-frying weight W per unit time is “large” is greater than the slope of the correlation graph in the case where the deep-frying weight W per unit time is “medium”, and is the greatest among those of the three correlation graphs.

Accordingly, in the correlation of the acid value of the frying oil P relative to the total polar compounds contained in the frying oil P, the first-order coefficient α of PCn in Equation (1) and the second-order coefficient γ and the first-order coefficient δ of PCn in Equation (2) are set to be greater, respectively, as the deep-frying weight W per unit time increases.

The total polar compounds contained in the frying oil P increase as the heating time at the fryer 2 (the total heating time at the fryer 2, including the heating time in which the deep-frying material Q is deep fried and the empty heating time) increases, and the acid value of the frying oil P increases as the deep-frying weight of the deep-frying material Q increases, respectively. Accordingly, considering both the heating time at the fryer 2 and the deep-frying weight of the deep-frying material Q in the correlation of the acid value of the frying oil P relative to the total polar compounds contained in the frying oil P enables improvement in the accuracy.

The “unit time” does not necessarily have to be one hour, and may be set to any time. The deep-frying weight W per unit time at a store does not necessarily have to be categorized based on the thresholds “2,000 g” and “12,000 g” used in the correlations illustrated in FIG. 7 and FIG. 8, and any value may be used as the thresholds for each store.

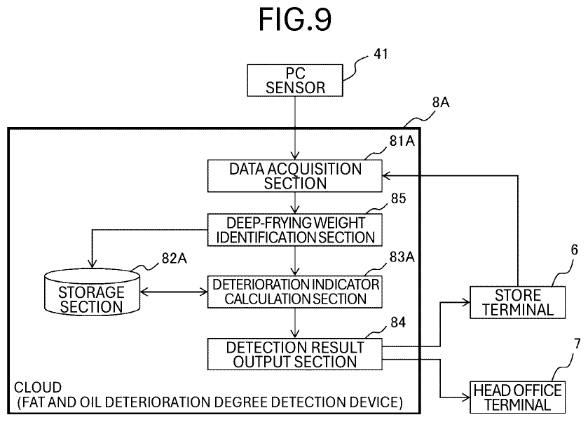

FIG. 9 is a functional block diagram illustrating functions provided in the cloud 8A according to the second embodiment.

The cloud 8A according to the present embodiment includes a data acquisition section 81A, a deep-frying weight identification section 85, a storage section 82A, a deterioration indicator calculation section 83A, and a detection result output section 84.

The data acquisition section 81A is configured to acquire, not only a measured value of the total polar compounds of the frying oil P output from PC sensor 41, but also deep-frying information output from the store terminal 6. The “deep-frying information” includes the heating time at the fryer 2 and the weight of the deep-frying material Q to be cooked in the fryer 2 (that is, the deep-frying weight of the deep-frying material Q). The deep-frying information does not necessarily have to be output from the store terminal 6. For example, the head office terminal 7 may output the information acquired from the store terminal 6, or a separate management terminal for managing the fryer 2 may output the deep-frying information.

The deep-frying weight identification section 85 is configured to calculate the deep-frying weight W per unit time (in the present embodiment, an hour) based on the deep-frying information acquired by the data acquisition section 81A, identify which category the deep-frying weight W per unit time at the store is included (in the present embodiment, “small”, “medium”, and “large” of the deep-frying weight W per unit time), and set the first-order coefficient α of PCn of Equation (1) or the second-order coefficient γ and the first-order coefficient δ of PCn of Equation (2), which is stored in the storage section 82A, to a value corresponding to the deep-frying weight W per unit time, respectively. Thus, Equation (1) or Equation (2) stored in the storage section 82A is updated.

In the present embodiment, the cloud 8A calculates the deep-frying weight W per unit time (an hour) based on the deep-frying information output from the store terminal 6 to identify which category the deep-frying weight W per unit time at the store is included, however, the store terminal 6 may identify which category the deep-frying weight W per unit time is included. In this case, the cloud 8A sets the first-order coefficient α of PCn of Equation (1) or the second-order coefficient γ and the first-order coefficient δ of PCn of Equation (2), respectively, based on the category information output from the store terminal 6. That is, the cloud 8A does not necessarily have to have the function of determining which category the deep-frying weight W per unit time is included.

The deterioration indicator calculation section 83A is configured to calculate the acid value of the frying oil P based on the measured value of the total polar compounds of the frying oil P acquired by the data acquisition section 81A, and Equation (1) in which the first-order coefficient α of PCn has been set by the deep-frying weight identification section 85 or Equation (2) in which the second-order coefficient γ and the first-order coefficient δ of PCn have been set by the deep-frying weight identification section 85.

In the same manner as the first embodiment, the detection result output section 84 is configured to output the acid value of the frying oil P calculated by the deterioration indicator calculation section 83A to the store terminal 6 and the head office terminal 7, respectively, as the result of detection of the deterioration degree of the frying oil P.

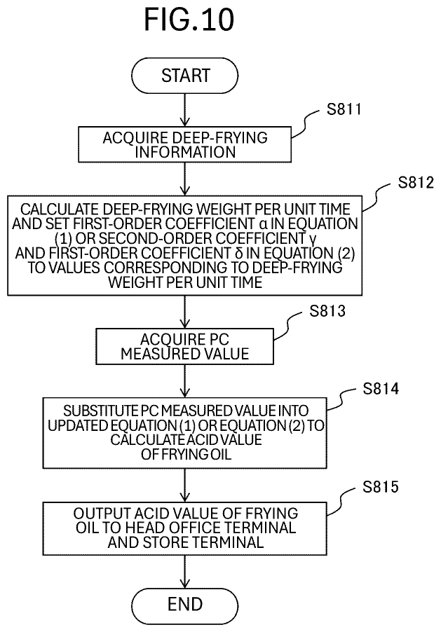

FIG. 10 illustrates a flowchart of a flow of the processing to be executed by the cloud 8A according to the second embodiment.

In the cloud 8A, firstly, the data acquisition section 81A acquires the deep-frying information output from the store terminal 6 (step S811; deep-frying information acquisition step).

Subsequently, the deep-frying weight identification section 85 calculates the deep-frying weight W per unit time based on the deep-frying information acquired in step S811 to identify which category the deep-frying weight W per unit time at the store is included (step S812; deep-frying weight determination step), and sets the first-order coefficient α of PCn of Equation (1) or the second-order coefficient γ and the first-order coefficient δ of PCn of Equation (2), which is stored in the storage section 82A, to values corresponding to the deep-frying weight W per unit time, respectively (step S812; parameter setting step). Thus, Equation (1) or Equation (2) stored in the storage section 82A is updated.

Next, the data acquisition section 81A acquires a measured value of the total polar compounds of the frying oil P output from the PC sensor 41 (step S813; data acquisition step).

Next, the deterioration indicator calculation section 83A substitutes the measured value of the total polar compounds of the frying oil P acquired in step S813 into PCn of Equation (1) or Equation (2) which has been updated in step S812, so as to calculate the acid value AVn of the frying oil P (step S814; deterioration indicator calculation step).

Then, the detection result output section 84 outputs the acid value AVn of the frying oil P calculated in step S814 to the store terminal 6 and the head office terminal 7, respectively, as the result of detection of the deterioration degree of the frying oil P (step S815; detection result output step), whereby the processing in the cloud 8A is ended.

In the present embodiment, the cloud 8A calculates the acid value of the frying oil P using Equation (1) or Equation (2) which depends on the deep-frying weight W per unit time at a store, which enables the calculation of the acid value of the frying oil P with higher accuracy than the case of calculating it using the predetermined Equation (1) or Equation (2).

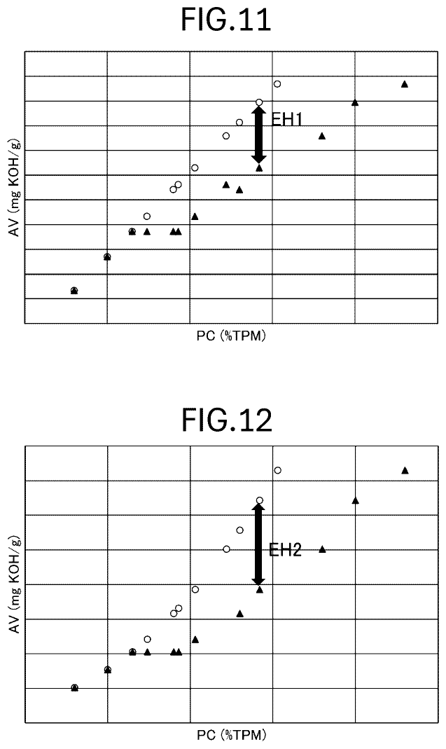

Next, a cloud 8B according to the third embodiment of the present invention will be described with reference to FIG. 11 to FIG. 14.

FIG. 11 illustrates a graph of a linear function showing the correlation of the acid value of the frying oil P relative to the total polar compounds contained in the frying oil P, which relates to whether empty heating has been performed. FIG. 12 illustrates a graph of a quadratic function showing the correlation of the acid value of the frying oil P relative to the total polar compounds contained in the frying oil P, which relates to whether empty heating has been performed.

As illustrated in FIG. 11 and FIG. 12, the graphs showing the correlations of the acid values of the frying oil P relative to the total polar compounds contained in the frying oil P vary depending on whether empty heating for the frying oil P has been performed. The “empty heating” is heating only the frying oil P without deep frying the deep-frying material Q, that is, with the deep-frying material Q not being placed in the frying oil P.

In each of FIG. 11 and FIG. 12, a correlation graph including a plurality of “∘” is indicative of the case where empty heating for the frying oil P has not been performed, and a correlation graph including a plurality of “▴” is indicative of the case where empty heating for the frying oil P has been performed.

In the correlation graph illustrated in FIG. 11, in the case where the empty heating for the frying oil P has been performed, the rate of increase in the acid value tends to be smaller than the rate of increase in the total polar compounds, and the acid value decreases by the amount of EH1. In the same manner, in the correlation graph illustrated in FIG. 12, in the case where the empty heating for the frying oil P has been performed, the rate of increase in the acid value tends to be smaller than the rate of increase in the total polar compounds, and the acid value decreases by the amount of EH2.

Here, each of “EH1” and “EH2” corresponds to an empty heating variable set in view of the empty heating for the frying oil P. The empty heating variable increases as the empty heating time increases.

Thus, in the case where the empty heating for the frying oil P has been performed, a correlation equation for the acid value of the frying oil P relative to the total polar compounds is the following Equation (3) expressed with a linear equation obtained by subtracting the empty heating variable EH1 from Equation (1), or the following Equation (4) expressed with a quadratic equation obtained by subtracting the empty heating variable EH2 from Equation (2)

$$AVn = α \times ( PCn ) + β - EH 1 \qquad ( 3 )$$

$$AVn = γ \times {( PCn )}^{2} + δ \times ( PCn ) + ε - EH 2 \qquad ( 4 )$$

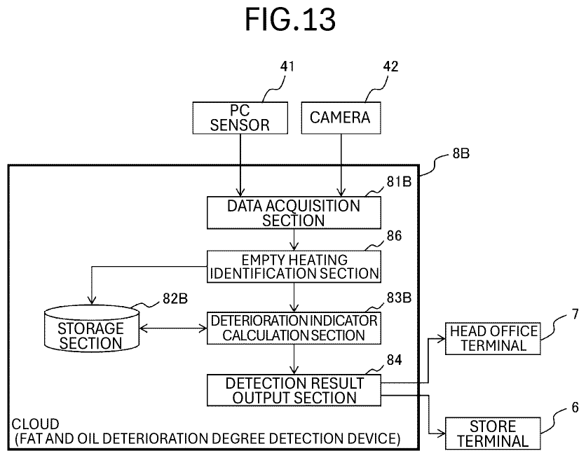

FIG. 13 illustrates a functional block diagram illustrating functions provided in the cloud 8B according to the third embodiment.

The cloud 8B according to the present embodiment includes a data acquisition section 81B, an empty heating identification section 86, a storage section 82B, a deterioration indicator calculation section 83B, and a detection result output section 84.

The data acquisition section 81B is configured to acquire not only a measured value of the total polar compounds of the frying oil P output from the PC sensor 41, but also a surface image of the frying oil P output from the camera 42.

The empty heating identification section 86 is configured to identify whether the empty heating for the frying oil P has been performed based on the surface image of the frying oil P acquired by the data acquisition section 81B. As described above, the empty heating is heating the frying oil P with the deep-frying material Q not being placed therein. The empty heating identification section 86 identifies how long the deep-frying material Q is not included in the surface image of the frying oil P captured by the camera 42, as the period of time during which the empty heating has been performed.

The method for determining whether the empty heating has been performed does not necessarily have to be based on whether the deep-frying material Q is included in the surface image of the frying oil P captured by the camera 42. For example, a temperature sensor may be attached to the fryer 2, so that it can be identified that the empty heating has been performed based on the temperature measured by the temperature sensor which falls below a predetermined temperature (empty heating temperature). Alternatively, for example, a weight sensor may be attached to the fryer 2, so that it can be identified that the empty heating has been performed based on the increase or decrease in the weight measured by the weight sensor.

Furthermore, for example, it may be identified that the empty heating has been performed based on the information on the type and number of the material Q for deep frying or the time for performing deep frying, which has been recorded at a store. Still further, it may be identified that the empty heating has been performed based on the empty heating time which is calculated based on a daily schedule of deep frying which has been recorded in advance at a store.

Still further, for example, it may be identified that the empty heating has been performed based on an operation made for the operation switches 22A, which starts the deep frying at the fryer 2. Still further, for example, it may be identified that the empty heating has been performed based on the consumption of power or gas at the fryer 2.

The storage 82B retains not only Equation (1) or Equation (2), but also retains Equation (3) or Equation (4) as the correlation equations between the acid values of the frying oil P and the total polar compounds contained in the frying oil P.

Upon determining that the empty heating for the frying oil P has been performed, the empty heating identification section 86 sets the empty heating variable EH1 of Equation (3) or the empty heating variable EH2 of Equation (4), which is stored in the storage section 82B.

In the present embodiment, the empty heating identification section 86 identifies whether the empty heating for the frying oil P has been performed, however, the data acquisition section 81B may acquire the information relating to whether the empty heating has been performed from the store terminal 6 or the head office terminal 7. In this case, based on the information (information indicating that the empty heating has been performed) acquired by the data acquisition section 81B, the empty heating identification section 86 uses and sets the empty heating variable EH1 of Equation (3) or the empty heating variable EH2 of Equation (4), which is stored in the storage section 82B. That is, the cloud 8B does not necessarily have to have the function of determining whether the empty heating for the frying oil P has been performed.

In the case where the empty heating identification section 86 identifies that the empty heating for the frying oil P has been performed, the deterioration indicator calculation section 83B calculates the acid value of the frying oil P based on the measured value of the total polar compounds acquired by the data acquisition section 81B and Equation (3) or Equation (4) stored in the storage section 82B.

In the case where the empty heating identification section 86 identifies that the empty heating for the frying oil P has not been performed, the deterioration indicator calculation section 83B calculates the acid value of the frying oil P based on the measured value of the total polar compounds acquired by the data acquisition section 81B and Equation (1) or Equation (2) stored in the storage section 82B.

The detection result output section 84 outputs the acid value of the frying oil P calculated by the deterioration indicator calculation section 83B to the store terminal 6 and the head office terminal 7, respectively, as the result of detection of the deterioration degree of the frying oil P.

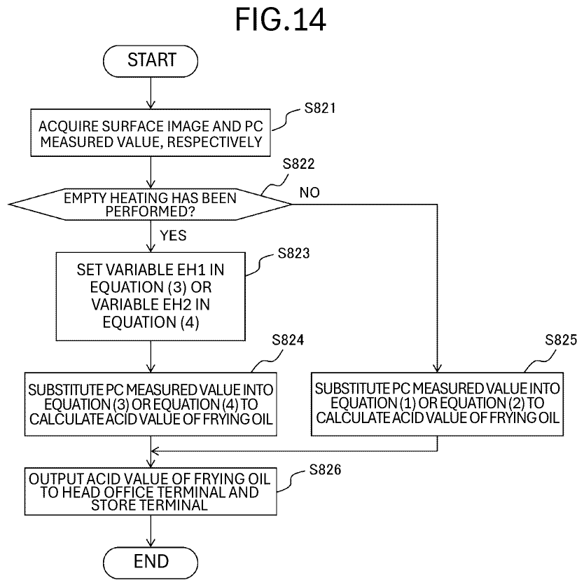

FIG. 14 illustrates a flowchart of a flow of the processing to be executed by the cloud 8B according to the third embodiment.

In the cloud 8B, firstly, the data acquisition section 81B acquires the measured value of the total polar compounds of the frying oil P output from the PC sensor 41 and the surface image of the frying oil P output from the camera 42 (step S821; data acquisition step).

Next, the empty heating identification section 86 identifies whether the empty heating for the frying oil P has been performed based on the surface image of the frying oil P acquired in step S821 (step S822; empty heating determination step).

In the case where it is identified in step S822 that the empty heating for the frying oil P has been performed (step 822/YES), the empty heating identification section 86 sets the empty heating variable EH1 of Equation (3) or the empty heating variable EH2 of Equation (4), which is stored in the storage section 82B (step S823; parameter setting step).

Next, the deterioration indicator calculation section 83B substitutes the measured value of the total polar compounds of the frying oil P acquired in step S821 into PCn of Equation (3) or PCn of Equation (4) to calculate the acid value AVn of the frying oil P (step S824; deterioration indicator calculation step).

On the other hand, in the case where it is identified in step S822 that the empty heating for the frying oil P has not been performed (step 822/NO), the deterioration indicator calculation section 83B substitutes the measured value of the total polar compounds of the frying oil P acquired in step S821 into PCn of Equation (1) or PCn of Equation (2), which is stored in the storage section 82B, to calculate the acid value AVn of the frying oil P (step S825; deterioration indicator calculation step).

Then, the detection result output section 84 outputs the acid value AVn of the frying oil P calculated in step S824 or step S825 to the store terminal 6 and the head office terminal 7, respectively, as the result of detection of the deterioration degree of the frying oil P (step S826; detection result output step), whereby the processing in the cloud 8B is ended.

According to the present embodiment, by using Equation (1) or Equation (2), or Equation (3) or Equation (4) appropriately depending on whether empty heating for the frying oil P has been performed so as to calculate the acid value of the frying oil P, the calculation of the acid value of the frying oil P can be carried out with higher accuracy than the case of calculating it using Equation (1) or Equation (2) in all cases without considering whether empty heating for the frying oil P has been performed.

### Fourth Embodiment

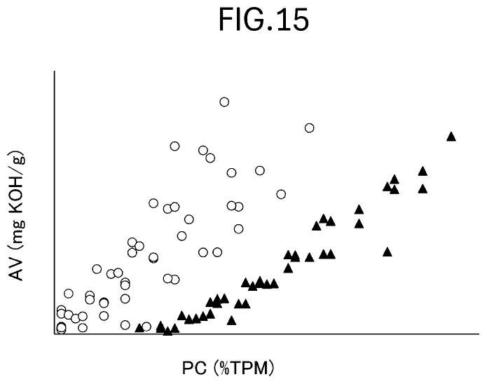

Next, a cloud 8C according to the fourth embodiment of the present invention will be described with reference to FIG. 15 to FIG. 19.

FIG. 15 illustrates a graph showing the correlation of the acid value of the frying oil P relative to the total polar compounds contained in the frying oil P relating to a first oil type and a second oil type. Specifically, this correlation graph shows the correlation of the acid value of the frying oil P relative to the total polar compounds of the frying oil P measured using the PC sensor 41.

The type of the frying oil P can be classified into a first oil type and a second oil type depending on the fatty acid composition of the frying oil P.

The first oil type is the type of oil indicative of a composition of the frying oil P in which the content of oleic acid is more than the content of linoleic acid (the content of oleic acid>the content of linoleic acid). The first oil type includes, for example, palm oil, olive oil, peanut oil, safflower oil, and rapeseed oil.

On the other hand, the second oil type is the type of oil indicative of a composition of the frying oil P in which the content of oleic acid is equal to or less than the content of linoleic acid (content of oleic acid 5 content of linoleic acid). The second oil type includes, for example, corn oil, soybean oil, and grape seed oil.

As illustrated in FIG. 15, the correlation graph of the acid value of the frying oil P relative to the total polar compounds contained in the frying oil P differs between the first oil type and the second oil type.

In FIG. 15, a correlation graph including a plurality of “∘” is indicative of the correlation graph of the acid value of the frying oil P corresponding to the first oil type relative to the total polar compounds contained in the frying oil P, and a correlation graph including a plurality of “▴” is indicative of the correlation graph of the acid value of the frying oil P corresponding to the second oil type relative to the total polar compounds contained in the frying oil P, respectively.

In the case where the type of the frying oil P is the first oil type, the correlation equation for the acid value of the frying oil P relative to the total polar compounds of the frying oil P is a linear equation expressed with the following Equation (5) including each of α1 as the first-order coefficient α of PCn and β1 as the constant β in Equation (1), or a quadratic equation expressed with the following Equation (6) including each of γ1 as the second-order coefficient γ of PCn, δ1 as the first-order coefficient δ of PCn, and ε1 as the constant ε in Equation (2).

$$AVn = α 1 \times ( PCn ) + β 1 \qquad ( 5 )$$

$$AVn = γ 1 \times {( PCn )}^{2} + δ 1 \times ( PCn ) + ε 1 \qquad ( 6 )$$

In the case where the type of the frying oil P is the second oil type, the correlation equation for the acid value of the frying oil P relative to the total polar compounds of the frying oil P is a linear equation expressed with the following Equation (7) including each of α2 as the first-order coefficient α of PCn and β2 as the constant β in Equation (1), or a quadratic equation expressed with the following Equation (8) including each of γ2 as the second-order coefficient γ of PCn, δ2 as the first-order coefficient δ of PCn, and ε2 as the constant ε in Equation (2).

$$AVn = α 2 \times ( PCn ) + β 2 \qquad ( 7 )$$

$$AVn = γ 2 \times {( PCn )}^{2} + δ 2 \times ( PCn ) + ε 2 \qquad ( 8 )$$

Here, the first-order coefficient α2 of PCn in Equation (7) is smaller than the first-order coefficient α1 of PCn in Equation (5) (α2<α1), and the constant β2 in Equation (7) is smaller than the constant β1 in Equation (5) (β2<β1). Furthermore, the second-order coefficient γ2 of PCn in Equation (8) is smaller than the second-order coefficient γ1 of PCn in Equation (6) (γ2<γ1), the first-order coefficient δ2 of PCn in Equation (8) is smaller than the first-order coefficient δ1 of PCn in Equation (6) (δ2<δ1), and the constant ε2 in Equation (8) is smaller than the constant ε1 in Equation (6) (ε2<ε1).

As described above, especially in the case of measuring the total polar compounds of the frying oil P using the PC sensor 41, a difference is found in the correlation of the acid value of the frying oil P relative to the total polar compounds of the frying oil P between the first oil type and the second oil type. Thus, using the equation in which the difference therebetween is considered (Equation (5) or Equation (6) in the case of the first oil type and Equation (7) or Equation (8) in the case of the second oil type) enables the calculation of the acid value of the frying oil P with higher accuracy.

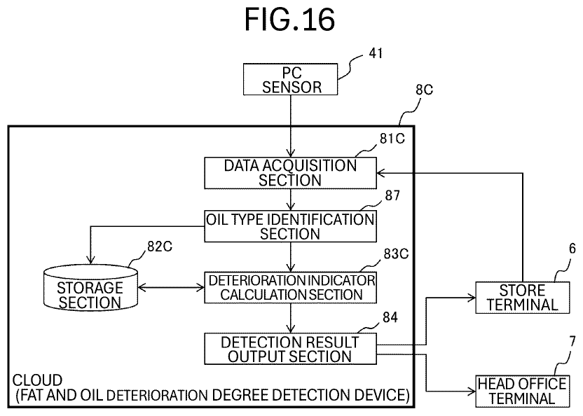

FIG. 16 is a functional block diagram illustrating functions provided in the cloud 8C according to the fourth embodiment.

The cloud 8C according to the present embodiment includes a data acquisition section 81C, an oil type identification section 87, a storage section 82C, a deterioration indicator calculation section 83C, and a detection result output section 84.

The data acquisition section 81C is configured to acquire not only a measured value of the total polar compounds of the frying oil P output from the PC sensor 41, but also the information relating to the fatty acid composition of the frying oil P output from the store terminal 6. The information relating to the fatty acid composition of the frying oil P includes, for example, the information indicative of the specific names of oil types such as palm oil, corn oil, and olive oil, the information indicative of the content of oleic acid and the content of linoleic acid of the frying oil P, and the like. The information relating to the fatty acid composition of the frying oil P does not necessarily have to be output from the store terminal 6. It may be output, for example, from the head office terminal 7, both the store terminal 6 and the head office terminal 7, or an external terminal which manages the frying oil P.

The oil type identification section 87 is configured to identify the type of the frying oil P, in other words, whether it is the first type oil or the second type oil, based on the information relating to the fatty acid composition of the frying oil P acquired by the data acquisition section 81C. Upon determining that the type of the frying oil P is the first oil type, the oil type identification section 87 selects Equation (5) or Equation (6) stored in the storage section 82C, and upon determining that the type of the frying oil P is the second oil type, the oil type identification section 87 selects Equation (7) or Equation (8) stored in the storage section 82C.

The deterioration indicator calculation section 83C is configured to calculate the acid value of the frying oil P based on the measured value of the total polar compounds of the frying oil P acquired by the data acquisition section 81C and Equation (5) or Equation (6) stored in the storage section 82C in the case where the oil type identification section 87 identifies that the type of the frying oil P is the first oil type.

The deterioration indicator calculation section 83C is configured to calculate the acid value of the frying oil P based on the measured value of the total polar compounds of the frying oil P acquired by the data acquisition section 81C and Equation (7) or Equation (8) stored in the storage section 82C in the case where the oil type identification section 87 identifies that the type of the frying oil P is the second oil type.

In the present embodiment, the oil type identification section 87 identifies the type of the frying oil P (whether it is the first oil type or the second oil type), however, for example, the data acquisition section 81C may acquire the information relating to the type of the frying oil P itself, such as the information indicative of the “the first oil type” or “the second oil type”, from the store terminal 6 or the head office terminal 7. In this case, based on the information relating to the type of the frying oil P acquired by the data acquisition section 81C, the deterioration indicator calculation section 83C selects an equation to be used for calculation of the acid value of the frying oil P. That is, the cloud 8C does not necessarily have to have the function of determining the type of the frying oil P (oil type). The same applies to the cloud 8C according to each of the fifth to seventh embodiments.

In the same manner as in the first to third embodiments, the detection result output section 84 outputs the acid value of the frying oil P calculated by the deterioration indicator calculation section 83C to the store terminal 6 and the head office terminal 7, respectively, as the result of detection of the deterioration degree of the frying oil P.

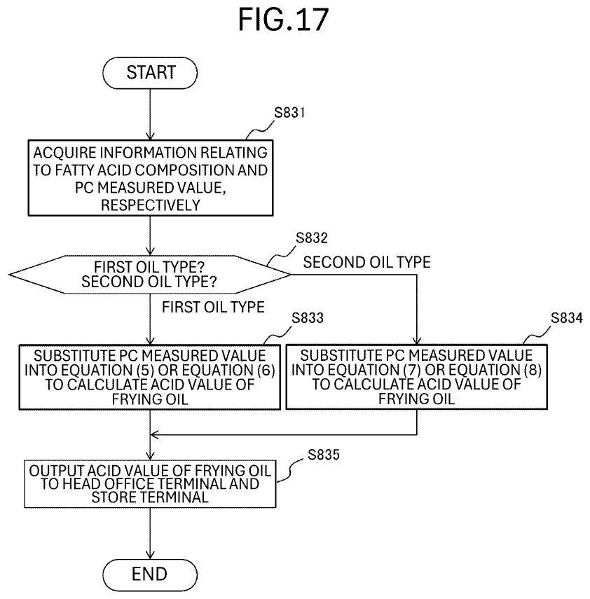

FIG. 17 illustrates a flowchart of a flow of the processing to be executed by the cloud 8C according to the fourth embodiment.

In the cloud 8C, firstly, the data acquisition section 81C acquires the measured value of the total polar compounds of the frying oil P output from the PC sensor 41 and the information relating to the fatty acid composition of the frying oil P output from the store terminal 6 (step S831; data acquisition step).

Next, the oil type identification section 87 identifies whether the type of the frying oil P is the first oil type or the second oil type based on the information relating to the fatty acid composition of the frying oil P acquired in step S831 (step S832; oil type identification step).

In the case where it is identified in step S832 that the type of the frying oil P is the first oil type (step S832/first oil type), the deterioration indicator calculation section 83C substitutes the measured value of the total polar compounds of the frying oil P acquired in step S831 into PCn of Equation (5) or PCn of Equation (6), which is stored in the storage section 82C, to calculate the acid value AVn of the frying oil P (step S833; deterioration indicator calculation step).

On the other hand, in the case where it is identified in step S832 that the type of the frying oil P is the second oil type (step S832/second oil type), the deterioration indicator calculation section 83C substitutes the measured value of the total polar compounds of the frying oil P acquired in step S831 into PCn of Equation (7) or PCn of Equation (8), which is stored in the storage section 82C, to calculate the acid value AVn of the frying oil P (step S834; deterioration indicator calculation step).

Then, the detection result output section 84 outputs the acid value AVn of the frying oil P calculated in step S833 or step S834 to the store terminal 6 and the head office terminal 7, respectively, as the result of detection of the deterioration degree of the frying oil P (step S835; detection result output step), whereby the processing in the cloud 8C is ended.

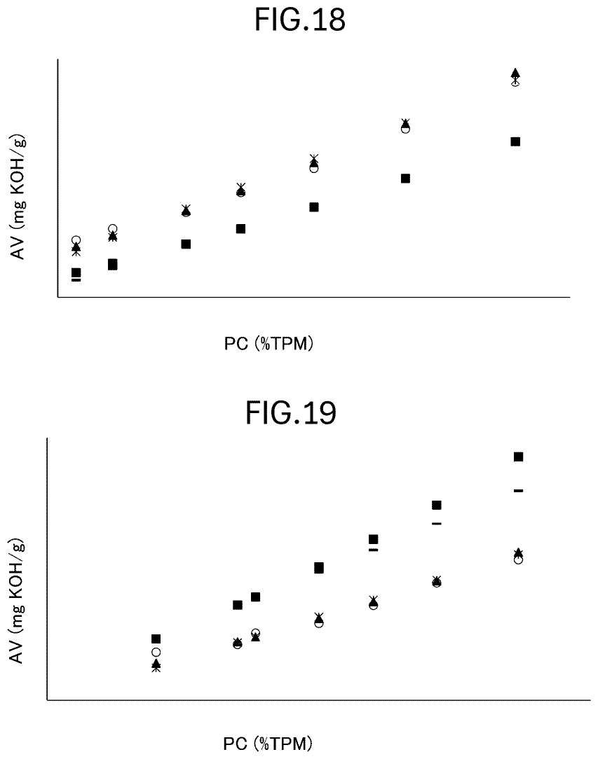

FIG. 18 illustrates a graph showing the correlation of the acid value of the frying oil P relating to the first oil type relative to the total polar compounds (measured using the PC sensor 41) contained in the frying oil P, in which the actually measured acid values are compared with the acid values calculated using Equation (1), Equation (2), Equation (5), and Equation (6), respectively. FIG. 19 illustrates a graph showing the correlation of the acid value of the frying oil P relating to the second oil type relative to the total polar compounds (measured using the PC sensor 41) contained in the frying oil P, in which the actually measured acid values are compared with the acid values calculated using Equation (1), Equation (2), Equation (7), and Equation (8), respectively.

In FIG. 18 relating to the case where the type of the frying oil P is the first oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (5), and a graph including a plurality of “▴” is indicative of the correlation graph for Equation (6), respectively.

In FIG. 19 relating to the case where the type of the frying oil P is the second oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (7), and a graph including a plurality of “▴” is indicative of the correlation graph for Equation (8), respectively.

As illustrated in FIG. 18, the correlation graph for Equation (5) and the correlation graph for Equation (6) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (5) and the correlation graph for Equation (6).

In the same manner, as illustrated in FIG. 19, the correlation graph for Equation (7) and the correlation graph for Equation (8) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (7) and the correlation graph for Equation (8).

Thus, in the case where the PC sensor 41 measures the total polar compounds of the frying oil P, the cloud 8C identifies whether the type of the frying oil P is the first oil type or the second oil type based on the fatty acid composition of the frying oil P and calculates the acid value of the frying oil P using the correlation equation for the oil type as identified. This enables the calculation of the acid value of the frying oil P with higher accuracy than the case of calculating it using Equation (1) or Equation (2) in all cases without considering the type of oil classified based on the fatty acid composition of the frying oil P.

### Fifth Embodiment

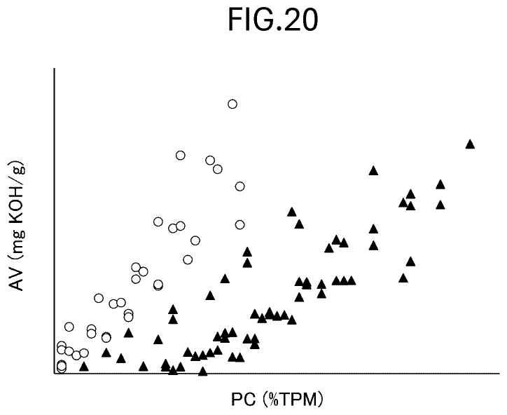

Next, the cloud 8C according to the fifth embodiment of the present invention will be described with reference to FIG. 20 to FIG. 23. The functional block of the functions provided in the cloud 8C according to the present embodiment is not illustrated herein as it is the same as that of the cloud 8C according to the fourth embodiment, and the components which are common therebetween are provided with the same reference signs. In the following, the same applies to the sixth embodiment and the seventh embodiment.

FIG. 20 illustrates a graph showing the correlation of the acid value of the frying oil P relating to a third oil type and a fourth oil type relative to the total polar compounds contained in the frying oil P. Specifically, this correlation graph shows the correlation of the acid value of the frying oil P relative to the total polar compounds of the frying oil P measured using the PC sensor 41.

The type of the frying oil P is classified into the third oil type and the fourth oil type depending on the iodine value (IV) of the frying oil P.

The third oil type is the type of oil for which the iodine value of the frying oil P is less than a predetermined iodine value threshold (for example, 100) (IV<IVth), and includes, for example, sunflower oil, rapeseed oil, olive oil, peanut oil, and safflower oil.

On the other hand, the fourth oil type is the type of oil for which the iodine value of the frying oil P is equal to or more than the predetermined iodine value threshold (for example, 100) (IV≥IVth), and includes, for example, rice oil, refined sesame oil, cottonseed oil, corn oil, soybean oil, and grape seed oil.

As illustrated in FIG. 20, the correlation graph of the acid value of the frying oil P relative to the total polar compounds contained in the frying oil P differs between the third oil type and the fourth oil type.

In FIG. 20, a correlation graph including a plurality of “∘” is indicative of the correlation graph of the acid value of the frying oil P corresponding to the third oil type relative to the total polar compounds contained in the frying oil P, and a correlation graph including a plurality of “▴” is indicative of the correlation graph of the acid value of the frying oil P corresponding to the fourth oil type relative to the total polar compounds contained in the frying oil P, respectively.

In the case where the type of the frying oil P is the third oil type, the correlation equation for the acid value of the frying oil P relative to the total polar compounds of the frying oil P is a linear equation expressed with the following Equation (9) including each of α3 as the first-order coefficient α of PCn and β3 as the constant β in Equation (1), or a quadratic equation expressed with the following Equation (10) including each of γ3 as the second-order coefficient γ of PCn, δ3 as the first-order coefficient δ of PCn, and ε3 as the constant ε in Equation (2)

$$AVn = α 3 \times ( PCn ) + β 3 \qquad ( 9 )$$

$$AVn = γ 3 \times {( PCn )}^{2} + δ 3 \times ( PCn ) + ε 3 \qquad ( 10 )$$

In the case where the type of the frying oil P is the fourth oil type, the correlation equation for the acid value of the frying oil P relative to the total polar compounds of the frying oil P is a linear equation expressed with the following Equation (11) including each of α4 as the first-order coefficient α of PCn and β4 as the constant β in Equation (1), or a quadratic equation expressed with the following Equation (12) including each of γ4 as the second-order coefficient γ of PCn, δ4 as the first-order coefficient δ of PCn, and ε4 as the constant ε in Equation (2).

$$AVn = α 4 \times ( PCn ) + β 4 \qquad ( 11 )$$

$$AVn = γ 4 \times {( PCn )}^{2} + δ 4 \times ( PCn ) + ε 4 \qquad ( 12 )$$

Here, the first-order coefficient α4 of PCn in Equation (11) is smaller than the first-order coefficient α3 of PCn in Equation (9) (α4<α3), and the constant β4 in Equation (11) is smaller than the constant β3 in Equation (9) (β4<β3). Furthermore, the second-order coefficient γ4 of PCn in Equation (12) is smaller than the second-order coefficient γ3 of PCn in Equation (10) (γ4<γ3), the first-order coefficient δ4 of PCn in Equation (12) is smaller than the first-order coefficient δ3 of PCn in Equation (10) (δ4<δ3), and the constant ε4 in Equation (12) is smaller than the constant ε3 in Equation (10) (ε4<ε3).

As described above, especially in the case of measuring the total polar compounds of the frying oil P using the PC sensor 41, a difference is found in the correlation of the acid value of the frying oil P relative to the total polar compounds of the frying oil P between the third oil type and the fourth oil type. Thus, using the equation in which the difference therebetween is considered (Equation (9) or Equation (10) in the case of the third oil type and Equation (11) or Equation (12) in the case of the fourth oil type) enables the calculation of the acid value of the frying oil P with higher accuracy.

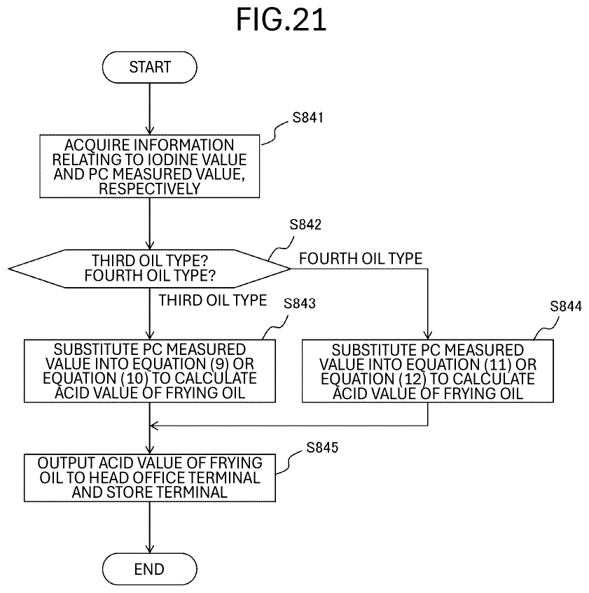

FIG. 21 illustrates a flowchart of a flow of the processing to be executed by the cloud 8C according to the fifth embodiment.

In the cloud 8C according to the present embodiment, firstly, the data acquisition section 81C acquires the measured value of the total polar compounds of the frying oil P output from the PC sensor 41 and the information relating to the iodine value of the frying oil P output from the store terminal 6 (step S841; data acquisition step).

The information relating to the iodine value of the frying oil P includes, for example, the information indicative of the specific names of oil types, the information indicative of the iodine value of the frying oil P, and the like. The information relating to the iodine value of the frying oil P does not necessarily have to be output from the store terminal 6 to the cloud 8C, but may be output from, for example, the head office terminal 7 to the cloud 8C.

Next, the oil type identification section 87 identifies whether the type of the frying oil P is the third oil type or the fourth oil type based on the information relating to the iodine value of the frying oil P acquired in step S841 (step S842; oil type identification step).

In the case where it is identified in step S842 that the type of the frying oil P is the third oil type (step S842/third oil type), the deterioration indicator calculation section 83C substitutes the measured value of the total polar compounds of the frying oil P acquired in step S841 into PCn of Equation (9) or PCn of Equation (10), which is stored in the storage section 82C, to calculate the acid value AVn of the frying oil P (step S843; deterioration indicator calculation step).

On the other hand, in the case where it is identified in step S842 that the type of the frying oil P is the fourth oil type (step S842/fourth oil type), the deterioration indicator calculation section 83C substitutes the measured value of the total polar compounds of the frying oil P acquired in step S841 into PCn of Equation (11) or PCn of Equation (12), which is stored in the storage section 82C, to calculate the acid value AVn of the frying oil P (step S844; deterioration indicator calculation step).

Then, the detection result output section 84 outputs the acid value AVn of the frying oil P calculated in step S843 or step S844 to the store terminal 6 and the head office terminal 7, respectively, as the result of detection of the deterioration degree of the frying oil P (step S845; detection result output step), whereby the processing in the cloud 8C is ended.

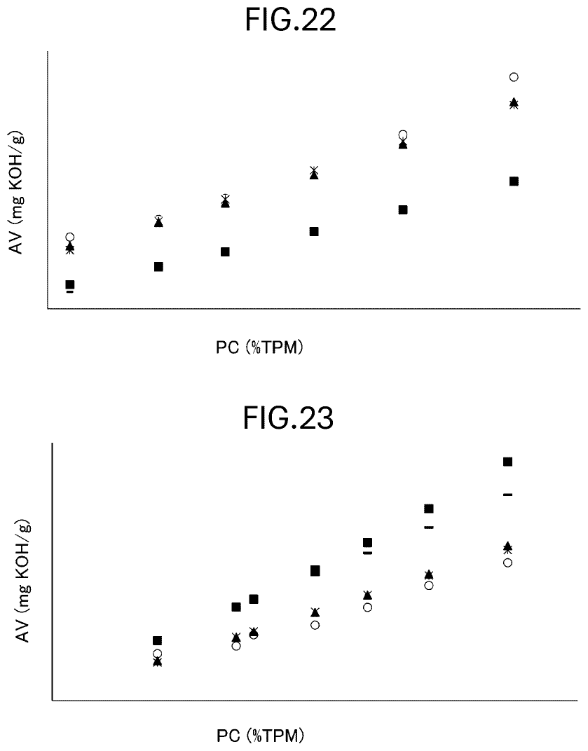

FIG. 22 illustrates a graph showing the correlation of the acid value of the frying oil P relating to the third oil type relative to the total polar compounds (measured using the PC sensor 41) contained in the frying oil P, in which the actually measured acid values are compared with the acid values calculated using Equation (1), Equation (2), Equation (9), and Equation (10), respectively. FIG. 23 illustrates a graph showing the correlation of the acid value of the frying oil P relating to the fourth oil type relative to the total polar compounds (measured using the PC sensor 41) contained in the frying oil P, in which the actually measured acid values are compared with the acid values calculated using Equation (1), Equation (2), Equation (11), and Equation (12), respectively.

In FIG. 22 relating to the case where the type of the frying oil P is the third oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (9), and a graph including a plurality of “▴” is indicative of the correlation graph for Equation (10), respectively.

In FIG. 23 relating to the case where the type of the frying oil P is the fourth oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (11), and a graph including a plurality of “▴” is indicative of the correlation graph for Equation (12), respectively.

As illustrated in FIG. 22, the correlation graph for Equation (9) and the correlation graph for Equation (10) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (9) and the correlation graph for Equation (10).

In the same manner, as illustrated in FIG. 23, the correlation graph for Equation (11) and the correlation graph for Equation (12) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (11) and the correlation graph for Equation (12).

Thus, in the case where the PC sensor 41 measures the total polar compounds of the frying oil P, the cloud 8C identifies whether the type of the frying oil P is the third oil type or the fourth oil type based on the iodine value of the frying oil P and calculates the acid value of the frying oil P using the correlation equation for the oil type as identified. This enables the calculation of the acid value of the frying oil P with higher accuracy than the case of calculating it using Equation (1) or Equation (2) in all cases without considering the type of oil classified based on the iodine value of the frying oil P.

### Sixth Embodiment

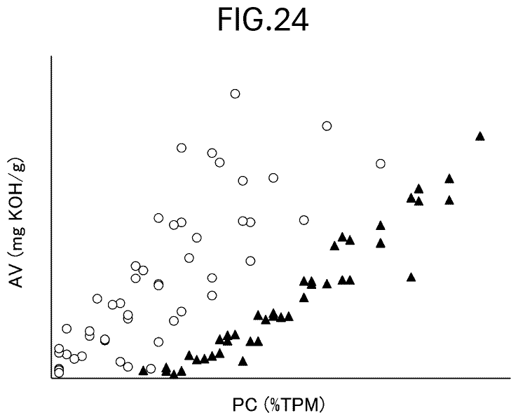

Next, the cloud 8C according to the sixth embodiment of the present invention will be described with reference to FIG. 24 to FIG. 27.

FIG. 24 illustrates a graph showing the correlation of the acid value of the frying oil P relating to a fifth oil type and a sixth oil type relative to the total polar compounds contained in the frying oil P. Specifically, this correlation graph shows the correlation of the acid value of the frying oil P relative to the total polar compounds of the frying oil P measured using the PC sensor 41.

The type of the frying oil P is classified into the fifth oil type and the sixth oil type depending on the value obtained by the conductometric determination method (CDM) test, which is one of the tests for evaluating the oxidative stability of fat and oil (hereinafter, simply referred to as “CDM value”).

The fifth oil type is the type of oil for which the CDM value of the frying oil P is equal to or more than a predetermined CDM threshold (for example, 26 at measurement temperature of 97.8° C.) (CDM value≥predetermined CDM threshold), and includes, for example, palm oil, olive oil, peanut oil, refined sesame oil, and safflower oil.

On the other hand, the sixth oil type is the type of oil for which the CDM value of the frying oil P is less than the predetermined CDM threshold (for example, 26 at measurement temperature of 97.8° C.) (CDM value<predetermined CDM threshold), and includes, for example, cottonseed oil, corn oil, soybean oil, and grape seed oil.

As illustrated in FIG. 24, the correlation graph of the acid value of the frying oil P relative to the total polar compounds contained in the frying oil P differs between the fifth oil type and the sixth oil type.

In FIG. 24, a correlation graph including a plurality of “∘” is indicative of the correlation graph of the acid value of the frying oil P corresponding to the fifth oil type relative to the total polar compounds contained in the frying oil P, and a correlation graph including a plurality of “▴” is indicative of the correlation graph of the acid value of the frying oil P corresponding to the sixth oil type relative to the total polar compounds contained in the frying oil P, respectively.

In the case where the type of the frying oil P is the fifth oil type, the correlation equation for the acid value of the frying oil P relative to the total polar compounds of the frying oil P is a linear equation expressed with the following Equation (13) including each of α5 as the first-order coefficient α of PCn and β5 as the constant β in Equation (1), or a quadratic equation expressed with the following Equation (14) including each of γ5 as the second-order coefficient γ of PCn, δ5 as the first-order coefficient δ of PCn, and ε5 as the constant ε in Equation (2)

$$AVn = α 5 \times ( P C n ) + β5 \qquad ( 13 )$$

$$A Vn = γ5 \times {( PCn )}^{2} + δ5 \times ( PCn ) + ε5 \qquad ( 14 )$$

In the case where the type of the frying oil P is the sixth oil type, the correlation equation for the acid value of the frying oil P relative to the total polar compounds of the frying oil P is a linear equation expressed with the following Equation (15) including each of α6 as the first-order coefficient α of PCn and β6 as the constant β in Equation (1), or a quadratic equation expressed with the following Equation (16) including each of γ6 as the second-order coefficient γ of PCn, δ6 as the first-order coefficient δ of PCn, and ε6 as the constant ε in Equation (2).

$$AVn = α 6 \times ( P C n ) + β6 \qquad ( 15 )$$

$$A Vn = γ6 \times {( PCn )}^{2} + δ6 \times ( PCn ) + ε6 \qquad ( 16 )$$

Here, the first-order coefficient α6 of PCn in Equation (15) is smaller than the first-order coefficient α5 of PCn in Equation (13) (α6<α5), and the constant β6 in Equation (15) is smaller than the constant β5 in Equation (13) (β6<β5). Furthermore, the second-order coefficient γ6 of PCn in Equation (16) is smaller than the second-order coefficient γ5 of PCn in Equation (14) (γ6<γ5), the first-order coefficient δ6 of PCn in Equation (16) is smaller than the first-order coefficient δ5 of PCn in Equation (14) (66<65), and the constant ε6 in Equation (16) is smaller than the constant ε5 in Equation (14) (ε6<ε5).

As described above, especially in the case of measuring the total polar compounds of the frying oil P using the PC sensor 41, a difference is found in the correlation of the acid value of the frying oil P relative to the total polar compounds of the frying oil P between the fifth oil type and the sixth oil type. Thus, using the equation in which the difference therebetween is considered (Equation (13) or Equation (14) in the case of the fifth oil type and Equation (15) or Equation (16) in the case of the sixth oil type) enables the calculation of the acid value of the frying oil P with higher accuracy.

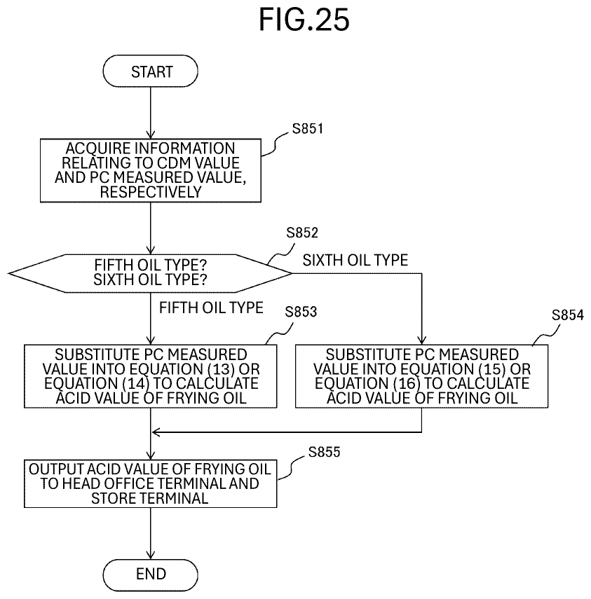

FIG. 25 illustrates a flowchart of a flow of the processing to be executed by the cloud 8C according to the sixth embodiment.

In the cloud 8C according to the present embodiment, firstly, the data acquisition section 81C acquires the measured value of the total polar compounds of the frying oil P output from the PC sensor 41 and the information relating to the CDM value of the frying oil P output from the store terminal 6 (step S851; data acquisition step).

The information relating to the CDM value of the frying oil P includes, for example, the information indicative of the specific names of oil types, the information indicative of the CDM value of the frying oil P, and the like. The information relating to the CDM value of the frying oil P does not necessarily have to be output from the store terminal 6 to the cloud 8C, but may be output from, for example, the head office terminal 7 to the cloud 8C.

Next, the oil type identification section 87 identifies whether the type of the frying oil P is the fifth oil type or the sixth oil type based on the information relating to the CDM value of the frying oil P acquired in step S851 (step S852; oil type identification step).

In the case where it is identified in step S852 that the type of the frying oil P is the fifth oil type (step S852/fifth oil type), the deterioration indicator calculation section 83C substitutes the measured value of the total polar compounds of the frying oil P acquired in step S851 into PCn of Equation (13) or PCn of Equation (14), which is stored in the storage section 82C, to calculate the acid value AVn of the frying oil P (step S853; deterioration indicator calculation step).

On the other hand, in the case where it is identified in step S852 that the type of the frying oil P is the sixth oil type (step S852/sixth oil type), the deterioration indicator calculation section 83C substitutes the measured value of the total polar compounds of the frying oil P acquired in step S851 into PCn of Equation (15) or PCn of Equation (16), which is stored in the storage section 82C, to calculate the acid value AVn of the frying oil P (step S854; deterioration indicator calculation step).

Then, the detection result output section 84 outputs the acid value AVn of the frying oil P calculated in step S853 or step S854 to the store terminal 6 and the head office terminal 7, respectively, as the result of detection of the deterioration degree of the frying oil P (step S855; detection result output step), whereby the processing in the cloud 8C is ended.

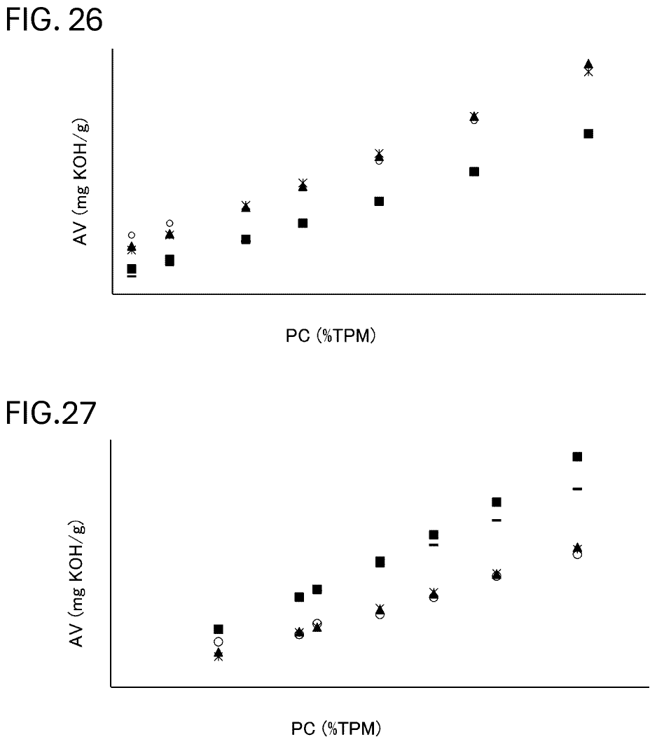

FIG. 26 illustrates a graph showing the correlation of the acid value of the frying oil P relating to the fifth oil type relative to the total polar compounds (measured using the PC sensor 41) contained in the frying oil P, in which the actually measured acid values are compared with the acid values calculated using Equation (1), Equation (2), Equation (13), and Equation (14), respectively. FIG. 27 illustrates a graph showing the correlation of the acid value of the frying oil P relating to the sixth oil type relative to the total polar compounds (measured using the PC sensor 41) contained in the frying oil P, in which the actually measured acid values are compared with the acid values calculated using Equation (1), Equation (2), Equation (15), and Equation (16), respectively.

In FIG. 26 relating to the case where the type of the frying oil P is the fifth oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (13), and a graph including a plurality of “▴” is indicative of the correlation graph for Equation (14), respectively.

In FIG. 27 relating to the case where the type of the frying oil P is the sixth oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (15), and a graph including a plurality of “▴” is indicative of the correlation graph for Equation (16), respectively.

As illustrated in FIG. 26, the correlation graph for Equation (13) and the correlation graph for Equation (14) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (13) and the correlation graph for Equation (14).

In the same manner, as illustrated in FIG. 27, the correlation graph for Equation (15) and the correlation graph for Equation (16) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (15) and the correlation graph for Equation (16).

Thus, in the case where the PC sensor 41 measures the total polar compounds of the frying oil P, the cloud 8C identifies whether the type of the frying oil P is the fifth oil type or the sixth oil type based on the CDM value of the frying oil P and calculates the acid value of the frying oil P using the correlation equation for the oil type as identified. This enables the calculation of the acid value of the frying oil P with higher accuracy than the case of calculating it using Equation (1) or Equation (2) in all cases without considering the type of oil classified based on the CDM value of the frying oil P.

### Seventh Embodiment

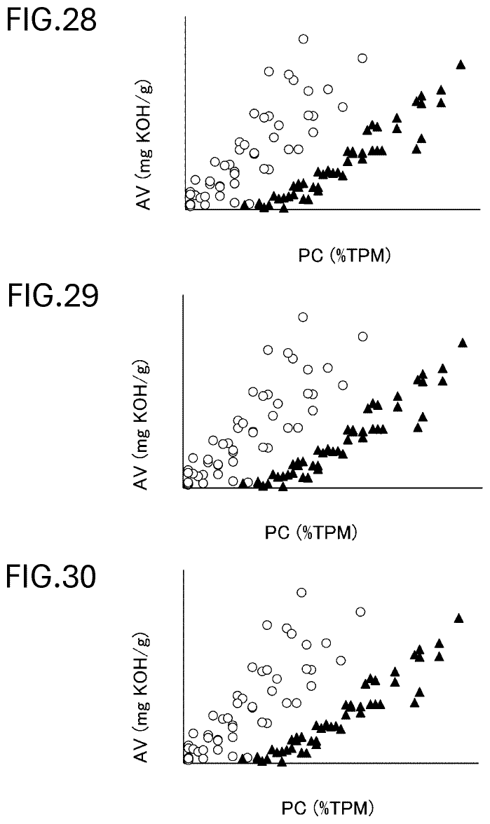

Next, the cloud 8C according to the seventh embodiment of the present invention will be described with reference to FIG. 28 to FIG. 49.

The frying oil P (vegetable oil) is mainly composed of triacylglycerol (TG). When heated, a part of TG is broken down into diacylglycerol (DG), monoacylglycerol (MG), and free fatty acid (FFA).

In general, TG, DG, MG, and FFA are collectively referred to as “lipid molecular species”, and are contained in both new oil (oil that has not been heated and is still fresh since its production) and heated oil. A part of TG is broken down during its production and storage processes, and thus even new oil contains a small amount of DG, MG, and FFA.

In new oil and heated oil, the content ratio of each of TG, DG, MG, and FFA differs depending on the type of oil, and thus the type of the frying oil P can be classified into a seventh oil type and an eighth oil type depending on the lipid molecular species in the frying oil P.

The seventh oil type is the type of oil for which the content of the lipid molecular species in the frying oil P is more than a predetermined content. Specifically, it is the type of oil for which the content of MG in new oil is more than a predetermined MG content threshold for new oil (for example, MG content in new oil>0.1 g/100 g), the type of oil for which the content of FFA in new oil is more than a predetermined FFA content threshold for new oil (for example, FFA content in new oil>0.07 g/100 g), the type of oil for which the content of MG in heated oil is more than a predetermined MG content threshold for heated oil (for example, MG content in heated oil>0.2 g/100 g), and the type of oil for which the content of TG in heated oil is more than a predetermined TG content threshold for heated oil (for example, TG content in heated oil>70 g/100 g).

Furthermore, the seventh oil type is the type of oil for which the rate of increase in the content of DG in the frying oil P due to heating is equal to or less than a predetermined DG increase rate threshold (first increase rate threshold) (for example, rate of increase in DG content due to heating≤1.4 g/100 g), the type of oil for which the rate of increase in the content of FFA in the frying oil P due to heating is equal to or less than a predetermined FFA increase rate threshold (second increase rate threshold) (for example, rate of increase in FFA content due to heating≤0.1/100 g), and the type of oil for which the rate of decrease in the content of TG in the frying oil P due to heating is equal to or less than a predetermined decrease rate threshold (for example, rate of decrease in TG content due to heating≤13 g/100 g).

The seventh oil type includes, for example, palm oil, sunflower oil, safflower oil, and rapeseed oil.

On the other hand, the eighth oil type is the type of oil for which the content of the lipid molecular species in the frying oil P is equal to or less than the predetermined content threshold. Specifically, it is the type of oil for which the content of MG in new oil is equal to or less than the predetermined MG content threshold for new oil (for example, MG content in new oil≤0.1 g/100 g), the type of oil for which the content of FFA in new oil is equal to or less than the predetermined FFA content threshold for new oil (for example, FFA content in new oil≤0.07 g/100 g), the type of oil for which the content of MG in heated oil is equal to or less than the predetermined MG content threshold for heated oil (for example, MG content in heated oil≤0.2 g/100 g), and the type of oil for which the content of TG in heated oil is equal to or less than the predetermined TG content threshold for heated oil (for example, TG content in heated oil≤70 g/100 g).

Furthermore, the eighth oil type is the type of oil for which the rate of increase in the content of DG in the frying oil P due to heating is more than the predetermined DG increase rate threshold (first increase rate threshold) (for example, rate of increase in DG content due to heating>1.4 g/100 g), the type of oil for which the rate of increase in the content of FFA in the frying oil P due to heating is more than the predetermined FFA increase rate threshold (second increase rate threshold) (for example, rate of increase in FFA content due to heating>0.1 g/100 g), and the type of oil for which the rate of decrease in the content of TG in the frying oil P due to heating is more than the predetermined decrease rate threshold (for example, rate of decrease in TG content due to heating>13 g/100 g).

The eighth oil type includes, for example, cottonseed oil, corn oil, soybean oil, and grape seed oil.

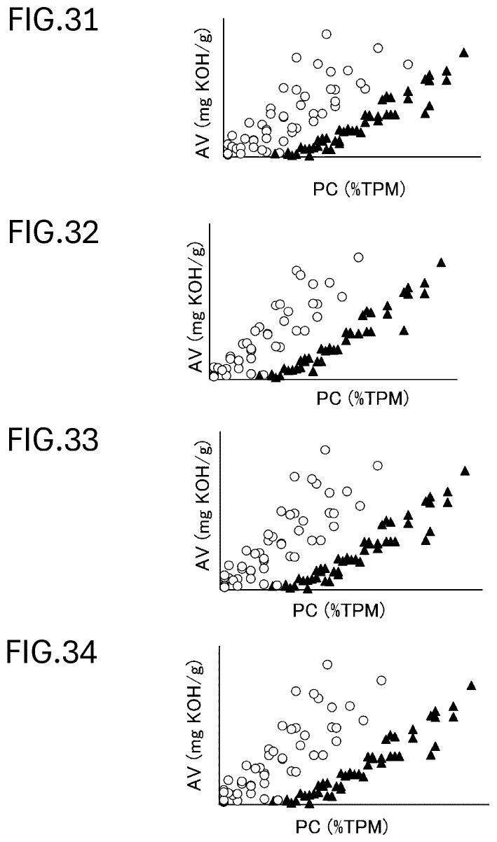

FIG. 28 illustrates a graph showing the correlation of the acid value of the frying oil P relating to the seventh oil type and the eighth oil type, which are classified based on the MG content in new oil, relative to the total polar compounds contained in the frying oil P. FIG. 29 illustrates a graph showing the correlation of the acid value of the frying oil P relating to the seventh oil type and the eighth oil type, which are classified based on the FFA content in new oil, relative to the total polar compounds contained in the frying oil P. FIG. 30 illustrates a graph showing the correlation of the acid value of the frying oil P relating to the seventh oil type and the eighth oil type, which are classified based on the MG content in heated oil, relative to the total polar compounds contained in the frying oil P. FIG. 31 illustrates a graph showing the correlation of the acid value of the frying oil P relating to the seventh oil type and the eighth oil type, which are classified based on the TG content in heated oil, relative to the total polar compounds contained in the frying oil P.

FIG. 32 illustrates a graph showing the correlation of the acid value of the frying oil P relating to the seventh oil type and the eighth oil type, which are classified based on the rate of increase in the DG content due to heating, relative to the total polar compounds contained in the frying oil P. FIG. 33 illustrates a graph showing the correlation of the acid value of the frying oil P relating to the seventh oil type and the eighth oil type, which are classified based on the rate of increase in the FFA content due to heating, relative to the total polar compounds contained in the frying oil P. FIG. 34 illustrates a graph showing the correlation of the acid value of the frying oil P relating to the seventh oil type and the eighth oil type, which are classified based on the rate of decrease in the TG content due to heating, relative to the total polar compounds contained in the frying oil P.

Specifically, each of the correlation graphs in FIG. 28 to FIG. 34 shows the correlation of the acid value of the frying oil P relative to the total polar compounds of the frying oil P measured using the PC sensor 41.

As illustrated in each of FIG. 28 to FIG. 34, the correlation graph of the acid value of the frying oil P relative to the total polar compounds contained in the frying oil P differs between the seventh oil type and the eighth oil type.

In each of FIG. 28 to FIG. 34, a correlation graph including a plurality of “∘” is indicative of the correlation graph of the acid value of the frying oil P corresponding to the seventh oil type relative to the total polar compounds contained in the frying oil P, and a correlation graph including a plurality of “▴” is indicative of the correlation graph of the acid value of the frying oil P corresponding to the eighth oil type relative to the total polar compounds contained in the frying oil P, respectively.

In the case where the type of the frying oil P is the seventh oil type, the correlation equation for the acid value of the frying oil P relative to the total polar compounds of the frying oil P is a linear equation expressed with the following Equation (17) including each of α7 as the first-order coefficient α of PCn and β7 as the constant β in Equation (1), or a quadratic equation expressed with the following Equation (18) including each of γ7 as the second-order coefficient γ of PCn, δ7 as the first-order coefficient δ of PCn, and ε7 as the constant ε in Equation (2).

$$AVn = α 7 \times ( P C n ) + β7 \qquad ( 17 )$$

$$A Vn = γ7 \times {( PCn )}^{2} + δ7 \times ( PCn ) + ε7 \qquad ( 18 )$$

In the case where the type of the frying oil P is the eighth oil type, the correlation equation for the acid value of the frying oil P relative to the total polar compounds of the frying oil P is a linear equation expressed with the following Equation (19) including each of α8 as the first-order coefficient α of PCn and β8 as the constant β in Equation (1), or a quadratic equation expressed with the following Equation (20) including each of γ8 as the second-order coefficient γ of PCn, δ8 as the first-order coefficient δ of PCn, and ε8 as the constant ε in Equation (2).

$$AVn = α 8 \times ( P C n ) + β8 \qquad ( 19 )$$

$$A Vn = γ8 \times {( PCn )}^{2} + δ8 \times ( PCn ) + ε8 \qquad ( 20 )$$

Here, the first-order coefficient α8 of PCn in Equation (19) is smaller than the first-order coefficient α7 of PCn in Equation (17) (α8<α7), and the constant β8 in Equation (19) is smaller than the constant β7 in Equation (17) (38<β7). Furthermore, the second-order coefficient γ8 of PCn in Equation (20) is smaller than the second-order coefficient γ7 of PCn in Equation (18) (γ8<γ7), the first-order coefficient δ8 of PCn in Equation (20) is smaller than the first-order coefficient δ7 of PCn in Equation (18) (δ8<δ7), and the constant ε8 in Equation (20) is smaller than the constant ε7 in Equation (18) (ε8<ε7).

As described above, especially in the case of measuring the total polar compounds of the frying oil P using the PC sensor 41, a difference is found in the correlation of the acid value of the frying oil P relative to the total polar compounds of the frying oil P between the seventh oil type and the eighth oil type. Thus, using the equation in which the difference therebetween is considered (Equation (17) or Equation (18) in the case of the seventh oil type and Equation (19) or Equation (20) in the case of the eighth oil type) enables the calculation of the acid value of the frying oil P with higher accuracy.

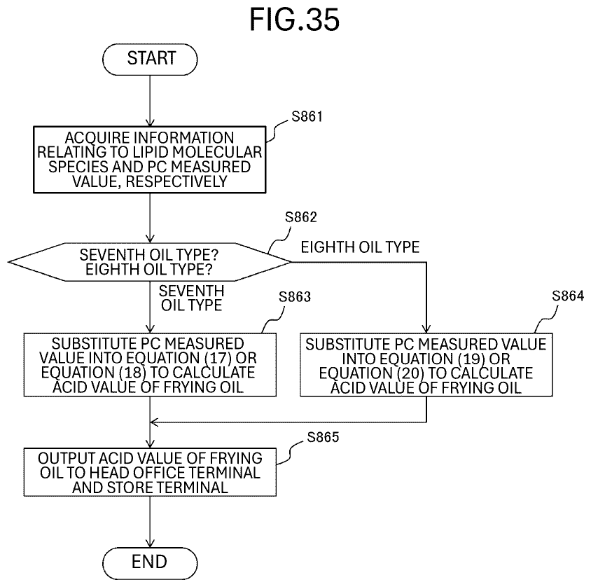

FIG. 35 illustrates a flowchart of a flow of the processing to be executed by the cloud 8C according to the seventh embodiment.

In the cloud 8C according to the present embodiment, firstly, the data acquisition section 81C acquires the measured value of the total polar compounds of the frying oil P output from the PC sensor 41 and the information relating to the lipid molecular species in the frying oil P output from the store terminal 6 (step S861; data acquisition step).

The information relating to the lipid molecular species in the frying oil P includes, for example, the information indicative of the specific names of oil types, and the like. The information relating to the lipid molecular species in the frying oil P does not necessarily have to be output from the store terminal 6 to the cloud 8C, but may be output from, for example, the head office terminal 7 to the cloud 8C.

Next, the oil type identification section 87 identifies whether the type of the frying oil P is the seventh oil type or the eighth oil type based on the information relating to the lipid molecular species in the frying oil P acquired in step S861 (step S862; oil type identification step).

In the case where it is identified in step S862 that the type of the frying oil P is the seventh oil type (step S862/seventh oil type), the deterioration indicator calculation section 83C substitutes the measured value of the total polar compounds of the frying oil P acquired in step S861 into PCn of Equation (17) or PCn of Equation (18), which is stored in the storage section 82C, to calculate the acid value AVn of the frying oil P (step S863; deterioration indicator calculation step).

On the other hand, in the case where it is identified in step S862 that the type of the frying oil P is the eighth oil type (step S862/eighth oil type), the deterioration indicator calculation section 83C substitutes the measured value of the total polar compounds of the frying oil P acquired in step S861 into PCn of Equation (19) or PCn of Equation (20), which is stored in the storage section 82C, to calculate the acid value AVn of the frying oil P (step S864; deterioration indicator calculation step).

Then, the detection result output section 84 outputs the acid value AVn of the frying oil P calculated in step S863 or step S864 to the store terminal 6 and the head office terminal 7, respectively, as the result of detection of the deterioration degree of the frying oil P (step S865; detection result output step), whereby the processing in the cloud 8C is ended.

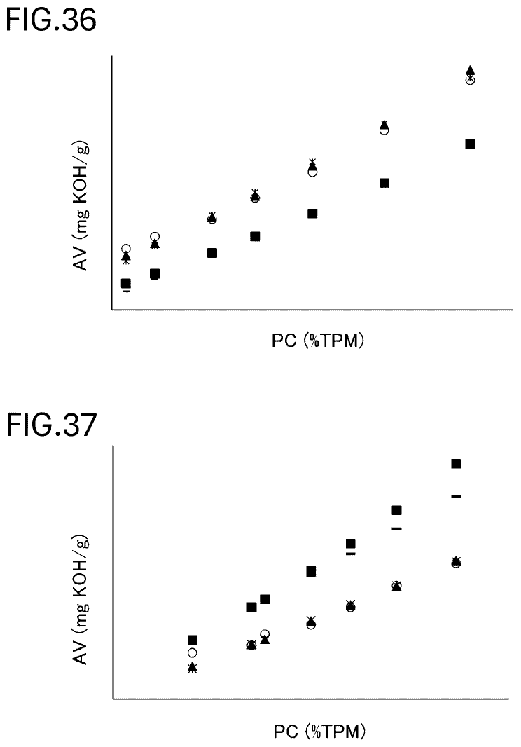

FIG. 36 illustrates a graph showing the correlation of the acid value of the frying oil P relating to the seventh oil type, which is classified based on the MG content in new oil, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 37 illustrates a graph showing the correlation of the acid value of the frying oil P relating to the eighth oil type, which is classified based on the MG content in new oil, relative to the total polar compounds (measured using the PC sensor 41) contained in the frying oil P, in which the actually measured acid values are compared with the acid values calculated using Equation (1), Equation (2), Equation (19), and Equation (20), respectively.

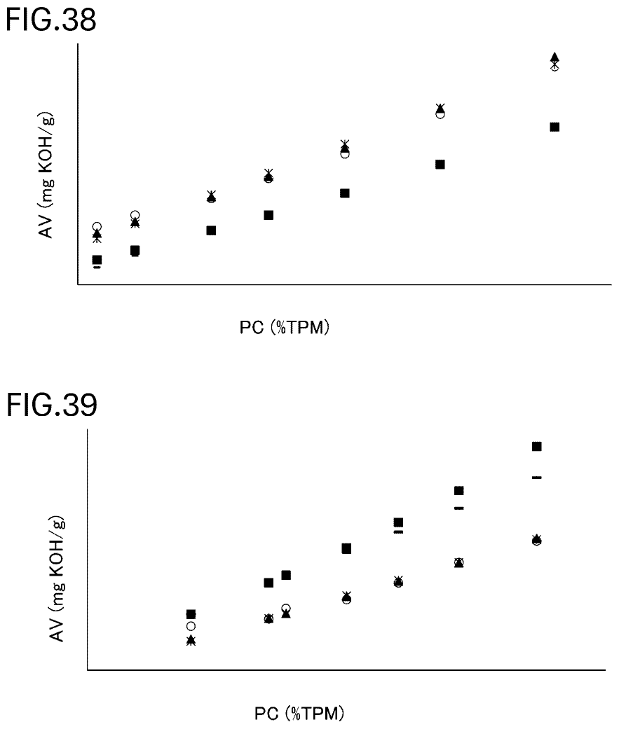

FIG. 38 illustrates a graph showing the correlation of the acid value of the frying oil P relating to the seventh oil type, which is classified based on the FFA content in new oil, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 39 illustrates a graph showing the correlation of the acid value of the frying oil P relating to the eighth oil type, which is classified based on the FFA content in new oil, relative to the total polar compounds (measured using the PC sensor 41) contained in the frying oil P, in which the actually measured acid values are compared with the acid values calculated using Equation (1), Equation (2), Equation (19), and Equation (20), respectively.

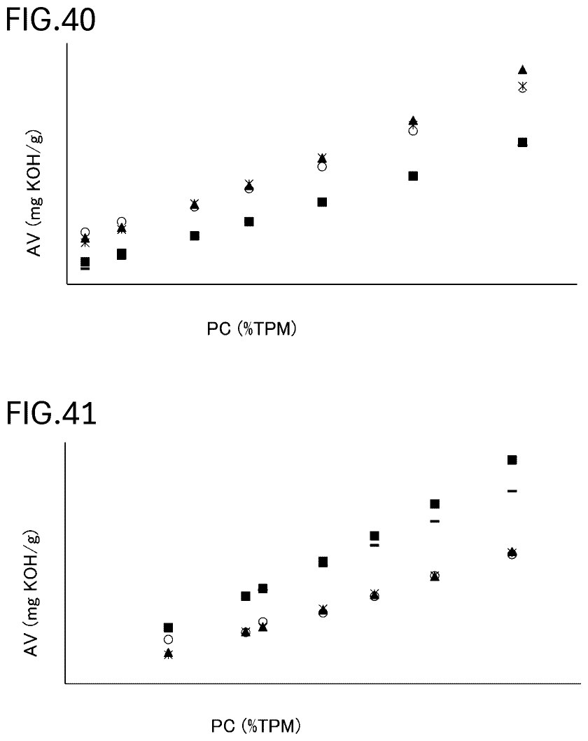

FIG. 40 illustrates a graph showing the correlation of the acid value of the frying oil P relating to the seventh oil type, which is classified based on the MG content in heated oil, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 41 illustrates a graph showing the correlation of the acid value of the frying oil P relating to the eighth oil type, which is classified based on the MG content in heated oil, relative to the total polar compounds (measured using the PC sensor 41) contained in the frying oil P, in which the actually measured acid values are compared with the acid values calculated using Equation (1), Equation (2), Equation (19), and Equation (20), respectively.

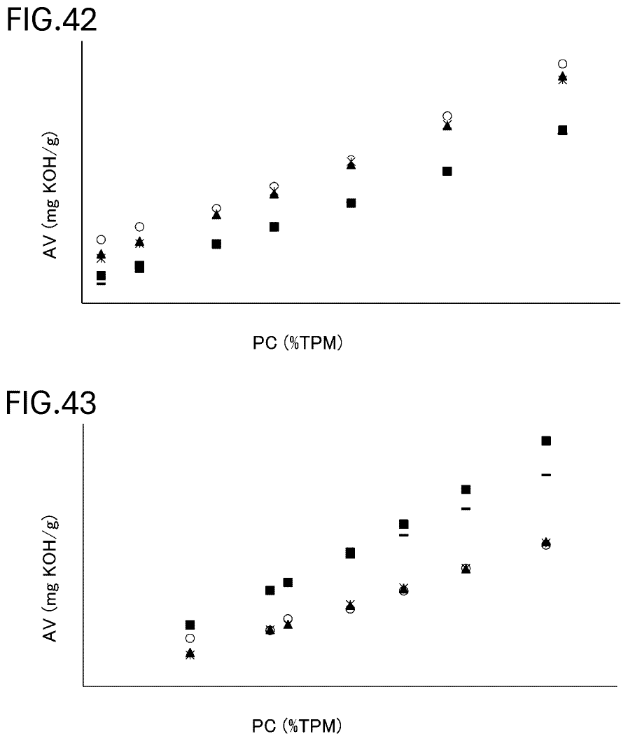

FIG. 42 illustrates a graph showing the correlation of the acid value of the frying oil P relating to the seventh oil type, which is classified based on the TG content in heated oil, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 43 illustrates a graph showing the correlation of the acid value of the frying oil P relating to the eighth oil type, which is classified based on the TG content in heated oil, relative to the total polar compounds (measured using the PC sensor 41) contained in the frying oil P, in which the actually measured acid values are compared with the acid values calculated using Equation (1), Equation (2), Equation (19), and Equation (20), respectively.

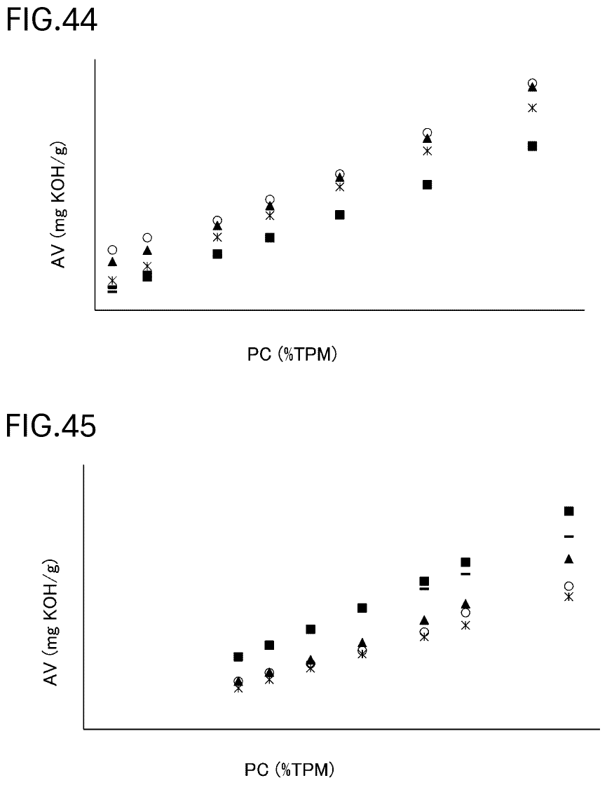

FIG. 44 illustrates a graph showing the correlation of the acid value of the frying oil P relating to the seventh oil type, which is classified based on the rate of increase in the DG content due to heating, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 45 illustrates a graph showing the correlation of the acid value of the frying oil P relating to the eighth oil type, which is classified based on the rate of increase in the DG content due to heating, relative to the total polar compounds (measured using the PC sensor 41) contained in the frying oil P, in which the actually measured acid values are compared with the acid values calculated using Equation (1), Equation (2), Equation (19), and Equation (20), respectively.

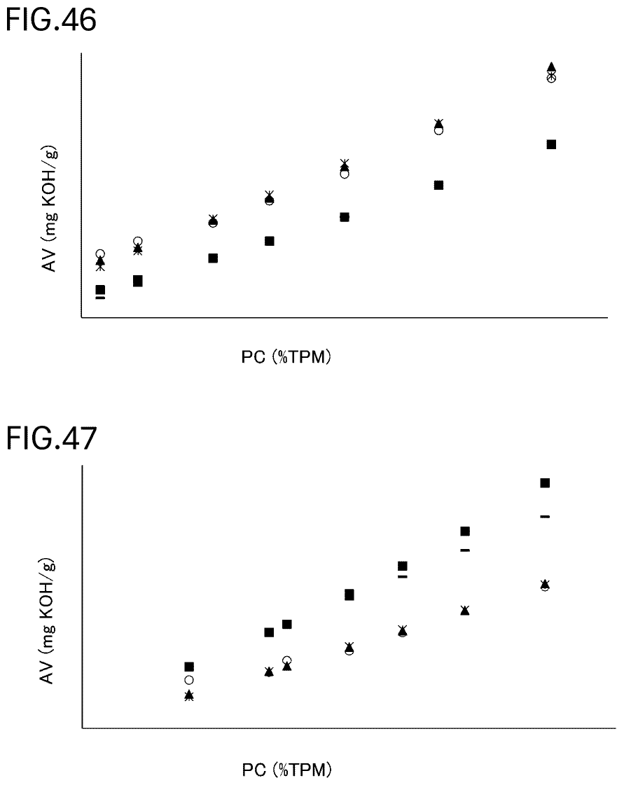

FIG. 46 illustrates a graph showing the correlation of the acid value of the frying oil P relating to the seventh oil type, which is classified based on the rate of increase in the FFA content due to heating, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 47 illustrates a graph showing the correlation of the acid value of the frying oil P relating to the eighth oil type, which is classified based on the rate of increase in the FFA content due to heating, relative to the total polar compounds (measured using the PC sensor 41) contained in the frying oil P, in which the actually measured acid values are compared with the acid values calculated using Equation (1), Equation (2), Equation (19), and Equation (20), respectively.

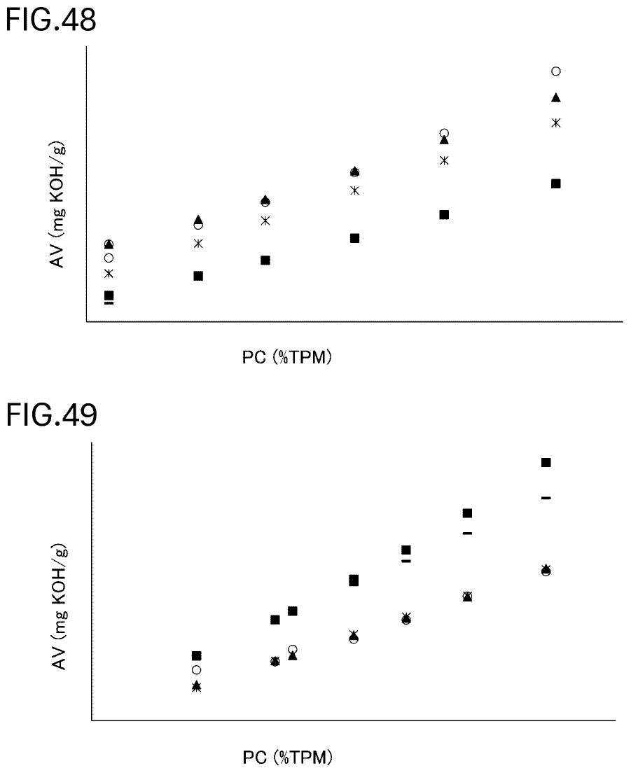

FIG. 48 illustrates a graph showing the correlation of the acid value of the frying oil P relating to the seventh oil type, which is classified based on the rate of decrease in the TG content due to heating, relative to the total polar compounds (measured using the PC sensor 41) contained in the frying oil P, in which the actually measured acid values are compared with the acid values calculated using Equation (1), Equation (2), Equation (17), and Equation (18), respectively. FIG. 49 illustrates a graph showing the correlation of the acid value of the frying oil P relating to the eighth oil type, which is classified based on the rate of decrease in the TG content due to heating, relative to the total polar compounds (measured using the PC sensor 41) contained in the frying oil P, in which the actually measured acid values are compared with the acid values calculated using Equation (1), Equation (2), Equation (19), and Equation (20), respectively.

In each of FIG. 36, FIG. 38, FIG. 40, FIG. 42, FIG. 44, FIG. 46, and FIG. 48 which relates to the case where the type of the frying oil P is the seventh oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (17), and a graph including a plurality of “▴” is indicative of the correlation graph for Equation (18), respectively.

In each of FIG. 37, FIG. 39, FIG. 41, FIG. 43, FIG. 45, FIG. 47, and FIG. 49 which relates to the case where the type of the frying oil P is the eighth oil type, a graph including a plurality of “∘” is indicative of the correlation graph for the actually measured acid value of the frying oil P, a graph including a plurality of “-” is indicative of the correlation graph for Equation (1), a graph including a plurality of “▪” is indicative of the correlation graph for Equation (2), a graph including a plurality of “*” is indicative of the correlation graph for Equation (19), and a graph including a plurality of “▴” is indicative of the correlation graph for Equation (20), respectively.

As illustrated in each of FIG. 36, FIG. 38, FIG. 40, FIG. 42, FIG. 44, FIG. 46, and FIG. 48, the correlation graph for Equation (17) and the correlation graph for Equation (18) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (17) and the correlation graph for Equation (18).

In the same manner, as illustrated in each of FIG. 37, FIG. 39, FIG. 41, FIG. 43, FIG. 45, FIG. 47, and FIG. 49, the correlation graph for Equation (19) and the correlation graph for Equation (20) are positioned closer to the correlation graph for the actually measured acid value of the frying oil P than the correlation graph for Equation (1) and the correlation graph for Equation (2). In other words, the correlation graph for Equation (1) and the correlation graph for Equation (2) are deviated from the correlation graph for the actually measured acid value of the frying oil P further than the correlation graph for Equation (19) and the correlation graph for Equation (20).

Thus, in the case where the PC sensor 41 measures the total polar compounds of the frying oil P, the cloud 8C identifies whether the type of the frying oil P is the seventh oil type or the eighth oil type based on the lipid molecular species of the frying oil P and calculates the acid value of the frying oil P using the correlation equation for the oil type as identified. This enables the calculation of the acid value of the frying oil P with higher accuracy than the case of calculating it using Equation (1) or Equation (2) in all cases without considering the type of oil classified based on the lipid molecular species of the frying oil P.

### Eighth Embodiment

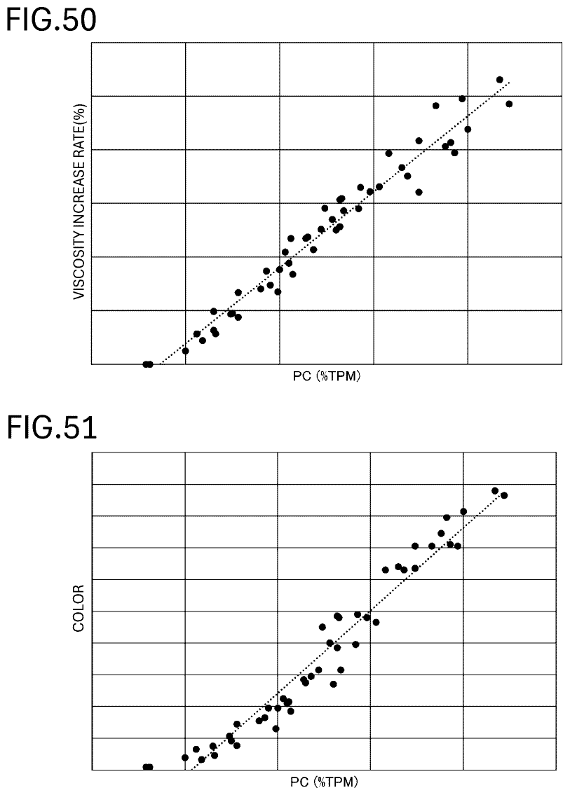

Next, a cloud 9 according to the eighth embodiment will be described with reference to FIG. 50 to FIG. 73.

In the first to seventh embodiments, the present invention has been described with the example of the correlation of the acid value of the frying oil P relative to the total polar compounds contained in the frying oil P.

In the eighth embodiment and thereafter, the present invention will be described with the examples of the correlations other than the above. The cloud according to the eighth embodiment and those according to the embodiments thereafter are provided with the reference number “9” and the reference numbers derived from the reference number “9”, respectively, and the hardware configurations thereof will not be described in detail as they are the same as those according to the first to seventh embodiments.

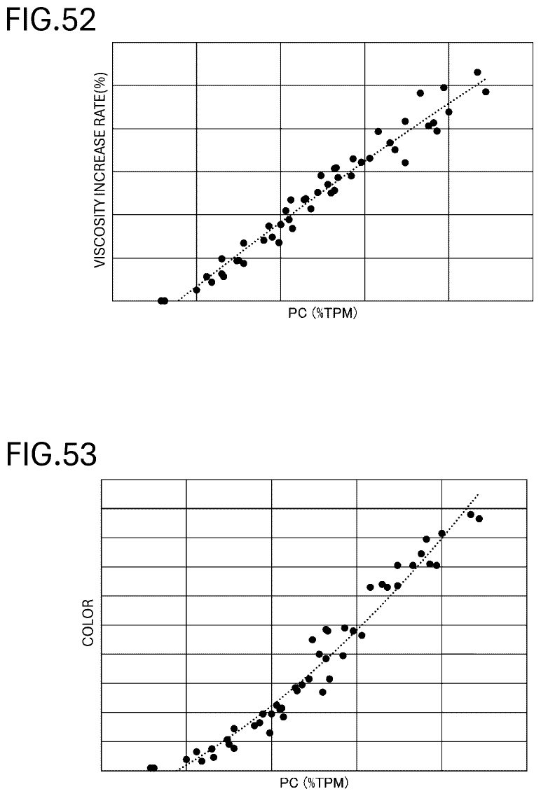

FIG. 50 illustrates a graph of a linear function showing the correlation of the viscosity increase rate of the frying oil P relative to the total polar compounds contained in the frying oil P. FIG. 51 illustrates a graph of a linear function showing the correlation of the color of the frying oil P relative to the total polar compounds contained in the frying oil P. FIG. 52 illustrates a graph of a quadratic function showing the correlation of the viscosity increase rate of the frying oil P relative to the total polar compounds contained in the frying oil P. FIG. 53 illustrates a graph of a quadratic function showing the correlation of the color of the frying oil P relative to the total polar compounds contained in the frying oil P.

The total polar compounds contained in the frying oil P and the rate of increase in the viscosity of the frying oil P are correlated with each other as illustrated in the graphs of FIG. 50 and FIG. 52. Specifically, as the total polar compounds contained in the frying oil P increase, the viscosity increase rate of the frying oil P increases.

That is, the correlation of the viscosity increase rate of the frying oil P relative to the total polar compounds contained in the frying oil P can be expressed with a correlation equation in the form of a polynomial in which the viscosity increase rate of the frying oil P is expressed with the total polar compounds.

Furthermore, the total polar compounds contained in the frying oil P and the color (practically, a numerical value indicative of the darkness of color, which applies below in the same manner) of the frying oil P are correlated with each other as illustrated in the graphs of FIG. 51 and FIG. 53. Specifically, as the total polar compounds contained in the frying oil P increase, the color of the frying oil P increases (the frying oil P darkens in color). That is, the correlation of the color of the frying oil P relative to the total polar compounds contained in the frying oil P can be expressed with a correlation equation in the form of a polynomial in which the color of the frying oil P is expressed with the total polar compounds.

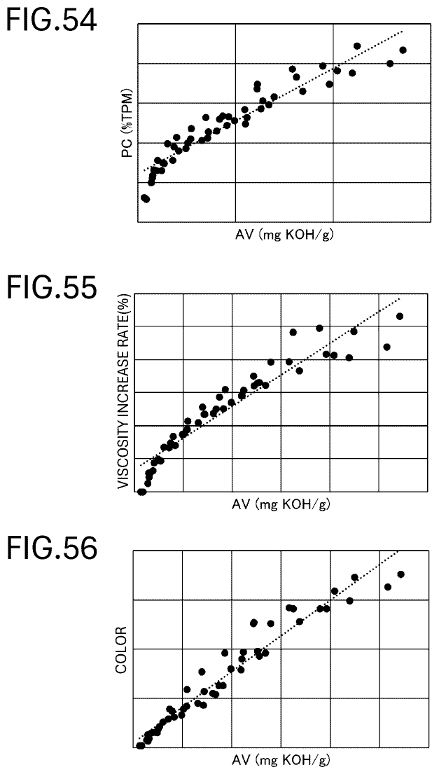

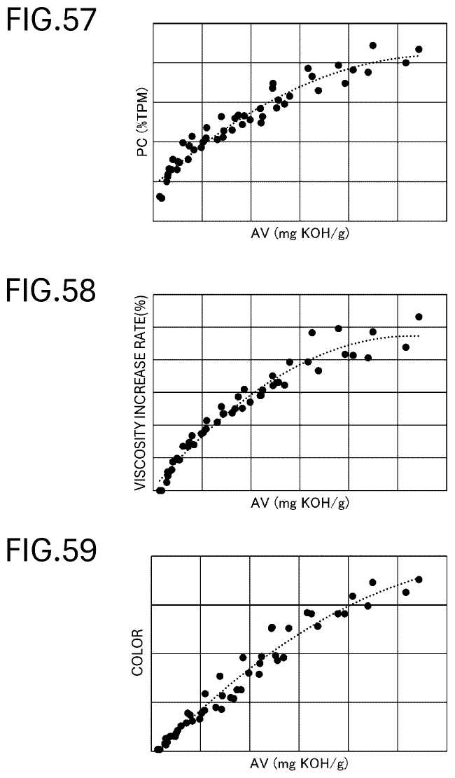

FIG. 54 illustrates a graph of a linear function showing the correlation of the total polar compounds contained in the frying oil P relative to the acid value of the frying oil P. FIG. 55 illustrates a graph of a linear function showing the correlation of the viscosity increase rate of the frying oil P relative to the acid value of the frying oil P. FIG. 56 illustrates a graph of a linear function showing the correlation of the color of the frying oil P relative to the acid value of the frying oil P. FIG. 57 illustrates a graph of a quadratic function showing the correlation of the total polar compounds contained in the frying oil P relative to the acid value of the frying oil P. FIG. 58 illustrates a graph of a quadratic function showing the correlation of the viscosity increase rate of the frying oil P relative to the acid value of the frying oil P. FIG. 59 illustrates a graph of a quadratic function showing the correlation of the color of the frying oil P relative to the acid value of the frying oil P.

The acid value of the frying oil P and the total polar compounds contained in the frying oil P are correlated with each other as illustrated in the graphs of FIG. 54 and FIG. 57. Specifically, as the acid value of the frying oil P increases, the total polar compounds contained in the frying oil P increase. That is, the correlation of the total polar compounds contained in the frying oil P relative to the acid value of the frying oil P can be expressed with a correlation equation in the form of a polynomial in which the total polar compounds contained in the frying oil P are expressed with the acid value.

The acid value of the frying oil P and the viscosity increase rate of the frying oil P are correlated with each other as illustrated in the graphs of FIG. 55 and FIG. 58. Specifically, as the acid value of the frying oil P increases, the viscosity increase rate of the frying oil P increases. That is, the correlation of the viscosity increase rate of the frying oil P relative to the acid value of the frying oil P can be expressed with a correlation equation in the form of a polynomial in which the viscosity increase rate of the frying oil P is expressed with the acid value.

Still further, the acid value of the frying oil P and the color of the frying oil P are correlated with each other as illustrated in the graphs of FIG. 56 and FIG. 59. Specifically, as the acid value of the frying oil P increases, a numerical value relating to the color of the frying oil P increases. That is, the correlation of the color of the frying oil P relative to the acid value of the frying oil P can be expressed with a correlation equation in the form of a polynomial in which the color of the frying oil P is expressed with the acid value.

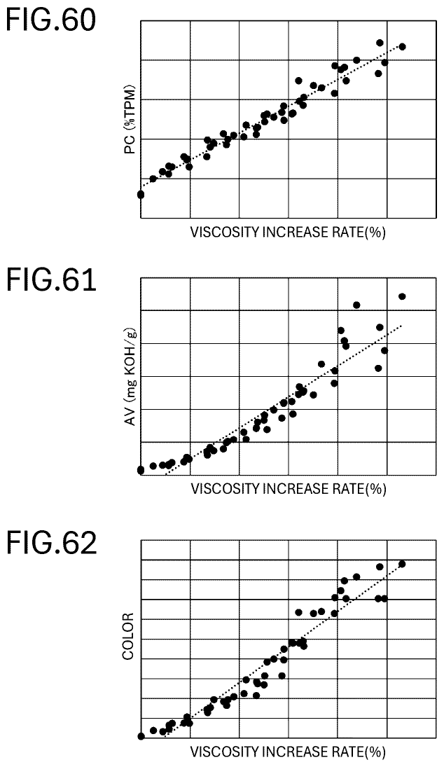

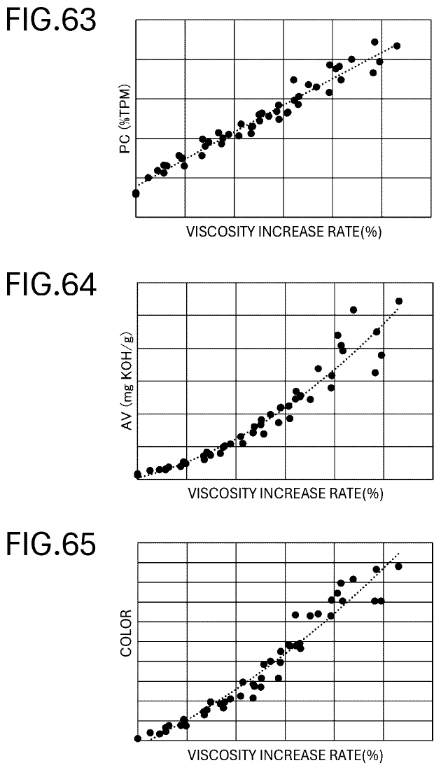

FIG. 60 illustrates a graph of a linear function showing the correlation of the total polar compounds contained in the frying oil P relative to the viscosity increase rate of the frying oil P. FIG. 61 illustrates a graph of a linear function showing the correlation of the acid value of the frying oil P relative to the viscosity increase rate of the frying oil P. FIG. 62 illustrates a graph of a linear function showing the correlation of the color of the frying oil P relative to the viscosity increase rate of the frying oil P. FIG. 63 illustrates a graph of a quadratic function showing the correlation of the total polar compounds contained in the frying oil P relative to the viscosity increase rate of the frying oil P. FIG. 64 illustrates a graph of a quadratic function showing the correlation of the acid value of the frying oil P relative to the viscosity increase rate of the frying oil P. FIG. 65 illustrates a graph of a quadratic function showing the correlation of the color of the frying oil P relative to the viscosity increase rate of the frying oil P.

The viscosity increase rate of the frying oil P and the total polar compounds contained in the frying oil P are correlated with each other as illustrated in the graphs of FIG. 60 and FIG. 63. Specifically, as the viscosity increase rate of the frying oil P increases, the total polar compounds contained in the frying oil P increase. That is, the correlation of the total polar compounds contained in the frying oil P relative to the viscosity increase rate of the frying oil P can be expressed with a correlation equation in the form of a polynomial in which the total polar compounds contained in the frying oil P are expressed with the viscosity increase rate.

Furthermore, the viscosity increase rate of the frying oil P and the acid value of the frying oil P are correlated with each other as illustrated in the graphs of FIG. 61 and FIG. 64. Specifically, as the viscosity increase rate of the frying oil P increases, the acid value of the frying oil P increases. That is, the correlation of the acid value of the frying oil P relative to the viscosity increase rate of the frying oil P can be expressed with a correlation equation in the form of a polynomial in which the acid value of the frying oil is expressed with the viscosity increase rate.

Still further, the viscosity increase rate of the frying oil P and the color of the frying oil P are correlated with each other as illustrated in the graphs of FIG. 62 and FIG. 65. Specifically, as the viscosity increase rate of the frying oil P increases, a numerical value relating to the color of the frying oil P increases. That is, the correlation of the color of the frying oil P relative to the viscosity increase rate of the frying oil P can be expressed with a correlation equation in the form of a polynomial in which the color of the frying oil P is expressed with the viscosity increase rate.

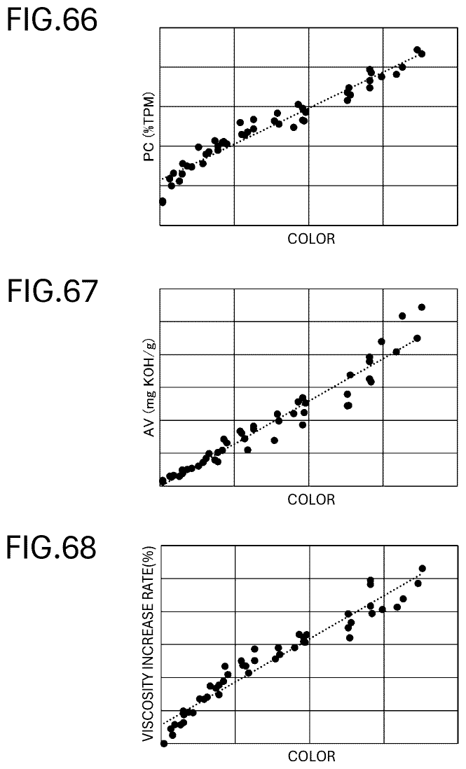

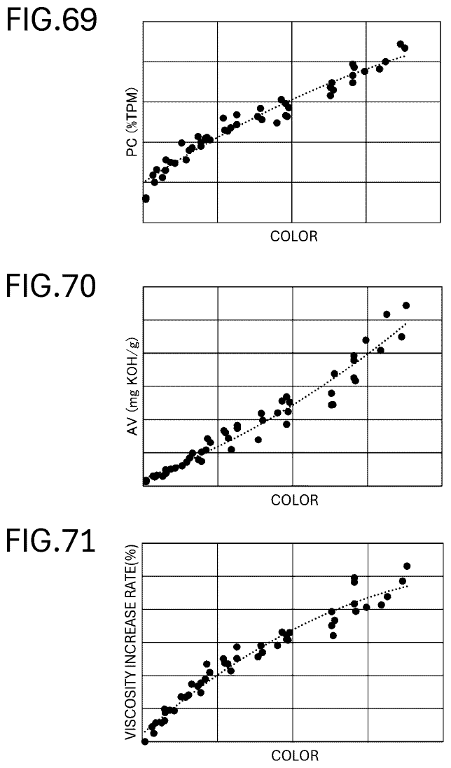

FIG. 66 illustrates a graph of a linear function showing the correlation of the total polar compounds contained in the frying oil P relative to the color of the frying oil P. FIG. 67 illustrates a graph of a linear function showing the correlation of the acid value of the frying oil P relative to the color of the frying oil P. FIG. 68 illustrates a graph of a linear function showing the correlation of the viscosity increase rate of the frying oil P relative to the color of the frying oil P. FIG. 69 illustrates a graph of a quadratic function showing the correlation of the total polar compounds contained in the frying oil P relative to the color of the frying oil P. FIG. 70 illustrates a graph of a quadratic function showing the correlation of the acid value of the frying oil P relative to the color of the frying oil P. FIG. 71 illustrates a graph of a quadratic function showing the correlation of the viscosity increase rate of the frying oil P relative to the color of the frying oil P.

The color of the frying oil P and the total polar compounds contained in the frying oil P are correlated with each other as illustrated in the graphs of FIG. 66 and FIG. 69. Specifically, as a numerical value relating to the color of the frying oil P increases, the total polar compounds contained in the frying oil P increase. That is, the correlation of the total polar compounds contained in the frying oil P relative to the color of the frying oil P can be expressed with a correlation equation in the form of a polynomial in which the total polar compounds contained in the frying oil P are expressed with the color.

Furthermore, the color of the frying oil P and the acid value of the frying oil P are correlated with each other as illustrated in the graphs of FIG. 67 and FIG. 70. Specifically, as a numerical value relating to the color of the frying oil P increases, the acid value of the frying oil P increases. That is, the correlation of the acid value of the frying oil P relative to the color of the frying oil P can be expressed with a correlation equation in the form of a polynomial in which the acid value of the frying oil is expressed with the color.

Still further, the color of the frying oil P and the viscosity increase rate of the frying oil P are correlated with each other as illustrated in the graphs of FIG. 68 and FIG. 71. Specifically, as a numerical value relating to the color of the frying oil P increases, the viscosity increase rate of the frying oil P increases. That is, the correlation of the viscosity increase rate of the frying oil P relative to the color of the frying oil P can be expressed with a correlation equation in the form of a polynomial in which the viscosity increase rate of the frying oil P is expressed with the color.

Here, each of the acid value, total polar materials, color, and viscosity increase rate of the frying oil P corresponds to a first deterioration indicator. The first deterioration indicator is defined based on the a substance produced by heating the frying oil P, namely, the fat and oil. The acid value of the frying oil P is a value corresponding to the free fatty acid produced by heating the frying oil P. The total polar compounds in the frying oil P are the ratio of the polar compounds produced by heating the frying oil P relative to the oil and fat. The color (depth of color) of the frying oil P is a value that varies depending the oxides, polymers, and eluates from the deep-frying material Q and reaction products thereof, which are produced by heating the frying oil P. The viscosity of the frying oil P varies depending on the progress of the polymerization reaction caused by heating the frying oil P and the eluates from the deep-frying material Q, and the rate of increase in viscosity, expressed as a percentage based on the viscosity of new oil.

Where the first deterioration indicator is Di1, a second deterioration indicator, which is a deterioration indicator other than the first deterioration indicator, is Di2, and the arbitrary heating time of the frying oil P is n, the correlation equation for the second deterioration indicator of the frying oil P relative to the first deterioration indicator of the frying oil P at the arbitrary heating time n is expressed with a linear equation of the following Equation (31) or a quadratic equation of the following Equation (32).

$$Di 2 = α \times ( Di 1 n ) + β \qquad ( 31 )$$

$$Di 2 = γ \times {( Di 1 n )}^{2} + δ \times ( Di 1 n ) + ε \qquad ( 32 )$$

Equation (31) is a correlation equation for each of the correlation graphs illustrated in FIG. 50, FIG. 51, FIG. 54 to FIG. 56, FIG. 60 to FIG. 62, and FIG. 66 to FIG. 68, and Equation (32) is a correlation equation for each of the correlation graphs illustrated in FIG. 52, FIG. 53, FIG. 57 to FIG. 59, FIG. 63 to FIG. 65, and FIG. 69 to FIG. 71. By substituting a measured value relating to the first deterioration indicator of the frying oil P at the arbitrary heating time n into Di1n of each of Equation (31) and Equation (32), the second deterioration indicator Di2n of the frying oil P at the arbitrary heating time n can be calculated.

In the following, various kinds of measurement device for measuring the acid value, total polar materials, color, and viscosity increase rate of the frying oil P, which are the first deterioration indicators, respectively, are collectively referred to as “measurement device 4”. For example, in the case where the first deterioration indicator is the total polar compounds, the measurement device 4 is the PC sensor 42, and in the case where the first deterioration indicator is the color, the measurement device 4 is the camera 42.

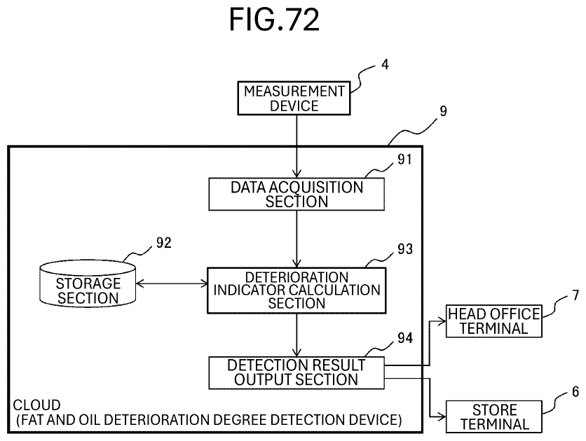

Next, a functional configuration of the cloud 9 according to the eighth embodiment will be described with reference to FIG. 72.

FIG. 72 is a functional block diagram illustrating functions provided in the cloud 9 according to the eighth embodiment.

As illustrated in FIG. 72, the cloud 9 includes a data acquisition section 91, a storage section 92, a deterioration indicator calculation section 93, and a detection result output section 94.

The data acquisition section 91 is configured to acquire a measured value of the first deterioration indicator of the frying oil P output from the measurement device 4.

The storage section 92 retains the correlation equation of the second deterioration indicator of the frying oil P relative to the first deterioration indicator which has been described above, specifically, Equation (31) or Equation (32). The storage section 92 may retain both Equation (31) and Equation (32), or only one of Equation (31) and Equation (32).

The deterioration indicator calculation section 93 is configured to calculate the second deterioration indicator of the frying oil P based on the measured value of the first deterioration indicator of the frying oil P acquired by the data acquisition section 91 and the correlation equation for the second deterioration indicator of the frying oil P relative to the first deterioration indicator of the frying oil P read from the storage section 92.

Specifically, the deterioration indicator calculation section 93 substitutes the measured value of the first deterioration indicator of the frying oil P acquired by the data acquisition section 91 into Di1n of Equation (31) or Equation (32) read out from the storage section 92 to calculate the second deterioration indicator Di2n of the frying oil P.

In the case where both Equations (31) and (32) are stored in the storage section 92, the deterioration indicator calculation section 93 selects one of Equations (31) and (32), substitutes the measured value of the first deterioration indicator of the frying oil P into Di1n of the selected equation to calculate the second deterioration indicator of the frying oil P.

The detection result output section 94 is configured to output the second deterioration indicator of the frying oil P calculated by the deterioration indicator calculation section 93 to each of the store terminal 6 and the head office terminal 7 as the result of detection of the deterioration degree of the frying oil P. In the present embodiment, the detection result output section 94 outputs the result of detection of the deterioration degree of the frying oil P to both the store terminal 6 and the head office terminal 7, respectively, however, may output it to only one of the store terminal 6 and the head office terminal 7.

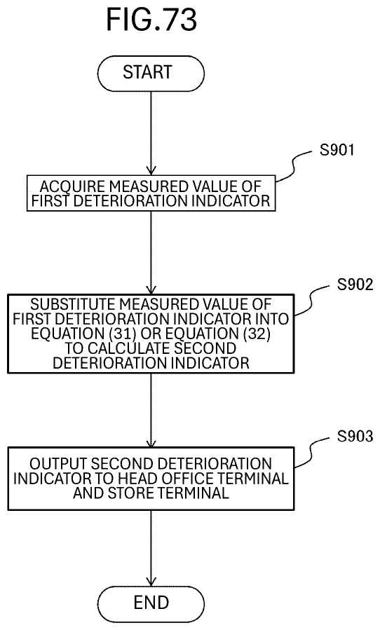

Next, a flow of the processing to be executed in the cloud 9 will be described with reference to FIG. 73.

FIG. 73 illustrates a flowchart of a flow of the processing to be executed in the cloud 9 according to the eighth embodiment.

As illustrated in FIG. 73, in the cloud 9, firstly, the data acquisition section 91 acquires a measured value of the first deterioration indicator of the frying oil P measured by the measurement device 4 in a measurement step (step S901; data acquisition step).

Next, the deterioration indicator calculation section 93 substitutes the measured value of the first deterioration indicator of the frying oil P acquired in step S901 into Di1n of the correlation equation for the second deterioration indicator of the frying oil P relative to the first deterioration indicator of the frying oil P stored in the storage section 92, that is, Equation (31) or Equation (32), so as to calculate the second deterioration indicator Di2n of the frying oil P (step S902; deterioration indicator calculation step).

Then, the detection result output section 94 outputs the second deterioration indicator Di2n of the frying oil P calculated in step S902 to the store terminal 6 and the head office terminal 7, respectively, as the result of detection of the deterioration degree of the frying oil P (step S903; detection result output step), whereby the processing in the cloud 9 is ended.

As described above, the cloud 9 enables conversion of the second deterioration indicator of the frying oil P into the first deterioration indicator of the frying oil P using the correlation equation for the second deterioration indicator of the frying oil P relative to the first deterioration indicator of the frying oil P. This enables a staff of a store to obtain a result of detection of the deterioration degree of the frying oil P, which is expressed with the second deterioration indicator different from the first deterioration indicator, merely by measuring the first deterioration indicator of the frying oil P using the measurement device 4, and thus, depending on the situation in the cooking area 1 or in response to the demands from the head office detect various deterioration indicators indicative of the deterioration degree of the frying oil P in a simple manner and with high accuracy.

### Ninth Embodiment

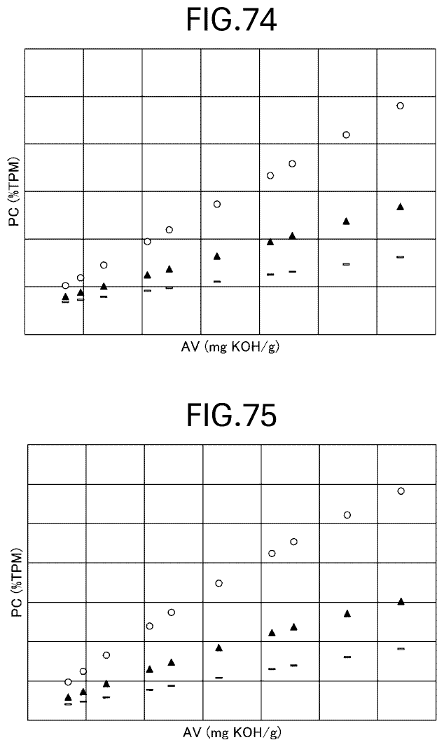

Next, a cloud 9A according to the ninth embodiment of the present invention will be described with reference to FIG. 74 to FIG. 81.

In the present embodiment, the coefficient and constant included in the correlation equation for the second deterioration indicator of the frying oil P relative to the first deterioration indicator of the frying oil P are set to values corresponding to a weight W per unit heating time (hereinafter, referred to as “deep-frying weight W per unit time”) of the deep-frying material Q (ingredient) to be deep fried using the frying oil P.

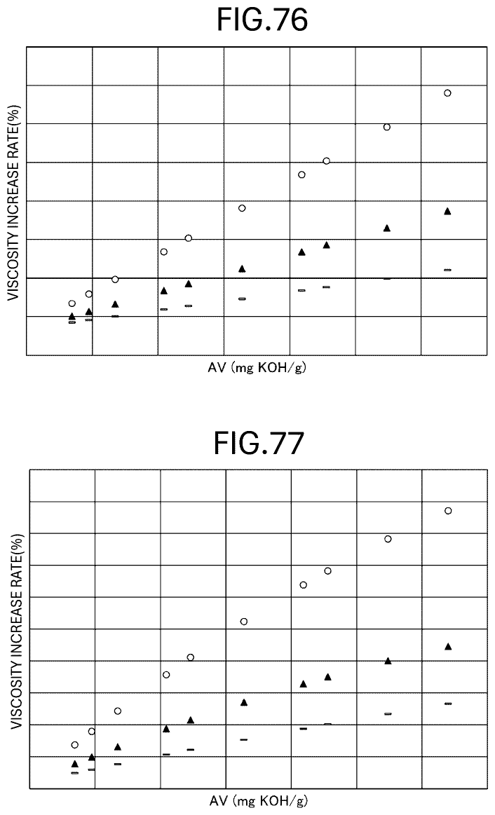

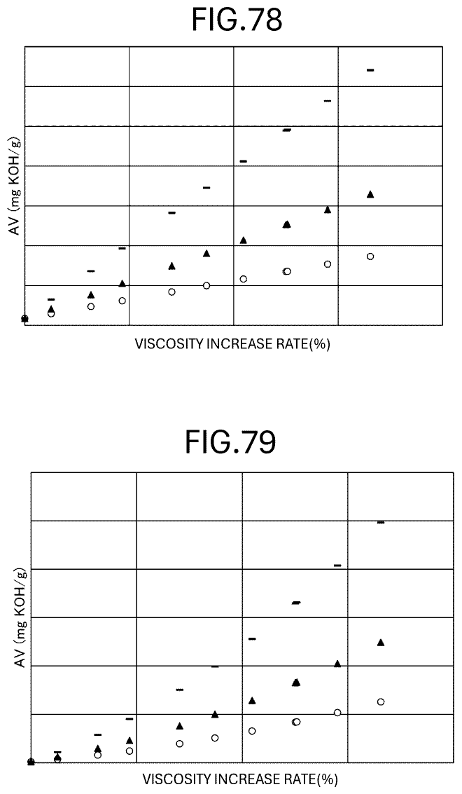

FIG. 74 illustrates a graph of a linear function showing the correlation of the total polar compounds contained in the frying oil P relative to the acid value of the frying oil P, in which the deep-frying weight W per unit time is considered. FIG. 75 illustrates a graph of a quadratic function showing the correlation of the total polar compounds contained in the frying oil P relative to the acid value of the frying oil P, in which the deep-frying weight W per unit time is considered. FIG. 76 illustrates a graph of a linear function showing the correlation of the viscosity increase rate of the frying oil P relative to the acid value of the frying oil P, in which the deep-frying weight W per unit time is considered. FIG. 77 illustrates a graph of a quadratic function showing the correlation of the viscosity increase rate of the frying oil P relative to the acid value of the frying oil P, in which the deep-frying weight W per unit time is considered. FIG. 78 illustrates a graph of a linear function showing the correlation of the acid value of the frying oil P relative to the viscosity increase rate of the frying oil P, in which the deep-frying weight W per unit time is considered. FIG. 79 illustrates a graph of a quadratic function showing the correlation of the acid value of the frying oil P relative to the viscosity increase rate of the frying oil P, in which the deep-frying weight W per unit time is considered.

As illustrated in each of FIG. 74 and FIG. 75, the slope of the graph showing the correlation of the total polar compounds of the frying oil P relative to the acid value of the frying oil P varies depending on the deep-frying weight W per unit time of the deep-frying material Q to be cooked using the frying oil P.

In the same manner, as illustrated in each of FIG. 76 and FIG. 77, the slope of the graph showing the correlation of the viscosity increase rate of the frying oil P relative to the acid value of the frying oil P varies depending on the deep-frying weight W per unit time of the deep-frying material Q to be cooked using the frying oil P.

In the same manner, as illustrated in each of FIG. 78 and FIG. 79, the slope of the graph showing the correlation of the acid value of the frying oil P relative to the viscosity increase rate of the frying oil P varies depending on the deep-frying weight W per unit time of the deep-frying material Q to be cooked using the frying oil P.

Accordingly, the first-order coefficient α of Di1n in Equation (31) and the second-order coefficient γ and the first-order coefficient δ of Di1n in Equation (32) are set to values corresponding to the deep-frying weight W per unit time of the deep-frying material Q to be cooked using the frying oil P, respectively.

Here, the deep-frying weight W of the deep-frying material Q per unit time at a store can also be calculated based on the sales per day at the store in which the frying oil P is used, and thus values corresponding to the sales per day at the store in which the frying oil P is used may be employed for the first-order coefficient α of Di1n in Equation (31) and the second-order coefficient γ and the first-order coefficient δ of Di1n in Equation (32). The sales per day at the store are obtained, for example, by calculating an average value per day based on the total sales at the store in the past year, or by calculating an average value per day based on the total sales at the store in a predetermined period (for example, every season). The sales per day at the store is preferably the amount obtained by extracting only the sales of the fried food cooked using the frying oil P.

In each of FIG. 74 to FIG. 79, the deep-frying weight W per unit time at a store is categorized into the three ranges of “large”, “medium”, and “small”, and a correlation graph including a plurality of “∘” is indicative of the case where the deep-frying weight W per unit time is less than 2,000 g (W<2000), a correlation graph including a plurality of “▴” is indicative of the case where the deep-frying weight W per unit time is equal to or more than 2,000 g and less than 12,000 g (20005W<12000), and a correlation graph including a plurality of “-” is indicative of the case where the deep-frying weight W per unit time is equal to or more than 12,000 g (W≥12000), respectively.

In each of FIG. 74 to FIG. 79, the slope of the correlation graph in the case where the deep-frying weight W per unit time is “large” is the smallest among those of the three correlation graphs. The slope of the correlation graph in the case where the deep-frying weight W per unit time is “medium” is greater than the slope of the correlation graph in the case where the deep-frying weight W per unit time is “large”. The slope of the correlation graph in the case where the deep-frying weight W per unit time is “small” is greater than the slope of the correlation graph in the case where the deep-frying weight W per unit time is “medium”, and is the greatest among those of the three correlation graphs.

Accordingly, in the correlation of the total polar compounds contained in the frying oil P relative to the acid value of the frying oil P and the correlation of the viscosity increase rate of the frying oil P relative to the acid value of the frying oil P (that is, each correlation shown in the correlation graph with the acid value on the horizontal axis), the first-order coefficient α of Di1n in Equation (31) and the second-order coefficient γ and the first-order coefficient δ of Di1n in Equation (32) are set to be greater, respectively, as the deep-frying weight W per unit time decreases.

On the other hand, in FIG. 78 and FIG. 79, the slope of the correlation graph in the case where the deep-frying weight W per unit time is “small” is the smallest among those of the three correlation graphs. The slope of the correlation graph in the case where the deep-frying weight W per unit time is “medium” is greater than the slope of the correlation graph in the case where the deep-frying weight W per unit time is “small”. The slope of the correlation graph in the case where the deep-frying weight W per unit time is “large” is greater than the slope of the correlation graph in the case where the deep-frying weight W per unit time is “medium”, and is the greatest among those of the three correlation graphs.

Accordingly, in the correlation of the acid value of the frying oil P relative to the viscosity increase rate of the frying oil P, the first-order coefficient α of Di1n in Equation (31) and the second-order coefficient γ and the first-order coefficient δ of Di1n in Equation (32) are set to be greater, respectively, as the deep-frying weight W per unit time increases.

The total polar compounds contained in the frying oil P and the viscosity increase rate increase as the heating time at the fryer 2 (the total heating time at the fryer 2, including the heating time in which the deep-frying material Q is deep fried and the empty heating time) increases, and the acid value of the frying oil P increases as the deep-frying weight of the deep-frying material Q increases, respectively. Accordingly, considering both the heating time at the fryer 2 and the deep-frying weight of the deep-frying material Q in the correlation of the total polar compounds contained in the frying oil P or the viscosity increase rate of the frying oil P relative to the acid value of the frying oil P enables improvement in the accuracy.

The “unit time” does not necessarily have to be one hour, and may be set to any time. The deep-frying weight W per unit time at a store does not necessarily have to be categorized based on the thresholds “2,000 g” and “12,000 g” used in the correlations illustrated in FIG. 74 and FIG. 79, and any value may be used as the thresholds for each store.

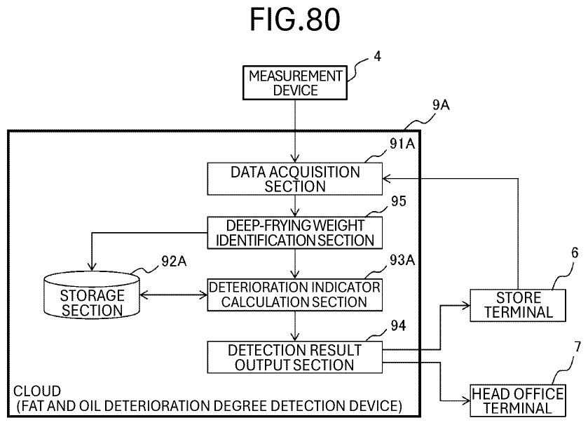

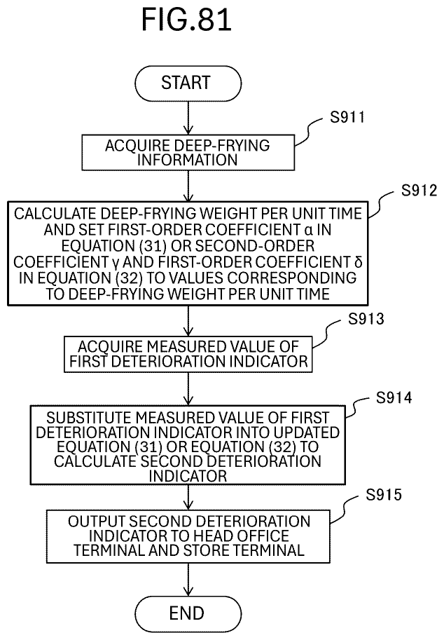

FIG. 80 is a functional block diagram illustrating functions provided in the cloud 9A according to the eighth embodiment. FIG. 81 illustrates a flowchart of a flow of the processing to be executed by the cloud 9A according to the eighth embodiment.

As illustrated in FIG. 80, the cloud 9A includes a data acquisition section 91A, a deep-frying weight identification section 95, a storage section 92A, a deterioration indicator calculation section 93A, and a detection result output section 94.

As illustrated in FIG. 81, in the cloud 9A, firstly, the data acquisition section 91A acquires the deep-frying information output from the store terminal 6 (step S911). The “deep-frying information” includes the heating time at the fryer 2 and the weight of the deep-frying material Q to be cooked in the fryer 2 (that is, the deep-frying weight of the deep-frying material Q).

Subsequently, the deep-frying weight identification section 95 calculates the deep-frying weight W per unit time based on the deep-frying information acquired in step S911 to determine which category the deep-frying weight W per unit time (in the present embodiment, an hour) at the store is included (step S912), and sets the first-order coefficient α of Di1n of Equation (31) or the second-order coefficient γ and the first-order coefficient δ of Di1n of Equation (32), which is stored in the storage section 92A, to values corresponding to the deep-frying weight W per unit time, respectively (step S912). Thus, Equation (31) or Equation (32) stored in the storage section 92A is updated.

Next, the data acquisition section 91A acquires a measured value of the first deterioration indicator of the frying oil P output from the measurement device 4 (step S913).

Next, the deterioration indicator calculation section 93A substitutes the measured value of the first deterioration indicator of the frying oil P acquired in step S913 into Di1 of Equation (31) or Equation (32) which has been updated in step S912, so as to calculate the second deterioration indicator Di2n of the frying oil P (step S914).

Then, the detection result output section 94 outputs the second deterioration indicator Di2n of the frying oil P calculated in step S914 to the store terminal 6 and the head office terminal 7, respectively, as the result of detection of the deterioration degree of the frying oil P (step S915), whereby the processing in the cloud 9A is ended.

In the present embodiment, the cloud 9A calculates the second deterioration indicator of the frying oil P using Equation (31) or Equation (32) which depends on the deep-frying weight W per unit time at a store, which enables the calculation of the second deterioration indicator of the frying oil P with higher accuracy than the case of calculating it using the predetermined Equation (31) or Equation (32).

### Tenth Embodiment

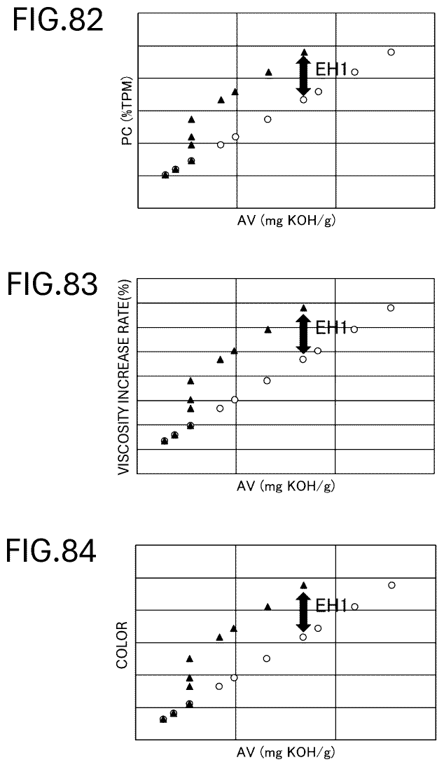

Next, a cloud 9B according to the tenth embodiment of the present invention will be described with reference to FIG. 82 to FIG. 93.

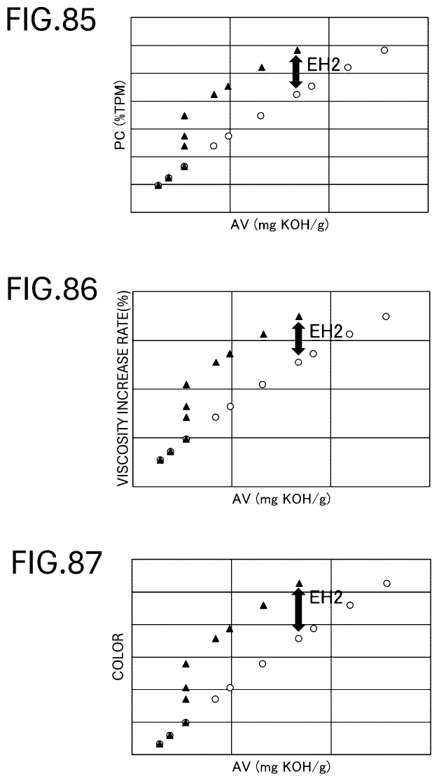

FIG. 82 illustrates a graph of a linear function showing the correlation of the total polar compounds contained in the frying oil P relative to the acid value of the frying oil P, which relates to whether empty heating has been performed. FIG. 83 illustrates a graph of a linear function showing the correlation of the viscosity increase rate of the frying oil P relative to the acid value of the frying oil P, which relates to whether empty heating has been performed. FIG. 84 illustrates a graph of a linear function showing the correlation of the color of the frying oil P relative to the acid value of the frying oil P, which relates to whether empty heating has been performed. FIG. 85 illustrates a graph of a quadratic function showing the correlation of the total polar compounds contained in the frying oil P relative to the acid value of the frying oil P, which relates to whether empty heating has been performed. FIG. 86 illustrates a graph of a quadratic function showing the correlation of the viscosity increase rate of the frying oil P relative to the acid value of the frying oil P, which relates to whether empty heating has been performed. FIG. 87 illustrates a graph of a quadratic function showing the correlation of the color of the frying oil P relative to the acid value of the frying oil P, which relates to whether empty heating has been performed.

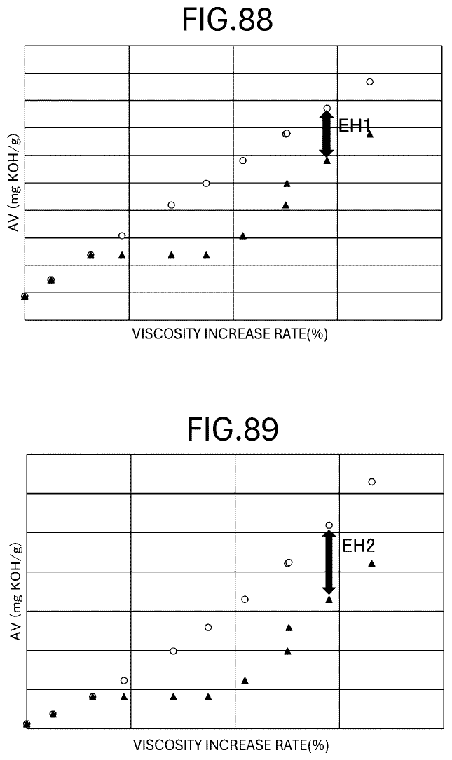

FIG. 88 illustrates a graph of a linear function showing the correlation of the acid value of the frying oil P relative to the viscosity increase rate of the frying oil P, which relates to whether empty heating has been performed. FIG. 89 illustrates a graph of a quadratic function showing the correlation of the acid value of the frying oil P relative to the viscosity increase rate of the frying oil P, which relates to whether empty heating has been performed.

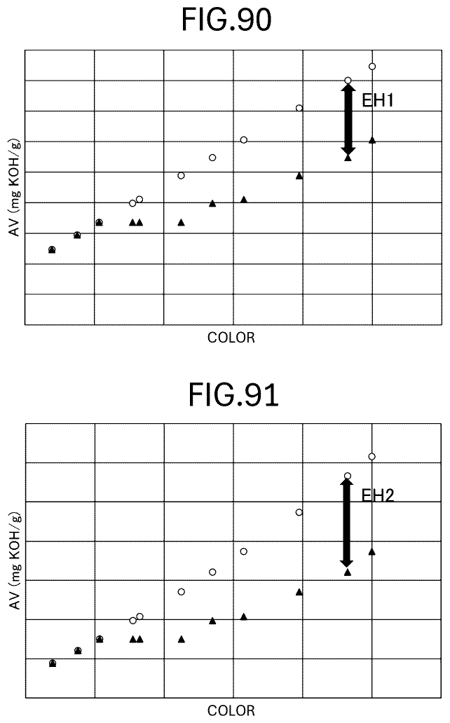

FIG. 90 illustrates a graph of a linear function showing the correlation of the acid value of the frying oil P relative to the color of the frying oil P, which relates to whether empty heating has been performed. FIG. 91 illustrates a graph of a quadratic function showing the correlation of the acid value of the frying oil P relative to the color of the frying oil P, which relates to whether empty heating has been performed.

As illustrated in each of FIG. 82 t0 FIG. 91, the graph showing the correlation of the second deterioration indicator of the frying oil P relative to the first deterioration indicator of the frying oil P varies depending on whether empty heating for the frying oil P has been performed. The “empty heating” is heating only the frying oil P without deep frying the deep-frying material Q, that is, with the deep-frying material Q not being placed in the frying oil P.

In each of FIG. 82 to FIG. 91, a correlation graph including a plurality of “∘” is indicative of the case where empty heating for the frying oil P has not been performed, and a correlation graph including a plurality of “A” is indicative of the case where empty heating for the frying oil P has been performed.

In the correlation graph illustrated in each of FIG. 82 to FIG. 84, in the case where the empty heating for the frying oil P has been performed, the rate of increase in the acid value as the first deterioration indicator tends to be greater than the rate of increase in the second deterioration indicator (at least one of the total polar compounds, the viscosity increase rate, or the color), and the second deterioration indicator increases by the amount of EH1. In the same manner, in the correlation graph illustrated in each of FIG. 85 to FIG. 87, in the case where the empty heating for the frying oil P has been performed, the rate of increase in the acid value as the first deterioration indicator tends to be greater than the rate of increase in the second deterioration indicator (at least one of the total polar compounds, the viscosity increase rate, and the color), and the second deterioration indicator increases by the amount of EH2.

On the other hand, in the correlation graph illustrated in each of FIG. 88 and FIG. 90, in the case where the empty heating for the frying oil P has been performed, the rate of increase in the acid value as the second deterioration indicator tends to be smaller than the rate of increase in the first deterioration indicator (viscosity increase rate or color), and the second deterioration indicator decreases by the amount of EH1. In the same manner, in the correlation graph illustrated in each of FIG. 89 and FIG. 91, in the case where the empty heating for the frying oil P has been performed, the rate of increase in the acid value as the second deterioration indicator tends to be smaller than the rate of increase in the first deterioration indicator (viscosity increase rate or color), and the acid value decreases by the amount of EH2.

Here, each of “EH1” and “EH2” corresponds to an empty heating variable set in view of the empty heating for the frying oil P, and in the case of using the acid value as the first deterioration indicator of the frying oil P, it becomes a positive variable (EH1>0, EH2>0) (see FIG. 82 to FIG. 87) while, in the case of using the acid value as the second deterioration indicator of the frying oil P, it becomes a negative variable (EH1<0, EH2<0) (see FIG. 88 to FIG. 91). The absolute value of the empty heating variable increases as the empty heating time increases.

Thus, in the case where the empty heating for the frying oil P has been performed, a correlation equation for the second deterioration indicator of the frying oil P relative to the first deterioration indicator of the frying oil P is the following Equation (33) expressed with a linear equation obtained by adding a member of the empty heating variable EH1 to Equation (31), or the following Equation (34) expressed with a quadratic equation obtained by adding a member of the empty heating variable EH2 to Equation (32).

$$D i 2 n = α \times ( Di 1 n ) + β + EH 1 \qquad ( 33 )$$

$$Di 2 n = γ \times {( Di 1 n )}^{2} + δ \times ( Di 1 n ) + ε + EH 2 \qquad ( 34 )$$

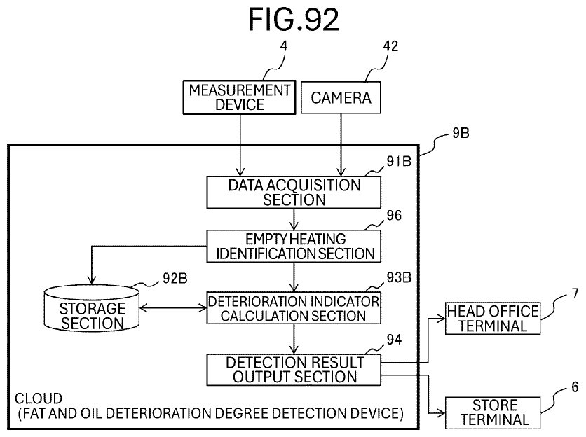

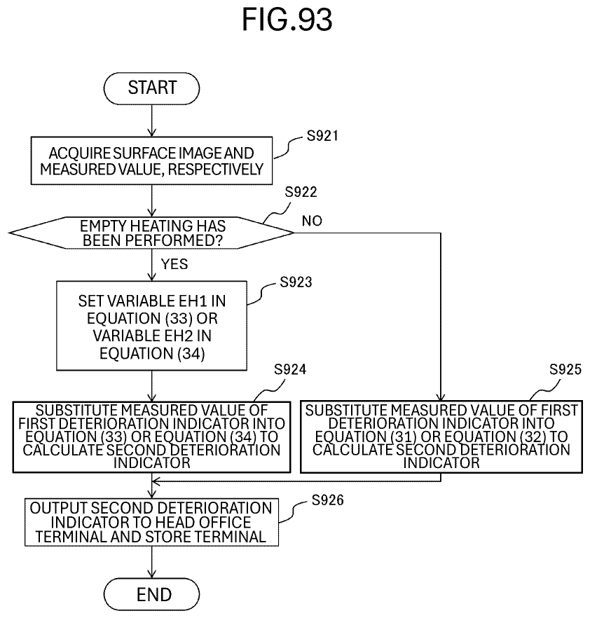

FIG. 92 illustrates a functional block diagram illustrating functions provided in the cloud 9B according to the tenth embodiment. FIG. 93 illustrates a flowchart of a flow of the processing to be executed by the cloud 9B according to the tenth embodiment.

As illustrated in FIG. 92, the cloud 9B according to the present embodiment includes a data acquisition section 91B, an empty heating identification section 96, a storage section 92B, a deterioration indicator calculation section 93B, and a detection result output section 94.

As illustrated in FIG. 93, in the cloud 9B, firstly, the data acquisition section 91B acquires the measured value of the first deterioration indicator of the frying oil P output from the measurement device 4 and the surface image of the frying oil P output from the camera 42 (step S921).

Next, the empty heating identification section 96 identifies whether the empty heating for the frying oil P has been performed based on the surface image of the frying oil P acquired in step S921 (step S922). As described above, the empty heating is heating the frying oil P with the deep-frying material Q not being placed therein. The empty heating identification section 96 identifies how long the deep-frying material Q is not included in the surface image of the frying oil P captured by the camera 42, as the period of time during which the empty heating has been performed.

The method for determining whether the empty heating has been performed does not necessarily have to be based on whether the deep-frying material Q is included in the surface image of the frying oil P captured by the camera 42. For example, a temperature sensor may be attached to the fryer 2, so that it can be identified that the empty heating has been performed based on the temperature measured by the temperature sensor which falls below a predetermined temperature (empty heating temperature). Alternatively, for example, a weight sensor may be attached to the fryer 2, so that it can be identified that the empty heating has been performed based on the increase or decrease in the weight measured by the weight sensor.

Furthermore, for example, it may be identified that the empty heating has been performed based on the information on the type and number of the material Q for deep frying or the time for performing deep frying, which has been recorded at a store. Still further, it may be identified that the empty heating has been performed based on the empty heating time which is calculated based on a daily schedule of deep frying which has been recorded in advance at a store.

Still further, for example, it may be identified that the empty heating has been performed based on an operation made for the operation switches 22A, which starts the deep frying at the fryer 2. Still further, for example, it may be identified that the empty heating has been performed based on the consumption of power or gas at the fryer 2.

In the case where it is identified in step S922 that the empty heating for the frying oil P has been performed (step 922/YES), the empty heating identification section 96 sets the empty heating variable EH1 of Equation (33) or the empty heating variable EH2 of Equation (34), which is stored in the storage section 92B (step S923).

Next, in the case where it is identified in step S922 that the empty heating for the frying oil P has not been performed (step 922/NO), the deterioration indicator calculation section 93B substitutes the measured value of the first deterioration indicator of the frying oil P acquired in step S921 into Di1n of Equation (31) or Di1n of Equation (32), which is stored in the storage section 92B, to calculate the second deterioration indicator Di2n of the frying oil P (step S925).

Then, the detection result output section 94 outputs the second deterioration indicator Di2n of the frying oil P calculated in step S924 or step S925 to the store terminal 6 and the head office terminal 7, respectively, as the result of detection of the deterioration degree of the frying oil P (step S926), whereby the processing in the cloud 9B is ended.

In the present embodiment, the empty heating identification section 96 identifies whether the empty heating for the frying oil P has been performed, however, the data acquisition section 91B may acquire the information relating to whether the empty heating has been performed from the store terminal 6 or the head office terminal 7. In this case, based on the information (information indicating that the empty heating has been performed) acquired by the data acquisition section 91B, the empty heating identification section 96 uses and sets the empty heating variable EH1 of Equation (33) or the empty heating variable EH2 of Equation (34), which is stored in the storage section 92B. That is, the cloud 9B does not necessarily have to have the function of determining whether the empty heating for the frying oil P has been performed.

According to the present embodiment, by using Equation (31) or Equation (32), or Equation (33) or Equation (34) appropriately depending on whether empty heating for the frying oil P has been performed so as to calculate the second deterioration indicator of the frying oil P, the calculation of the second deterioration indicator of the frying oil P can be carried out with higher accuracy than the case of calculating it using Equation (31) or Equation (32) in all cases without considering whether empty heating for the frying oil P has been performed.

### Eleventh Embodiment

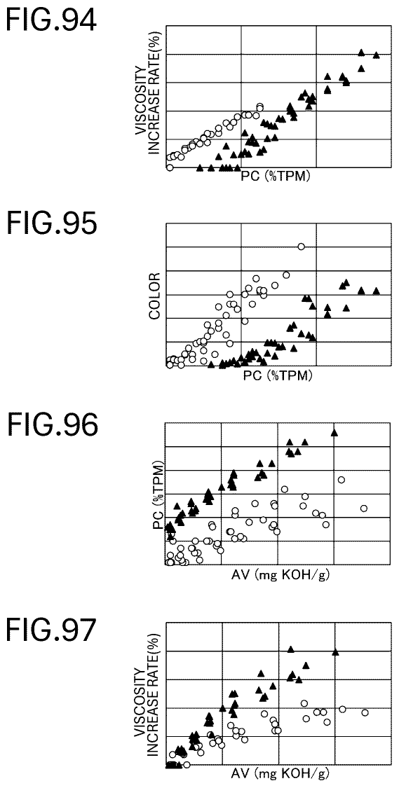

Next, a cloud 9C according to the eleventh embodiment of the present invention will be described with reference to FIG. 94 to FIG. 122.

FIG. 94 illustrates a graph showing the correlation of the viscosity increase rate of the frying oil P relative to the total polar compounds contained in the frying oil P relating to the first oil type and the second oil type.

FIG. 95 illustrates a graph showing the correlation of the color of the frying oil P relative to the total polar compounds contained in the frying oil P relating to the first oil type and the second oil type. FIG. 96 illustrates a graph showing the correlation of the total polar compounds contained in the frying oil P relative to the acid value of the frying oil P relating to the first oil type and the second oil type. FIG. 97 illustrates a graph showing the correlation of the viscosity increase rate of the frying oil P relative to the acid value of the frying oil P relating to the first oil type and the second oil type.

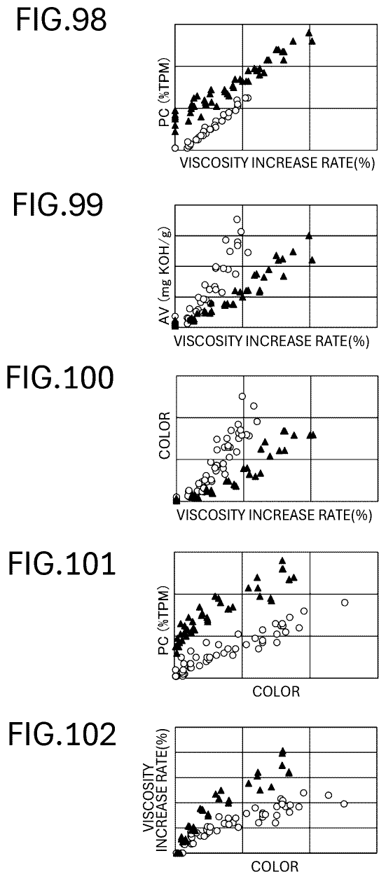

FIG. 98 illustrates a graph showing the correlation of the total polar compounds contained in the frying oil P relative to the viscosity increase rate of the frying oil P relating to the first oil type and the second oil type.

FIG. 99 illustrates a graph showing the correlation of the acid value of the frying oil P relative to the viscosity increase rate of the frying oil P relating to the first oil type and the second oil type. FIG. 100 illustrates a graph showing the correlation of the color of the frying oil P relative to the viscosity increase rate of the frying oil P relating to the first oil type and the second oil type.

FIG. 101 illustrates a graph showing the correlation of the total polar compounds contained in the frying oil P relative to the color of the frying oil P relating to the first oil type and the second oil type. FIG. 102 illustrates a graph showing the correlation of the viscosity increase rate of the frying oil P relative to the color of the frying oil P relating to the first oil type and the second oil type.

In each of the correlation graphs illustrated in FIG. 94 to FIG. 102, the first deterioration indicator corresponding to the horizontal axis is the value measured using the measurement device 4.

As described in the fourth embodiment, the type of the frying oil P can be classified into the first oil type and the second oil type depending on the fatty acid composition of the frying oil P.

As illustrated in each of FIG. 94 to FIG. 102, the correlation graph of the second deterioration indicator of the frying oil P relative to the first deterioration indicator of the frying oil P differs between the first oil type and the second oil type. In each of FIG. 94 to FIG. 102, a correlation graph including a plurality of “∘” is indicative of the correlation graph of the second deterioration indicator of the frying oil P corresponding to the first oil type relative to the first deterioration indicator of the frying oil P, and a correlation graph including a plurality of “▴” is indicative of the correlation graph of the second deterioration indicator of the frying oil P corresponding to the second oil type relative to the first deterioration indicator of the frying oil P, respectively.

In the case where the type of the frying oil P is the first oil type, the correlation equation for the acid value of the frying oil P relative to the total polar compounds of the frying oil P is a linear equation expressed with the following Equation (35) including each of α1 as the first-order coefficient α of Di1n and β1 as the constant β in Equation (31), or a quadratic equation expressed with the following Equation (36) including each of γ1 as the second-order coefficient γ of Di1n, δ1 as the first-order coefficient δ of Di1n, and ε1 as the constant ε in Equation (32).

$$Di 2 n = α 1 \times ( Di 1 n ) + β 1 \qquad ( 35 )$$

$$Di 2 n = γ 1 \times {( Di 1 n )}^{2} + δ 1 \times ( PCn ) + ε 1 \qquad ( 36 )$$

In the case where the type of the frying oil P is the second oil type, the correlation equation for the second deterioration indicator of the frying oil P relative to the first deterioration indicator of the frying oil P is a linear equation expressed with the following Equation (37) including each of α2 as the first-order coefficient α of Di1n and β2 as the constant β in Equation (31), or a quadratic equation expressed with the following Equation (38) including each of γ2 as the second-order coefficient γ of Di1n, δ2 as the first-order coefficient δ of Di1n, and ε2 as the constant ε in Equation (32).

$$Di 2 n = α 2 \times ( Di 1 n ) + β 2 \qquad ( 37 )$$

$$Di 2 n = γ 2 \times {( Di 1 n )}^{2} + δ 2 \times ( Di 1 n ) + ε 2 \qquad ( 38 )$$

Here, in each of FIG. 94, FIG. 95, FIG. 99, and FIG. 100, the first-order coefficient α2 of Di1n in Equation (37) is smaller than the first-order coefficient α1 of Di1n in Equation (35) (α2<α1), and the constant β2 in Equation (37) is smaller than the constant β1 in Equation (35) (β2<β1). Furthermore, the second-order coefficient γ2 of Di1n in Equation (38) is smaller than the second-order coefficient γ1 of Di1n in Equation (36) (γ2<γ1), the first-order coefficient δ2 of Di1n in Equation (38) is smaller than the first-order coefficient δ1 of Di1n in Equation (36) (62<61), and the constant ε2 in Equation (38) is smaller than the constant ε1 in Equation (36) (E2<ε1).

On the other hand, in each of FIG. 96 to FIG. 98, FIG. 101, and FIG. 102, the first-order coefficient α2 of Di1n in Equation (37) is greater than the first-order coefficient α1 of Di1n in Equation (35) (α2>α1), and the constant β2 in Equation (37) is greater than the constant β1 in Equation (35) (β2>β1). Furthermore, the second-order coefficient γ2 of Di1n in Equation (38) is greater than the second-order coefficient γ1 of Di1n in Equation (36) (γ2>γ1), the first-order coefficient δ2 of Di1n in Equation (38) is greater than the first-order coefficient δ1 of Di1n in Equation (36) (62>61), and the constant ε2 in Equation (38) is greater than the constant ε1 in Equation (36) (ε2>ε1).

As described above, especially in the case of measuring the first deterioration indicator of the frying oil P using the measurement device 4, a difference is found in the correlation of the second deterioration indicator of the frying oil P relative to the first deterioration indicator of the frying oil P between the first oil type and the second oil type. Thus, using the equation in which the difference therebetween is considered (Equation (35) or Equation (36) in the case of the first oil type and Equation (37) or Equation (38) in the case of the second oil type) enables the calculation of the second deterioration indicator of the frying oil P with higher accuracy.

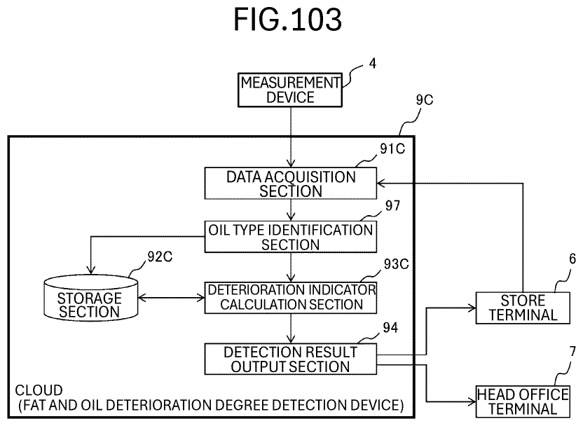

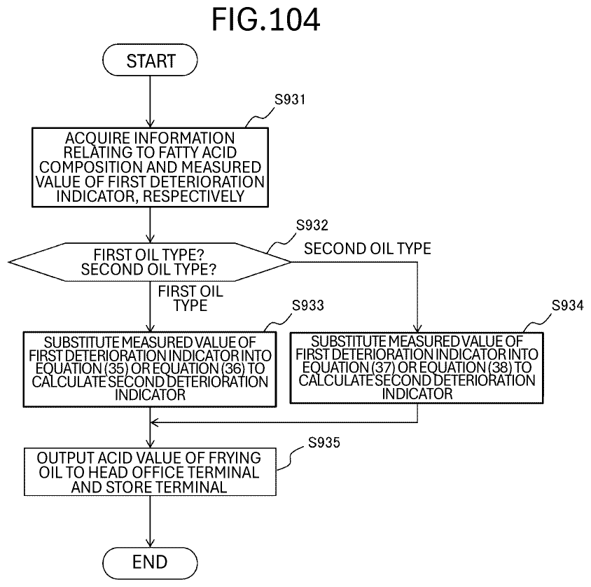

FIG. 103 is a functional block diagram illustrating functions provided in the cloud 9C according to the eleventh embodiment. FIG. 104 illustrates a flowchart of a flow of the processing to be executed by the cloud 9C according to the eleventh embodiment.

As illustrated in FIG. 103, the cloud 9C according to the present embodiment includes a data acquisition section 91C, an oil type identification section 97, a storage section 92C, a deterioration indicator calculation section 93C, and a detection result output section 94.

As illustrated in FIG. 104, in the cloud 9C, firstly, the data acquisition section 91C acquires the measured value of the first deterioration indicator of the frying oil P output from the measurement device 4 and the information relating to the fatty acid composition of the frying oil P output from the store terminal 6 (step S931).

Next, the oil type identification section 97 identifies whether the type of the frying oil P is the first oil type or the second oil type based on the information relating to the fatty acid composition of the frying oil P acquired in step S931 (step S932).

In the case where it is identified in step S932 that the type of the frying oil P is the first oil type (step S932/first oil type), the deterioration indicator calculation section 93C substitutes the measured value of the first deterioration indicator of the frying oil P acquired in step S931 into Di1n of Equation (35) or Di1n of Equation (36), which is stored in the storage section 92C, to calculate the second deterioration indicator Di2n of the frying oil P (step S933).

On the other hand, in the case where it is identified in step S932 that the type of the frying oil P is the second oil type (step S932/second oil type), the deterioration indicator calculation section 93C substitutes the measured value of the first deterioration indicator of the frying oil P acquired in step S931 into Di1n of Equation (37) or Di1n of Equation (38), which is stored in the storage section 92C, to calculate the second deterioration indicator Di2n of the frying oil P (step S934).

Then, the detection result output section 94 outputs the second deterioration indicator Di2n of the frying oil P calculated in step S933 or step S934 to the store terminal 6 and the head office terminal 7, respectively, as the result of detection of the deterioration degree of the frying oil P (step S935), whereby the processing in the cloud 9C is ended.

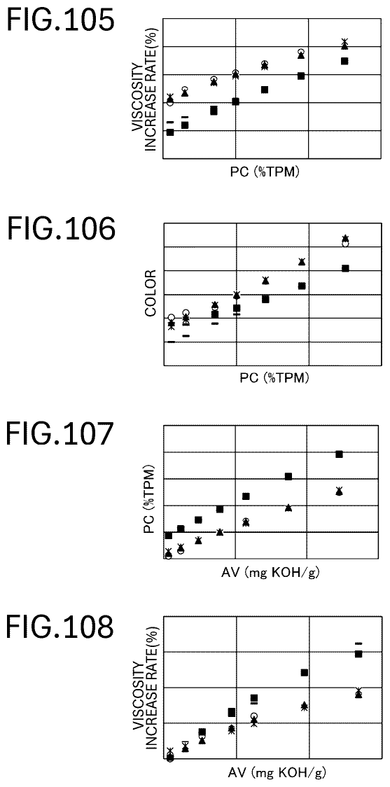

FIG. 105 illustrates a graph showing the correlation of the viscosity increase rate of the frying oil P relating to the first oil type relative to the total polar compounds (measured using the measurement device 4) contained in the frying oil P, in which the actually measured viscosity increase rates are compared with the viscosity increase rates calculated using Equation (31), Equation (32), Equation (35), and Equation (36), respectively. FIG. 106 illustrates a graph showing the correlation of the color of the frying oil P relating to the first oil type relative to the total polar compounds (measured using the measurement device 4) contained in the frying oil P, in which the actually measured values of the color are compared with the colors calculated using Equation (31), Equation (32), Equation (35), and Equation (36), respectively.

FIG. 107 illustrates a graph showing the correlation of the total polar compounds contained in the frying oil P relating to the first oil type relative to the acid value (measured using the measurement device 4) of the frying oil P, in which the actually measured total polar compounds are compared with the total polar compounds calculated using Equation (31), Equation (32), Equation (35), and Equation (36), respectively. FIG. 108 illustrates a graph showing the correlation of the viscosity increase rate of the frying oil P relating to the first oil type relative to the acid value (measured using the measurement device 4) of the frying oil P, in which the actually measured viscosity increase rates are compared with the viscosity increase rates calculated using Equation (31), Equation (32), Equation (35), and Equation (36), respectively.

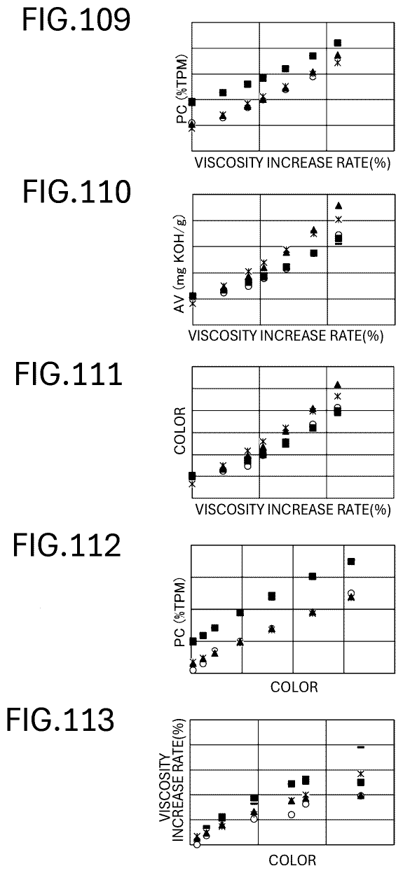

FIG. 109 illustrates a graph showing the correlation of the total polar compounds contained in the frying oil P relating to the first oil type relative to the viscosity increase rate (measured using the measurement device 4) of the frying oil P, in which the actually measured total polar compounds are compared with the total polar compounds calculated using Equation (31), Equation (32), Equation (35), and Equation (36), respectively. FIG. 110 illustrates a graph showing the correlation of the acid value of the frying oil P relating to the first oil type relative to the viscosity increase rate (measured using the measurement device 4) of the frying oil P, in which the actually measured acid values are compared with the acid values calculated using Equation (31), Equation (32), Equation (35), and Equation (36), respectively. FIG. 111 illustrates a graph showing the correlation of the color of the frying oil P relating to the first oil type relative to the viscosity increase rate (measured using the measurement device 4) of the frying oil P, in which the actually measured values of the color are compared with the colors calculated using Equation (31), Equation (32), Equation (35), and Equation (36), respectively.

FIG. 112 illustrates a graph showing the correlation of the total polar compounds contained in the frying oil P relating to the first oil type relative to the color (measured using the measurement device 4) of the frying oil P, in which the actually measured total polar compounds are compared with the total polar compounds calculated using Equation (31), Equation (32), Equation (35), and Equation (36), respectively. FIG. 113 illustrates a graph showing the correlation of the viscosity increase rate of the frying oil P relating to the first oil type relative to the color (measured using the measurement device 4) of the frying oil P, in which the actually measured viscosity increase rates are compared with the viscosity increase rates calculated using Equation (31), Equation (32), Equation (35), and Equation (36), respectively.

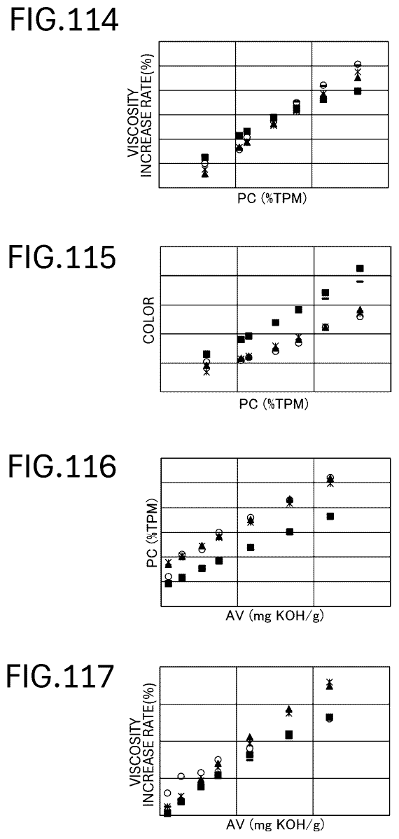

FIG. 114 illustrates a graph showing the correlation of the viscosity increase rate of the frying oil P relating to the second oil type relative to the total polar compounds (measured using the measurement device 4) contained in the frying oil P, in which the actually measured viscosity increase rates are compared with the viscosity increase rates calculated using Equation (31), Equation (32), Equation (37), and Equation (38), respectively. FIG. 115 illustrates a graph showing the correlation of the color of the frying oil P relating to the second oil type relative to the total polar compounds (measured using the measurement device 4) contained in the frying oil P, in which the actually measured values of the color are compared with the colors calculated using Equation (31), Equation (32), Equation (37), and Equation (38), respectively.

FIG. 116 illustrates a graph showing the correlation of the total polar compounds contained in the frying oil P relating to the second oil type relative to the acid value (measured using the measurement device 4) of the frying oil P, in which the actually measured total polar compounds are compared with the total polar compounds calculated using Equation (31), Equation (32), Equation (37), and Equation (38), respectively. FIG. 117 illustrates a graph showing the correlation of the viscosity increase rate of the frying oil P relating to the second oil type relative to the acid value (measured using the measurement device 4) of the frying oil P, in which the actually measured viscosity increase rates are compared with the viscosity increase rates calculated using Equation (31), Equation (32), Equation (37), and Equation (38), respectively.

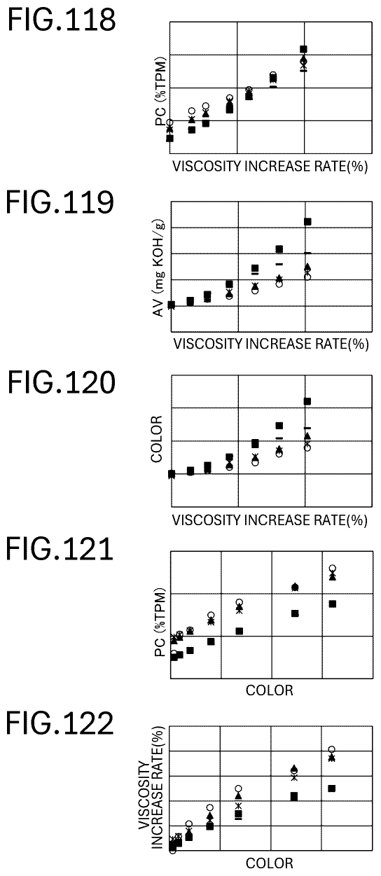

FIG. 118 illustrates a graph showing the correlation of the total polar compounds contained in the frying oil P relating to the second oil type relative to the viscosity increase rate (measured using the measurement device 4) of the frying oil P, in which the actually measured total polar compounds are compared with the total polar compounds calculated using Equation (31), Equation (32), Equation (37), and Equation (38), respectively. FIG. 119 illustrates a graph showing the correlation of the acid value of the frying oil P relating to the second oil type relative to the viscosity increase rate (measured using the measurement device 4) of the frying oil P, in which the actually measured acid values are compared with the acid values calculated using Equation (31), Equation (32), Equation (37), and Equation (38), respectively. FIG. 120 illustrates a graph showing the correlation of the color of the frying oil P relating to the second oil type relative to the viscosity increase rate (measured using the measurement device 4) of the frying oil P, in which the actually measured values of the color are compared with the colors calculated using Equation (31), Equation (32), Equation (37), and Equation (38), respectively.

FIG. 121 illustrates a graph showing the correlation of the total polar compounds contained in the frying oil P relating to the second oil type relative to the color (measured using the measurement device 4) of the frying oil P, in which the actually measured total polar compounds are compared with the total polar compounds calculated using Equation (31), Equation (32), Equation (37), and Equation (38), respectively. FIG. 122 illustrates a graph showing the correlation of the viscosity increase rate of the frying oil P relating to the second oil type relative to the color (measured using the measurement device 4) of the frying oil P, in which the actually measured viscosity increase rates are compared with the viscosity increase rates calculated using Equation (31), Equation (32), Equation (37), and Equation (38), respectively.

In each of FIG. 105 to FIG. 113 relating to the case where the type of the frying oil P is the first oil type, a graph including a plurality of “∘” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (35), and a graph including a plurality of “▴” is indicative of the correlation graph for Equation (36), respectively.

In each of FIG. 114 to FIG. 122 relating to the case where the type of the frying oil P is the second oil type, a graph including a plurality of “∘” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (37), and a graph including a plurality of “▴” is indicative of the correlation graph for Equation (38), respectively.

As illustrated in each of FIG. 105 to FIG. 113, the correlation graph for Equation (35) and the correlation graph for Equation (36) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually measured second deterioration indicator of the frying oil P further than the correlation graph for Equation (35) and the correlation graph for Equation (36).

In the same manner, as illustrated in each of FIG. 114 to FIG. 122, the correlation graph for Equation (37) and the correlation graph for Equation (38) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually measured second deterioration indicator of the frying oil P further than the correlation graph for Equation (37) and the correlation graph for Equation (38).

Thus, in the case where the measurement device 4 measures the first deterioration indicator of the frying oil P, the cloud 9C identifies whether the type of the frying oil P is the first oil type or the second oil type based on the fatty acid composition of the frying oil P and calculates the second deterioration indicator of the frying oil P using the correlation equation for the oil type as identified. This enables the calculation of the second deterioration indicator of the frying oil P with higher accuracy than the case of calculating it using Equation (31) or Equation (32) in all cases without considering the type of oil classified based on the fatty acid composition of the frying oil P.

### Twelfth Embodiment

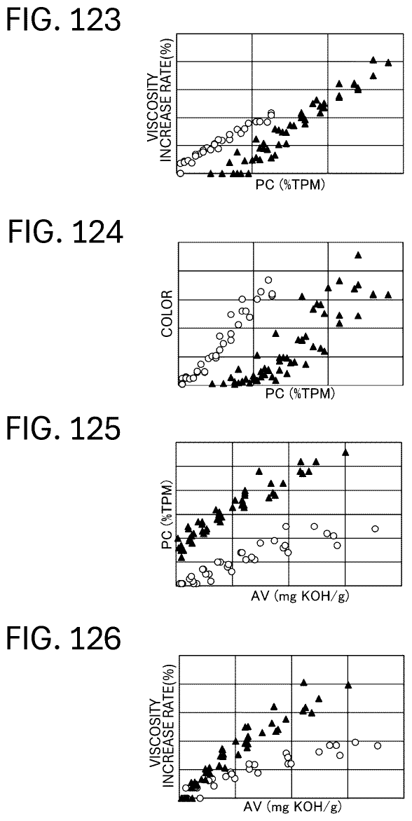

Next, the cloud 9C according to the twelfth embodiment of the present invention will be described with reference to FIG. 123 to FIG. 150. The functional block of the functions provided in the cloud 9C according to the present embodiment is not illustrated herein as it is the same as that of the cloud 9C according to the eleventh embodiment, and the components which are common therebetween are provided with the same reference signs. In the following, the same applies to the thirteenth embodiment and the fourteenth embodiment.

FIG. 123 illustrates a graph showing the correlation of the viscosity increase rate of the frying oil P relating to the third oil type and the fourth oil type relative to the total polar compounds contained in the frying oil P. FIG. 124 illustrates a graph showing the correlation of the color of the frying oil P relating to the third oil type and the fourth oil type relative to the total polar compounds contained in the frying oil P. FIG. 125 illustrates a graph showing the correlation of the total polar compounds contained in the frying oil P relating to the third oil type and the fourth oil type relative to the acid value of the frying oil P. FIG. 126 illustrates a graph showing the correlation of the viscosity increase rate of the frying oil P relating to the third oil type and the fourth oil type relative to the acid value of the frying oil P.

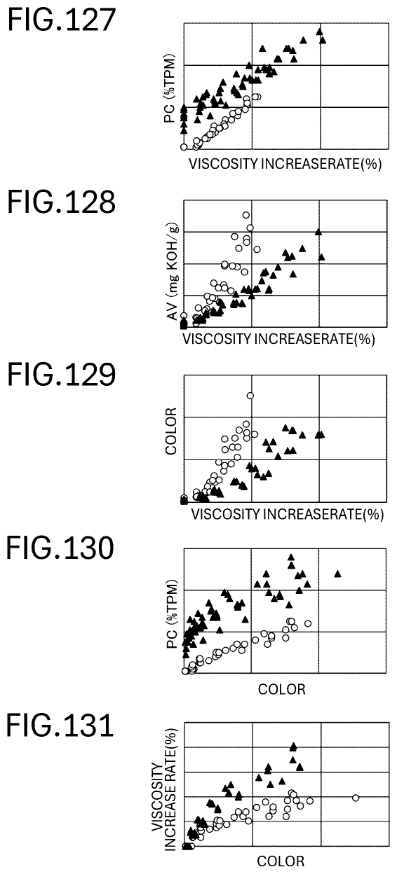

FIG. 127 illustrates a graph showing the correlation of the total polar compounds contained in the frying oil P relating to the third oil type and the fourth oil type relative to the viscosity increase rate of the frying oil P. FIG. 128 illustrates a graph showing the correlation of the acid value of the frying oil P relating to the third oil type and the fourth oil type relative to the viscosity increase rate of the frying oil P. FIG. 129 illustrates a graph showing the correlation of the color of the frying oil P relating to the third oil type and the fourth oil type relative to the viscosity increase rate of the frying oil P. FIG. 130 illustrates a graph showing the correlation of the total polar compounds contained in the frying oil P relating to the third oil type and the fourth oil type relative to the color of the frying oil P. FIG. 131 illustrates a graph showing the correlation of the viscosity increase rate of the frying oil P relating to the third oil type and the fourth oil type relative to the color of the frying oil P.

In each of the correlation graphs illustrated in FIG. 123 to FIG. 131, the first deterioration indicator corresponding to the horizontal axis is the value measured using the measurement device 4.

The type of the frying oil P is classified into the third oil type and the fourth oil type depending on the iodine value (IV) of the frying oil P.

As illustrated in each of FIG. 123 to FIG. 131, the correlation graph of the second deterioration indicator of the frying oil P relative to the first deterioration indicator of the frying oil P differs between the third oil type and the fourth oil type. In each of FIG. 123 to FIG. 131, a correlation graph including a plurality of “∘” is indicative of the correlation graph of the second deterioration indicator of the frying oil P corresponding to the third oil type relative to the first deterioration indicator of the frying oil P, and a correlation graph including a plurality of “▴” is indicative of the correlation graph of the second deterioration indicator of the frying oil P corresponding to the fourth oil type relative to the first deterioration indicator of the frying oil P, respectively.

In the case where the type of the frying oil P is the third oil type, the correlation equation for the second deterioration indicator of the frying oil P relative to the first deterioration indicator of the frying oil P is a linear equation expressed with the following Equation (39) including each of α3 as the first-order coefficient α of Di1n and β3 as the constant β in Equation (31), or a quadratic equation expressed with the following Equation (40) including each of γ3 as the second-order coefficient γ of Di1n, δ3 as the first-order coefficient δ of Di1n, and ε3 as the constant ε in Equation (32).

Di2n=α3×(Di1n)+β3 (39)

Di2n=γ3×(Di1n)²+δ3×(Di1n)+ε3 (40)

In the case where the type of the frying oil P is the fourth oil type, the correlation equation for the second deterioration indicator of the frying oil P relative to the first deterioration indicator of the frying oil P is a linear equation expressed with the following Equation (41) including each of α4 as the first-order coefficient α of Di1n and β4 as the constant β in Equation (31), or a quadratic equation expressed with the following Equation (42) including each of γ4 as the second-order coefficient γ of Di1n, δ4 as the first-order coefficient δ of Di1n, and ε4 as the constant ε in Equation (32).

$$Di 2 n = α 4 \times ( Di 1 n ) + β 4 \qquad ( 41 )$$

$${Di 2 n = γ 4 \times Di 1 n )}^{2} + δ 4 \times ( Di 1 n ) + ε 4 \qquad ( 42 )$$

Here, in each of FIG. 123, FIG. 124, FIG. 128, and FIG. 129, the first-order coefficient α4 of Di1n in Equation (41) is smaller than the first-order coefficient α3 of Di1n in Equation (39) (α4<α3), and the constant β4 in Equation (41) is smaller than the constant β3 in Equation (39) (β4<β3). Furthermore, the second-order coefficient γ4 of Di1n in Equation (42) is smaller than the second-order coefficient γ3 of Di1n in Equation (40) (γ4<γ3), the first-order coefficient δ4 of Di1n in Equation (42) is smaller than the first-order coefficient δ3 of Di1n in Equation (40) (δ4<δ3), and the constant ε4 in Equation (42) is smaller than the constant ε3 in Equation (40) (ε4<ε3).

On the other hand, in each of FIG. 125 to FIG. 127, FIG. 130, and FIG. 131, the first-order coefficient α4 of Di1n in Equation (41) is greater than the first-order coefficient α3 of Di1n in Equation (39) (α4>α3), and the constant β4 in Equation (41) is greater than the constant β3 in Equation (39) (β4>β3). Furthermore, the second-order coefficient γ4 of Di1n in Equation (42) is greater than the second-order coefficient γ3 of Di1n in Equation (40) (γ4>γ3), the first-order coefficient δ4 of Di1n in Equation (42) is greater than the first-order coefficient δ3 of Di1n in Equation (40) (δ4>δ3), and the constant ε4 in Equation (42) is greater than the constant ε3 in Equation (40) (ε4>ε3).

As described above, especially in the case of measuring the first deterioration indicator of the frying oil P using the measurement device 4, a difference is found in the correlation of the second deterioration indicator of the frying oil P relative to the first deterioration indicator of the frying oil P between the third oil type and the fourth oil type. Thus, by using the equation in which the difference therebetween is considered (Equation (39) or Equation (40) in the case of the third oil type and Equation (41) or Equation (42) in the case of the fourth oil type), the calculation of the second deterioration indicator of the frying oil P can be carried out with higher accuracy.

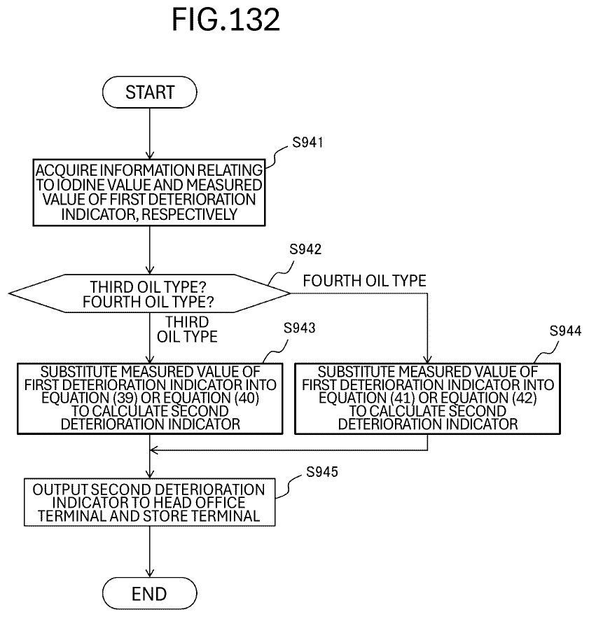

FIG. 132 illustrates a flowchart of a flow of the processing to be executed by the cloud 9C according to the twelfth embodiment.

In the cloud 9C according to the present embodiment, firstly, the data acquisition section 91C acquires the measured value of the first deterioration indicator of the frying oil P output from the measurement device 4 and the information relating to the iodine value of the frying oil P output from the store terminal 6 (step S941).

The information relating to the iodine value of the frying oil P includes, for example, the information indicative of the specific names of oil types, the information indicative of the iodine value of the frying oil P, and the like. The information relating to the iodine value of the frying oil P does not necessarily have to be output from the store terminal 6 to the cloud 9C, but may be output from, for example, the head office terminal 7 to the cloud 8C.

Next, the oil type identification section 97 identifies whether the type of the frying oil P is the third oil type or the fourth oil type based on the information relating to the iodine value of the frying oil P acquired in step S941 (step S942).

In the case where it is identified in step S942 that the type of the frying oil P is the third oil type (step S942/third oil type), the deterioration indicator calculation section 93C substitutes the measured value of the first deterioration indicator of the frying oil P acquired in step S941 into Di1n of Equation (39) or Di1n of Equation (40), which is stored in the storage section 92C, to calculate the second deterioration indicator of the frying oil P (step S943).

On the other hand, in the case where it is identified in step S942 that the type of the frying oil P is the fourth oil type (step S942/fourth oil type), the deterioration indicator calculation section 93C substitutes the measured value of the first deterioration indicator of the frying oil P acquired in step S941 into Di1n of Equation (41) or Di1n of Equation (42), which is stored in the storage section 92C, to calculate the second deterioration indicator of the frying oil P (step S944).

Then, the detection result output section 94 outputs the second deterioration indicator of the frying oil P calculated in step S943 or step S944 to the store terminal 6 and the head office terminal 7, respectively, as the result of detection of the deterioration degree of the frying oil P (step S945), whereby the processing in the cloud 9C is ended.

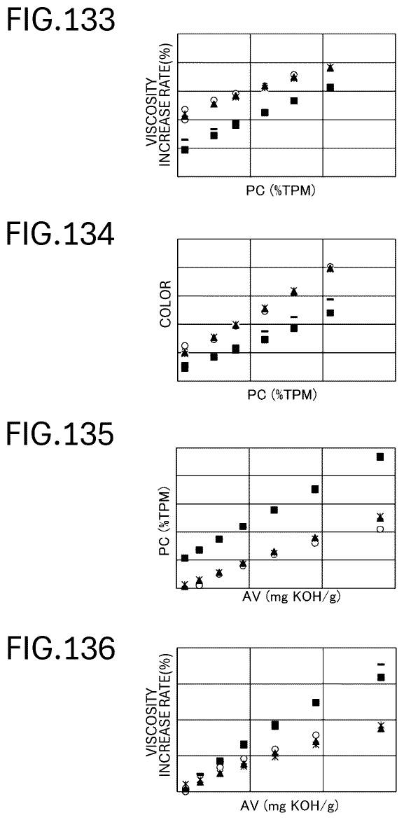

FIG. 133 illustrates a graph showing the correlation of the viscosity increase rate of the frying oil P relating to the third oil type relative to the total polar compounds (measured using the measurement device 4) contained in the frying oil P, in which the actually measured viscosity increase rates are compared with the viscosity increase rates calculated using Equation (31), Equation (32), Equation (39), and Equation (40), respectively. FIG. 134 illustrates a graph showing the correlation of the color of the frying oil P relating to the third oil type relative to the total polar compounds (measured using the measurement device 4) contained in the frying oil P, in which the actually measured values of the color are compared with the colors calculated using Equation (31), Equation (32), Equation (39), and Equation (40), respectively.

FIG. 135 illustrates a graph showing the correlation of the total polar compounds contained in the frying oil P relating to the third oil type relative to the acid value (measured using the measurement device 4) of the frying oil P, in which the actually measured total polar compounds are compared with the total polar compounds calculated using Equation (31), Equation (32), Equation (39), and Equation (40), respectively. FIG. 136 illustrates a graph showing the correlation of the viscosity increase rate of the frying oil P relating to the third oil type relative to the acid value (measured using the measurement device 4) of the frying oil P, in which the actually measured viscosity increase rates are compared with the viscosity increase rates calculated using Equation (31), Equation (32), Equation (39), and Equation (40), respectively.

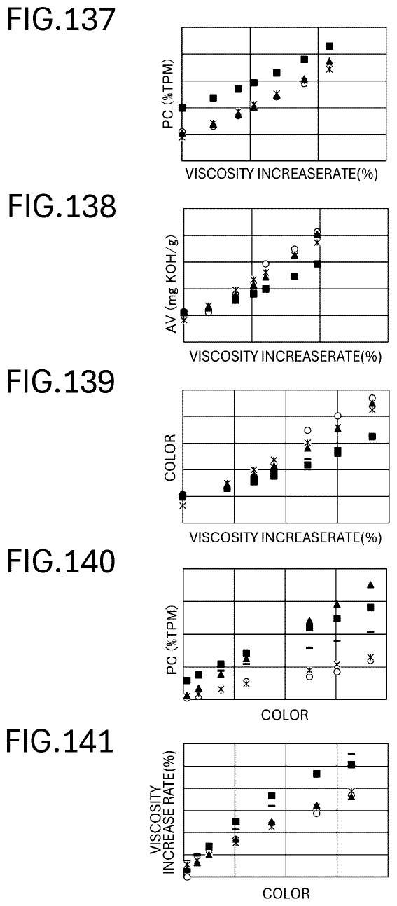

FIG. 137 illustrates a graph showing the correlation of the total polar compounds contained in the frying oil P relating to the third oil type relative to the viscosity increase rate (measured using the measurement device 4) of the frying oil P, in which the actually measured total polar compounds are compared with the total polar compounds calculated using Equation (31), Equation (32), Equation (39), and Equation (40), respectively. FIG. 138 illustrates a graph showing the correlation of the acid value of the frying oil P relating to the third oil type relative to the viscosity increase rate (measured using the measurement device 4) of the frying oil P, in which the actually measured acid values are compared with the acid values calculated using Equation (31), Equation (32), Equation (39), and Equation (40), respectively. FIG. 139 illustrates a graph showing the correlation of the color of the frying oil P relating to the third oil type relative to the viscosity increase rate (measured using the measurement device 4) of the frying oil P, in which the actually measured values of the color are compared with the colors calculated using Equation (31), Equation (32), Equation (39), and Equation (40), respectively.

FIG. 140 illustrates a graph showing the correlation of the total polar compounds contained in the frying oil P relating to the third oil type relative to the color (measured using the measurement device 4) of the frying oil P, in which the actually measured total polar compounds are compared with the total polar compounds calculated using Equation (31), Equation (32), Equation (39), and Equation (40), respectively. FIG. 141 illustrates a graph showing the correlation of the viscosity increase rate of the frying oil P relating to the third oil type relative to the color (measured using the measurement device 4) of the frying oil P, in which the actually measured viscosity increase rates are compared with the viscosity increase rates calculated using Equation (31), Equation (32), Equation (39), and Equation (40), respectively.

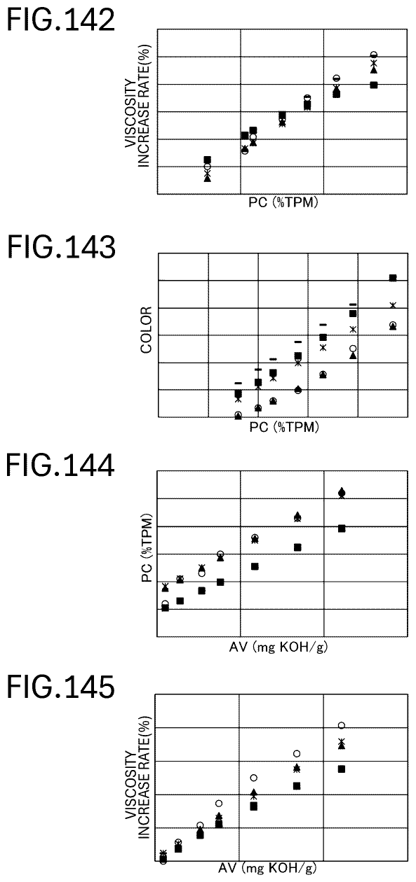

FIG. 142 illustrates a graph showing the correlation of the viscosity increase rate of the frying oil P relating to the fourth oil type relative to the total polar compounds (measured using the measurement device 4) contained in the frying oil P, in which the actually measured viscosity increase rates are compared with the viscosity increase rates calculated using Equation (31), Equation (32), Equation (41), and Equation (42), respectively. FIG. 143 illustrates a graph showing the correlation of the color of the frying oil P relating to the fourth oil type relative to the total polar compounds (measured using the measurement device 4) contained in the frying oil P, in which the actually measured values of the color are compared with the colors calculated using Equation (31), Equation (32), Equation (41), and Equation (42), respectively.

FIG. 144 illustrates a graph showing the correlation of the total polar compounds contained in the frying oil P relating to the fourth oil type relative to the acid value (measured using the measurement device 4) of the frying oil P, in which the actually measured total polar compounds are compared with the total polar compounds calculated using Equation (31), Equation (32), Equation (41), and Equation (42), respectively. FIG. 145 illustrates a graph showing the correlation of the viscosity increase rate of the frying oil P relating to the fourth oil type relative to the acid value (measured using the measurement device 4) of the frying oil P, in which the actually measured viscosity increase rates are compared with the viscosity increase rates calculated using Equation (31), Equation (32), Equation (41), and Equation (42), respectively.

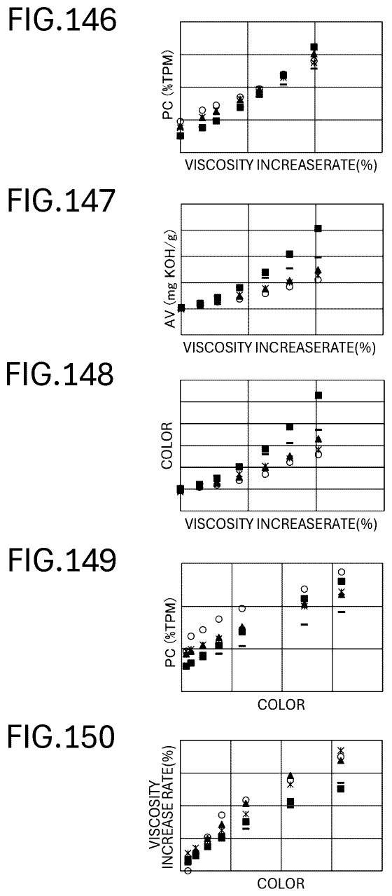

FIG. 146 illustrates a graph showing the correlation of the total polar compounds contained in the frying oil P relating to the fourth oil type relative to the viscosity increase rate (measured using the measurement device 4) of the frying oil P, in which the actually measured total polar compounds are compared with the total polar compounds calculated using Equation (31), Equation (32), Equation (41), and Equation (42), respectively. FIG. 147 illustrates a graph showing the correlation of the acid value of the frying oil P relating to the fourth oil type relative to the viscosity increase rate (measured using the measurement device 4) of the frying oil P, in which the actually measured acid values are compared with the acid values calculated using Equation (31), Equation (32), Equation (41), and Equation (42), respectively. FIG. 148 illustrates a graph showing the correlation of the color of the frying oil P relating to the fourth oil type relative to the viscosity increase rate (measured using the measurement device 4) of the frying oil P, in which the actually measured values of the color are compared with the colors calculated using Equation (31), Equation (32), Equation (41), and Equation (42), respectively.

FIG. 149 illustrates a graph showing the correlation of the total polar compounds contained in the frying oil P relating to the fourth oil type relative to the color (measured using the measurement device 4) of the frying oil P, in which the actually measured total polar compounds are compared with the total polar compounds calculated using Equation (31), Equation (32), Equation (41), and Equation (42), respectively. FIG. 150 illustrates a graph showing the correlation of the viscosity increase rate of the frying oil P relating to the fourth oil type relative to the color (measured using the measurement device 4) of the frying oil P, in which the actually measured viscosity increase rates are compared with the viscosity increase rates calculated using Equation (31), Equation (32), Equation (41), and Equation (42), respectively.

In each of FIG. 133 to FIG. 141 relating to the case where the type of the frying oil P is the third oil type, a graph including a plurality of “∘” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (39), and a graph including a plurality of “▴” is indicative of the correlation graph for Equation (40), respectively.

In each of FIG. 142 to FIG. 150 relating to the case where the type of the frying oil P is the fourth oil type, a graph including a plurality of “∘” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (41), and a graph including a plurality of “▴” is indicative of the correlation graph for Equation (42), respectively.

As illustrated in each of FIG. 133 to FIG. 141, the correlation graph for Equation (39) and the correlation graph for Equation (40) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually measured second deterioration indicator of the frying oil P further than the correlation graph for Equation (39) and the correlation graph for Equation (40).

In the same manner, as illustrated in each of FIG. 142 to FIG. 150, the correlation graph for Equation (41) and the correlation graph for Equation (42) are positioned closer to the correlation graph for the actually measured acid value of the frying oil P than the correlation graph for Equation (31) and the correlation graph for Equation (32). In other words, the correlation graph for Equation (31) and the correlation graph for Equation (32) are deviated from the correlation graph for the actually measured acid value of the frying oil P further than the correlation graph for Equation (41) and the correlation graph for Equation (42).

Thus, in the case where the measurement device 4 measures the first deterioration indicator of the frying oil P, the cloud 9C identifies whether the type of the frying oil P is the third oil type or the fourth oil type based on the iodine value of the frying oil P and calculates the second deterioration indicator of the frying oil P using the correlation equation for the oil type as identified. This enables the calculation of the second deterioration indicator of the frying oil P with higher accuracy than the case of calculating it using Equation (31) or Equation (32) in all cases without considering the type of oil classified based on the iodine value of the frying oil P.

### Thirteenth Embodiment

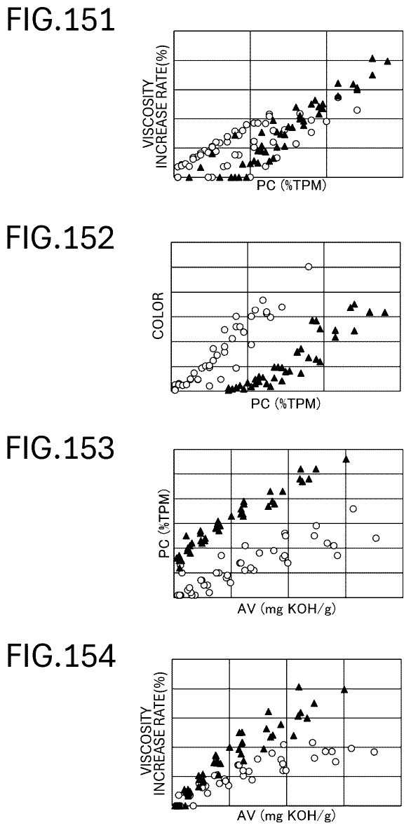

Next, the cloud 9C according to the thirteenth embodiment of the present invention will be described with reference to FIG. 151 to FIG. 178.

FIG. 151 illustrates a graph showing the correlation of the viscosity increase rate of the frying oil P relating to the fifth oil type and the sixth oil type relative to the total polar compounds contained in the frying oil P.

FIG. 152 illustrates a graph showing the correlation of the color of the frying oil P relating to the fifth oil type and the sixth oil type relative to the total polar compounds contained in the frying oil P. FIG. 153 illustrates a graph showing the correlation of the total polar compounds contained in the frying oil P relating to the fifth oil type and the sixth oil type relative to the acid value of the frying oil P. FIG. 154 illustrates a graph showing the correlation of the viscosity increase rate of the frying oil P relating to the fifth oil type and the sixth oil type relative to the acid value of the frying oil P.

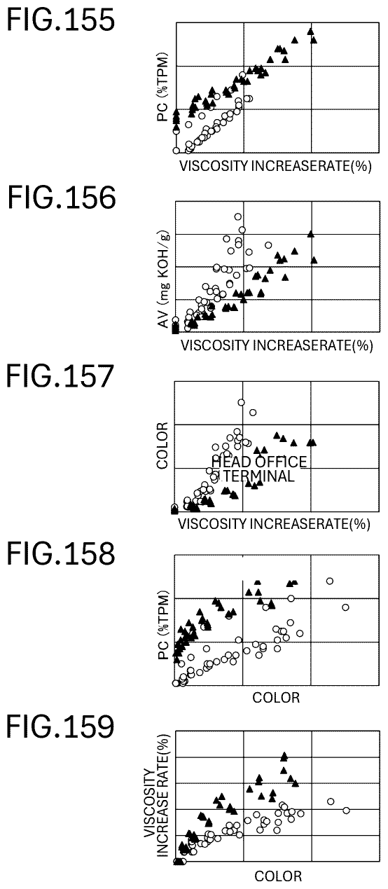

FIG. 155 illustrates a graph showing the correlation of the total polar compounds contained in the frying oil P relating to the fifth oil type and the sixth oil type relative to the viscosity increase rate of the frying oil P. FIG. 156 illustrates a graph showing the correlation of the acid value of the frying oil P relating to the fifth oil type and the sixth oil type relative to the viscosity increase rate of the frying oil P. FIG. 157 illustrates a graph showing the correlation of the color of the frying oil P relating to the fifth oil type and the sixth oil type relative to the viscosity increase rate of the frying oil P. FIG. 158 illustrates a graph showing the correlation of the total polar compounds contained in the frying oil P relating to the fifth oil type and the sixth oil type relative to the color of the frying oil P. FIG. 159 illustrates a graph showing the correlation of the viscosity increase rate of the frying oil P relating to the fifth oil type and the sixth oil type relative to the color of the frying oil P.

In each of the correlation graphs illustrated in FIG. 151 to FIG. 159, the first deterioration indicator corresponding to the horizontal axis is the value measured using the measurement device 4.

As described in the sixth embodiment, the type of the frying oil P is classified into the fifth oil type and the sixth oil type depending on the value obtained by the conductometric determination method (CDM) test, which is one of the tests for evaluating the oxidative stability of fat and oil (hereinafter, simply referred to as “CDM value”).

As illustrated in each of FIG. 151 to FIG. 159, the correlation graph of the second deterioration indicator of the frying oil P relative to the first deterioration indicator of the frying oil P differs between the fifth oil type and the sixth oil type. In each of FIG. 151 to FIG. 159, a correlation graph including a plurality of “∘” is indicative of the correlation graph of the second deterioration indicator of the frying oil P corresponding to the fifth oil type relative to the first deterioration indicator of the frying oil P, and a correlation graph including a plurality of “▴” is indicative of the correlation graph of the second deterioration indicator of the frying oil P corresponding to the sixth oil type relative to the first deterioration indicator of the frying oil P, respectively.

In the case where the type of the frying oil P is the fifth oil type, the correlation equation for the second deterioration indicator of the frying oil P relative to the first deterioration indicator of the frying oil P is a linear equation expressed with the following Equation (43) including each of α5 as the first-order coefficient α of Di1n and β5 as the constant β in Equation (31), or a quadratic equation expressed with the following Equation (44) including each of γ5 as the second-order coefficient γ of Di1n, δ5 as the first-order coefficient δ of Di1n, and ε5 as the constant ε in Equation (32).

$$Di 2 n = α 5 \times ( Di 1 n ) + β 5 \qquad ( 43 )$$

$$Di 2 n = γ 5 \times {( Di 1 n )}^{2} + δ 5 \times ( Di 1 n ) + ε 5 \qquad ( 44 )$$

In the case where the type of the frying oil P is the sixth oil type, the correlation equation for the second deterioration indicator of the frying oil P relative to the first deterioration indicator of the frying oil P is a linear equation expressed with the following Equation (45) including each of α6 as the first-order coefficient α of Di1n and β6 as the constant β in Equation (31), or a quadratic equation expressed with the following Equation (46) including each of γ6 as the second-order coefficient γ of Di1n, δ6 as the first-order coefficient δ of Di1n, and ε6 as the constant ε in Equation (32).

$$Di 2 n = α 6 \times ( Di 1 n ) + β 6 \qquad ( 45 )$$

$$Di 2 n = γ 6 \times {( Di 1 n )}^{2} + δ 6 \times ( Di 1 n ) + ε6 \qquad ( 46 )$$

Here, in each of FIG. 151, FIG. 152, FIG. 156, and FIG. 157, the first-order coefficient α6 of Di1n in Equation (45) is smaller than the first-order coefficient α5 of Di1n in Equation (43) (α6<α5), and the constant β6 in Equation (45) is smaller than the constant β5 in Equation (43) (β6<β5). Furthermore, the second-order coefficient γ6 of Di1n in Equation (46) is smaller than the second-order coefficient γ5 of Di1n in Equation (14) (γ6<γ5), the first-order coefficient δ6 of Di1n in Equation (46) is smaller than the first-order coefficient δ5 of Di1n in Equation (44) (δ6<δ5), and the constant ε6 in Equation (16) is smaller than the constant ε5 in Equation (44) (ε6<ε5).

On the other hand, in each of FIG. 153 to FIG. 155, FIG. 158, and FIG. 159, the first-order coefficient α6 of Di1n in Equation (45) is greater than the first-order coefficient α5 of Di1n in Equation (43) (α6>α5), and the constant β6 in Equation (45) is greater than the constant β5 in Equation (43) (β6>β5). Furthermore, the second-order coefficient γ6 of Di1n in Equation (46) is greater than the second-order coefficient γ5 of Di1n in Equation (14) (γ6>γ5), the first-order coefficient δ6 of Di1n in Equation (46) is greater than the first-order coefficient δ5 of Di1n in Equation (44) (δ6>δ5), and the constant ε6 in Equation (16) is greater than the constant ε5 in Equation (44) (ε6>ε5).

As described above, especially in the case of measuring the first deterioration indicator of the frying oil P using the measurement device 4, a difference is found in the correlation of the second deterioration indicator of the frying oil P relative to the first deterioration indicator of the frying oil P between the fifth oil type and the sixth oil type. Thus, by using the equation in which the difference therebetween is considered (Equation (43) or Equation (44) in the case of the fifth oil type and Equation (45) or Equation (46) in the case of the sixth oil type), the calculation of the second deterioration indicator of the frying oil P can be carried out with higher accuracy.

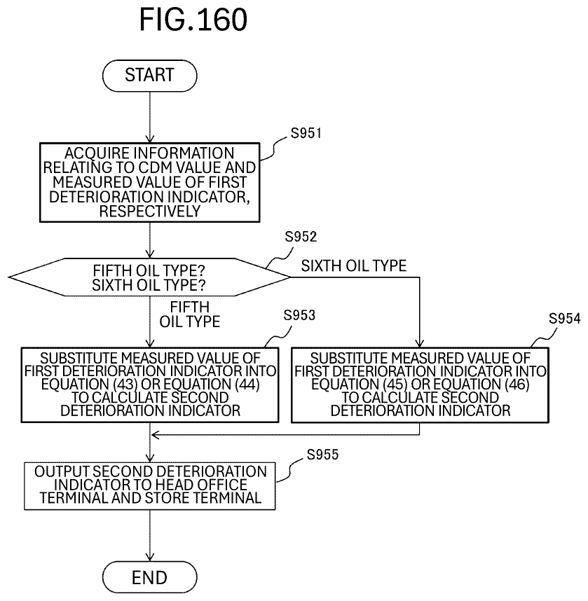

FIG. 160 illustrates a flowchart of a flow of the processing to be executed by the cloud 9C according to the thirteenth embodiment.

In the cloud 9C according to the present embodiment, firstly, the data acquisition section 91C acquires the measured value of the first deterioration indicator of the frying oil P output from the measurement device 4 and the information relating to the CDM value of the frying oil P output from the store terminal 6 (step S951).

The information relating to the CDM value of the frying oil P includes, for example, the information indicative of the specific names of oil types, the information indicative of the CDM value of the frying oil P, and the like. The information relating to the CDM value of the frying oil P does not necessarily have to be output from the store terminal 6 to the cloud 9C, but may be output from, for example, the head office terminal 7 to the cloud 9C.

Next, the oil type identification section 97 identifies whether the type of the frying oil P is the fifth oil type or the sixth oil type based on the information relating to the CDM value of the frying oil P acquired in step S951 (step S952).

In the case where it is identified in step S952 that the type of the frying oil P is the fifth oil type (step S952/fifth oil type), the deterioration indicator calculation section 93C substitutes the measured value of the first deterioration indicator of the frying oil P acquired in step S951 into Di1n of Equation (43) or Di1n of Equation (44), which is stored in the storage section 92C, to calculate the second deterioration indicator Di2n of the frying oil P (step S953).

On the other hand, in the case where it is identified in step S952 that the type of the frying oil P is the sixth oil type (step S952/sixth oil type), the deterioration indicator calculation section 93C substitutes the measured value of the first deterioration indicator of the frying oil P acquired in step S951 into Di1n of Equation (45) or Di1n of Equation (46), which is stored in the storage section 92C, to calculate the second deterioration indicator Di2n of the frying oil P (step S954).

Then, the detection result output section 94 outputs the second deterioration indicator Di2n of the frying oil P calculated in step S953 or step S954 to the store terminal 6 and the head office terminal 7, respectively, as the result of detection of the deterioration degree of the frying oil P (step S955), whereby the processing in the cloud 9C is ended.

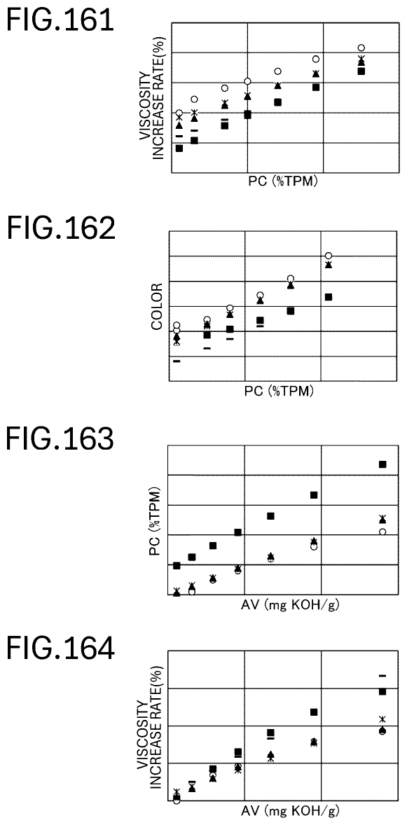

FIG. 161 illustrates a graph showing the correlation of the viscosity increase rate of the frying oil P relating to the fifth oil type relative to the total polar compounds (measured using the measurement device 4) contained in the frying oil P, in which the actually measured viscosity increase rates are compared with the viscosity increase rates calculated using Equation (31), Equation (32), Equation (43), and Equation (44), respectively. FIG. 162 illustrates a graph showing the correlation of the color of the frying oil P relating to the fifth oil type relative to the total polar compounds (measured using the measurement device 4) contained in the frying oil P, in which the actually measured values of the color are compared with the colors calculated using Equation (31), Equation (32), Equation (43), and Equation (44), respectively.

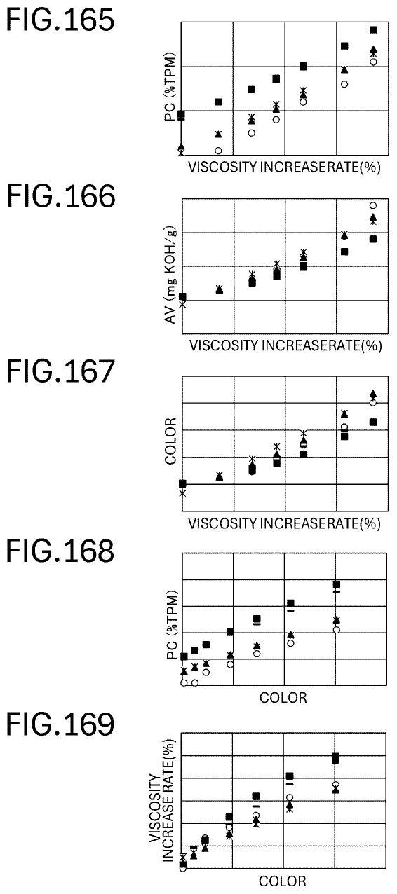

FIG. 163 illustrates a graph showing the correlation of the total polar compounds contained in the frying oil P relating to the fifth oil type relative to the acid value (measured using the measurement device 4) of the frying oil P, in which the actually measured total polar compounds are compared with the total polar compounds calculated using Equation (31), Equation (32), Equation (43), and Equation (44), respectively. FIG. 164 illustrates a graph showing the correlation of the viscosity increase rate of the frying oil P relating to the fifth oil type relative to the acid value (measured using the measurement device 4) of the frying oil P, in which the actually measured viscosity increase rates are compared with the viscosity increase rates calculated using Equation (31), Equation (32), Equation (43), and Equation (44), respectively. FIG. 165 illustrates a graph showing the correlation of the total polar compounds contained in the frying oil P relating to the fifth oil type relative to the viscosity increase rate (measured using the measurement device 4) of the frying oil P, in which the actually measured total polar compounds are compared with the total polar compounds calculated using Equation (31), Equation (32), Equation (43), and Equation (44), respectively.

FIG. 166 illustrates a graph showing the correlation of the acid value of the frying oil P relating to the fifth oil type relative to the viscosity increase rate (measured using the measurement device 4) of the frying oil P, in which the actually measured acid values are compared with the acid values calculated using Equation (31), Equation (32), Equation (43), and Equation (44), respectively. FIG. 167 illustrates a graph showing the correlation of the color of the frying oil P relating to the fifth oil type relative to the viscosity increase rate (measured using the measurement device 4) of the frying oil P, in which the actually measured values of the color are compared with the colors calculated using Equation (31), Equation (32), Equation (43), and Equation (44), respectively.

FIG. 168 illustrates a graph showing the correlation of the total polar compounds contained in the frying oil P relating to the fifth oil type relative to the color (measured using the measurement device 4) of the frying oil P, in which the actually measured total polar compounds are compared with the total polar compounds calculated using Equation (31), Equation (32), Equation (43), and Equation (44), respectively. FIG. 169 illustrates a graph showing the correlation of the viscosity increase rate of the frying oil P relating to the fifth oil type relative to the color (measured using the measurement device 4) of the frying oil P, in which the actually measured viscosity increase rates are compared with the viscosity increase rates calculated using Equation (31), Equation (32), Equation (43), and Equation (44), respectively.

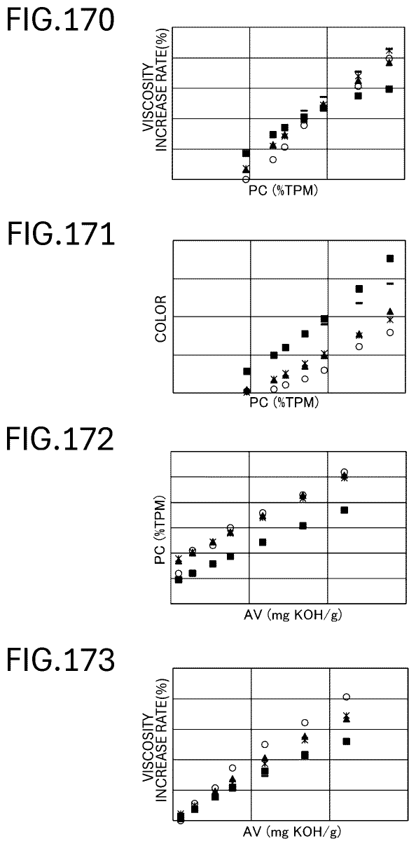

FIG. 170 illustrates a graph showing the correlation of the viscosity increase rate of the frying oil P relating to the sixth oil type relative to the total polar compounds (measured using the measurement device 4) contained in the frying oil P, in which the actually measured viscosity increase rates are compared with the viscosity increase rates calculated using Equation (31), Equation (32), Equation (45), and Equation (46), respectively. FIG. 171 illustrates a graph showing the correlation of the color of the frying oil P relating to the sixth oil type relative to the total polar compounds (measured using the measurement device 4) contained in the frying oil P, in which the actually measured values of the color are compared with the colors calculated using Equation (31), Equation (32), Equation (45), and Equation (46), respectively.

FIG. 172 illustrates a graph showing the correlation of the total polar compounds contained in the frying oil P relating to the sixth oil type relative to the acid value (measured using the measurement device 4) of the frying oil P, in which the actually measured total polar compounds are compared with the total polar compounds calculated using Equation (31), Equation (32), Equation (45), and Equation (46), respectively. FIG. 173 illustrates a graph showing the correlation of the viscosity increase rate of the frying oil P relating to the sixth oil type relative to the acid value (measured using the measurement device 4) of the frying oil P, in which the actually measured viscosity increase rates are compared with the viscosity increase rates calculated using Equation (31), Equation (32), Equation (45), and Equation (46), respectively.

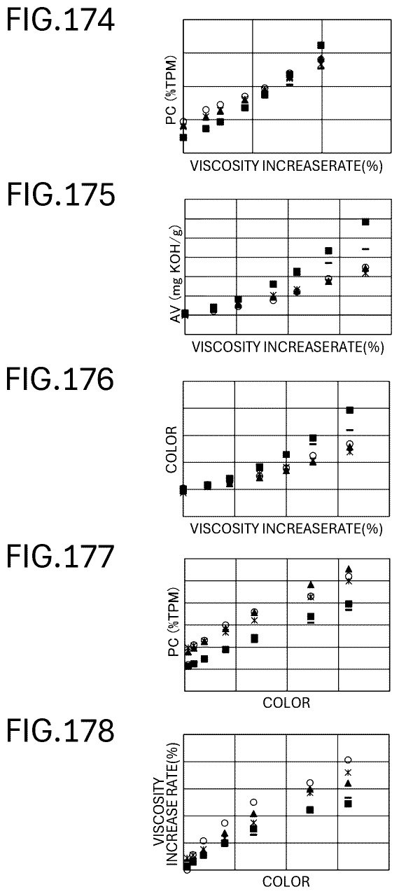

FIG. 174 illustrates a graph showing the correlation of the total polar compounds contained in the frying oil P relating to the sixth oil type relative to the viscosity increase rate (measured using the measurement device 4) of the frying oil P, in which the actually measured total polar compounds are compared with the total polar compounds calculated using Equation (31), Equation (32), Equation (45), and Equation (46), respectively. FIG. 175 illustrates a graph showing the correlation of the acid value of the frying oil P relating to the sixth oil type relative to the viscosity increase rate (measured using the measurement device 4) of the frying oil P, in which the actually measured acid values are compared with the acid values calculated using Equation (31), Equation (32), Equation (45), and Equation (46), respectively. FIG. 176 illustrates a graph showing the correlation of the color of the frying oil P relating to the sixth oil type relative to the viscosity increase rate (measured using the measurement device 4) of the frying oil P, in which the actually measured values of the color are compared with the colors calculated using Equation (31), Equation (32), Equation (45), and Equation (46), respectively.

FIG. 177 illustrates a graph showing the correlation of the total polar compounds contained in the frying oil P relating to the sixth oil type relative to the color (measured using the measurement device 4) of the frying oil P, in which the actually measured total polar compounds are compared with the total polar compounds calculated using Equation (31), Equation (32), Equation (45), and Equation (46), respectively. FIG. 178 illustrates a graph showing the correlation of the viscosity increase rate of the frying oil P relating to the sixth oil type relative to the color (measured using the measurement device 4) of the frying oil P, in which the actually measured viscosity increase rates are compared with the viscosity increase rates calculated using Equation (31), Equation (32), Equation (45), and Equation (46), respectively.

In each of FIG. 161 to FIG. 169 relating to the case where the type of the frying oil P is the fifth oil type, a graph including a plurality of “∘” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (43), and a graph including a plurality of “▴” is indicative of the correlation graph for Equation (44), respectively.

In each of FIG. 170 to FIG. 178 relating to the case where the type of the frying oil P is the sixth oil type, a graph including a plurality of “∘” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (45), and a graph including a plurality of “▴” is indicative of the correlation graph for Equation (46), respectively.

As illustrated in each of FIG. 161 to FIG. 169, the correlation graph for Equation (43) and the correlation graph for Equation (44) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually measured second deterioration indicator of the frying oil P further than the correlation graph for Equation (43) and the correlation graph for Equation (44).

In the same manner, as illustrated in each of FIG. 170 to FIG. 178, the correlation graph for Equation (45) and the correlation graph for Equation (46) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually second deterioration indicator of the frying oil P further than the correlation graph for Equation (45) and the correlation graph for Equation (46).

Thus, in the case where the measurement device 4 measures the first deterioration indicator of the frying oil P, the cloud 9C identifies whether the type of the frying oil P is the fifth oil type or the sixth oil type based on the CDM value of the frying oil P and calculates the second deterioration indicator of the frying oil P using the correlation equation for the oil type as identified. This enables the calculation of the second deterioration indicator of the frying oil P with higher accuracy than the case of calculating it using Equation (31) or Equation (32) in all cases without considering the type of oil classified based on the CDM value of the frying oil P.

### Fourteenth Embodiment

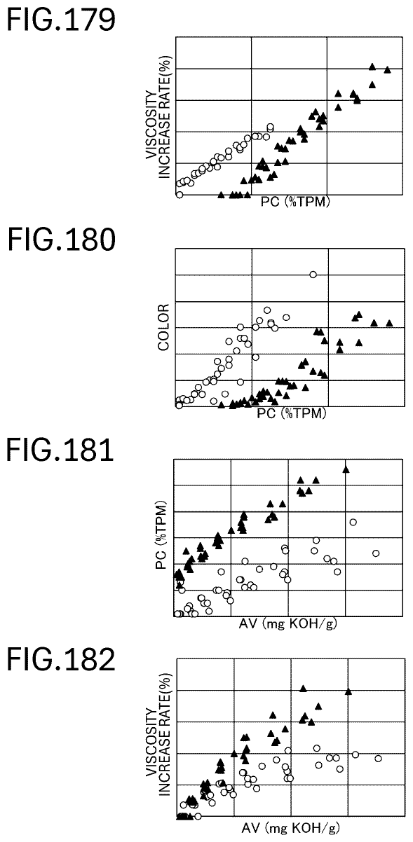

Next, the cloud 9C according to the fourteenth embodiment of the present invention will be described with reference to FIG. 179 to FIG. 368.

As described in the seventh embodiment, the frying oil P (vegetable oil) is mainly composed of TG (triacylglycerol). When heated, a part of TG is broken down into DG (diacylglycerol), MG (monoacylglycerol), and FFA (free fatty acid).

In new oil and heated oil, the content ratio of each of TG, DG, MG, and FFA differs depending on the type of oil, and thus the type of the frying oil P can be classified into the seventh oil type and the eighth oil type depending on the lipid molecular species in the frying oil P.

FIG. 179 illustrates a graph showing the correlation of the viscosity increase rate of the frying oil P relating to the seventh oil type and the eighth oil type, which are classified based on the MG content in new oil, relative to the total polar compounds contained in the frying oil P. FIG. 180 illustrates a graph showing the correlation of the color of the frying oil P relating to the seventh oil type and the eighth oil type, which are classified based on the MG content in new oil, relative to the total polar compounds contained in the frying oil P. FIG. 181 illustrates a graph showing the correlation of the total polar compounds contained in the frying oil P relating to the seventh oil type and the eighth oil type, which are classified based on the MG content in new oil, relative to the acid value of the frying oil P. FIG. 182 illustrates a graph showing the correlation of the viscosity increase rate of the frying oil P relating to the seventh oil type and the eighth oil type, which are classified based on the MG content in new oil, relative to the acid value of the frying oil P.

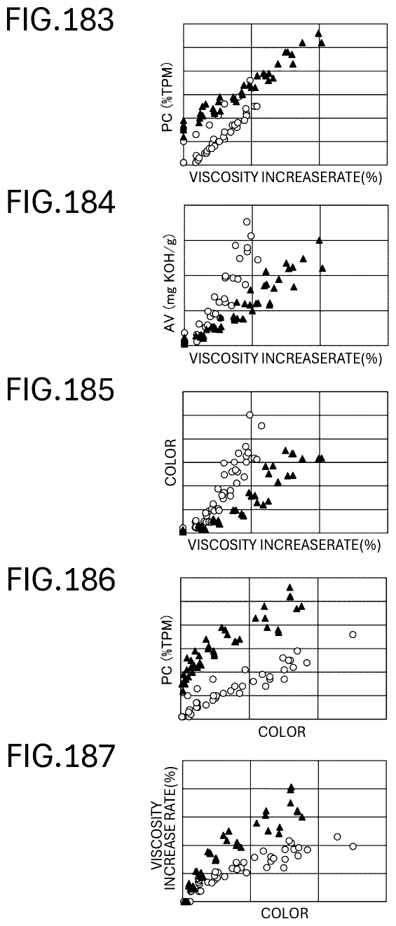

FIG. 183 illustrates a graph showing the correlation of the total polar compounds contained in the frying oil P relating to the seventh oil type and the eighth oil type, which are classified based on the MG content in new oil, relative to the viscosity increase rate of the frying oil P. FIG. 184 illustrates a graph showing the correlation of the acid value of the frying oil P relating to the seventh oil type and the eighth oil type, which are classified based on the MG content in new oil, relative to the viscosity increase rate of the frying oil P. FIG. 185 illustrates a graph showing the correlation of the color of the frying oil P relating to the seventh oil type and the eighth oil type, which are classified based on the MG content in new oil, relative to the viscosity increase rate of the frying oil P. FIG. 186 illustrates a graph showing the correlation of the total polar compounds contained in the frying oil P relating to the seventh oil type and the eighth oil type, which are classified based on the MG content in new oil, relative to the color of the frying oil P. FIG. 187 illustrates a graph showing the correlation of the viscosity increase rate of the frying oil P relating to the seventh oil type and the eighth oil type, which are classified based on the MG content in new oil, relative to the color of the frying oil P.

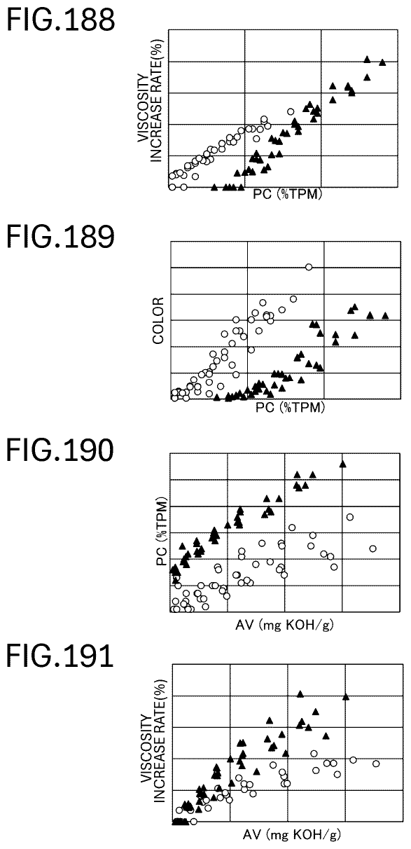

FIG. 188 illustrates a graph showing the correlation of the viscosity increase rate of the frying oil P relating to the seventh oil type and the eighth oil type, which are classified based on the FFA content in new oil, relative to the total polar compounds contained in the frying oil P. FIG. 189 illustrates a graph showing the correlation of the color of the frying oil P relating to the seventh oil type and the eighth oil type, which are classified based on the FFA content in new oil, relative to the total polar compounds contained in the frying oil P. FIG. 190 illustrates a graph showing the correlation of the total polar compounds contained in the frying oil P relating to the seventh oil type and the eighth oil type, which are classified based on the FFA content in new oil, relative to the acid value of the frying oil P. FIG. 191 illustrates a graph showing the correlation of the viscosity increase rate of the frying oil P relating to the seventh oil type and the eighth oil type, which are classified based on the FFA content in new oil, relative to the acid value of the frying oil P.

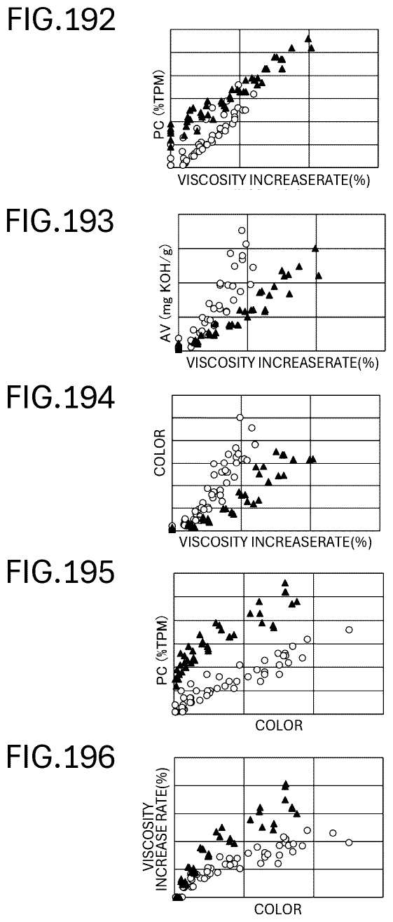

FIG. 192 illustrates a graph showing the correlation of the total polar compounds contained in the frying oil P relating to the seventh oil type and the eighth oil type, which are classified based on the FFA content in new oil, relative to the viscosity increase rate of the frying oil P. FIG. 193 illustrates a graph showing the correlation of the acid value of the frying oil P relating to the seventh oil type and the eighth oil type, which are classified based on the FFA content in new oil, relative to the viscosity increase rate of the frying oil P. FIG. 194 illustrates a graph showing the correlation of the color of the frying oil P relating to the seventh oil type and the eighth oil type, which are classified based on the FFA content in new oil, relative to the viscosity increase rate of the frying oil P. FIG. 195 illustrates a graph showing the correlation of the total polar compounds contained in the frying oil P relating to the seventh oil type and the eighth oil type, which are classified based on the FFA content in new oil, relative to the color of the frying oil P. FIG. 196 illustrates a graph showing the correlation of the viscosity increase rate of the frying oil P relating to the seventh oil type and the eighth oil type, which are classified based on the FFA content in new oil, relative to the color of the frying oil P.

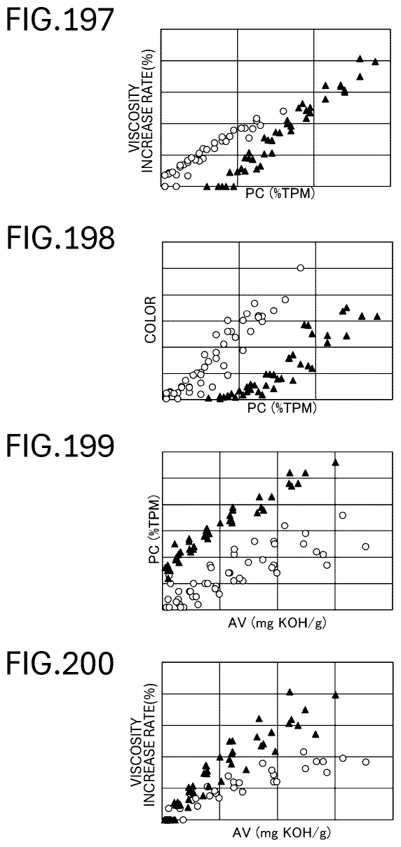

FIG. 197 illustrates a graph showing the correlation of the viscosity increase rate of the frying oil P relating to the seventh oil type and the eighth oil type, which are classified based on the MG content in heated oil, relative to the total polar compounds contained in the frying oil P. FIG. 198 illustrates a graph showing the correlation of the color of the frying oil P relating to the seventh oil type and the eighth oil type, which are classified based on the MG content in heated oil, relative to the total polar compounds contained in the frying oil P. FIG. 199 illustrates a graph showing the correlation of the total polar compounds contained in the frying oil P relating to the seventh oil type and the eighth oil type, which are classified based on the MG content in heated oil, relative to the acid value of the frying oil P. FIG. 200 illustrates a graph showing the correlation of the viscosity increase rate of the frying oil P relating to the seventh oil type and the eighth oil type, which are classified based on the MG content in heated oil, relative to the acid value of the frying oil P.

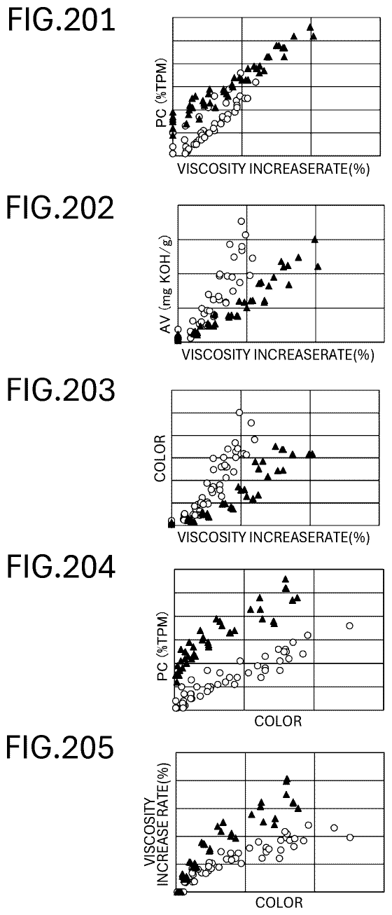

FIG. 201 illustrates a graph showing the correlation of the total polar compounds contained in the frying oil P relating to the seventh oil type and the eighth oil type, which are classified based on the MG content in heated oil, relative to the viscosity increase rate of the frying oil P. FIG. 202 illustrates a graph showing the correlation of the acid value of the frying oil P relating to the seventh oil type and the eighth oil type, which are classified based on the MG content in heated oil, relative to the viscosity increase rate of the frying oil P. FIG. 203 illustrates a graph showing the correlation of the color of the frying oil P relating to the seventh oil type and the eighth oil type, which are classified based on the MG content in heated oil, relative to the viscosity increase rate of the frying oil P. FIG. 204 illustrates a graph showing the correlation of the total polar compounds contained in the frying oil P relating to the seventh oil type and the eighth oil type, which are classified based on the MG content in heated oil, relative to the color of the frying oil P. FIG. 205 illustrates a graph showing the correlation of the viscosity increase rate of the frying oil P relating to the seventh oil type and the eighth oil type, which are classified based on the MG content in heated oil, relative to the color of the frying oil P.

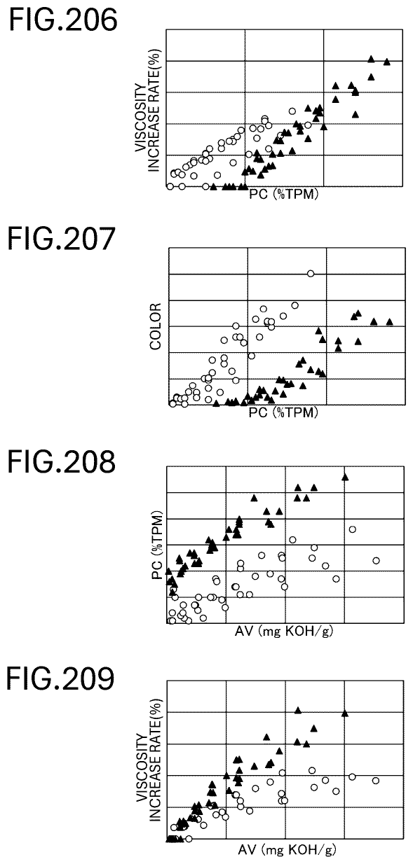

FIG. 206 illustrates a graph showing the correlation of the viscosity increase rate of the frying oil P relating to the seventh oil type and the eighth oil type, which are classified based on the TG content in heated oil, relative to the total polar compounds contained in the frying oil P. FIG. 207 illustrates a graph showing the correlation of the color of the frying oil P relating to the seventh oil type and the eighth oil type, which are classified based on the TG content in heated oil, relative to the total polar compounds contained in the frying oil P. FIG. 208 illustrates a graph showing the correlation of the total polar compounds contained in the frying oil P relating to the seventh oil type and the eighth oil type, which are classified based on the TG content in heated oil, relative to the acid value of the frying oil P. FIG. 209 illustrates a graph showing the correlation of the viscosity increase rate of the frying oil P relating to the seventh oil type and the eighth oil type, which are classified based on the TG content in heated oil, relative to the acid value of the frying oil P.

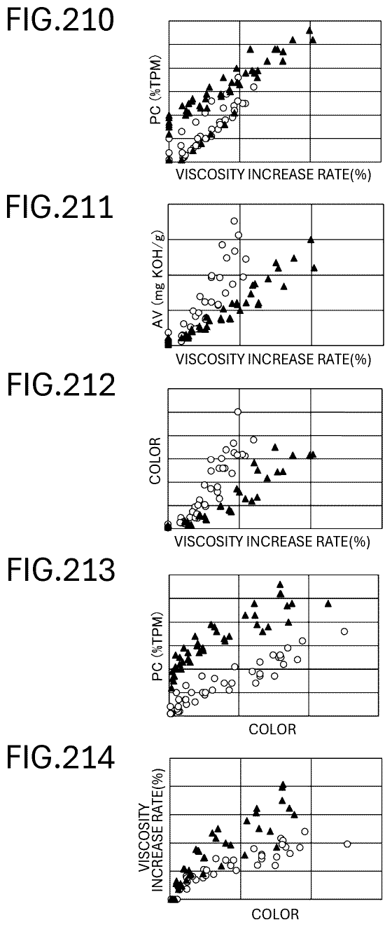

FIG. 210 illustrates a graph showing the correlation of the total polar compounds contained in the frying oil P relating to the seventh oil type and the eighth oil type, which are classified based on the TG content in heated oil, relative to the viscosity increase rate of the frying oil P. FIG. 211 illustrates a graph showing the correlation of the acid value of the frying oil P relating to the seventh oil type and the eighth oil type, which are classified based on the TG content in heated oil, relative to the viscosity increase rate of the frying oil P. FIG. 212 illustrates a graph showing the correlation of the color of the frying oil P relating to the seventh oil type and the eighth oil type, which are classified based on the TG content in heated oil, relative to the viscosity increase rate of the frying oil P. FIG. 213 illustrates a graph showing the correlation of the total polar compounds contained in the frying oil P relating to the seventh oil type and the eighth oil type, which are classified based on the TG content in heated oil, relative to the color of the frying oil P. FIG. 214 illustrates a graph showing the correlation of the viscosity increase rate of the frying oil P relating to the seventh oil type and the eighth oil type, which are classified based on the TG content in heated oil, relative to the color of the frying oil P.

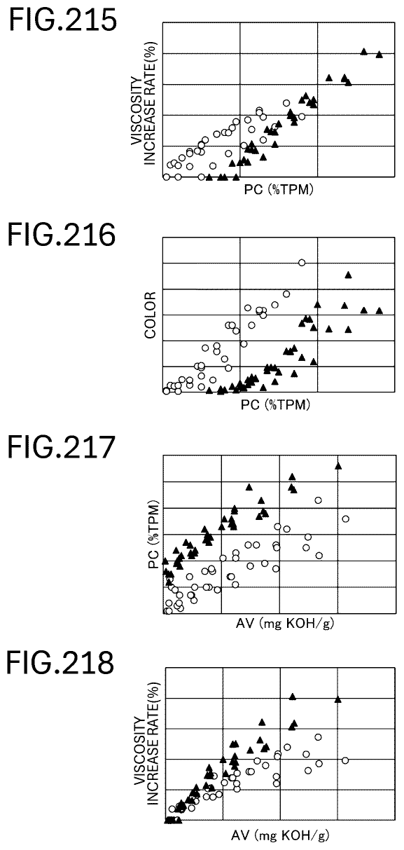

FIG. 215 illustrates a graph showing the correlation of the viscosity increase rate of the frying oil P relating to the seventh oil type and the eighth oil type, which are classified based on the rate of increase in the DG content due to heating, relative to the total polar compounds contained in the frying oil P. FIG. 216 illustrates a graph showing the correlation of the color of the frying oil P relating to the seventh oil type and the eighth oil type, which are classified based on the rate of increase in the DG content due to heating, relative to the total polar compounds contained in the frying oil P. FIG. 217 illustrates a graph showing the correlation of the total polar compounds contained in the frying oil P relating to the seventh oil type and the eighth oil type, which are classified based on the rate of increase in the DG content due to heating, relative to the acid value of the frying oil P. FIG. 218 illustrates a graph showing the correlation of the viscosity increase rate of the frying oil P relating to the seventh oil type and the eighth oil type, which are classified based on the rate of increase in the DG content due to heating, relative to the acid value of the frying oil P.

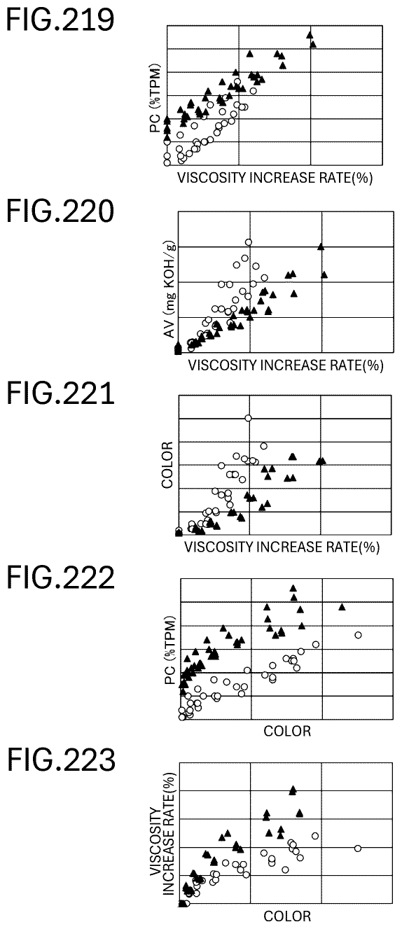

FIG. 219 illustrates a graph showing the correlation of the total polar compounds contained in the frying oil P relating to the seventh oil type and the eighth oil type, which are classified based on the rate of increase in the DG content due to heating, relative to the viscosity increase rate of the frying oil P. FIG. 220 illustrates a graph showing the correlation of the acid value of the frying oil P relating to the seventh oil type and the eighth oil type, which are classified based on the rate of increase in the DG content due to heating, relative to the viscosity increase rate of the frying oil P. FIG. 221 illustrates a graph showing the correlation of the color of the frying oil P relating to the seventh oil type and the eighth oil type, which are classified based on the rate of increase in the DG content due to heating, relative to the viscosity increase rate of the frying oil P. FIG. 222 illustrates a graph showing the correlation of the total polar compounds contained in the frying oil P relating to the seventh oil type and the eighth oil type, which are classified based on the rate of increase in the DG content due to heating, relative to the color of the frying oil P. FIG. 223 illustrates a graph showing the correlation of the viscosity increase rate of the frying oil P relating to the seventh oil type and the eighth oil type, which are classified based on the rate of increase in the DG content due to heating, relative to the color of the frying oil P.

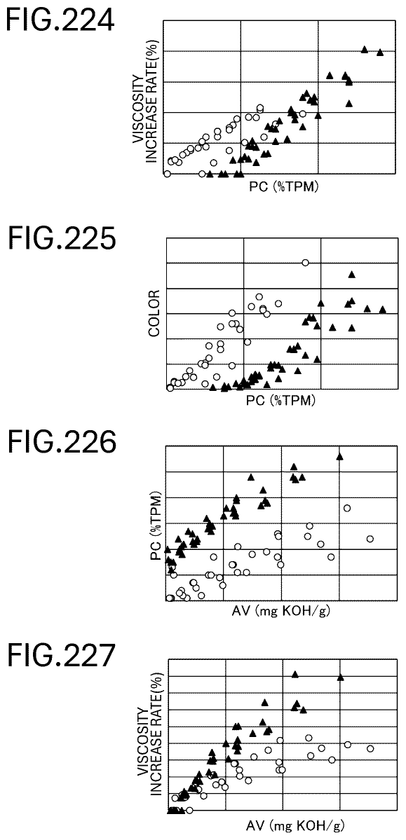

FIG. 224 illustrates a graph showing the correlation of the viscosity increase rate of the frying oil P relating to the seventh oil type and the eighth oil type, which are classified based on the rate of increase in the FFA content due to heating, relative to the total polar compounds contained in the frying oil P. FIG. 225 illustrates a graph showing the correlation of the color of the frying oil P relating to the seventh oil type and the eighth oil type, which are classified based on the rate of increase in the FFA content due to heating, relative to the total polar compounds contained in the frying oil P. FIG. 226 illustrates a graph showing the correlation of the total polar compounds contained in the frying oil P relating to the seventh oil type and the eighth oil type, which are classified based on the rate of increase in the FFA content due to heating, relative to the acid value of the frying oil P. FIG. 227 illustrates a graph showing the correlation of the viscosity increase rate of the frying oil P relating to the seventh oil type and the eighth oil type, which are classified based on the rate of increase in the FFA content due to heating, relative to the acid value of the frying oil P.

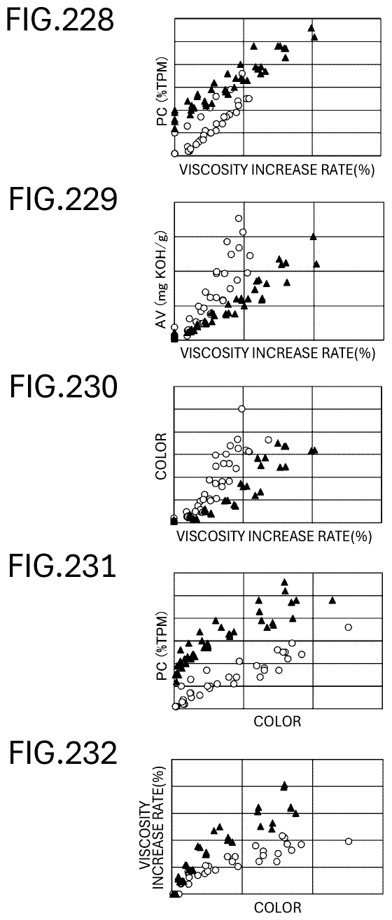

FIG. 228 illustrates a graph showing the correlation of the total polar compounds contained in the frying oil P relating to the seventh oil type and the eighth oil type, which are classified based on the rate of increase in the FFA content due to heating, relative to the viscosity increase rate of the frying oil P. FIG. 229 illustrates a graph showing the correlation of the acid value of the frying oil P relating to the seventh oil type and the eighth oil type, which are classified based on the rate of increase in the FFA content due to heating, relative to the viscosity increase rate of the frying oil P. FIG. 230 illustrates a graph showing the correlation of the color of the frying oil P relating to the seventh oil type and the eighth oil type, which are classified based on the rate of increase in the FFA content due to heating, relative to the viscosity increase rate of the frying oil P. FIG. 231 illustrates a graph showing the correlation of the total polar compounds contained in the frying oil P relating to the seventh oil type and the eighth oil type, which are classified based on the rate of increase in the FFA content due to heating, relative to the color of the frying oil P. FIG. 232 illustrates a graph showing the correlation of the viscosity increase rate of the frying oil P relating to the seventh oil type and the eighth oil type, which are classified based on the rate of increase in the FFA content due to heating, relative to the color of the frying oil P.

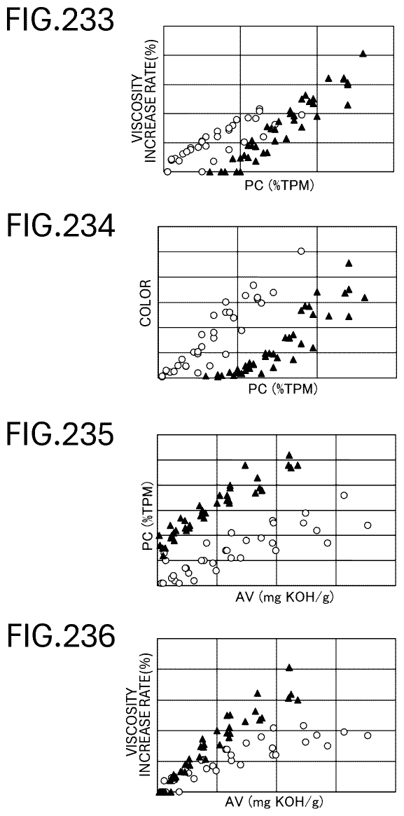

FIG. 233 illustrates a graph showing the correlation of the viscosity increase rate of the frying oil P relating to the seventh oil type and the eighth oil type, which are classified based on the rate of decrease in the TG content due to heating, relative to the total polar compounds contained in the frying oil P. FIG. 234 illustrates a graph showing the correlation of the color of the frying oil P relating to the seventh oil type and the eighth oil type, which are classified based on the rate of decrease in the TG content due to heating, relative to the total polar compounds contained in the frying oil P. FIG. 235 illustrates a graph showing the correlation of the total polar compounds contained in the frying oil P relating to the seventh oil type and the eighth oil type, which are classified based on the rate of decrease in the TG content due to heating, relative to the acid value of the frying oil P. FIG. 236 illustrates a graph showing the correlation of the viscosity increase rate of the frying oil P relating to the seventh oil type and the eighth oil type, which are classified based on the rate of decrease in the TG content due to heating, relative to the acid value of the frying oil P.

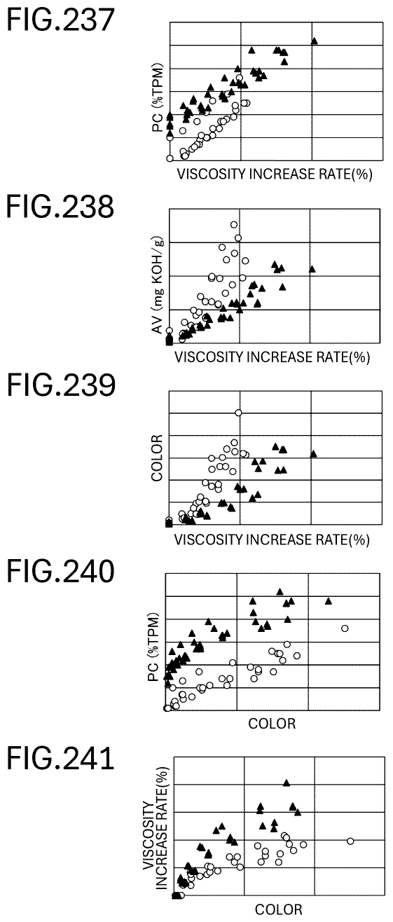

FIG. 237 illustrates a graph showing the correlation of the total polar compounds contained in the frying oil P relating to the seventh oil type and the eighth oil type, which are classified based on the rate of decrease in the TG content due to heating, relative to the viscosity increase rate of the frying oil P. FIG. 238 illustrates a graph showing the correlation of the acid value of the frying oil P relating to the seventh oil type and the eighth oil type, which are classified based on the rate of decrease in the TG content due to heating, relative to the viscosity increase rate of the frying oil P. FIG. 239 illustrates a graph showing the correlation of the color of the frying oil P relating to the seventh oil type and the eighth oil type, which are classified based on the rate of decrease in the TG content due to heating, relative to the viscosity increase rate of the frying oil P. FIG. 240 illustrates a graph showing the correlation of the total polar compounds contained in the frying oil P relating to the seventh oil type and the eighth oil type, which are classified based on the rate of decrease in the TG content due to heating, relative to the color of the frying oil P. FIG. 241 illustrates a graph showing the correlation of the viscosity increase rate of the frying oil P relating to the seventh oil type and the eighth oil type, which are classified based on the rate of decrease in the TG content due to heating, relative to the color of the frying oil P.

In each of the correlation graphs illustrated in FIG. 179 to FIG. 241, the first deterioration indicator corresponding to the horizontal axis is the value measured using the measurement device 4.

As illustrated in each of FIG. 179 to FIG. 241, the correlation graph of the second deterioration indicator of the frying oil P relative to the first deterioration indicator of the frying oil P differs between the seventh oil type and the eighth oil type. In each of FIG. 179 to FIG. 241, a correlation graph including a plurality of “∘” is indicative of the correlation graph of the first deterioration indicator of the frying oil P corresponding to the seventh oil type relative to the second deterioration indicator of the frying oil P, and a correlation graph including a plurality of “▴” is indicative of the correlation graph of the first deterioration indicator of the frying oil P corresponding to the eighth oil type relative to the second deterioration indicator of the frying oil P, respectively.

In the case where the type of the frying oil P is the seventh oil type, the correlation equation for the second deterioration indicator of the frying oil P relative to the first deterioration indicator of the frying oil P is a linear equation expressed with the following Equation (47) including each of α7 as the first-order coefficient α of Di1n and β7 as the constant β in Equation (31), or a quadratic equation expressed with the following Equation (48) including each of γ7 as the second-order coefficient γ of Di1n, δ7 as the first-order coefficient δ of Di1n, and ε7 as the constant ε in Equation (32).

$$Di 2 n = α 7 \times ( Di 1 n ) + β 7 \qquad ( 47 )$$

$$Di 2 n = γ 7 \times {( Di 1 n )}^{2} + δ 7 \times ( PCn ) + ε 7 \qquad ( 48 )$$

In the case where the type of the frying oil P is the eighth oil type, the correlation equation for the second deterioration indicator of the frying oil P relative to the first deterioration indicator of the frying oil P is a linear equation expressed with the following Equation (49) including each of α8 as the first-order coefficient α of Di1n and β8 as the constant β in Equation (31), or a quadratic equation expressed with the following Equation (50) including each of γ8 as the second-order coefficient γ of Di1n, δ8 as the first-order coefficient δ of Di1n, and ε8 as the constant ε in Equation (32).

$$Di 2 n = α 8 \times ( Di 1 n ) + β8 \qquad ( 49 )$$

$$Di 2 n = γ 8 \times {( Di 1 n )}^{2} + δ 8 \times ( Di 1 n ) + ε8 \qquad ( 50 )$$

Here, in each of FIG. 179, FIG. 180, FIG. 184, FIG. 185, FIG. 188, FIG. 189, FIG. 193, FIG. 194, FIG. 197, FIG. 198, FIG. 202, FIG. 203, FIG. 206, FIG. 207, FIG. 211, FIG. 212, FIG. 215, FIG. 216, FIG. 220, FIG. 221, FIG. 224, FIG. 225, FIG. 229, FIG. 230, FIG. 233, FIG. 234, FIG. 238, and FIG. 239, the first-order coefficient α8 of Di1n in Equation (49) is smaller than the first-order coefficient α7 of Di1n in Equation (47) (α6<α5), and the constant β8 in Equation (49) is smaller than the constant β7 in Equation (47) (β8<β7). Furthermore, the second-order coefficient γ8 of Di1n in Equation (50) is smaller than the second-order coefficient γ7 of Di1n in Equation (48) (γ8<γ7), the first-order coefficient δ8 of Di1n in Equation (50) is smaller than the first-order coefficient δ7 of Di1n in Equation (48) (δ8<δ7), and the constant ε8 in Equation (50) is smaller than the constant ε7 in Equation (48) (ε8<ε7).

On the other hand, in each of FIG. 181 to FIG. 183, FIG. 186, FIG. 187, FIG. 190 to FIG. 192, FIG. 195, FIG. 196, FIG. 199 to FIG. 201, FIG. 204, FIG. 205, FIG. 208 to FIG. 210, FIG. 213, FIG. 214, FIG. 217 to FIG. 219, FIG. 222, FIG. 223, FIG. 226 to FIG. 228, FIG. 231, FIG. 232, FIG. 235 to FIG. 237, FIG. 240, and FIG. 241, the first-order coefficient α8 of Di1n in Equation (49) is greater than the first-order coefficient α7 of Di1n in Equation (47) (α8>α7), and the constant β7 in Equation (47) is greater than the constant β8 in Equation (49) (β8>β7).

Furthermore, the second-order coefficient γ8 of Di1n in Equation (50) is greater than the second-order coefficient γ7 of Di1n in Equation (48) (γ8>γ7), the first-order coefficient δ8 of Di1n in Equation (50) is greater than the first-order coefficient δ7 of Di1n in Equation (48) (δ8>δ7), and the constant ε8 in Equation (50) is greater than the constant ε7 in Equation (48) (ε8>ε7).

As described above, especially in the case of measuring the first deterioration indicator of the frying oil P using the measurement device 4, a difference is found in the correlation of the second deterioration indicator of the frying oil P relative to the first deterioration indicator of the frying oil P between the seventh oil type and the eighth oil type. Thus, by using the equation in which the difference therebetween is considered (Equation (47) or Equation (48) in the case of the seventh oil type and Equation (49) or Equation (50) in the case of the eighth oil type), the calculation of the second deterioration indicator of the frying oil P can be carried out with higher accuracy.

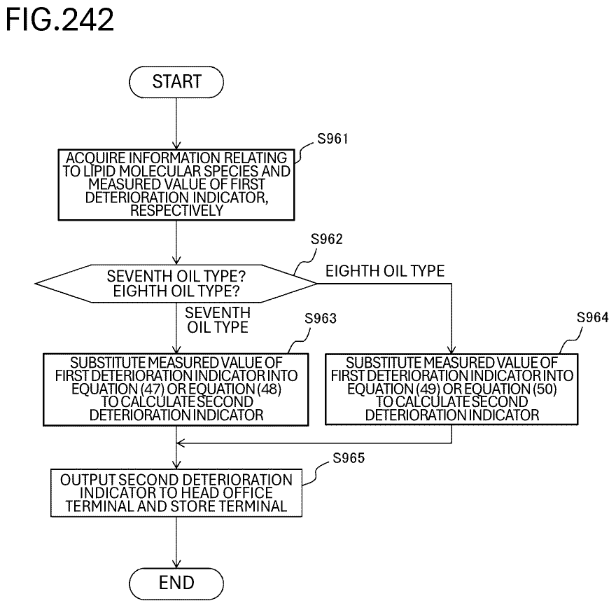

FIG. 242 illustrates a flowchart of a flow of the processing to be executed by the cloud 9C according to the fourteenth embodiment.

In the cloud 9C according to the present embodiment, firstly, the data acquisition section 91C acquires the measured value of the first deterioration indicator of the frying oil P output from the measurement device 4 and the information relating to the lipid molecular species of the frying oil P output from the store terminal 6 (step S961).

The information relating to the lipid molecular species of the frying oil P includes, for example, the information indicative of the specific names of oil types, and the like. The information relating to the lipid molecular species of the frying oil P does not necessarily have to be output from the store terminal 6 to the cloud 9C, but may be output from, for example, the head office terminal 7 to the cloud 9C.

Next, the oil type identification section 97 identifies whether the type of the frying oil P is the seventh oil type or the eighth oil type based on the information relating to the lipid molecular species of the frying oil P acquired in step S961 (step S962).

In the case where it is identified in step S962 that the type of the frying oil P is the seventh oil type (step S962/seventh oil type), the deterioration indicator calculation section 93C substitutes the measured value of the first deterioration indicator of the frying oil P acquired in step S961 into Di1n of Equation (47) or Di1n of Equation (48), which is stored in the storage section 92C, to calculate the second deterioration indicator Di2n of the frying oil P (step S963).

On the other hand, in the case where it is identified in step S962 that the type of the frying oil P is the eighth oil type (step S962/eighth oil type), the deterioration indicator calculation section 93C substitutes the measured value of the first deterioration indicator of the frying oil P acquired in step S961 into Di1n of Equation (49) or Di1n of Equation (50), which is stored in the storage section 92C, to calculate the second deterioration indicator Di2n of the frying oil P (step S964).

Then, the detection result output section 94 outputs the second deterioration indicator Di2n of the frying oil P calculated in step S963 or step S964 to the store terminal 6 and the head office terminal 7, respectively, as the result of detection of the deterioration degree of the frying oil P (step S965), whereby the processing in the cloud 9C is ended.

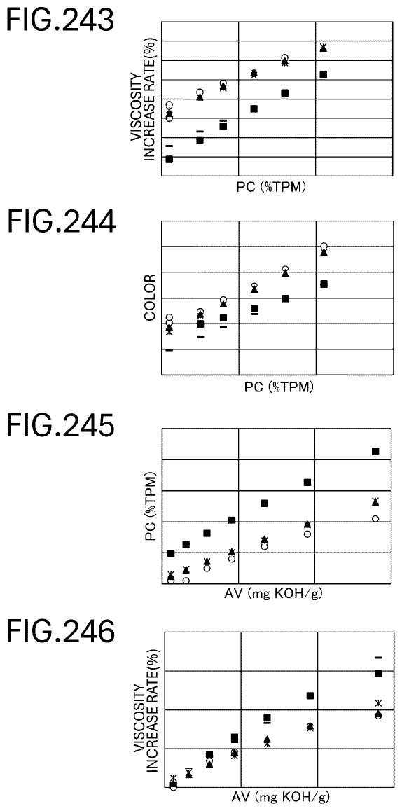

FIG. 243 illustrates a graph showing the correlation of the viscosity increase rate of the frying oil P relating to the seventh oil type, which is classified based on the MG content in new oil, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 244 illustrates a graph showing the correlation of the color of the frying oil P relating to the seventh oil type, which is classified based on the MG content in new oil, relative to the total polar compounds (measured using the measurement device 4) contained in the frying oil P, in which the actually measured values of the color are compared with the colors calculated using Equation (31), Equation (32), Equation (47), and Equation (48), respectively. FIG. 245 illustrates a graph showing the correlation of the total polar compounds contained in the frying oil P relating to the seventh oil type, which is classified based on the MG content in new oil, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 246 illustrates a graph showing the correlation of the viscosity increase rate of the frying oil P relating to the seventh oil type, which is classified based on the MG content in new oil, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

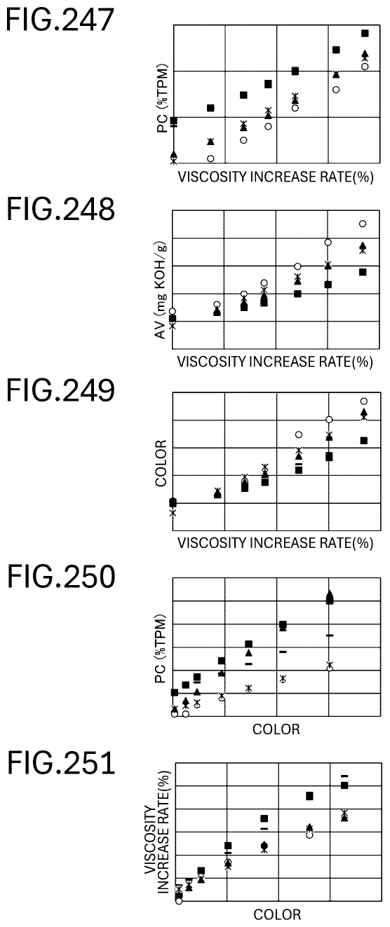

FIG. 247 illustrates a graph showing the correlation of the total polar compounds contained in the frying oil P relating to the seventh oil type, which is classified based on the MG content in new oil, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 248 illustrates a graph showing the correlation of the acid value of the frying oil P relating to the seventh oil type, which is classified based on the MG content in new oil, relative to the viscosity increase rate (measured using the measurement device 4) of the frying oil P, in which the actually measured acid values are compared with the acid values calculated using Equation (31), Equation (32), Equation (47), and Equation (48), respectively. FIG. 249 illustrates a graph showing the correlation of the color of the frying oil P relating to the seventh oil type, which is classified based on the MG content in new oil, relative to the viscosity increase rate (measured using the measurement device 4) of the frying oil P, in which the actually measured values of the color are compared with the colors calculated using Equation (31), Equation (32), Equation (47), and Equation (48), respectively. FIG. 250 illustrates a graph showing the correlation of the total polar compounds contained in the frying oil P relating to the seventh oil type, which is classified based on the MG content in new oil, relative to the color (measured using the measurement device 4) of the frying oil P, in which the actually measured total polar compounds are compared with the total polar compounds calculated using Equation (31), Equation (32), Equation (47), and Equation (48), respectively. FIG. 251 illustrates a graph showing the correlation of the viscosity increase rate of the frying oil P relating to the seventh oil type, which is classified based on the MG content in new oil, relative to the color (measured using the measurement device 4) of the frying oil P, in which the actually measured viscosity increase rates are compared with the viscosity increase rates calculated using Equation (31), Equation (32), Equation (47), and Equation (48), respectively.

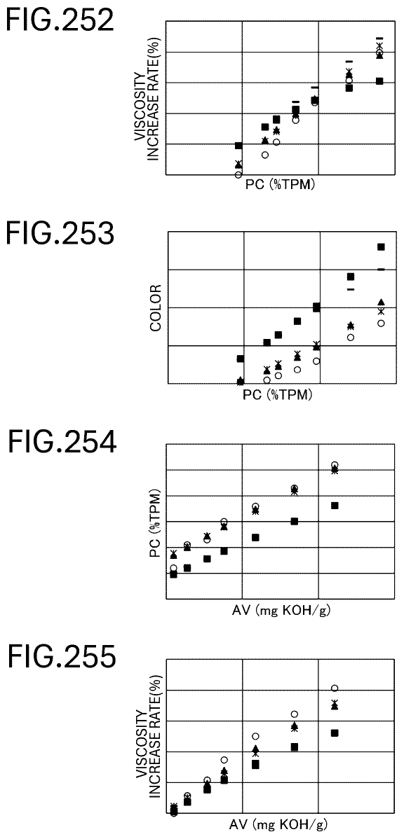

FIG. 252 illustrates a graph showing the correlation of the viscosity increase rate of the frying oil P relating to the eighth oil type, which is classified based on the MG content in new oil, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 253 illustrates a graph showing the correlation of the color of the frying oil P relating to the eighth oil type, which is classified based on the MG content in new oil, relative to the total polar compounds (measured using the measurement device 4) contained in the frying oil P, in which the actually measured values of the color are compared with the colors calculated using Equation (31), Equation (32), Equation (49), and Equation (50), respectively. FIG. 254 illustrates a graph showing the correlation of the total polar compounds contained in the frying oil P relating to the eighth oil type, which is classified based on the MG content in new oil, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 255 illustrates a graph showing the correlation of the viscosity increase rate of the frying oil P relating to the eighth oil type, which is classified based on the MG content in new oil, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

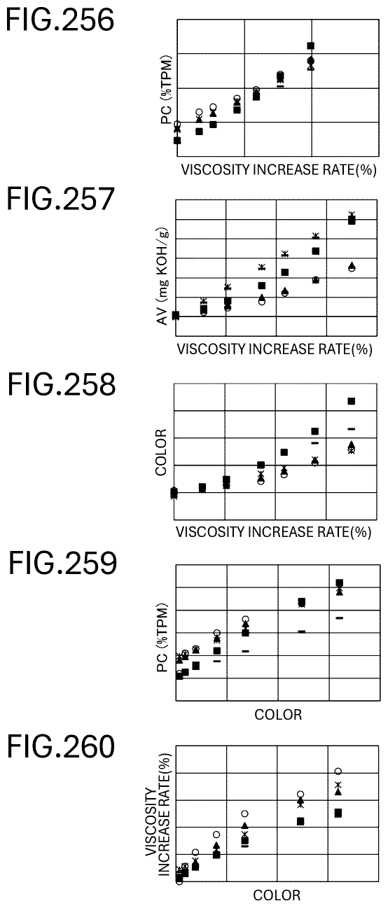

FIG. 256 illustrates a graph showing the correlation of the total polar compounds contained in the frying oil P relating to the eighth oil type, which is classified based on the MG content in new oil, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 257 illustrates a graph showing the correlation of the acid value of the frying oil P relating to the eighth oil type, which is classified based on the MG content in new oil, relative to the viscosity increase rate (measured using the measurement device 4) of the frying oil P, in which the actually measured acid values are compared with the acid values calculated using Equation (31), Equation (32), Equation (49), and Equation (50), respectively. FIG. 258 illustrates a graph showing the correlation of the color of the frying oil P relating to the eighth oil type, which is classified based on the MG content in new oil, relative to the viscosity increase rate (measured using the measurement device 4) of the frying oil P, in which the actually measured values of the color are compared with the colors calculated using Equation (31), Equation (32), Equation (49), and Equation (50), respectively. FIG. 259 illustrates a graph showing the correlation of the total polar compounds contained in the frying oil P relating to the eighth oil type, which is classified based on the MG content in new oil, relative to the color (measured using the measurement device 4) of the frying oil P, in which the actually measured total polar compounds are compared with the total polar compounds calculated using Equation (31), Equation (32), Equation (49), and Equation (50), respectively. FIG. 260 illustrates a graph showing the correlation of the viscosity increase rate of the frying oil P relating to the eighth oil type, which is classified based on the MG content in new oil, relative to the color (measured using the measurement device 4) of the frying oil P, in which the actually measured viscosity increase rates are compared with the viscosity increase rates calculated using Equation (31), Equation (32), Equation (49), and Equation (50), respectively.

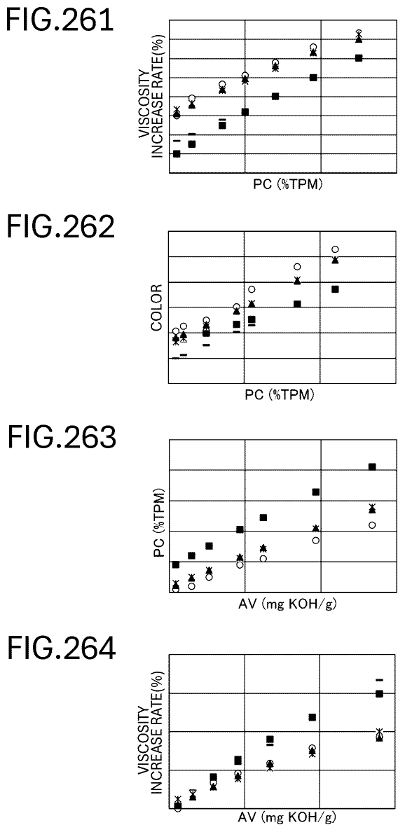

FIG. 261 illustrates a graph showing the correlation of the viscosity increase rate of the frying oil P relating to the seventh oil type, which is classified based on the FFA content in new oil, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 262 illustrates a graph showing the correlation of the color of the frying oil P relating to the seventh oil type, which is classified based on the FFA content in new oil, relative to the total polar compounds (measured using the measurement device 4) contained in the frying oil P, in which the actually measured values of the color are compared with the colors calculated using Equation (31), Equation (32), Equation (47), and Equation (48), respectively. FIG. 263 illustrates a graph showing the correlation of the total polar compounds contained in the frying oil P relating to the seventh oil type, which is classified based on the FFA content in new oil, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 264 illustrates a graph showing the correlation of the viscosity increase rate of the frying oil P relating to the seventh oil type, which is classified based on the FFA content in new oil, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

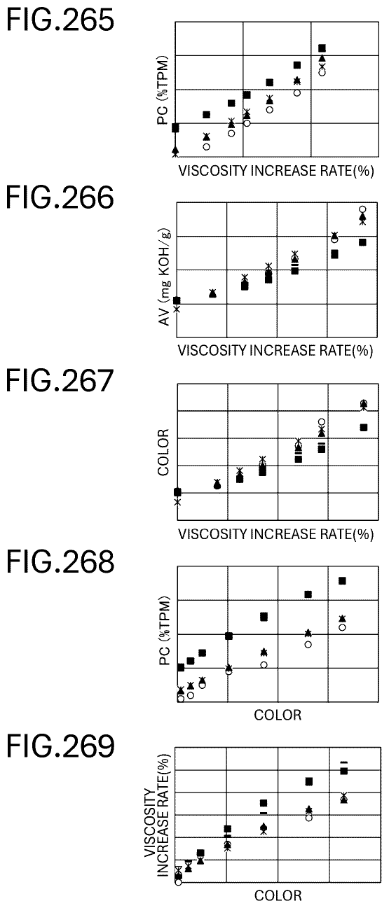

FIG. 265 illustrates a graph showing the correlation of the total polar compounds contained in the frying oil P relating to the seventh oil type, which is classified based on the FFA content in new oil, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 266 illustrates a graph showing the correlation of the acid value of the frying oil P relating to the seventh oil type, which is classified based on the FFA content in new oil, relative to the viscosity increase rate (measured using the measurement device 4) of the frying oil P, in which the actually measured acid values are compared with the acid values calculated using Equation (31), Equation (32), Equation (47), and Equation (48), respectively. FIG. 267 illustrates a graph showing the correlation of the color of the frying oil P relating to the seventh oil type, which is classified based on the FFA content in new oil, relative to the viscosity increase rate (measured using the measurement device 4) of the frying oil P, in which the actually measured values of the color are compared with the colors calculated using Equation (31), Equation (32), Equation (47), and Equation (48), respectively. FIG. 268 illustrates a graph showing the correlation of the total polar compounds contained in the frying oil P relating to the seventh oil type, which is classified based on the FFA content in new oil, relative to the color (measured using the measurement device 4) of the frying oil P, in which the actually measured total polar compounds are compared with the total polar compounds calculated using Equation (31), Equation (32), Equation (47), and Equation (48), respectively. FIG. 269 illustrates a graph showing the correlation of the viscosity increase rate of the frying oil P relating to the seventh oil type, which is classified based on the FFA content in new oil, relative to the color (measured using the measurement device 4) of the frying oil P, in which the actually measured viscosity increase rates are compared with the viscosity increase rates calculated using Equation (31), Equation (32), Equation (47), and Equation (48), respectively.

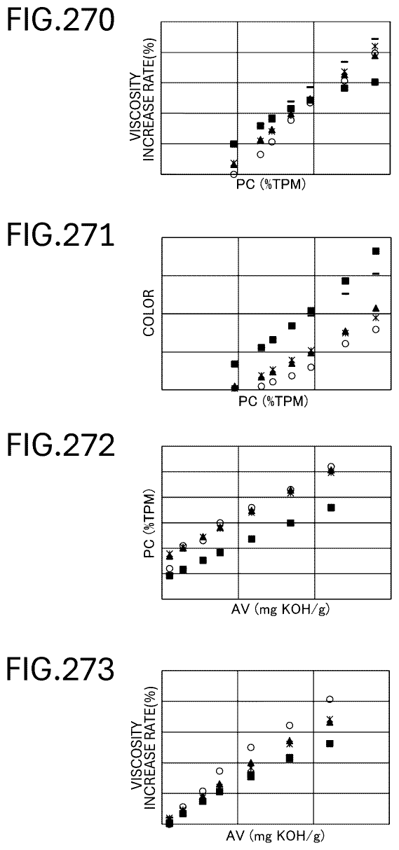

FIG. 270 illustrates a graph showing the correlation of the viscosity increase rate of the frying oil P relating to the eighth oil type, which is classified based on the FFA content in new oil, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 271 illustrates a graph showing the correlation of the color of the frying oil P relating to the eighth oil type, which is classified based on the FFA content in new oil, relative to the total polar compounds (measured using the measurement device 4) contained in the frying oil P, in which the actually measured values of the color are compared with the colors calculated using Equation (31), Equation (32), Equation (49), and Equation (50), respectively. FIG. 272 illustrates a graph showing the correlation of the total polar compounds contained in the frying oil P relating to the eighth oil type, which is classified based on the FFA content in new oil, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 273 illustrates a graph showing the correlation of the viscosity increase rate of the frying oil P relating to the eighth oil type, which is classified based on the FFA content in new oil, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

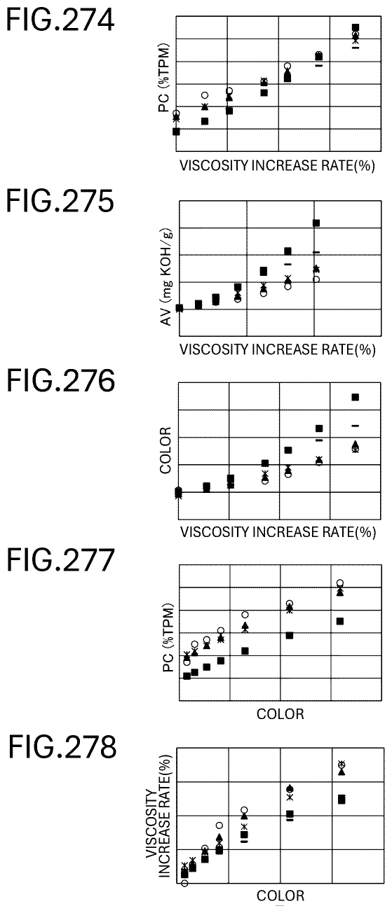

FIG. 274 illustrates a graph showing the correlation of the total polar compounds contained in the frying oil P relating to the eighth oil type, which is classified based on the FFA content in new oil, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 275 illustrates a graph showing the correlation of the acid value of the frying oil P relating to the eighth oil type, which is classified based on the FFA content in new oil, relative to the viscosity increase rate (measured using the measurement device 4) of the frying oil P, in which the actually measured acid values are compared with the acid values calculated using Equation (31), Equation (32), Equation (49), and Equation (50), respectively. FIG. 276 illustrates a graph showing the correlation of the color of the frying oil P relating to the eighth oil type, which is classified based on the FFA content in new oil, relative to the viscosity increase rate (measured using the measurement device 4) of the frying oil P, in which the actually measured values of the color are compared with the colors calculated using Equation (31), Equation (32), Equation (49), and Equation (50), respectively. FIG. 277 illustrates a graph showing the correlation of the total polar compounds contained in the frying oil P relating to the eighth oil type, which is classified based on the FFA content in new oil, relative to the color (measured using the measurement device 4) of the frying oil P, in which the actually measured total polar compounds are compared with the total polar compounds calculated using Equation (31), Equation (32), Equation (49), and Equation (50), respectively. FIG. 278 illustrates a graph showing the correlation of the viscosity increase rate of the frying oil P relating to the eighth oil type, which is classified based on the FFA content in new oil, relative to the color (measured using the measurement device 4) of the frying oil P, in which the actually measured viscosity increase rates are compared with the viscosity increase rates calculated using Equation (31), Equation (32), Equation (49), and Equation (50), respectively.

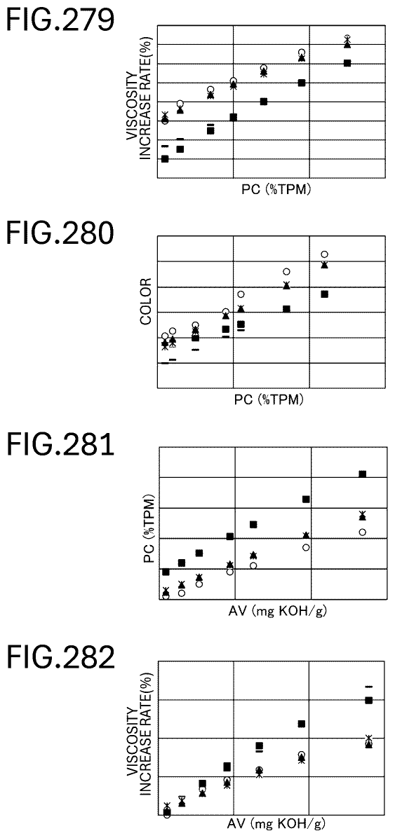

FIG. 279 illustrates a graph showing the correlation of the viscosity increase rate of the frying oil P relating to the seventh oil type, which is classified based on the MG content in heated oil, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 280 illustrates a graph showing the correlation of the color of the frying oil P relating to the seventh oil type, which is classified based on the MG content in heated oil, relative to the total polar compounds (measured using the measurement device 4) contained in the frying oil P, in which the actually measured values of the color are compared with the colors calculated using Equation (31), Equation (32), Equation (47), and Equation (48), respectively. FIG. 281 illustrates a graph showing the correlation of the total polar compounds contained in the frying oil P relating to the seventh oil type, which is classified based on the MG content in heated oil, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 282 illustrates a graph showing the correlation of the viscosity increase rate of the frying oil P relating to the seventh oil type, which is classified based on the MG content in heated oil, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

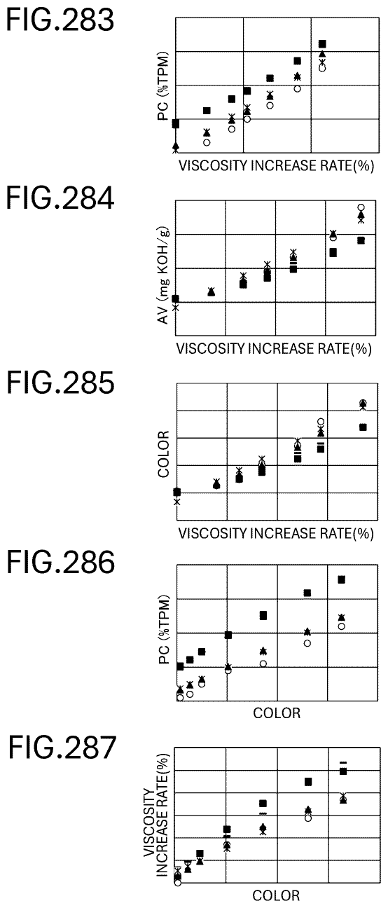

FIG. 283 illustrates a graph showing the correlation of the total polar compounds contained in the frying oil P relating to the seventh oil type, which is classified based on the MG content in heated oil, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 284 illustrates a graph showing the correlation of the acid value of the frying oil P relating to the seventh oil type, which is classified based on the MG content in heated oil, relative to the viscosity increase rate (measured using the measurement device 4) of the frying oil P, in which the actually measured acid values are compared with the acid values calculated using Equation (31), Equation (32), Equation (47), and Equation (48), respectively. FIG. 285 illustrates a graph showing the correlation of the color of the frying oil P relating to the seventh oil type, which is classified based on the MG content in heated oil, relative to the viscosity increase rate (measured using the measurement device 4) of the frying oil P, in which the actually measured values of the color are compared with the colors calculated using Equation (31), Equation (32), Equation (47), and Equation (48), respectively. FIG. 286 illustrates a graph showing the correlation of the total polar compounds contained in the frying oil P relating to the seventh oil type, which is classified based on the MG content in heated oil, relative to the color (measured using the measurement device 4) of the frying oil P, in which the actually measured total polar compounds are compared with the total polar compounds calculated using Equation (31), Equation (32), Equation (47), and Equation (48), respectively. FIG. 287 illustrates a graph showing the correlation of the viscosity increase rate of the frying oil P relating to the seventh oil type, which is classified based on the MG content in heated oil, relative to the color (measured using the measurement device 4) of the frying oil P, in which the actually measured viscosity increase rates are compared with the viscosity increase rates calculated using Equation (31), Equation (32), Equation (47), and Equation (48), respectively.

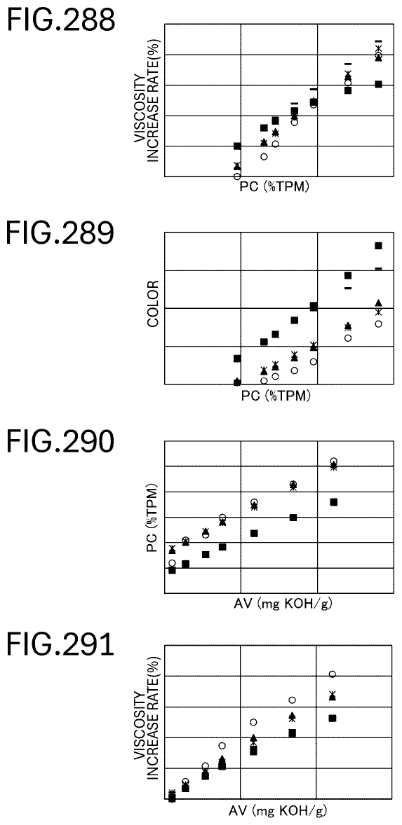

FIG. 288 illustrates a graph showing the correlation of the viscosity increase rate of the frying oil P relating to the eighth oil type, which is classified based on the MG content in heated oil, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 289 illustrates a graph showing the correlation of the color of the frying oil P relating to the eighth oil type, which is classified based on the MG content in heated oil, relative to the total polar compounds (measured using the measurement device 4) contained in the frying oil P, in which the actually measured values of the color are compared with the colors calculated using Equation (31), Equation (32), Equation (49), and Equation (50), respectively. FIG. 290 illustrates a graph showing the correlation of the total polar compounds contained in the frying oil P relating to the eighth oil type, which is classified based on the MG content in heated oil, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 291 illustrates a graph showing the correlation of the viscosity increase rate of the frying oil P relating to the eighth oil type, which is classified based on the MG content in heated oil, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

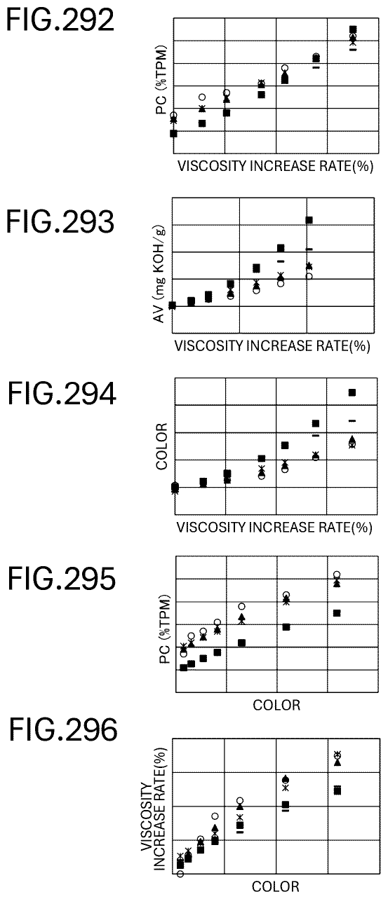

FIG. 292 illustrates a graph showing the correlation of the total polar compounds contained in the frying oil P relating to the eighth oil type, which is classified based on the MG content in heated oil, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 293 illustrates a graph showing the correlation of the acid value of the frying oil P relating to the eighth oil type, which is classified based on the MG content in heated oil, relative to the viscosity increase rate (measured using the measurement device 4) of the frying oil P, in which the actually measured acid values are compared with the acid values calculated using Equation (31), Equation (32), Equation (49), and Equation (50), respectively. FIG. 294 illustrates a graph showing the correlation of the color of the frying oil P relating to the eighth oil type, which is classified based on the MG content in heated oil, relative to the viscosity increase rate (measured using the measurement device 4) of the frying oil P, in which the actually measured values of the color are compared with the colors calculated using Equation (31), Equation (32), Equation (49), and Equation (50), respectively. FIG. 295 illustrates a graph showing the correlation of the total polar compounds contained in the frying oil P relating to the eighth oil type, which is classified based on the MG content in heated oil, relative to the color (measured using the measurement device 4) of the frying oil P, in which the actually measured total polar compounds are compared with the total polar compounds calculated using Equation (31), Equation (32), Equation (49), and Equation (50), respectively. FIG. 296 illustrates a graph showing the correlation of the viscosity increase rate of the frying oil P relating to the eighth oil type, which is classified based on the MG content in heated oil, relative to the color (measured using the measurement device 4) of the frying oil P, in which the actually measured viscosity increase rates are compared with the viscosity increase rates calculated using Equation (31), Equation (32), Equation (49), and Equation (50), respectively.

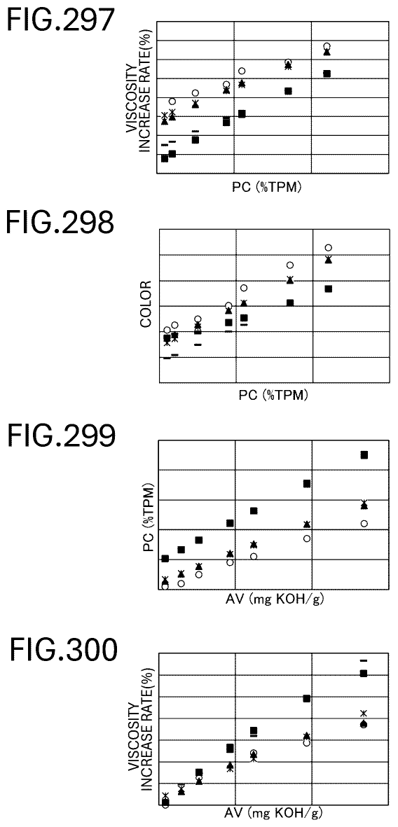

FIG. 297 illustrates a graph showing the correlation of the viscosity increase rate of the frying oil P relating to the seventh oil type, which is classified based on the TG content in heated oil, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 298 illustrates a graph showing the correlation of the color of the frying oil P relating to the seventh oil type, which is classified based on the TG content in heated oil, relative to the total polar compounds (measured using the measurement device 4) contained in the frying oil P, in which the actually measured values of the color are compared with the colors calculated using Equation (31), Equation (32), Equation (47), and Equation (48), respectively. FIG. 299 illustrates a graph showing the correlation of the total polar compounds contained in the frying oil P relating to the seventh oil type, which is classified based on the TG content in heated oil, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 300 illustrates a graph showing the correlation of the viscosity increase rate of the frying oil P relating to the seventh oil type, which is classified based on the TG content in heated oil, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

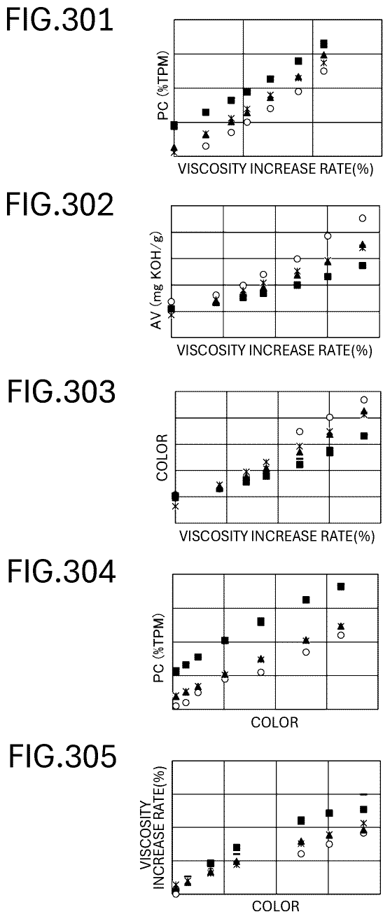

FIG. 301 illustrates a graph showing the correlation of the total polar compounds contained in the frying oil P relating to the seventh oil type, which is classified based on the TG content in heated oil, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 302 illustrates a graph showing the correlation of the acid value of the frying oil P relating to the seventh oil type, which is classified based on the TG content in heated oil, relative to the viscosity increase rate (measured using the measurement device 4) of the frying oil P, in which the actually measured acid values are compared with the acid values calculated using Equation (31), Equation (32), Equation (47), and Equation (48), respectively. FIG. 303 illustrates a graph showing the correlation of the color of the frying oil P relating to the seventh oil type, which is classified based on the TG content in heated oil, relative to the viscosity increase rate (measured using the measurement device 4) of the frying oil P, in which the actually measured values of the color are compared with the colors calculated using Equation (31), Equation (32), Equation (47), and Equation (48), respectively. FIG. 304 illustrates a graph showing the correlation of the total polar compounds contained in the frying oil P relating to the seventh oil type, which is classified based on the TG content in heated oil, relative to the color (measured using the measurement device 4) of the frying oil P, in which the actually measured total polar compounds are compared with the total polar compounds calculated using Equation (31), Equation (32), Equation (47), and Equation (48), respectively. FIG. 305 illustrates a graph showing the correlation of the viscosity increase rate of the frying oil P relating to the seventh oil type, which is classified based on the TG content in heated oil, relative to the color (measured using the measurement device 4) of the frying oil P, in which the actually measured viscosity increase rates are compared with the viscosity increase rates calculated using Equation (31), Equation (32), Equation (47), and Equation (48), respectively.

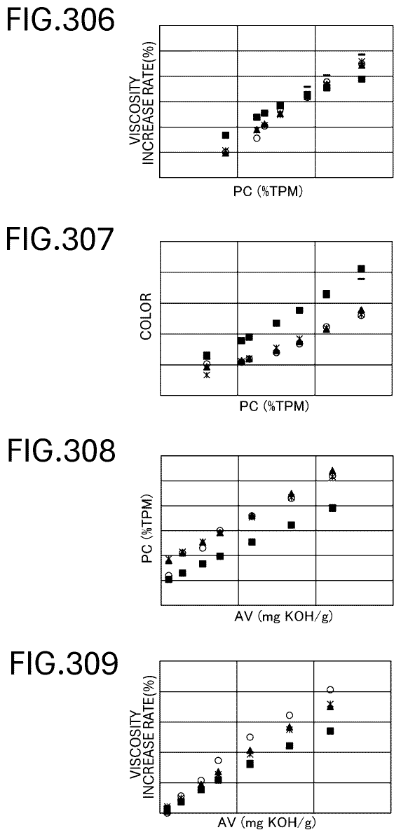

FIG. 306 illustrates a graph showing the correlation of the viscosity increase rate of the frying oil P relating to the eighth oil type, which is classified based on the TG content in heated oil, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 307 illustrates a graph showing the correlation of the color of the frying oil P relating to the eighth oil type, which is classified based on the TG content in heated oil, relative to the total polar compounds (measured using the measurement device 4) contained in the frying oil P, in which the actually measured values of the color are compared with the colors calculated using Equation (31), Equation (32), Equation (49), and Equation (50), respectively. FIG. 308 illustrates a graph showing the correlation of the total polar compounds contained in the frying oil P relating to the eighth oil type, which is classified based on the TG content in heated oil, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 309 illustrates a graph showing the correlation of the viscosity increase rate of the frying oil P relating to the eighth oil type, which is classified based on the TG content in heated oil, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

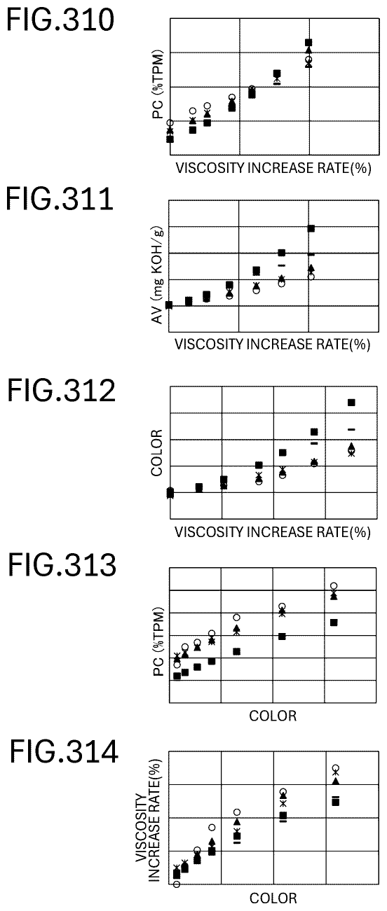

FIG. 310 illustrates a graph showing the correlation of the total polar compounds contained in the frying oil P relating to the eighth oil type, which is classified based on the TG content in heated oil, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 311 illustrates a graph showing the correlation of the acid value of the frying oil P relating to the eighth oil type, which is classified based on the TG content in heated oil, relative to the viscosity increase rate (measured using the measurement device 4) of the frying oil P, in which the actually measured acid values are compared with the acid values calculated using Equation (31), Equation (32), Equation (49), and Equation (50), respectively. FIG. 312 illustrates a graph showing the correlation of the color of the frying oil P relating to the eighth oil type, which is classified based on the TG content in heated oil, relative to the viscosity increase rate (measured using the measurement device 4) of the frying oil P, in which the actually measured values of the color are compared with the colors calculated using Equation (31), Equation (32), Equation (49), and Equation (50), respectively. FIG. 313 illustrates a graph showing the correlation of the total polar compounds contained in the frying oil P relating to the eighth oil type, which is classified based on the TG content in heated oil, relative to the color (measured using the measurement device 4) of the frying oil P, in which the actually measured total polar compounds are compared with the total polar compounds calculated using Equation (31), Equation (32), Equation (49), and Equation (50), respectively. FIG. 314 illustrates a graph showing the correlation of the viscosity increase rate of the frying oil P relating to the eighth oil type, which is classified based on the TG content in heated oil, relative to the color (measured using the measurement device 4) of the frying oil P, in which the actually measured viscosity increase rates are compared with the viscosity increase rates calculated using Equation (31), Equation (32), Equation (49), and Equation (50), respectively.

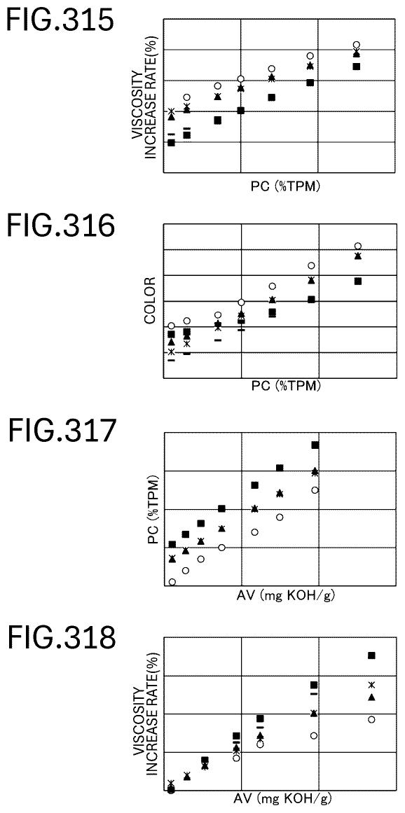

FIG. 315 illustrates a graph showing the correlation of the viscosity increase rate of the frying oil P relating to the seventh oil type, which is classified based on the rate of increase in the DG content due to heating, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 316 illustrates a graph showing the correlation of the color of the frying oil P relating to the seventh oil type, which is classified based on the rate of increase in the DG content due to heating, relative to the total polar compounds (measured using the measurement device 4) contained in the frying oil P, in which the actually measured values of the color are compared with the colors calculated using Equation (31), Equation (32), Equation (47), and Equation (48), respectively. FIG. 317 illustrates a graph showing the correlation of the total polar compounds contained in the frying oil P relating to the seventh oil type, which is classified based on the rate of increase in the DG content due to heating, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 318 illustrates a graph showing the correlation of the viscosity increase rate of the frying oil P relating to the seventh oil type, which is classified based on the rate of increase in the DG content due to heating, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

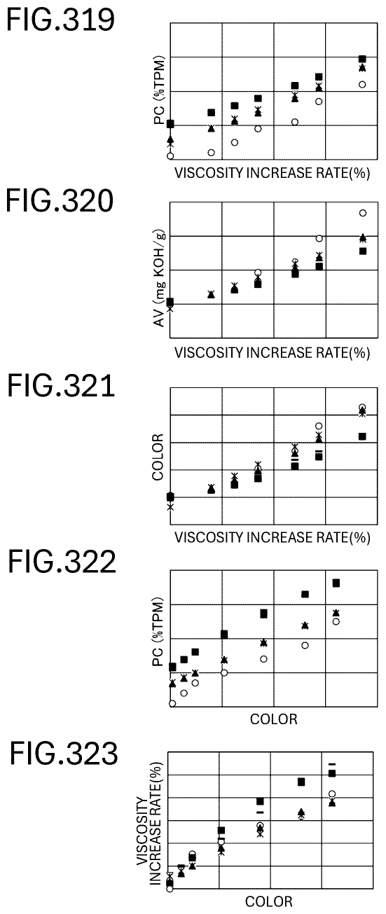

FIG. 319 illustrates a graph showing the correlation of the total polar compounds contained in the frying oil P relating to the seventh oil type, which is classified based on the rate of increase in the DG content due to heating, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 320 illustrates a graph showing the correlation of the acid value of the frying oil P relating to the seventh oil type, which is classified based on the rate of increase in the DG content due to heating, relative to the viscosity increase rate (measured using the measurement device 4) of the frying oil P, in which the actually measured acid values are compared with the acid values calculated using Equation (31), Equation (32), Equation (47), and Equation (48), respectively. FIG. 321 illustrates a graph showing the correlation of the color of the frying oil P relating to the seventh oil type, which is classified based on the rate of increase in the DG content due to heating, relative to the viscosity increase rate (measured using the measurement device 4) of the frying oil P, in which the actually measured values of the color are compared with the colors calculated using Equation (31), Equation (32), Equation (47), and Equation (48), respectively. FIG. 322 illustrates a graph showing the correlation of the total polar compounds contained in the frying oil P relating to the seventh oil type, which is classified based on the rate of increase in the DG content due to heating, relative to the color (measured using the measurement device 4) of the frying oil P, in which the actually measured total polar compounds are compared with the total polar compounds calculated using Equation (31), Equation (32), Equation (47), and Equation (48), respectively. FIG. 323 illustrates a graph showing the correlation of the viscosity increase rate of the frying oil P relating to the seventh oil type, which is classified based on the rate of increase in the DG content due to heating, relative to the color (measured using the measurement device 4) of the frying oil P, in which the actually measured viscosity increase rates are compared with the viscosity increase rates calculated using Equation (31), Equation (32), Equation (47), and Equation (48), respectively.

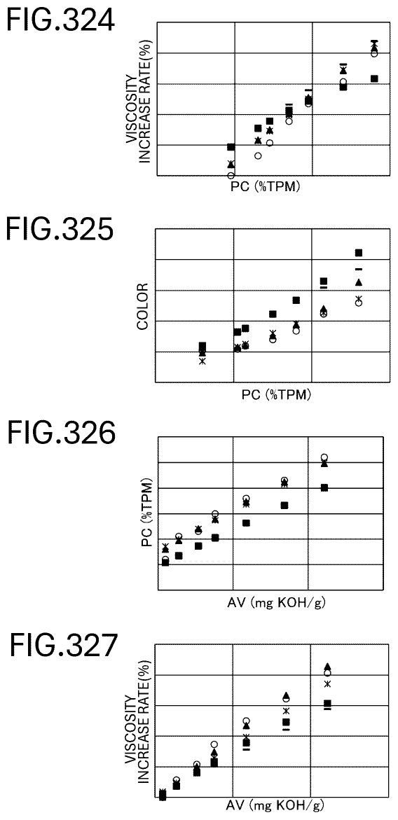

FIG. 324 illustrates a graph showing the correlation of the viscosity increase rate of the frying oil P relating to the eighth oil type, which is classified based on the rate of increase in the DG content due to heating, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 325 illustrates a graph showing the correlation of the color of the frying oil P relating to the eighth oil type, which is classified based on the rate of increase in the DG content due to heating, relative to the total polar compounds (measured using the measurement device 4) contained in the frying oil P, in which the actually measured values of the color are compared with the colors calculated using Equation (31), Equation (32), Equation (49), and Equation (50), respectively. FIG. 326 illustrates a graph showing the correlation of the total polar compounds contained in the frying oil P relating to the eighth oil type, which is classified based on the rate of increase in the DG content due to heating, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 327 illustrates a graph showing the correlation of the viscosity increase rate of the frying oil P relating to the eighth oil type, which is classified based on the rate of increase in the DG content due to heating, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

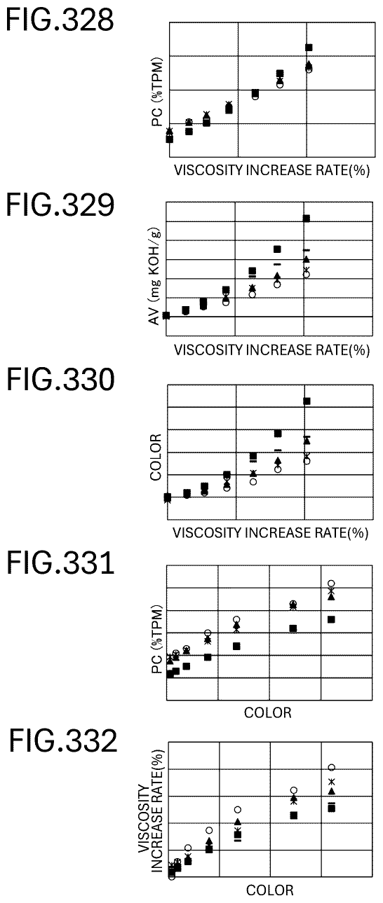

FIG. 328 illustrates a graph showing the correlation of the total polar compounds contained in the frying oil P relating to the eighth oil type, which is classified based on the rate of increase in the DG content due to heating, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 329 illustrates a graph showing the correlation of the acid value of the frying oil P relating to the eighth oil type, which is classified based on the rate of increase in the DG content due to heating, relative to the viscosity increase rate (measured using the measurement device 4) of the frying oil P, in which the actually measured acid values are compared with the acid values calculated using Equation (31), Equation (32), Equation (49), and Equation (50), respectively. FIG. 330 illustrates a graph showing the correlation of the color of the frying oil P relating to the eighth oil type, which is classified based on the rate of increase in the DG content due to heating, relative to the viscosity increase rate (measured using the measurement device 4) of the frying oil P, in which the actually measured values of the color are compared with the colors calculated using Equation (31), Equation (32), Equation (49), and Equation (50), respectively. FIG. 331 illustrates a graph showing the correlation of the total polar compounds contained in the frying oil P relating to the eighth oil type, which is classified based on the rate of increase in the DG content due to heating, relative to the color (measured using the measurement device 4) of the frying oil P, in which the actually measured total polar compounds are compared with the total polar compounds calculated using Equation (31), Equation (32), Equation (49), and Equation (50), respectively. FIG. 332 illustrates a graph showing the correlation of the viscosity increase rate of the frying oil P relating to the eighth oil type, which is classified based on the rate of increase in the DG content due to heating, relative to the color (measured using the measurement device 4) of the frying oil P, in which the actually measured viscosity increase rates are compared with the viscosity increase rates calculated using Equation (31), Equation (32), Equation (49), and Equation (50), respectively.

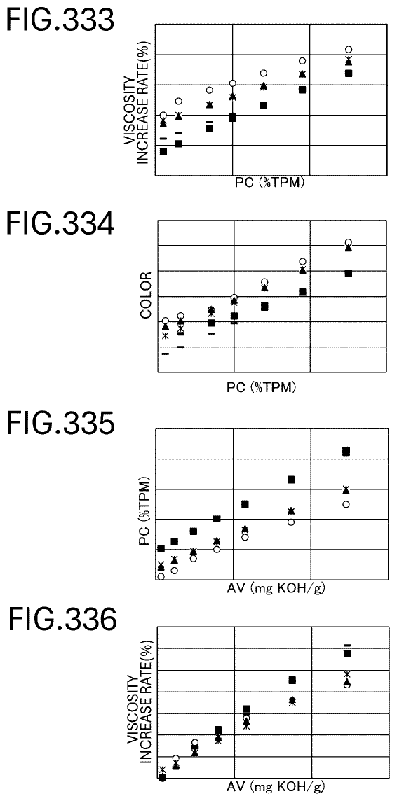

FIG. 333 illustrates a graph showing the correlation of the viscosity increase rate of the frying oil P relating to the seventh oil type, which is classified based on the rate of increase in the FFA content due to heating, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 334 illustrates a graph showing the correlation of the color of the frying oil P relating to the seventh oil type, which is classified based on the rate of increase in the FFA content due to heating, relative to the total polar compounds (measured using the measurement device 4) contained in the frying oil P, in which the actually measured values of the color are compared with the colors calculated using Equation (31), Equation (32), Equation (47), and Equation (48), respectively. FIG. 335 illustrates a graph showing the correlation of the total polar compounds contained in the frying oil P relating to the seventh oil type, which is classified based on the rate of increase in the FFA content due to heating, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 336 illustrates a graph showing the correlation of the viscosity increase rate of the frying oil P relating to the seventh oil type, which is classified based on the rate of increase in the FFA content due to heating, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

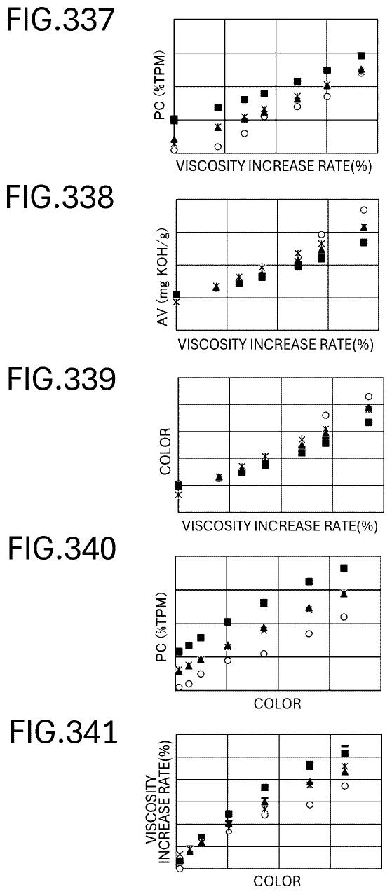

FIG. 337 illustrates a graph showing the correlation of the total polar compounds contained in the frying oil P relating to the seventh oil type, which is classified based on the rate of increase in the FFA content due to heating, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 338 illustrates a graph showing the correlation of the acid value of the frying oil P relating to the seventh oil type, which is classified based on the rate of increase in the FFA content due to heating, relative to the viscosity increase rate (measured using the measurement device 4) of the frying oil P, in which the actually measured acid values are compared with the acid values calculated using Equation (31), Equation (32), Equation (47), and Equation (48), respectively. FIG. 339 illustrates a graph showing the correlation of the color of the frying oil P relating to the seventh oil type, which is classified based on the rate of increase in the FFA content due to heating, relative to the viscosity increase rate (measured using the measurement device 4) of the frying oil P, in which the actually measured values of the color are compared with the colors calculated using Equation (31), Equation (32), Equation (47), and Equation (48), respectively. FIG. 340 illustrates a graph showing the correlation of the total polar compounds contained in the frying oil P relating to the seventh oil type, which is classified based on the rate of increase in the FFA content due to heating, relative to the color (measured using the measurement device 4) of the frying oil P, in which the actually measured total polar compounds are compared with the total polar compounds calculated using Equation (31), Equation (32), Equation (47), and Equation (48), respectively. FIG. 341 illustrates a graph showing the correlation of the viscosity increase rate of the frying oil P relating to the seventh oil type, which is classified based on the rate of increase in the FFA content due to heating, relative to the color (measured using the measurement device 4) of the frying oil P, in which the actually measured viscosity increase rates are compared with the viscosity increase rates calculated using Equation (31), Equation (32), Equation (47), and Equation (48), respectively.

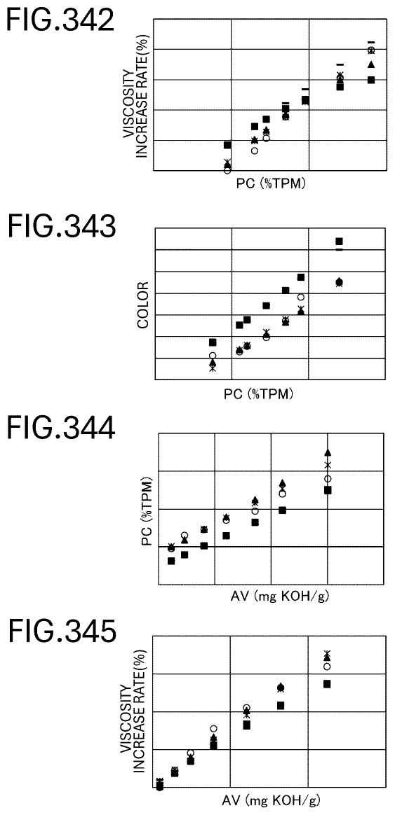

FIG. 342 illustrates a graph showing the correlation of the viscosity increase rate of the frying oil P relating to the eighth oil type, which is classified based on the rate of increase in the FFA content due to heating, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 343 illustrates a graph showing the correlation of the color of the frying oil P relating to the eighth oil type, which is classified based on the rate of increase in the FFA content due to heating, relative to the total polar compounds (measured using the measurement device 4) contained in the frying oil P, in which the actually measured values of the color are compared with the colors calculated using Equation (31), Equation (32), Equation (49), and Equation (50), respectively. FIG. 344 illustrates a graph showing the correlation of the total polar compounds contained in the frying oil P relating to the eighth oil type, which is classified based on the rate of increase in the FFA content due to heating, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 345 illustrates a graph showing the correlation of the viscosity increase rate of the frying oil P relating to the eighth oil type, which is classified based on the rate of increase in the FFA content due to heating, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

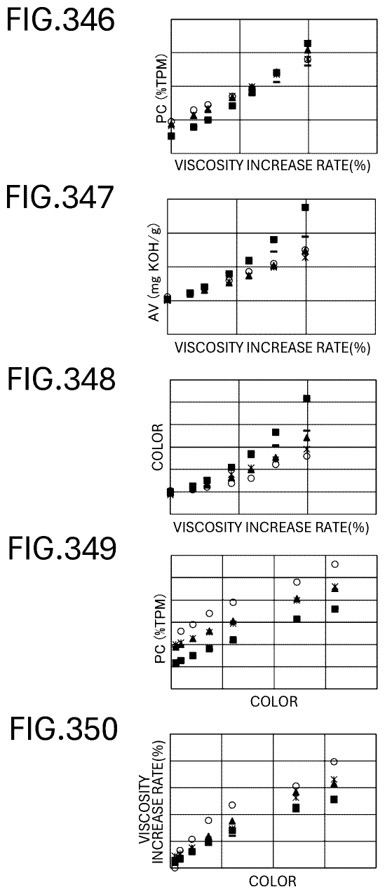

FIG. 346 illustrates a graph showing the correlation of the total polar compounds contained in the frying oil P relating to the eighth oil type, which is classified based on the rate of increase in the FFA content due to heating, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 347 illustrates a graph showing the correlation of the acid value of the frying oil P relating to the eighth oil type, which is classified based on the rate of increase in the FFA content due to heating, relative to the viscosity increase rate (measured using the measurement device 4) of the frying oil P, in which the actually measured acid values are compared with the acid values calculated using Equation (31), Equation (32), Equation (49), and Equation (50), respectively. FIG. 348 illustrates a graph showing the correlation of the color of the frying oil P relating to the eighth oil type, which is classified based on the rate of increase in the FFA content due to heating, relative to the viscosity increase rate (measured using the measurement device 4) of the frying oil P, in which the actually measured values of the color are compared with the colors calculated using Equation (31), Equation (32), Equation (49), and Equation (50), respectively. FIG. 350 illustrates a graph showing the correlation of the viscosity increase rate of the frying oil P relating to the eighth oil type, which is classified based on the rate of increase in the FFA content due to heating, relative to the color (measured using the measurement device 4) of the frying oil P, in which the actually measured viscosity increase rates are compared with the viscosity increase rates calculated using Equation (31), Equation (32), Equation (49), and Equation (50), respectively.

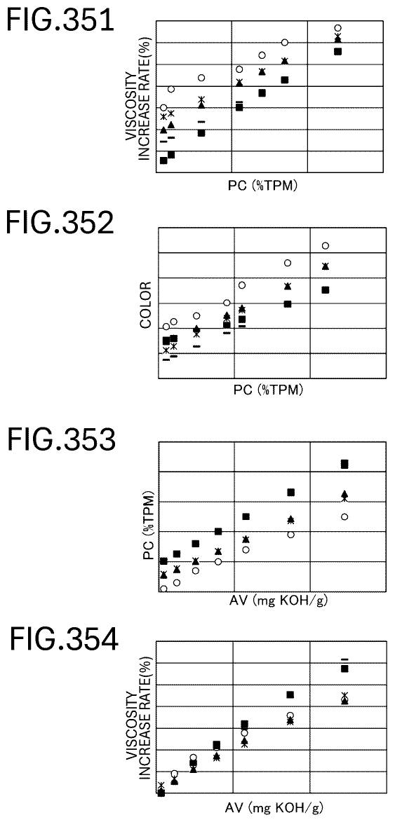

FIG. 351 illustrates a graph showing the correlation of the viscosity increase rate of the frying oil P relating to the seventh oil type, which is classified based on the rate of decrease in the TG content due to heating, relative to the total polar compounds (measured using the measurement device 4) contained in the frying oil P, in which the actually measured viscosity increase rates are compared with the viscosity increase rates calculated using Equation (31), Equation (32), Equation (47), and Equation (48), respectively. FIG. 352 illustrates a graph showing the correlation of the color of the frying oil P relating to the seventh oil type, which is classified based on the rate of decrease in the TG content due to heating, relative to the total polar compounds (measured using the measurement device 4) contained in the frying oil P, in which the actually measured values of the color are compared with the colors calculated using Equation (31), Equation (32), Equation (47), and Equation (48), respectively. FIG. 353 illustrates a graph showing the correlation of the total polar compounds contained in the frying oil P relating to the seventh oil type, which is classified based on the rate of decrease in the TG content due to heating, relative to the acid value (measured using the measurement device 4) of the frying oil P, in which the actually measured total polar compounds are compared with the total polar compounds calculated using Equation (31), Equation (32), Equation (47), and Equation (48), respectively. FIG. 354 illustrates a graph showing the correlation of the viscosity increase rate of the frying oil P relating to the seventh oil type, which is classified based on the rate of decrease in the TG content due to heating, relative to the acid value (measured using the measurement device 4) of the frying oil P, in which the actually measured viscosity increase rates are compared with the viscosity increase rates calculated using Equation (31), Equation (32), Equation (47), and Equation (48), respectively.

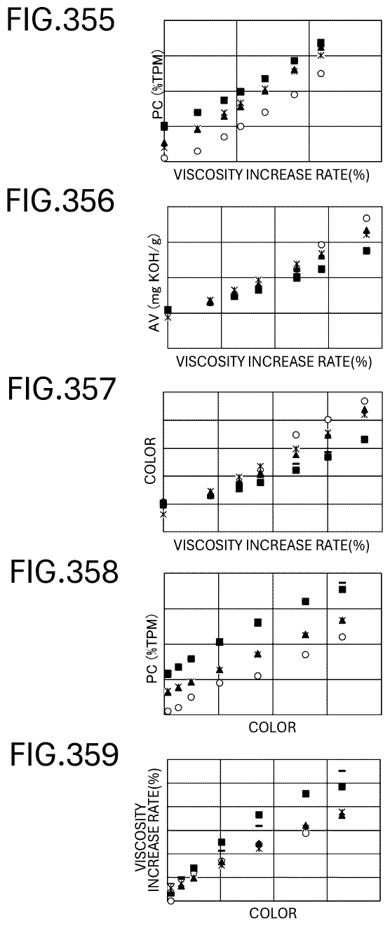

FIG. 355 illustrates a graph showing the correlation of the total polar compounds contained in the frying oil P relating to the seventh oil type, which is classified based on the rate of decrease in the TG content due to heating, relative to the viscosity increase rate (measured using the measurement device 4) of the frying oil P, in which the actually measured total polar compounds are compared with the total polar compounds calculated using Equation (31), Equation (32), Equation (47), and Equation (48), respectively. FIG. 356 illustrates a graph showing the correlation of the acid value of the frying oil P relating to the seventh oil type, which is classified based on the rate of decrease in the TG content due to heating, relative to the viscosity increase rate (measured using the measurement device 4) of the frying oil P, in which the actually measured acid values are compared with the acid values calculated using Equation (31), Equation (32), Equation (47), and Equation (48), respectively. FIG. 357 illustrates a graph showing the correlation of the color of the frying oil P relating to the seventh oil type, which is classified based on the rate of decrease in the TG content due to heating, relative to the viscosity increase rate (measured using the measurement device 4) of the frying oil P, in which the actually measured values of the color are compared with the colors calculated using Equation (31), Equation (32), Equation (47), and Equation (48), respectively. FIG. 358 illustrates a graph showing the correlation of the total polar compounds contained in the frying oil P relating to the seventh oil type, which is classified based on the rate of decrease in the TG content due to heating, relative to the color (measured using the measurement device 4) of the frying oil P, in which the actually measured total polar compounds are compared with the total polar compounds calculated using Equation (31), Equation (32), Equation (47), and Equation (48), respectively. FIG. 359 illustrates a graph showing the correlation of the viscosity increase rate of the frying oil P relating to the seventh oil type, which is classified based on the rate of decrease in the TG content due to heating, relative to the color (measured using the measurement device 4) of the frying oil P, in which the actually measured viscosity increase rates are compared with the viscosity increase rates calculated using Equation (31), Equation (32), Equation (47), and Equation (48), respectively.

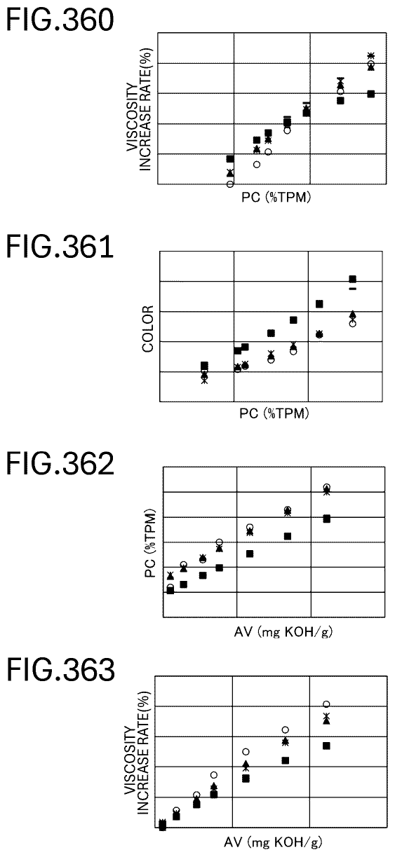

FIG. 360 illustrates a graph showing the correlation of the viscosity increase rate of the frying oil P relating to the eighth oil type, which is classified based on the rate of decrease in the TG content due to heating, relative to the total polar compounds (measured using the measurement device 4) contained in the frying oil P, in which the actually measured viscosity increase rates are compared with the viscosity increase rates calculated using Equation (31), Equation (32), Equation (49), and Equation (50), respectively. FIG. 361 illustrates a graph showing the correlation of the color of the frying oil P relating to the eighth oil type, which is classified based on the rate of decrease in the TG content due to heating, relative to the total polar compounds (measured using the measurement device 4) contained in the frying oil P, in which the actually measured values of the color are compared with the colors calculated using Equation (31), Equation (32), Equation (49), and Equation (50), respectively. FIG. 362 illustrates a graph showing the correlation of the total polar compounds contained in the frying oil P relating to the eighth oil type, which is classified based on the rate of decrease in the TG content due to heating, relative to the acid value (measured using the measurement device 4) of the frying oil P, in which the actually measured total polar compounds are compared with the total polar compounds calculated using Equation (31), Equation (32), Equation (49), and Equation (50), respectively. FIG. 363 illustrates a graph showing the correlation of the viscosity increase rate of the frying oil P relating to the eighth oil type, which is classified based on the rate of decrease in the TG content due to heating, relative to the acid value (measured using the measurement device 4) of the frying oil P, in which the actually measured viscosity increase rates are compared with the viscosity increase rates calculated using Equation (31), Equation (32), Equation (49), and Equation (50), respectively.

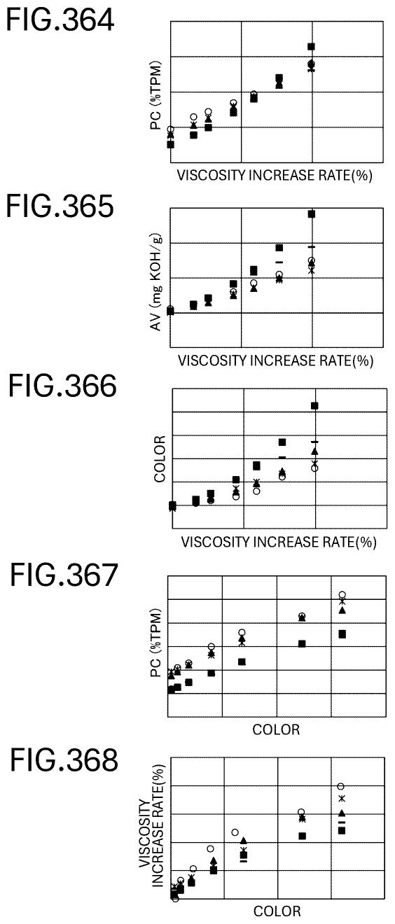

FIG. 364 illustrates a graph showing the correlation of the total polar compounds contained in the frying oil P relating to the eighth oil type, which is classified based on the rate of decrease in the TG content due to heating, relative to the viscosity increase rate (measured using the measurement device 4) of the frying oil P, in which the actually measured total polar compounds are compared with the total polar compounds calculated using Equation (31), Equation (32), Equation (49), and Equation (50), respectively. FIG. 365 illustrates a graph showing the correlation of the acid value of the frying oil P relating to the eighth oil type, which is classified based on the rate of decrease in the TG content due to heating, relative to the viscosity increase rate (measured using the measurement device 4) of the frying oil P, in which the actually measured acid values are compared with the acid values calculated using Equation (31), Equation (32), Equation (49), and Equation (50), respectively. FIG. 366 illustrates a graph showing the correlation of the color of the frying oil P relating to the eighth oil type, which is classified based on the rate of decrease in the TG content due to heating, relative to the viscosity increase rate (measured using the measurement device 4) of the frying oil P, in which the actually measured values of the color are compared with the colors calculated using Equation (31), Equation (32), Equation (49), and Equation (50), respectively. FIG. 367 illustrates a graph showing the correlation of the total polar compounds contained in the frying oil P relating to the eighth oil type, which is classified based on the rate of decrease in the TG content due to heating, relative to the color (measured using the measurement device 4) of the frying oil P, in which the actually measured total polar compounds are compared with the total polar compounds calculated using Equation (31), Equation (32), Equation (49), and Equation (50), respectively. FIG. 368 illustrates a graph showing the correlation of the viscosity increase rate of the frying oil P relating to the eighth oil type, which is classified based on the rate of decrease in the TG content due to heating, relative to the color (measured using the measurement device 4) of the frying oil P, in which the actually measured viscosity increase rates are compared with the viscosity increase rates calculated using Equation (31), Equation (32), Equation (49), and Equation (50), respectively.

In each of FIG. 243 to FIG. 251, FIG. 261 to FIG. 269, FIG. 279 to FIG. 287, FIG. 297 to FIG. 305, FIG. 315 to FIG. 323, FIG. 333 to FIG. 341, and FIG. 351 to FIG. 359 which relates to the case where the type of the frying oil P is the seventh oil type, a graph including a plurality of “o” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (47), and a graph including a plurality of “▴” is indicative of the correlation graph for Equation (48), respectively.

In each of FIG. 252 to FIG. 260, FIG. 270 to FIG. 278, FIG. 288 to FIG. 296, FIG. 306 to FIG. 314, FIG. 324 to FIG. 332, FIG. 342 to FIG. 350, and FIG. 360 to FIG. 368 which relates to the case where the type of the frying oil P is the eighth oil type, a graph including a plurality of “o” is indicative of the correlation graph for the actually measured second deterioration indicator of the frying oil P, a graph including a plurality of “-” is indicative of the correlation graph for Equation (31), a graph including a plurality of “▪” is indicative of the correlation graph for Equation (32), a graph including a plurality of “*” is indicative of the correlation graph for Equation (49), and a graph including a plurality of “▴” is indicative of the correlation graph for Equation (50), respectively.

As illustrated in each of FIG. 243 to FIG. 251, FIG. 261 to FIG. 269, FIG. 279 to FIG. 287, FIG. 297 to FIG. 305, FIG. 315 to FIG. 323, FIG. 333 to FIG. 341, and FIG. 351 to FIG. 359, the correlation graph for Equation (47) and the correlation graph for Equation (48) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually measured second deterioration indicator of the frying oil P further than the correlation graph for Equation (47) and the correlation graph for Equation (48).

In the same manner, as illustrated in each of FIG. 252 to FIG. 260, FIG. 270 to FIG. 278, FIG. 288 to FIG. 296, FIG. 306 to FIG. 314, FIG. 324 to FIG. 332, FIG. 342 to FIG. 350, and FIG. 360 to FIG. 368, the correlation graph for Equation (49) and the correlation graph for Equation (50) are positioned closer to the correlation graph for the actually measured second deterioration indicator of the frying oil P than the correlation graph for Equation (31) and the correlation graph for Equation (32). In other words, the correlation graph for Equation (31) and the correlation graph for Equation (32) are deviated from the correlation graph for the actually measured second deterioration indicator of the frying oil P further than the correlation graph for Equation (49) and the correlation graph for Equation (50).

Thus, in the case where the measurement device 4 measures the first deterioration indicator of the frying oil P, the cloud 9C identifies whether the type of the frying oil P is the seventh oil type or the eighth oil type based on the lipid molecular species of the frying oil P and calculates the second deterioration indicator of the frying oil P using the correlation equation for the oil type as identified. This enables the calculation of the second deterioration indicator of the frying oil P with higher accuracy than the case of calculating it using Equation (31) or Equation (32) in all cases without considering the type of oil classified based on the lipid molecular species of the frying oil P.

### Fifteenth Embodiment

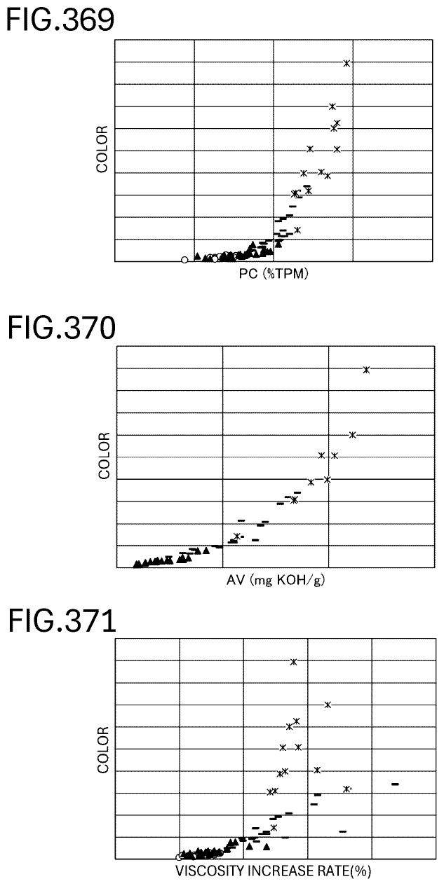

Next, a cloud 9D according to the fifteenth embodiment of the present invention will be described with reference to FIG. 369 to FIG. 373.

FIG. 369 illustrates a graph of the correlation of the color of the frying oil P relative to the total polar compounds contained in the frying oil P, in which the type of a deep-frying material is considered. FIG. 370 illustrates a graph of the correlation of the color of the frying oil P relative to the acid value of the frying oil P, in which the type of a deep-frying material is considered. FIG. 371 illustrates a graph of the correlation of the color of the frying oil P relative to the viscosity increase rate contained in the frying oil P, in which the type of a deep-frying material is considered.

As illustrated in each of FIG. 369 to FIG. 371, in the case where the second deterioration indicator of the frying oil P is the color, the correlation graph varies depending on the type of the deep-frying material Q. In each of FIG. 369 to FIG. 371, the correlation graphs relate to four types of deep-frying materials Q1, Q2, Q3, Q4 which are different from each other in their types, in which the one including a plurality of “∘” is indicative of the correlation graph for the deep-frying material Q1, the one including a plurality of “-” is indicative of the correlation graph for the deep-frying material Q2, the one including a plurality of “▪” is indicative of the correlation graph for the deep-frying material Q3, and the one including a plurality of “▴” is indicative of the correlation graph for the deep-frying material Q4, respectively.

For example, in the case where the deep-frying material Q is the deep-frying material Q1, the correlation equation for the second deterioration indicator of the frying oil P relative to the first deterioration indicator of the frying oil P is a linear equation expressed with the following Equation (51) including each of α9 as the first-order coefficient α of Di1n and β9 as the constant β in Equation (31), or a quadratic equation expressed with the following Equation (52) including each of γ9 as the second-order coefficient γ of Di1n, δ9 as the first-order coefficient δ of Di1n, and ε9 as the constant ε in Equation (32).

$$Di 2 n = α 9 \times ( Di 1 n ) + β 9 \qquad ( 51 )$$

$$Di 2 n = γ 9 \times {( Di 1 n )}^{2} + δ 9 \times ( Pcn ) + ε 9 \qquad ( 52 )$$

In the case where the deep-frying material Q is the deep-frying material Q2, the correlation equation for the second deterioration indicator of the frying oil P relative to the first deterioration indicator of the frying oil P is a linear equation expressed with the following Equation (53) including each of α10 as the first-order coefficient α of Di1n and β10 as the constant β in Equation (31), or a quadratic equation expressed with the following Equation (54) including each of γ10 as the second-order coefficient γ of Di1n, δ10 as the first-order coefficient δ of Di1n, and ε10 as the constant ε in Equation (32).

$$Di 2 n = α 10 \times ( Di 1 n ) + β 10 \qquad ( 53 )$$

$$Di 2 n = γ 10 \times {( Di 1 n )}^{2} + δ 10 \times ( PCn ) + ε 10 \qquad ( 52 )$$

In the case where the deep-frying material Q is the deep-frying material Q3, the correlation equation for the second deterioration indicator of the frying oil P relative to the first deterioration indicator of the frying oil P is a linear equation expressed with the following Equation (55) including each of all as the first-order coefficient α of Di1n and β11 as the constant β in Equation (31), or a quadratic equation expressed with the following Equation (56) including each of γ11 as the second-order coefficient γ of Di1n, δ11 as the first-order coefficient δ of Di1n, and Ell as the constant ε in Equation (32).

$$Di 2 n = α 11 \times ( Di 1 n ) + β 11 \qquad ( 55 )$$

$$Di 2 n = γ 11 \times {( Di 1 n )}^{2} + δ 11 \times ( PCn ) + ε 11 \qquad ( 56 )$$

In the case where the deep-frying material Q is the deep-frying material Q4, the correlation equation for the second deterioration indicator of the frying oil P relative to the first deterioration indicator of the frying oil P is a linear equation expressed with the following Equation (57) including each of α12 as the first-order coefficient α of Di1n and β12 as the constant β in Equation (31), or a quadratic equation expressed with the following Equation (58) including each of γ12 as the second-order coefficient γ of Di1n, δ12 as the first-order coefficient δ of Di1n, and ε12 as the constant ε in Equation (32).

$$Di 2 n = α 12 \times ( Di 1 n ) + β 12 \qquad ( 57 )$$

$$Di 2 n = γ 12 \times {( Di 1 n )}^{2} + δ 12 \times ( PCn ) + ε 12 \qquad ( 58 )$$

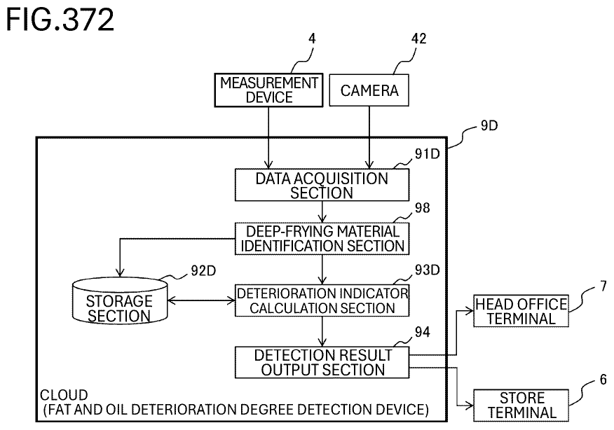

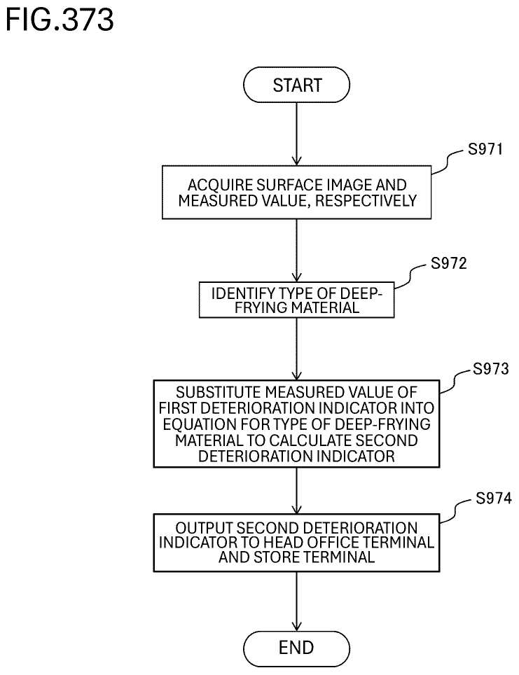

FIG. 372 is a functional block diagram illustrating functions provided in the cloud 9D according to the fifteenth embodiment. FIG. 373 illustrates a flowchart of a flow of the processing to be executed by the cloud 9D according to the fifteenth embodiment.

As illustrated in FIG. 372, the cloud 9D according to the present embodiment includes a data acquisition section 91D, a deep-frying material identification section 98, a storage section 92D, a deterioration indicator calculation section 93D, and a detection result output section 94.

As illustrated in FIG. 373, in the cloud 9D, firstly, the data acquisition section 91D acquires the measured value of the first deterioration indicator of the frying oil P output from the measurement device 4 and the surface image of the frying oil P output from the camera 4 (step S971).

Next, the deep-frying material identification section 98 identifies the type of the deep-frying material Q being cooked based on the surface image of the frying oil P acquired in step S971 (step S972). The method for determining the type of the deep-frying material Q does not necessarily have to be based on the surface image of the frying oil P captured by the camera 42. For example, inputting the type of the deep-frying material Q to be cooked in advance by a staff of a store allows the cloud 9D to identify the type of the deep-frying material Q based on the deep-frying material information output from the store terminal 6.

Next, the deterioration indicator calculation section 93D reads, from the storage section 92D, the equation for the type of the deep-frying material Q identified in step S972, and substitutes the measured value of the first deterioration indicator of the frying oil P acquired in step S971 into Di1n of the equation as read so as to calculate the second deterioration indicator Di2n of the frying oil P (step S973).

For example, in the case where it is identified in step S972 that the deep-frying material Q is the deep-frying material Q1, the deterioration indicator calculation section 93D substitutes the measured value of the first deterioration indicator of the frying oil P into Di1n of Equation (51) or Di1n of Equation (52), so as to calculate the second deterioration indicator Di2n of the frying oil P.

Then, the detection result output section 94 outputs the second deterioration indicator Di2n of the frying oil P calculated in step S973 to the store terminal 6 and the head office terminal 7, respectively, as the result of detection of the deterioration degree of the frying oil P (step S974), whereby the processing in the cloud 9D is ended.

In the present embodiment, the cloud 9D calculates the second deterioration indicator of the frying oil P using the equations appropriately depending on the type of the deep-frying material Q, which enables the calculation of the second deterioration indicator of the frying oil P with higher accuracy than the case of calculating it using Equation (31) or Equation (32) in all cases without considering the type of the deep-frying material Q.

The present invention has been described with reference to each of the embodiments of the present invention. However, the present invention is not limited to the embodiments described above but various modifications can be made therein. For example, each of the embodiments is described in detail herein for the purpose of clarity and concise description, and the present invention is not necessarily limited to those including all the features described above. Furthermore, some of the features according to a predetermined embodiment can be replaced with other features according to a separate embodiment, and other features can be added to the configuration of the predetermined embodiment. Still further, other features of a separate embodiment may be added to some of the features of each of the embodiments described above, and some of the features of each of the embodiments described above may be deleted or replaced.

For example, in each of the embodiments described above, the correlation between the first deterioration indicator of the frying oil P and the second deterioration indicator of the frying oil P is a correlation equation in which the second deterioration indicator of the frying oil P is expressed with a linear or quadratic equation of the first deterioration indicator, however, it does not necessarily have to be a linear or quadratic equation but may be any correlation equation as long as it is expressed with a polynomial. Furthermore, the correlation between the first deterioration indicator of the frying oil P and the second deterioration indicator of the frying oil P does not necessarily have to be a correlation equation but may be, for example, a correlation map, or a correlation model created by machine learning using all variables relating to the correlation between the first deterioration indicator of the frying oil P and the second deterioration indicator of the frying oil P.

Furthermore, in the first to seventh embodiments described above, each of the clouds 8, 8A, 8B, 8C is configured to calculate the acid value (AVn) of the frying oil P based on a measured value of the total polar compounds of the frying oil P measured by the PC sensor 41, however, it may be configured to calculate a deterioration indicator other than the acid value (AVn). In other words, each of the clouds 8, 8A, 8B, 8C may use a correlation between the total polar compounds (PCn) contained in the frying oil P and a predetermined deterioration indicator (DIn) other than the total polar compounds to convert a measured value of the total polar compounds of the frying oil P measured by the PC sensor 41 into a predetermined deterioration indicator (DIn) other than the total polar compounds.

Still further, in the embodiments described above, each of the clouds 8, 8A, 8B, 8C, 9, 9A, 9B, 9C, 9D has been described as one of the aspects of the fat and oil deterioration degree detection device, however, the functions of the fat and oil deterioration degree detection device may be provided in a frying oil management application installed in the store terminal 6. In this case, the store terminal 6 serves not only as an input terminal and a notification device, but also as a fat and oil deterioration degree detection device.

Still further, in the embodiments described above, the frying oil P has been exemplified as fat and oil, however, the fat and oil to which the present invention is to be applied does not have to be edible oil for use in deep fry cooking, but may be edible oil for use in other cooking, or fat and oil other than edible oil (for example, oil for industry use).

### REFERENCE SIGNS LIST

5: fat and oil deterioration degree detection system
41: PC sensor (measurement device)
8, 8A, 8B, 8C, 9, 9A, 9B, 9C, 9D: cloud (fat and oil deterioration degree detection device)
81, 81A, 81B, 81C, 91, 91A, 91B, 91C, 91D: data acquisition section
82, 82A, 82B, 82C, 92, 92A, 92B, 92C, 92D: storage section
83, 83A, 83B, 83C, 93, 93A, 93B, 93C, 93D: deterioration indicator calculation section
84, 94: detection result output section
P: frying oil (edible oil)
Q, Q1, Q2, Q3, Q4: deep-frying material (ingredient)

1. A fat and oil deterioration degree detection device for detecting a deterioration degree of a fat and oil based on total polar compounds of the fat and oil which is one of fat and oil deterioration indicators, the device comprising:
a storage section configured to retain a correlation between the total polar compounds and a predetermined deterioration indicator other than the total polar compounds;
a data acquisition section configured to acquire a measured value of the total polar compounds;
a deterioration indicator calculation section configured to calculate the predetermined deterioration indicator based on the measured value of the total polar compounds acquired by the data acquisition section and the correlation stored in the storage section; and
a detection result output section configured to output the predetermined deterioration indicator calculated by the deterioration indicator calculation section as a result of detection of the deterioration degree.

2. The fat and oil deterioration degree detection device according to claim 1, wherein
the correlation is a correlation equation in a form of a polynomial in which the predetermined deterioration indicator is expressed with the total polar compounds.

3. The fat and oil deterioration degree detection device according to claim 2, wherein
the correlation equation is at least one of a linear equation expressed with a following equation (1) or a quadratic equation expressed with a following equation (2), where the total polar compounds are defined as PC, the predetermined deterioration indicator is defined as DI, and an arbitrary heating time of the fat and oil is defined as n.
$$DIn = α \times ( PCn ) + β \qquad ( 1 )$$
α: first-order coefficient of PCn
β: constant
$$DIn = γ \times {( PCn )}^{2} + δ \times ( PCn ) + ε \qquad ( 2 )$$
γ: second-order coefficient of PCn
δ: first-order coefficient of PCn
ε: constant

4. The fat and oil deterioration degree detection device according to claim 3, wherein
the fat and oil are edible oil used for deep frying an ingredient, and
the first-order coefficient α and the constant β included in the equation (1) and the second-order coefficient γ, the first-order coefficient δ, and the constant β included in the equation (2) are set to values corresponding to a deep-frying weight per unit time of a deep-frying material to be cooked using the edible oil, respectively.

5. The fat and oil deterioration degree detection device according to claim 3, wherein
the fat and oil are edible oil used for deep frying an ingredient,
the storage section retains, as the correlation equation, a linear equation expressed with a following equation (3) obtained by subtracting an empty heating variable EH1 from the equation (1) or a quadratic equation expressed with a following equation (4) obtained by subtracting an empty heating variable EH2 from the equation (2), the empty heating variable EH1 being set considering empty heating in which only the fat and oil are heated without cooking the ingredient, and the empty heating variable EH2 being set considering the empty heating, and
$$DIn = α \times ( PCn ) + β - EH 1 \qquad ( 3 )$$
α: first-order coefficient of PCn
β: constant
EH1: empty heating variable
$$DIn = γ \times {( PCn )}^{2} + δ \times ( PCn ) + ε - EH 2 \qquad ( 4 )$$
γ: second-order coefficient of PCn
δ: first-order coefficient of PCn
ε: constant
EH2: empty heating variable
in a case where the empty heating for the fat and oil has been performed, the deterioration indicator calculation section uses the equation (3) or the equation (4) stored in the storage section to calculate the predetermined deterioration indicator.

6. The fat and oil deterioration degree detection device according to claim 3, wherein
the first-order coefficient α and the constant 3 included in the equation (1) and the second-order coefficient γ, the first-order coefficient δ, and the constant 3 included in the equation (2) are set to values corresponding to a type of the fat and oil, respectively.

7. The fat and oil deterioration degree detection device according to claim 6, wherein
the type of the fat and oil is classified into a first oil type and a second oil type depending on a fatty acid composition of the fat and oil,
the first oil type is an oil type indicative of a composition of the fat and oil in which a content of oleic acid is more than a content of linoleic acid,
the second oil type is an oil type indicative of a composition of the fat and oil in which the content of oleic acid is equal to or less than the content of linoleic acid,
the storage section retains, as the correlation equation, a linear equation expressed with a following equation (5) including each of α1 set to a value corresponding to the first oil type as the first-order coefficient α in the equation (1) and 31 set to a value corresponding to the first oil type as the constant β in the equation (1), or a quadratic equation expressed with a following equation (6) including each of γ1 set to a value corresponding to the first oil type as the second-order coefficient γ in the equation (2), 61 set to a value corresponding to the first oil type as the first-order coefficient δ in the equation (2), and ε1 set to a value corresponding to the first oil type as the constant ε in the equation (2),
$$DIn = α1 \times ( PCn ) + β1 \qquad ( 5 )$$
α1: first-order coefficient of PCn
β1: constant
$$DIn = γ 1 \times {( PCn )}^{2} + δ 1 \times ( PCn ) + ε 1 \qquad ( 6 )$$
γ1: second-order coefficient of PCn
δ1: first-order coefficient of PCn
ε1: constant
the storage section retains, as the correlation equation, a linear equation expressed with a following equation (7) including each of α2 set to a value corresponding to the second oil type as the first-order coefficient α in the equation (1) and β2 set to a value corresponding to the second oil type as the constant β in the equation (1), or a quadratic equation expressed with a following equation (8) including each of γ2 set to a value corresponding to the second oil type as the second-order coefficient γ in the equation (2), δ2 set to a value corresponding to the second oil type as the first-order coefficient δ in the equation (2), and ε2 set to a value corresponding to the second oil type as the constant ε in the equation (2),
$$DIn = α 2 \times ( PCn ) + β2 \qquad ( 7 )$$
α2: first-order coefficient of PCn
β2: constant
$$DIn = γ 2 \times {( PCn )}^{2} + δ2 \times ( PCn ) + ε2 \qquad ( 8 )$$
γ2: second-order coefficient of PCn
δ2: first-order coefficient of PCn
ε2: constant
in a case where the type of the oil and fat is the first oil type, the deterioration indicator calculation section uses the equation (5) or the equation (6) stored in the storage section to calculate the predetermined deterioration indicator, and
in a case where the type of the oil and fat is the second oil type, the deterioration indicator calculation section uses the equation (7) or the equation (8) stored in the storage section to calculate the predetermined deterioration indicator.

8. The fat and oil deterioration degree detection device according to claim 6, wherein
the type of the fat and oil is classified into a third oil type and a fourth oil type depending on an iodine value of the fat and oil,
the third oil type is an oil type for which the iodine value of the fat and oil is less than a predetermined iodine value threshold,
the fourth oil type is an oil type for which the iodine value of the fat and oil is equal to or more than the predetermined iodine value threshold,
the storage section retains, as the correlation equation, a linear equation expressed with a following equation (9) including each of α3 set to a value corresponding to the third oil type as the first-order coefficient α in the equation (1) and β3 set to a value corresponding to the third oil type as the constant β in the equation (1), or a quadratic equation expressed with a following equation (10) including each of γ3 set to a value corresponding to the third oil type as the second-order coefficient γ in the equation (2), 63 set to a value corresponding to the third oil type as the first-order coefficient δ in the equation (2), and ε3 set to a value corresponding to the third oil type as the constant ε in the equation (2),
$$DIn = α3 \times ( PCn ) + β3 \qquad ( 9 )$$
α3: first-order coefficient of PCn
β3: constant
$$DIn = γ3 \times {( PCn )}^{2} + δ3 \times ( PCn ) + ε3 \qquad ( 10 )$$
γ3: second-order coefficient of PCn
δ3: first-order coefficient of PCn
ε3: constant
the storage section retains, as the correlation equation, a linear equation expressed with a following equation (11) including each of α4 set to a value corresponding to the fourth oil type as the first-order coefficient α in the equation (1) and β4 set to a value corresponding to the fourth oil type as the constant β in the equation (1), or a quadratic equation expressed with a following equation (12) including each of γ4 set to a value corresponding to the fourth oil type as the second-order coefficient γ in the equation (2), 54 set to a value corresponding to the fourth oil type as the first-order coefficient δ in the equation (2), and ε4 set to a value corresponding to the fourth oil type as the constant ε in the equation (2),
$$DIn = α 4 \times ( PCn ) + β4 \qquad ( 11 )$$
α4: first-order coefficient of PCn
β4: constant
$$DIn = γ 4 \times {( PCn )}^{2} + δ 4 \times ( PCn ) + ε 4 \qquad ( 12 )$$
γ4: second-order coefficient of PCn
δ4: first-order coefficient of PCn
ε4: constant
in a case where the type of the oil and fat is the third oil type, the deterioration indicator calculation section uses the equation (9) or the equation (10) to calculate the predetermined deterioration indicator, and
in a case where the type of the oil and fat is the fourth oil type, the deterioration indicator calculation section uses the equation (11) or the equation (12) to calculate the predetermined deterioration indicator.

9. The fat and oil deterioration degree detection device according to claim 6, wherein
the type of the fat and oil is classified into a fifth oil type and a sixth oil type depending on a CDM value of the fat and oil,
the fifth oil type is an oil type for which the CDM value of the fat and oil is equal to or more than a predetermined CDM threshold,
the sixth oil type is an oil type for which the CDM value of the fat and oil is less than the predetermined CDM threshold,
the storage section retains, as the correlation equation, a linear equation expressed with a following equation (13) including each of α5 set to a value corresponding to the fifth oil type as the first-order coefficient α in the equation (1) and 135 set to a value corresponding to the fifth oil type as the constant β in the equation (1), or a quadratic equation expressed with a following equation (14) including each of γ5 set to a value corresponding to the fifth oil type as the second-order coefficient γ in the equation (2), 65 set to a value corresponding to the fifth oil type as the first-order coefficient δ in the equation (2), and ε5 set to a value corresponding to the fifth oil type as the constant ε in the equation (2),
$$DIn = α 5 \times ( PCn ) + β 5 \qquad ( 13 )$$
α5: first-order coefficient of PCn
β5: constant
$$DIn = γ 5 \times {( PCn )}^{2} + δ 5 \times ( PCn ) + ε 5 \qquad ( 14 )$$
γ5: second-order coefficient of PCn
δ5: first-order coefficient of PCn
ε5: constant
the storage section retains, as the correlation equation, a linear equation expressed with a following equation (15) including each of α6 set to a value corresponding to the sixth oil type as the first-order coefficient α in the equation (1) and β6 set to a value corresponding to the sixth oil type as the constant 3 in the equation (1), or a quadratic equation expressed with a following equation (16) including each of γ6 set to a value corresponding to the sixth oil type as the second-order coefficient γ in the equation (2), 66 set to a value corresponding to the sixth oil type as the first-order coefficient δ in the equation (2), and ε6 set to a value corresponding to the sixth oil type as the constant ε in the equation (2),
$$DIn = α 6 \times ( PCn ) + β 6 \qquad ( 15 )$$
α6: first-order coefficient of PCn
β6: constant
$$DIn = γ 6 \times {( PCn )}^{2} + δ 6 \times ( PCn ) + ε 6 \qquad ( 16 )$$
δ6: first-order coefficient of PCn
ε6: constant
in a case where the type of the oil and fat is the fifth oil type, the deterioration indicator calculation section uses the equation (13) or the equation (14) to calculate the predetermined deterioration indicator, and
in a case where the type of the oil and fat is the sixth oil type, the deterioration indicator calculation section uses the equation (15) or the equation (16) to calculate the predetermined deterioration indicator.

10. The fat and oil deterioration degree detection device according to claim 6, wherein
the type of the fat and oil is classified into a seventh oil type and an eighth oil type depending on lipid molecular species in the fat and oil,
the seventh oil type is an oil type for which a content of the lipid molecular species in the fat and oil is more than a predetermined content threshold, a rate of increase in a content of diacylglycerol in the fat and oil due to heating is equal to or less than a predetermined first increase rate threshold, a rate of increase in a content of free fatty acid in the fat and oil due to heating is equal to or less than a predetermined second increase rate threshold, and a rate of decrease in a content of triacylglycerol in the fat and oil due to heating is equal to or less than a predetermined decrease rate threshold,
the eighth oil type is an oil type for which the content of the lipid molecular species in the fat and oil is equal to or less than the predetermined content threshold, the rate of increase in the content of diacylglycerol in the fat and oil due to heating is more than the predetermined first increase rate threshold, the rate of increase in the content of free fatty acid in the fat and oil due to heating is more than the predetermined second increase rate threshold, and the rate of decrease in the content of triacylglycerol in the fat and oil due to heating is more than the predetermined decrease rate threshold,
the storage section retains, as the correlation equation, a linear equation expressed with a following equation (17) including each of α7 set to a value corresponding to the seventh oil type as the first-order coefficient α in the equation (1) and β7 set to a value corresponding to the seventh oil type as the constant β in the equation (1), or a quadratic equation expressed with a following equation (18) including each of γ7 set to a value corresponding to the seventh oil type as the second-order coefficient γ in the equation (2), 67 set to a value corresponding to the seventh oil type as the first-order coefficient δ in the equation (2), and ε7 set to a value corresponding to the seventh oil type as the constant ε in the equation (2),
$$DIn = α 7 \times ( PCn ) + β 7 \qquad ( 17 )$$
α7: first-order coefficient of PCn
ϵ7: constant
$$DIn = γ 7 \times {( PCn )}^{2} + δ 7 \times ( PCn ) + ε7 \qquad ( 18 )$$
γ7: second-order coefficient of PCn
δ7: first-order coefficient of PCn
ε7: constant
the storage section retains, as the correlation equation, a linear equation expressed with a following equation (19) including each of α8 set to a value corresponding to the eighth oil type as the first-order coefficient α in the equation (1) and 38 set to a value corresponding to the eighth oil type as the constant β in the equation (1), or a quadratic equation expressed with a following equation (20) including each of γ8 set to a value corresponding to the eighth oil type as the second-order coefficient γ in the equation (2), δ8 set to a value corresponding to the eighth oil type as the first-order coefficient δ in the equation (2), and ε8 set to a value corresponding to the eighth oil type as the constant ε in the equation (2),
$$DIn = α 8 \times ( PCn ) + β 8 \qquad ( 19 )$$
α8: first-order coefficient of PCn
β8: constant
$$DIn = γ 8 \times {( PCn )}^{2} + δ 8 \times ( PCn ) + ε 8 \qquad ( 20 )$$
γ8: second-order coefficient of PCn
γ8: first-order coefficient of PCn
ε8: constant
in a case where the type of the oil and fat is the seventh oil type, the deterioration indicator calculation section uses the equation (17) or the equation (18) to calculate the predetermined deterioration indicator, and
in a case where the type of the oil and fat is the eighth oil type, the deterioration indicator calculation section uses the equation (19) or the equation (20) to calculate the predetermined deterioration indicator.

11-19. (canceled)

20. A fat and oil deterioration degree detection device for detecting a deterioration degree of a fat and oil, the device comprising:
a storage section configured to retain a correlation between a first deterioration indicator and a second deterioration indicator, the first deterioration indicator being a deterioration indicator of the fat and oil and defined based on a substance produced by heating the fat and oil, and the second deterioration indicator being a deterioration indicator of the fat and oil other than the first deterioration indicator;
a data acquisition section configured to acquire a measured value of the first deterioration indicator;
a deterioration indicator calculation section configured to calculate the second deterioration indicator based on the measured value of the first deterioration indicator acquired by the data acquisition section and the correlation stored in the storage section; and
a detection result output section configured to output the second deterioration indicator calculated by the deterioration indicator calculation section as a result of detection of the deterioration degree.

21. The fat and oil deterioration degree detection device according to claim 20, wherein
the correlation is a correlation equation in a form of a polynomial in which the second deterioration indicator is expressed with the first deterioration indicator.

22. The fat and oil deterioration degree detection device according to claim 21, wherein
the correlation equation is at least one of a linear equation expressed with a following equation (31) or a quadratic equation expressed with a following equation (32), where the first deterioration indicator is Di1, the second deterioration indicator is Di2, and an arbitrary heating time of the fat and oil is n.
$$Di 2 n = α \times ( Di 1 n ) + β \qquad ( 31 )$$
α: first-order coefficient of Di1n
β: constant
$$Di 2 n = γ \times {( Di 1 n )}^{2} + δ \times ( Di 1 n ) + ε \qquad ( 32 )$$
γ: second-order coefficient of Di1n
δ: first-order coefficient of Di1n
ε: constant

23. The fat and oil deterioration degree detection device according to claim 22, wherein
the fat and oil are edible oil used for deep frying an ingredient, and
the first-order coefficient α and the constant β included in the equation (31) and the second-order coefficient γ, the first-order coefficient δ, and the constant β included in the equation (32) are set to values corresponding to a deep-frying weight per unit time of a deep-frying material to be cooked using the edible oil, respectively.

24. The fat and oil deterioration degree detection device according to claim 22, wherein
the fat and oil are edible oil used for deep frying an ingredient,
the storage section retains, as the correlation equation, a linear equation expressed with a following equation (33) obtained by adding a member of an empty heating variable EH1 to the equation (31) or a quadratic equation expressed with a following equation (34) obtained by adding a member of an empty heating variable EH2 to the equation (32), the empty heating variable EH1 being set considering empty heating in which only the fat and oil are heated without cooking the ingredient, and the empty heating variable EH2 being set considering the empty heating, and
$$Di 2 n = α \times ( Di 1 n ) + β + EH 1 \qquad ( 33 )$$
α: first-order coefficient of Di1n
β: constant
EH1: empty heating variable
$$Di 2 n = γ \times {( Di 1 n )}^{2} + δ \times ( Di 1 n ) + ε + EH 2 \qquad ( 34 )$$
γ: second-order coefficient of Di1n
δ: first-order coefficient of Di1n
ε: constant
EH2: empty heating variable
in a case where the empty heating for the fat and oil has been performed, the deterioration indicator calculation section uses the equation (33) or the equation (34) stored in the storage section to calculate the second deterioration indicator.

25. The fat and oil deterioration degree detection device according to claim 22, wherein
the first-order coefficient α and the constant β included in the equation (31) and the second-order coefficient γ, the first-order coefficient δ, and the constant β included in the equation (32) are set to values corresponding to a type of the fat and oil, respectively.

26. The fat and oil deterioration degree detection device according to claim 25, wherein
the type of the fat and oil is classified into a first oil type and a second oil type depending on a fatty acid composition of the fat and oil,
the first oil type is an oil type indicative of a composition of the fat and oil in which a content of oleic acid is more than a content of linoleic acid,
the second oil type is an oil type indicative of a composition of the fat and oil in which the content of oleic acid is equal to or less than the content of linoleic acid, the storage section retains, as the correlation equation, a linear equation expressed with a following equation (35) including each of α1 set to a value corresponding to the first oil type as the first-order coefficient α in the equation (31) and β1 set to a value corresponding to the first oil type as the constant β in the equation (31), or a quadratic equation expressed with a following equation (36) including each of γ1 set to a value corresponding to the first oil type as the second-order coefficient γ in the equation (32), δ1 set to a value corresponding to the first oil type as the first-order coefficient δ in the equation (32), and ε1 set to a value corresponding to the first oil type as the constant ε in the equation (32),
$$Di 2 n = α 1 \times ( Di 1 n ) + β 1 \qquad ( 35 )$$
α1: first-order coefficient of Di1n
β1: constant
$$Di 2 n = γ 1 \times {( Di 1 n )}^{2} + δ 1 \times ( Di 1 n ) + ε 1 \qquad ( 36 )$$
γ1: second-order coefficient of Di1n
δ1: first-order coefficient of Di1n
ε1: constant
the storage section retains, as the correlation equation, a linear equation expressed with a following equation (37) including each of α2 set to a value corresponding to the second oil type as the first-order coefficient α in the equation (31) and β2 set to a value corresponding to the second oil type as the constant β in the equation (31), or a quadratic equation expressed with a following equation (38) including each of γ2 set to a value corresponding to the second oil type as the second-order coefficient γ in the equation (32), 62 set to a value corresponding to the second oil type as the first-order coefficient δ in the equation (32), and ε2 set to a value corresponding to the second oil type as the constant ε in the equation (32),
$$Di 2 n = α 2 \times ( Di 1 n ) + β 2 \qquad ( 37 )$$
α2: first-order coefficient of Di1n
β2: constant
$$Di 2 n = γ 2 \times {( Di 1 n )}^{2} + δ 2 \times ( Di 1 n ) + ε 2 \qquad ( 38 )$$
γ2: second-order coefficient of Di1n
δ2: first-order coefficient of Di1n
ε2: constant
in a case where the type of the oil and fat is the first oil type, the deterioration indicator calculation section uses the equation (35) or the equation (36) stored in the storage section to calculate the second deterioration indicator, and
in a case where the type of the oil and fat is the second oil type, the deterioration indicator calculation section uses the equation (37) or the equation (38) stored in the storage section to calculate the second deterioration indicator.

27. The fat and oil deterioration degree detection device according to claim 25, wherein
the type of the fat and oil is classified into a third oil type and a fourth oil type depending on an iodine value of the fat and oil,
the third oil type is an oil type for which the iodine value of the fat and oil is less than a predetermined iodine value threshold,
the fourth oil type is an oil type for which the iodine value of the fat and oil is equal to or more than the predetermined iodine value threshold,
the storage section retains, as the correlation equation, a linear equation expressed with a following equation (39) including each of α3 set to a value corresponding to the third oil type as the first-order coefficient α in the equation (31) and β3 set to a value corresponding to the third oil type as the constant β in the equation (31), or a quadratic equation expressed with a following equation (40) including each of γ3 set to a value corresponding to the third oil type as the second-order coefficient γ in the equation (32), δ3 set to a value corresponding to the third oil type as the first-order coefficient δ in the equation (32), and ε3 set to a value corresponding to the third oil type as the constant ε in the equation (32),
$$Di 2 n = α 3 \times ( Di 1 n ) + β 3 \qquad ( 39 )$$
α3: first-order coefficient of Di1n
β3: constant
$$Di 2 n = γ 3 \times {( Di 1 n )}^{2} + δ 3 \times ( Di 1 n ) + ε 3 \qquad ( 40 )$$
γ3: second-order coefficient of Di1n
δ3: first-order coefficient of Di1n
ε3: constant
the storage section retains, as the correlation equation, a linear equation expressed with a following equation (41) including each of α4 set to a value corresponding to the fourth oil type as the first-order coefficient α in the equation (31) and β4 set to a value corresponding to the fourth oil type as the constant β in the equation (31), or a quadratic equation expressed with a following equation (42) including each of γ4 set to a value corresponding to the fourth oil type as the second-order coefficient γ in the equation (32), 54 set to a value corresponding to the fourth oil type as the first-order coefficient δ in the equation (32), and ε4 set toa value corresponding to the fourth oil type as the constant ε in the equation (32),
$$Di 2 n = α 4 \times ( Di 1 n ) + β 4 \qquad ( 41 )$$
α4: first-order coefficient of Di1n
β4: constant
$$Di 2 n = γ 4 \times {( Di 1 n )}^{2} + δ 4 \times ( Di 1 n ) + ε 4 \qquad ( 42 )$$
γ4: second-order coefficient of Di1n
δ4: first-order coefficient of Di1n
ε4: constant
in a case where the type of the oil and fat is the third oil type, the deterioration indicator calculation section uses the equation (39) or the equation (40) to calculate the second deterioration indicator, and
in a case where the type of the oil and fat is the fourth oil type, the deterioration indicator calculation section uses the equation (41) or the equation (42) to calculate the second deterioration indicator.

28. The fat and oil deterioration degree detection device according to claim 25, wherein
the type of the fat and oil is classified into a fifth oil type and a sixth oil type depending on a CDM value of the fat and oil,
the fifth oil type is an oil type for which the CDM value of the fat and oil is equal to or more than a predetermined CDM threshold,
the sixth oil type is an oil type for which the CDM value of the fat and oil is less than the predetermined CDM threshold,
the storage section retains, as the correlation equation, a linear equation expressed with a following equation (43) including each of α5 set to a value corresponding to the fifth oil type as the first-order coefficient α in the equation (31) and 135 set to a value corresponding to the fifth oil type as the constant β in the equation (31), or a quadratic equation expressed with a following equation (44) including each of γ5 set to a value corresponding to the fifth oil type as the second-order coefficient γ in the equation (32), δ5 set to a value corresponding to the fifth oil type as the first-order coefficient δ in the equation (32), and ε5 set to a value corresponding to the fifth oil type as the constant ε in the equation (32),
$$Di 2 n = α 5 \times ( Di 1 n ) + β 5 \qquad ( 43 )$$
α5: first-order coefficient of Di1n
β5: constant
$$Di 2 n = γ 5 \times {( Di 1 n )}^{2} + δ 5 \times ( Di 1 n ) + ε 5 \qquad ( 44 )$$
γ5: second-order coefficient of Di1n
δ5: first-order coefficient of Di1n
ε5: constant
the storage section retains, as the correlation equation, a linear equation expressed with a following equation (45) including each of α6 set to a value corresponding to the sixth oil type as the first-order coefficient α in the equation (31) and β6 set to a value corresponding to the sixth oil type as the constant β in the equation (31), or a quadratic equation expressed with a following equation (46) including each of γ6 set to a value corresponding to the sixth oil type as the second-order coefficient γ in the equation (32), δ6 set to a value corresponding to the sixth oil type as the first-order coefficient δ in the equation (32), and ε6 set to a value corresponding to the sixth oil type as the constant ε in the equation (32),
$$Di 2 n = α 6 \times ( Di 1 n ) + β 6 \qquad ( 45 )$$
α6: first-order coefficient of Di1n
β6: constant
$$Di 2 n = γ 6 \times {( Di 1 n )}^{2} + δ 6 \times ( Di 1 n ) + ε 6 \qquad ( 46 )$$
γ6: second-order coefficient of Di1n
δ6: first-order coefficient of Di1n
ε6: constant
in a case where the type of the oil and fat is the fifth oil type, the deterioration indicator calculation section uses the equation (43) or the equation (44) to calculate the second deterioration indicator, and
in a case where the type of the oil and fat is the sixth oil type, the deterioration indicator calculation section uses the equation (45) or the equation (46) to calculate the second deterioration indicator.

29. The fat and oil deterioration degree detection device according to claim 25, wherein
the type of the fat and oil is classified into a seventh oil type and an eighth oil type depending on lipid molecular species in the fat and oil,
the seventh oil type is an oil type for which a content of the lipid molecular species in the fat and oil is more than a predetermined content threshold, a rate of increase in a content of diacylglycerol in the fat and oil due to heating is equal to or less than a predetermined first increase rate threshold, a rate of increase in a content of free fatty acid in the fat and oil due to heating is equal to or less than a predetermined second increase rate threshold, and a rate of decrease in a content of triacylglycerol in the fat and oil due to heating is equal to or less than a predetermined decrease rate threshold,
the eighth oil type is an oil type for which the content of the lipid molecular species in the fat and oil is equal to or less than the predetermined content threshold, the rate of increase in the content of diacylglycerol in the fat and oil due to heating is more than the predetermined first increase rate threshold, the rate of increase in the content of free fatty acid in the fat and oil due to heating is more than the predetermined second increase rate threshold, and the rate of decrease in the content of triacylglycerol in the fat and oil due to heating is more than the predetermined decrease rate threshold,
the storage section retains, as the correlation equation, a linear equation expressed with a following equation (47) including each of α7 set to a value corresponding to the seventh oil type as the first-order coefficient α in the equation (31) and β7 set to a value corresponding to the seventh oil type as the constant β in the equation (31), or a quadratic equation expressed with a following equation (48) including each of γ7 set to a value corresponding to the seventh oil type as the second-order coefficient γ in the equation (32), 67 set to a value corresponding to the seventh oil type as the first-order coefficient δ in the equation (2), and ε7 set to a value corresponding to the seventh oil type as the constant ε in the equation (32),
$$Di 2 n = α 7 \times ( Di 1 n ) + β 7 \qquad ( 47 )$$
α7: first-order coefficient of Di1n
β7: constant
$$Di 2 n = γ 7 \times {( Di 1 n )}^{2} + δ 7 \times ( Di 1 n ) + ε 7 \qquad ( 48 )$$
γ7: second-order coefficient of Di1n
δ7: first-order coefficient of Di1n
ε7: constant
the storage section retains, as the correlation equation, a linear equation expressed with a following equation (49) including each of α8 set to a value corresponding to the eighth oil type as the first-order coefficient α in the equation (31) and 38 set to a value corresponding to the eighth oil type as the constant β in the equation (31), or a quadratic equation expressed with a following equation (50) including each of γ8 set to a value corresponding to the eighth oil type as the second-order coefficient γ in the equation (32), 58 set to a value corresponding to the eighth oil type as the first-order coefficient δ in the equation (32), and ε8 set to a value corresponding to the eighth oil type as the constant ε in the equation (32),
$$Di 2 n = α 8 \times ( Di 1 n ) + β 8 \qquad ( 49 )$$
α8: first-order coefficient of Di1n
β8: constant
$$Di 2 n = γ 8 \times {( Di 1 n )}^{2} + δ 8 \times ( Di 1 n ) + ε 8 \qquad ( 50 )$$
γ8: second-order coefficient of Di1n
δ8: first-order coefficient of Di1n
ε8: constant
in a case where the type of the oil and fat is the seventh oil type, the deterioration indicator calculation section uses the equation (47) or the equation (48) to calculate the second deterioration indicator, and
in a case where the type of the oil and fat is the eighth oil type, the deterioration indicator calculation section uses the equation (49) or the equation (50) to calculate the second deterioration indicator.

30. The fat and oil deterioration degree detection device according to claim 22, wherein
the fat and oil are edible oil used for deep frying an ingredient, and
in a case where the deterioration indicator calculation section calculates the color of the edible oil as the second deterioration indicator, the first-order coefficient α and the constant β included in the equation (31) and the second-order coefficient γ, the first-order coefficient δ, and the constant β included in the equation (32) are set to values corresponding to a type of a deep-frying material to be cooked using the edible oil, respectively.

31. The fat and oil deterioration degree detection device according to claim 20, wherein
the first deterioration indicator is at least one of an acid value of the fat and oil, total polar compounds of the fat and oil, a color of the fat and oil, or a rate of increase in viscosity of the fat and oil.

32-40. (canceled)
